SEQUENCE LISTING (216 sequences)

SEQ ID NO: 1 - protein, 832 aa, Thermus aquaticus [sequence omitted]

SEQ ID NO: 2 - DNA, 2626 bp, Thermus aquaticus
  source 1..2626: mol_type = genomic DNA
aagctcagatctacctgcctgagggcgtccggttccagctggcccttcccgagggggagagggaggcgtttctaaaagcccttcaggacgctacccgggggcgggtggtggaagggtaacatgagggggatgctgcccctctttgagcccaagggccgggtcctcctggtggacggccaccacctggcctaccgcaccttccacgccctgaagggcctcaccaccagccggggggagccggtgcaggcggtctacggcttcgccaagagcctcctcaaggccctcaaggaggacggggacgcggtgatcgtggtctttgacgccaaggccccctccttccgccacgaggcctacggggggtacaaggcgggccgggcccccacgccggaggactttccccggcaactcgccctcatcaaggagctggtggacctcctggggctggcgcgcctcgaggtcccgggctacgaggcggacgacgtcctggccagcctggccaagaaggcggaaaaggagggctacgaggtccgcatcctcaccgccgacaaagacctttaccagctcctttccgaccgcatccacgtcctccaccccgaggggtacctcatcaccccggcctggctttgggaaaagtacggcctgaggcccgaccagtgggccgactaccgggccctgaccggggacgagtccgacaaccttcccggggtcaagggcatcggggagaagacggcgaggaagcttctggaggagtgggggagcctggaagccctcctcaagaacctggaccggctgaagcccgccatccgggagaagatcctggcccacatggacgatctgaagctctcctgggacctggccaaggtgcgcaccgacctgcccctggaggtggacttcgccaaaaggcgggagcccgaccgggagaggcttagggcctttctggagaggcttgagtttggcagcctcctccacgagttcggccttctggaaagccccaaggccctggaggaggccccctggcccccgccggaagcagggggggccgggtccaccgggcccccgagccttataaagccctcaggggggccttcgtgggctttgtgctttcccgcaaggagcccatgtgggccgatcttctggccctggccgccgcacctgaaggaggcgcgggggcttctcgccaaagacctgagcgttctggccctgagggaaggccttggcctcccgcccggcgacgaccccatgctcctcgcctacctcctggacccttccaacaccacccccgagggggtggcccggcgctacggcggggagtggacggaggaggcgggggagcgggccgccctttccgagaggctcttcgccaacctgtgggggaggcttgagggggaggagaggctcctttggctttaccgggaggtggagaggcccctttccgctgtcctggcccacatggaggccacgggggtgcgcctggacgtggcctatctcagggccttgtccctggaggtggccgaggagatcgcccgcctcgaggccgaggtcttccgcctggccggccaccccttcaacctcaactcccgggaccagctggaaagggtcctctttgacgagctagggcttcccgccatcggcaagacggagaagaccggcaagcgctccaccagcgccgccgtcctggaggccctccgcgaggcccaccccatcgtggagaagatcctgcagtaccgggagctcaccaagctgaagagcacctacattgaccccttgccggacctcatccaccccaggacgggccgcctccacacccgcttcaaccagacggccacggccacgggcaggctaagtagctccgatcccaacctccagaacatccccgtccgcaccccgcttgggcagaggatccgccgggccttcatcgccgaggaggggtggctattggtggccctggactatagccagatagagctcagggtgctggcccacctctccggcgacgagaacctgatccgggtcttccaggaggggcgggacatccacacggagaccgccagctggatgttcggcgtcccccgggaggccgtggaccccctgatgcgccgggcggccaagaccatcaacttcggggtcctctacggcatgtcggcccaccgcctctcccaggagctagccatcccttacgaggaggcccaggccttcattgagcgctactttcagagcttccccaaggtgcgggcctggattgagaagaccctggaggagggcaggaggcgggggtacgtggagaccctcttcggccgccgccgctacgtgccagacctagaggcccgggtgaagagcgtgcgggaggcggccgagcgcatggccttcaacatgcccgtccagggcaccgccgccgacctcatgaagctggctatggtgaagctcttccccaggctggaggaaatgggggccaggatgctccttcaggtccacgacgagctggtcctcgaggccccaaaagagagggcggaggccgtggcccggctggccaaggaggtcatggagggggtgtatcccctggccgtgcccctggaggtggaggtggggataggggaggactggctctccgccaaggagtgataccacc

SEQ ID NO: 3 - protein, 861 aa, Thermus aquaticus
MRGMLPLFEPKGRVLLVDGHHLAYRTFHALKGLTTSRGRPVQAVYGFAKSLLKALKEDGDDVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 4 - DNA, 2583 bp, Thermus aquaticus
  source 1..2583: mol_type = genomic DNA
atgcgtggtatgctgccgttgttcgagcctaaaggccgcgtactgttagtcgatggtcatcacttggcctatcggacgttccatgcactcaaaggtctgacgaccagtcgtggccgtccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctaccccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 5 - protein, 861 aa, Thermus aquaticus
MRGMLPLFEPKGRVLLVDGHRLAYRTFHALKGLTTSRGEPVQAVYGFAKSLLKALKEDGDDVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 6 - DNA, 2583 bp, Thermus aquaticus
  source 1..2583: mol_type = genomic DNA
atgcgtggtatgctgccgttgttcgagcctaaaggccgcgtactgttagtcgatggtcatcgtttggcctatcggacgttccatgcactcaaaggtctgacgaccagtcgtggcgaaccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctaccccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 7 - protein, 861 aa, Thermus aquaticus
MRGMLPLFEPKGRVLLVDGHHLAYRTFHALKGLTTSRGEPVQAVYGFAKSLLKALKEDGDDVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGVPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 8 - DNA, 2583 bp, Thermus aquaticus
  source 1..2583: mol_type = genomic DNA
atgcgtggtatgctgccgttgttcgagcctaaaggccgcgtactgttagtcgatggtcatcacttggcctatcggacgttccatgcactcaaaggtctgacgaccagtcgtggcgaaccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctaccccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggagtcccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 9 - protein, 861 aa, Thermus aquaticus
MFGMLPLFEPKGRVLLVDGHHLAYRTFHALKGLTTSRGEPVQAVYGFAKSLLKALKEDGDDVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 10 - DNA, 2583 bp, Thermus aquaticus
  source 1..2583: mol_type = genomic DNA
atgtttggtatgctgccgttgttcgagcctaaaggccgcgtactgttagtcgatggtcatcacttggcctatcggacgttccatgcactcaaaggtctgacgaccagtcgtggcgaaccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctaccccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 11 - protein, 861 aa, Thermus aquaticus
MRGMLPLFEPKGRVLLVDGHHLAYRTFHALKGLTTSRGEPVQAVYGFAKSLLKALKEDGDDVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 12 - DNA, 2583 bp, Thermus aquaticus
  source 1..2583: mol_type = genomic DNA
atgcgtggtatgctgccgttgttcgagcctaaaggccgcgtactgttagtcgatggtcatcacttggcctatcggacgttccatgcactcaaaggtctgacgaccagtcgtggcgaaccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 13 - protein, 861 aa, Thermus aquaticus
MRGMLPLFEPKGRVLLVDGHHLAYRTFHALKGLTTSRGEPVQAVYGFAKSLLKALKEDGDDVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADSRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 14 - DNA, 2583 bp, Thermus aquaticus
  source 1..2583: mol_type = genomic DNA
atgcgtggtatgctgccgttgttcgagcctaaaggccgcgtactgttagtcgatggtcatcacttggcctatcggacgttccatgcactcaaaggtctgacgaccagtcgtggcgaaccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctaccccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggatagccgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 15 - protein, 861 aa, Thermus aquaticus
MRGMLPLFEPKGRVLLVDGHRLAYRTFHALKGLTTSRGRPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 16 - DNA, 2583 bp, Thermus aquaticus
  source 1..2583: mol_type = genomic DNA
atgcgtggtatgctgccgttgttcgagcctaaaggccgcgtactgttagtcgatggtcatcgtttggcctatcggacgttccatgcactcaaaggtctgacgaccagtcgtggccgtccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctaccccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 17 - protein, 861 aa, Thermus aquaticus
MRGMLPLFEPKGRVLLVDGHHLAYRTFHALKGLTTSRGRPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGVPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 18 - DNA, 2583 bp, Thermus aquaticus
  source 1..2583: mol_type = genomic DNA
atgcgtggtatgctgccgttgttcgagcctaaaggccgcgtactgttagtcgatggtcatcacttggcctatcggacgttccatgcactcaaaggtctgacgaccagtcgtggccgtccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctaccccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggagtcccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 19 - protein, 861 aa, Thermus aquaticus
MFGMLPLFEPKGRVLLVDGHHLAYRTFHALKGLTTSRGRPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 20 - DNA, 2583 bp, Thermus aquaticus
  source 1..2583: mol_type = genomic DNA
atgtttggtatgctgccgttgttcgagcctaaaggccgcgtactgttagtcgatggtcatcacttggcctatcggacgttccatgcactcaaaggtctgacgaccagtcgtggccgtccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctaccccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 21 - protein, 861 aa, Thermus aquaticus
MRGMLPLFEPKGRVLLVDGHHLAYRTFHALKGLTTSRGRPVQAVYGFAKSLLKALKEDGDAVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 22 - DNA, 2583 bp, Thermus aquaticus
  source 1..2583: mol_type = genomic DNA
atgcgtggtatgctgccgttgttcgagcctaaaggccgcgtactgttagtcgatggtcatcacttggcctatcggacgttccatgcactcaaaggtctgacgaccagtcgtggccgtccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 23 - protein, 861 aa, Thermus aquaticus
MRGMLPLFEPKGRVLLVDGHHLAYRTFHALKGLTTSRGRPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADSRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 24 - DNA, 2583 bp, Thermus aquaticus
  source 1..2583: mol_type = genomic DNA
atgcgtggtatgctgccgttgttcgagcctaaaggccgcgtactgttagtcgatggtcatcacttggcctatcggacgttccatgcactcaaaggtctgacgaccagtcgtggccgtccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctaccccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggatagccgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 25 - protein, 861 aa, Thermus aquaticus
MRGMLPLFEPKGRVLLVDGHRLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGVPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 26 - DNA, 2583 bp, Thermus aquaticus
  source 1..2583: mol_type = genomic DNA
atgcgtggtatgctgccgttgttcgagcctaaaggccgcgtactgttagtcgatggtcatcgtttggcctatcggacgttccatgcactcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctaccccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggagtcccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 27 - protein, 861 aa, Thermus aquaticus
MFGMLPLFEPKGRVLLVDGHRLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 28 - DNA, 2583 bp, Thermus aquaticus
  source 1..2583: mol_type = genomic DNA
atgtttggtatgctgccgttgttcgagcctaaaggccgcgtactgttagtcgatggtcatcgtttggcctatcggacgttccatgcactcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctaccccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 29 - protein, 861 aa, Thermus aquaticus
MRGMLPLFEPKGRVLLVDGHRLAYRTFHALKGLTTSRGEPVQAVYGFAKSLLKALKEDGDAVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 30 - DNA, 2583 bp, Thermus aquaticus
  source 1..2583: mol_type = genomic DNA
atgcgtggtatgctgccgttgttcgagcctaaaggccgcgtactgttagtcgatggtcatcgtttggcctatcggacgttccatgcactcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 31 - protein, 861 aa, Thermus aquaticus
MRGMLPLFEPKGRVLLVDGHRLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADSRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 32 - DNA, 2583 bp, Thermus aquaticus
  source 1..2583: mol_type = genomic DNA
atgcgtggtatgctgccgttgttcgagcctaaaggccgcgtactgttagtcgatggtcatcgtttggcctatcggacgttccatgcactcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctaccccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggatagccgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 33 - protein, 861 aa, Thermus aquaticus
MFGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGVPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 34 - DNA, 2583 bp, Thermus aquaticus
  source 1..2583: mol_type = genomic DNA
atgtttggtatgctgccgttgttcgagcctaaaggccgcgtactgttagtcgatggtcatcacttggcctatcggacgttccatgcactcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctaccccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggagtcccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 35 - protein, 861 aa, Thermus aquaticus
MRGMLPLFEPKGRVLLVDGHHLAYRTFHALKGLTTSRGEPVQAVYGFAKSLLKALKEDGDAVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGVPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 36 - DNA, 2583 bp, Thermus aquaticus
  source 1..2583: mol_type = genomic DNA
atgcgtggtatgctgccgttgttcgagcctaaaggccgcgtactgttagtcgatggtcatcacttggcctatcggacgttccatgcactcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggagtcccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 37 - protein, 861 aa, Thermus aquaticus
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADSRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGVPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 38 - DNA, 2583 bp, Thermus aquaticus
  source 1..2583: mol_type = genomic DNA
atgcgtggtatgctgccgttgttcgagcctaaaggccgcgtactgttagtcgatggtcatcacttggcctatcggacgttccatgcactcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctaccccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggatagccgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggagtcccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 39 - protein, 861 aa, Thermus aquaticus
MFGMLPLFEPKGRVLLVDGHHLAYRTFHALKGLTTSRGEPVQAVYGFAKSLLKALKEDGDAVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 40 - DNA, 2583 bp, Thermus aquaticus
  source 1..2583: mol_type = genomic DNA
atgtttggtatgctgccgttgttcgagcctaaaggccgcgtactgttagtcgatggtcatcacttggcctatcggacgttccatgcactcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 41 - protein, 861 aa, Thermus aquaticus
MFGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADSRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 42 - DNA, 2583 bp, Thermus aquaticus
  source 1..2583: mol_type = genomic DNA
atgtttggtatgctgccgttgttcgagcctaaaggccgcgtactgttagtcgatggtcatcacttggcctatcggacgttccatgcactcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctaccccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggatagccgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 43 - protein, 861 aa, Thermus aquaticus
MRGMLPLFEPKGRVLLVDGHHLAYRTFHALKGLTTSRGEPVQAVYGFAKSLLKALKEDGDAVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADSRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 44 - DNA, 2583 bp, Thermus aquaticus
  source 1..2583: mol_type = genomic DNA
atgcgtggtatgctgccgttgttcgagcctaaaggccgcgtactgttagtcgatggtcatcacttggcctatcggacgttccatgcactcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggatagccgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 45 - protein, 861 aa, Thermus aquaticus
MRGMLPLFEPKGRVLLVDGHRLAYRTFHALKGLTTSRGRPVQAVYGFAKSLLKALKEDGDAVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 46 - DNA, 2583 bp, Thermus aquaticus
  source 1..2583: mol_type = genomic DNA
atgcgtggtatgctgccgttgttcgagcctaaaggccgcgtactgttagtcgatggtcatcgtttggcctatcggacgttccatgcactcaaaggtctgacgaccagtcgtggccgtccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 47 - protein, 861 aa, Thermus aquaticus
MRGMLPLFEPKGRVLLVDGHRLAYRTFHALKGLTTSRGEPVQAVYGFAKSLLKALKEDGDAVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGVPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 48 - DNA, 2583 bp, Thermus aquaticus
  source 1..2583: mol_type = genomic DNA
atgcgtggtatgctgccgttgttcgagcctaaaggccgcgtactgttagtcgatggtcatcgtttggcctatcggacgttccatgcactcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggagtcccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 49 - protein, 861 aa, Thermus aquaticus
MFGMLPLFEPKGRVLLVDGHRLAYRTFHALKGLTTSRGEPVQAVYGFAKSLLKALKEDGDAVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 50 - DNA, 2583 bp, Thermus aquaticus
  source 1..2583: mol_type = genomic DNA
atgtttggtatgctgccgttgttcgagcctaaaggccgcgtactgttagtcgatggtcatcgtttggcctatcggacgttccatgcactcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 51 - protein, 861 aa, Thermus aquaticus
MRGMLPLFEPKGRVLLVDGHRLAYRTFHALKGLTTSRGEPVQAVYGFAKSLLKALKEDGDAVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADSRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 52 - DNA, 2583 bp, Thermus aquaticus
  source 1..2583: mol_type = genomic DNA
atgcgtggtatgctgccgttgttcgagcctaaaggccgcgtactgttagtcgatggtcatcgtttggcctatcggacgttccatgcactcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggatagccgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 53 - protein, 861 aa, Thermus aquaticus
MRGMLPLFEPKGRVLLVDGHRLAYRTFHALKGLTTSRGEPVQAVYGFAKSLLKALKEDGDDVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 54 - DNA, 2583 bp, Thermus aquaticus
  source 1..2583: mol_type = genomic DNA
atgcgtggtatgctgccgttgttcgagcctaaaggccgcgtactgttagtcgatggtcatcgtttggcctatcggacgttccatgcactcaaaggtctgacgaccagtcgtggcgaaccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 55 - protein, 861 aa, Thermus aquaticus
MRGMLPLFEPKGRVLLVDGHRLAYRTFHALKGLTTSRGRPVQAVYGFAKSLLKALKEDGDAVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGVPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 56 - DNA, 2583 bp, Thermus aquaticus
  source 1..2583: mol_type = genomic DNA
atgcgtggtatgctgccgttgttcgagcctaaaggccgcgtactgttagtcgatggtcatcgtttggcctatcggacgttccatgcactcaaaggtctgacgaccagtcgtggccgtccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggagtcccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 57 - protein, 861 aa, Thermus aquaticus
MFGMLPLFEPKGRVLLVDGHRLAYRTFHALKGLTTSRGRPVQAVYGFAKSLLKALKEDGDAVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 58 - DNA, 2583 bp, Thermus aquaticus
  source 1..2583: mol_type = genomic DNA
atgtttggtatgctgccgttgttcgagcctaaaggccgcgtactgttagtcgatggtcatcgtttggcctatcggacgttccatgcactcaaaggtctgacgaccagtcgtggccgtccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 59 - protein, 861 aa, Thermus aquaticus
MRGMLPLFEPKGRVLLVDGHRLAYRTFHALKGLTTSRGRPVQAVYGFAKSLLKALKEDGDAVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADSRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 60 - DNA, 2583 bp, Thermus aquaticus
  source 1..2583: mol_type = genomic DNA
atgcgtggtatgctgccgttgttcgagcctaaaggccgcgtactgttagtcgatggtcatcgtttggcctatcggacgttccatgcactcaaaggtctgacgaccagtcgtggccgtccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggatagccgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 61 - protein, 861 aa, Thermus aquaticus
MRGMLPLFEPKGRVLLVDGHRLAYRTFHALKGLTTSRGRPVQAVYGFAKSLLKALKEDGDDVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 62 - DNA, 2583 bp, Thermus aquaticus
  source 1..2583: mol_type = genomic DNA
atgcgtggtatgctgccgttgttcgagcctaaaggccgcgtactgttagtcgatggtcatcgtttggcctatcggacgttccatgcactcaaaggtctgacgaccagtcgtggccgtccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 63 - protein, 861 aa, Thermus aquaticus
MFGMLPLFEPKGRVLLVDGHRLAYRTFHALKGLTTSRGEPVQAVYGFAKSLLKALKEDGDAVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGVPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 64 - DNA, 2583 bp, Thermus aquaticus
  source 1..2583: mol_type = genomic DNA
atgtttggtatgctgccgttgttcgagcctaaaggccgcgtactgttagtcgatggtcatcgtttggcctatcggacgttccatgcactcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggagtcccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 65 - protein, 861 aa, Thermus aquaticus
MRGMLPLFEPKGRVLLVDGHRLAYRTFHALKGLTTSRGEPVQAVYGFAKSLLKALKEDGDAVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADSRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGVPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 66 - DNA, 2583 bp, Thermus aquaticus
  source 1..2583: mol_type = genomic DNA
atgcgtggtatgctgccgttgttcgagcctaaaggccgcgtactgttagtcgatggtcatcgtttggcctatcggacgttccatgcactcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggatagccgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggagtcccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 67 - protein, 861 aa, Thermus aquaticus
MRGMLPLFEPKGRVLLVDGHRLAYRTFHALKGLTTSRGEPVQAVYGFAKSLLKALKEDGDDVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGVPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 68 - DNA, 2583 bp, Thermus aquaticus
  source 1..2583: mol_type = genomic DNA
atgcgtggtatgctgccgttgttcgagcctaaaggccgcgtactgttagtcgatggtcatcgtttggcctatcggacgttccatgcactcaaaggtctgacgaccagtcgtggcgaaccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggagtcccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 69 - protein, 861 aa, Thermus aquaticus
MFGMLPLFEPKGRVLLVDGHRLAYRTFHALKGLTTSRGEPVQAVYGFAKSLLKALKEDGDAVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADSRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 70 - DNA, 2583 bp, Thermus aquaticus
  source 1..2583: mol_type = genomic DNA
atgtttggtatgctgccgttgttcgagcctaaaggccgcgtactgttagtcgatggtcatcgtttggcctatcggacgttccatgcactcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggatagccgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 71 - protein, 861 aa, Thermus aquaticus
MFGMLPLFEPKGRVLLVDGHRLAYRTFHALKGLTTSRGEPVQAVYGFAKSLLKALKEDGDDVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 72 - DNA, 2583 bp, Thermus aquaticus
  source 1..2583: mol_type = genomic DNA
atgtttggtatgctgccgttgttcgagcctaaaggccgcgtactgttagtcgatggtcatcgtttggcctatcggacgttccatgcactcaaaggtctgacgaccagtcgtggcgaaccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 73 - protein, 861 aa, Thermus aquaticus
MRGMLPLFEPKGRVLLVDGHRLAYRTFHALKGLTTSRGEPVQAVYGFAKSLLKALKEDGDDVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADSRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 74 - DNA, 2583 bp, Thermus aquaticus
  source 1..2583: mol_type = genomic DNA
atgcgtggtatgctgccgttgttcgagcctaaaggccgcgtactgttagtcgatggtcatcgtttggcctatcggacgttccatgcactcaaaggtctgacgaccagtcgtggcgaaccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggatagccgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 75 - protein, 861 aa, Thermus aquaticus
MRGMLPLFEPKGRVLLLDGHRLAYRTFHALKGLTTSRGEPVQAVYGFAKSLLKALKEDGDAVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 76 - DNA, 2583 bp, Thermus aquaticus
  source 1..2583: mol_type = genomic DNA
atgcgtggtatgctgccgttgttcgagcctaaaggccgcgtactgttacttgatggtcatcgtttggcctatcggacgttccatgcactcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 77 - protein, 861 aa, Thermus aquaticus
MRGMLPLFEPKGRVLLVDGHRLAHRTFHALKGLTTSRGEPVQAVYGFAKSLLKALKEDGDAVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 78 - DNA, 2583 bp, Thermus aquaticus
  source 1..2583: mol_type = genomic DNA
atgcgtggtatgctgccgttgttcgagcctaaaggccgcgtactgttagtcgatggtcatcgtttggcccatcggacgttccatgcactcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 79 - protein, 861 aa, Thermus aquaticus
MRGMLPLFEPKGRVLLVDGHRLAYRTRHALKGLTTSRGEPVQAVYGFAKSLLKALKEDGDAVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 80 - DNA, 2583 bp, Thermus aquaticus
  source 1..2583: mol_type = genomic DNA
atgcgtggtatgctgccgttgttcgagcctaaaggccgcgtactgttagtcgatggtcatcgtttggcctatcggacgcgtcatgcactcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 81 - protein, 861 aa, Thermus aquaticus
MRGMLPLFEPKGRVLLVDGHRLAYRTFHPLKGLTTSRGEPVQAVYGFAKSLLKALKEDGDAVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 82 - DNA, 2583 bp, Thermus aquaticus
  source 1..2583: mol_type = genomic DNA
atgcgtggtatgctgccgttgttcgagcctaaaggccgcgtactgttagtcgatggtcatcgtttggcctatcggacgttccatccgctcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 83 - protein, 861 aa, Thermus aquaticus
MRGMLPLFEPKGRVLLVDGHRLAYRTFHALKGLTTSRGEPVQAVYGFAKSLLKALKEDGDAVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLERWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 84 - DNA, 2583 bp, Thermus aquaticus
  source 1..2583: mol_type = genomic DNA
atgcgtggtatgctgccgttgttcgagcctaaaggccgcgtactgttagtcgatggtcatcgtttggcctatcggacgttccatgcactcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaacgttggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 85 - protein, 861 aa, Thermus aquaticus
MRGMLPLFEPKGRVLLVDGHRLAYRTFHALKGLTTSRGEPVQAVYGFAKSLLKALKEDGDAVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRAFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 86 - DNA, 2583 bp, Thermus aquaticus
  source 1..2583: mol_type = genomic DNA
atgcgtggtatgctgccgttgttcgagcctaaaggccgcgtactgttagtcgatggtcatcgtttggcctatcggacgttccatgcactcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgcgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 87 - protein, 861 aa, Thermus aquaticus
MRGMLPLFEPKGRVLLVDGHRLAYRTFHALKGLTTSRGEPVQAVYGFAKSLLKALKEDGDAVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIERTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 88 - DNA, 2583 bp, Thermus aquaticus
  source 1..2583: mol_type = genomic DNA
atgcgtggtatgctgccgttgttcgagcctaaaggccgcgtactgttagtcgatggtcatcgtttggcctatcggacgttccatgcactcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagcgtacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 89 - protein, 861 aa, Thermus aquaticus
MRGMLPLFEPKGRVLLVDGHRLAYRTFHALKGLTTSRGEPVQAVYGFAKSLLKALKEDGDAVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRGAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 90 - DNA, 2583 bp, Thermus aquaticus
  source 1..2583: mol_type = genomic DNA
atgcgtggtatgctgccgttgttcgagcctaaaggccgcgtactgttagtcgatggtcatcgtttggcctatcggacgttccatgcactcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacggggcgctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 91 - protein, 861 aa, Thermus aquaticus
MRGMLPLFEPKGRVLLVDGHRLAYRTFHALKGLTTSRGEPVQAVYGFAKSLLKALKEDGDAVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAEEAAALEIKRASQPELAPEDPEDVEHHHHHH

SEQ ID NO: 92 - DNA, 2583 bp, Thermus aquaticus
  source 1..2583: mol_type = genomic DNA
atgcgtggtatgctgccgttgttcgagcctaaaggccgcgtactgttagtcgatggtcatcgtttggcctatcggacgttccatgcactcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcagaggaagcggccgcactcgagatcaaacgggctagccagccagaactcgccccggaagaccccgaggatgtcgagcaccaccaccaccaccac

SEQ ID NO: 93 - protein, 861 aa, Thermus aquaticus
MRGMLPLFEPKGRVLLLDGHRLAYRTFHALKGLTTSRGRPVQAVYGFAKSLLKALKEDGDDVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 94 - DNA, 2583 bp, Thermus aquaticus
  source 1..2583: mol_type = genomic DNA
atgcgtggtatgctgccgttgttcgagcctaaaggccgcgtactgttacttgatggtcatcgtttggcctatcggacgttccatgcactcaaaggtctgacgaccagtcgtggccgtccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 95 - protein, 861 aa, Thermus aquaticus
MRGMLPLFEPKGRVLLVDGHRLAHRTFHALKGLTTSRGRPVQAVYGFAKSLLKALKEDGDDVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 96 - DNA, 2583 bp, Thermus aquaticus
  source 1..2583: mol_type = genomic DNA
atgcgtggtatgctgccgttgttcgagcctaaaggccgcgtactgttagtcgatggtcatcgtttggcccatcggacgttccatgcactcaaaggtctgacgaccagtcgtggccgtccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 97 - protein, 861 aa, Thermus aquaticus
MRGMLPLFEPKGRVLLVDGHRLAYRTRHALKGLTTSRGRPVQAVYGFAKSLLKALKEDGDDVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 98 - DNA, 2583 bp, Thermus aquaticus
  source 1..2583: mol_type = genomic DNA
atgcgtggtatgctgccgttgttcgagcctaaaggccgcgtactgttagtcgatggtcatcgtttggcctatcggacgcgtcatgcactcaaaggtctgacgaccagtcgtggccgtccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 99 - protein, 861 aa, Thermus aquaticus
MRGMLPLFEPKGRVLLVDGHRLAYRTFHPLKGLTTSRGRPVQAVYGFAKSLLKALKEDGDDVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 100 - DNA, 2583 bp, Thermus aquaticus
  source 1..2583: mol_type = genomic DNA
atgcgtggtatgctgccgttgttcgagcctaaaggccgcgtactgttagtcgatggtcatcgtttggcctatcggacgttccatccgctcaaaggtctgacgaccagtcgtggccgtccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 101 - protein, 861 aa, Thermus aquaticus
MRGMLPLFEPKGRVLLVDGHRLAYRTFHALKGLTTSRGRPVQAVYGFAKSLLKALKEDGDDVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLERWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 102 - DNA, 2583 bp, Thermus aquaticus
  source 1..2583: mol_type = genomic DNA
atgcgtggtatgctgccgttgttcgagcctaaaggccgcgtactgttagtcgatggtcatcgtttggcctatcggacgttccatgcactcaaaggtctgacgaccagtcgtggccgtccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaacgttggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 103 - protein, 861 aa, Thermus aquaticus
MRGMLPLFEPKGRVLLVDGHRLAYRTFHALKGLTTSRGRPVQAVYGFAKSLLKALKEDGDDVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRAFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 104 - DNA, 2583 bp, Thermus aquaticus
  source 1..2583: mol_type = genomic DNA
atgcgtggtatgctgccgttgttcgagcctaaaggccgcgtactgttagtcgatggtcatcgtttggcctatcggacgttccatgcactcaaaggtctgacgaccagtcgtggccgtccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgcgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 105 - protein, 861 aa, Thermus aquaticus
MRGMLPLFEPKGRVLLVDGHRLAYRTFHALKGLTTSRGRPVQAVYGFAKSLLKALKEDGDDVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIERTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 106 - DNA, 2583 bp, Thermus aquaticus
  source 1..2583: mol_type = genomic DNA
atgcgtggtatgctgccgttgttcgagcctaaaggccgcgtactgttagtcgatggtcatcgtttggcctatcggacgttccatgcactcaaaggtctgacgaccagtcgtggccgtccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagcgtacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 107 - protein, 861 aa, Thermus aquaticus
MRGMLPLFEPKGRVLLVDGHRLAYRTFHALKGLTTSRGRPVQAVYGFAKSLLKALKEDGDDVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVRGAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 108 - DNA, 2583 bp, Thermus aquaticus
  source 1..2583: mol_type = genomic DNA
atgcgtggtatgctgccgttgttcgagcctaaaggccgcgtactgttagtcgatggtcatcgtttggcctatcggacgttccatgcactcaaaggtctgacgaccagtcgtggccgtccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacggggcgctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 109 - protein, 861 aa, Thermus aquaticus
MRGMLPLFEPKGRVLLVDGHRLAYRTFHALKGLTTSRGRPVQAVYGFAKSLLKALKEDGDDVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAEEAAALEIKRASQPELAPEDPEDVEHHHHHH

SEQ ID NO: 110 - DNA, 2583 bp, Thermus aquaticus
  source 1..2583: mol_type = genomic DNA
atgcgtggtatgctgccgttgttcgagcctaaaggccgcgtactgttagtcgatggtcatcgtttggcctatcggacgttccatgcactcaaaggtctgacgaccagtcgtggccgtccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcagaggaagcggccgcactcgagatcaaacgggctagccagccagaactcgccccggaagaccccgaggatgtcgagcaccaccaccaccaccac

SEQ ID NO: 111 - protein, 861 aa, Thermus aquaticus
MRGMLPLFEPKGRVLLLDGHRLAYRTFHALKGLTTSRGEPVQAVYGFAKSLLKALKEDGDAVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLERWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 112 - DNA, 2583 bp, Thermus aquaticus
  source 1..2583: mol_type = genomic DNA
atgcgtggtatgctgccgttgttcgagcctaaaggccgcgtactgttacttgatggtcatcgtttggcctatcggacgttccatgcactcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaacgttggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 113 - protein, 861 aa, Thermus aquaticus
MRGMLPLFEPKGRVLLVDGHRLAYRTFHALKGLTTSRGRPVQAVYGFAKSLLKALKEDGDAVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLERWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 114 - DNA, 2583 bp, Thermus aquaticus
  source 1..2583: mol_type = genomic DNA
atgcgtggtatgctgccgttgttcgagcctaaaggccgcgtactgttagtcgatggtcatcgtttggcctatcggacgttccatgcactcaaaggtctgacgaccagtcgtggccgtccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaacgttggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 115 - protein, 861 aa, Thermus aquaticus
MRGMLPLFEPKGRVLLVDGHRLAYRTFHALKGLTTSRGEPVQAVYGFAKSLLKALKEDGDDVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLERWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 116 - DNA, 2583 bp, Thermus aquaticus
  source 1..2583: mol_type = genomic DNA
atgcgtggtatgctgccgttgttcgagcctaaaggccgcgtactgttagtcgatggtcatcgtttggcctatcggacgttccatgcactcaaaggtctgacgaccagtcgtggcgaaccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaacgttggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 117 - protein, 861 aa, Thermus aquaticus
MRGMLPLFEPKGRVLLLDGHRLAYRTFHALKGLTTSRGRPVQAVYGFAKSLLKALKEDGDAVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLERWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 118 - DNA, 2583 bp, Thermus aquaticus
  source 1..2583: mol_type = genomic DNA
atgcgtggtatgctgccgttgttcgagcctaaaggccgcgtactgttacttgatggtcatcgtttggcctatcggacgttccatgcactcaaaggtctgacgaccagtcgtggccgtccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaacgttggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 119 - protein, 861 aa, Thermus aquaticus
MRGMLPLFEPKGRVLLLDGHRLAYRTFHALKGLTTSRGEPVQAVYGFAKSLLKALKEDGDDVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLERWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 120 - DNA, 2583 bp, Thermus aquaticus
  source 1..2583: mol_type = genomic DNA
atgcgtggtatgctgccgttgttcgagcctaaaggccgcgtactgttacttgatggtcatcgtttggcctatcggacgttccatgcactcaaaggtctgacgaccagtcgtggcgaaccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaacgttggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 121 - protein, 861 aa, Thermus aquaticus
MRGMLPLFEPKGRVLLVDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLERWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTTKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 122 - DNA, 2583 bp, Thermus aquaticus
  source 1..2583: mol_type = genomic DNA
atgcgtggtatgctgccgttgttcgagcctaaaggccgcgtactgttagtcgatggtcatcacttggcctatcggacgttccatgcactcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctaccccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaacgttggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccaccaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 123 - protein, 861 aa, Thermus aquaticus
MRGMLPLFEPKGRVLLVDGHHLAYRTFHALKGLTTSRGEPVQAVYGFAKSLLKALKEDGDAVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTTKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 124 - DNA, 2583 bp, Thermus aquaticus
  source 1..2583: mol_type = genomic DNA
atgcgtggtatgctgccgttgttcgagcctaaaggccgcgtactgttagtcgatggtcatcacttggcctatcggacgttccatgcactcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccaccaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 125 - protein, 861 aa, Thermus aquaticus
MRGMLPLFEPKGRVLLVDGHHLAYRTFHALKGLTTSRGEPVQAVYGFAKSLLKALKEDGDAVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLERWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 126 - DNA, 2583 bp, Thermus aquaticus
  source 1..2583: mol_type = genomic DNA
atgcgtggtatgctgccgttgttcgagcctaaaggccgcgtactgttagtcgatggtcatcacttggcctatcggacgttccatgcactcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaacgttggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 127 - protein, 861 aa, Thermus aquaticus
MRGMLPLFEPKGRVLLVDGHHLAYRTFHALKGLTTSRGEPVQAVYGFAKSLLKALKEDGDDVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTTKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 128 - DNA, 2583 bp, Thermus aquaticus
  source 1..2583: mol_type = genomic DNA
atgcgtggtatgctgccgttgttcgagcctaaaggccgcgtactgttagtcgatggtcatcacttggcctatcggacgttccatgcactcaaaggtctgacgaccagtcgtggcgaaccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctaccccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccaccaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 129 - protein, 861 aa, Thermus aquaticus
MRGMLPLFEPKGRVLLVDGHHLAYRTFHALKGLTTSRGEPVQAVYGFAKSLLKALKEDGDDVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLERWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 130 - DNA, 2583 bp, Thermus aquaticus
  source 1..2583: mol_type = genomic DNA
atgcgtggtatgctgccgttgttcgagcctaaaggccgcgtactgttagtcgatggtcatcacttggcctatcggacgttccatgcactcaaaggtctgacgaccagtcgtggcgaaccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctaccccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaacgttggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 131 - protein, 861 aa, Thermus aquaticus
MRGMLPLFEPKGRVLLVDGHRLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTTKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 132 - DNA, 2583 bp, Thermus aquaticus
  source 1..2583: mol_type = genomic DNA
atgcgtggtatgctgccgttgttcgagcctaaaggccgcgtactgttagtcgatggtcatcgtttggcctatcggacgttccatgcactcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctaccccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccaccaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 133 - protein, 861 aa, Thermus aquaticus
MRGMLPLFEPKGRVLLVDGHRLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLERWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 134 - DNA, 2583 bp, Thermus aquaticus
  source 1..2583: mol_type = genomic DNA
atgcgtggtatgctgccgttgttcgagcctaaaggccgcgtactgttagtcgatggtcatcgtttggcctatcggacgttccatgcactcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctaccccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaacgttggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 135 - protein, 861 aa, Thermus aquaticus
MRGMLPLFEPKGRVLLLDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTTKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 136 - DNA, 2583 bp, Thermus aquaticus
  source 1..2583: mol_type = genomic DNA
atgcgtggtatgctgccgttgttcgagcctaaaggccgcgtactgttacttgatggtcatcacttggcctatcggacgttccatgcactcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctaccccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccaccaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 137 - protein, 861 aa, Thermus aquaticus
MRGMLPLFEPKGRVLLLDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLERWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 138 - DNA, 2583 bp, Thermus aquaticus
  source 1..2583: mol_type = genomic DNA
atgcgtggtatgctgccgttgttcgagcctaaaggccgcgtactgttacttgatggtcatcacttggcctatcggacgttccatgcactcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctaccccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaacgttggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 139 - protein, 861 aa, Thermus aquaticus
MRGMLPLFEPKGRVLLLDGHHLAYRTFHALKGLTTSRGEPVQAVYGFAKSLLKALKEDGDAVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 140 - DNA, 2583 bp, Thermus aquaticus
  source 1..2583: mol_type = genomic DNA
atgcgtggtatgctgccgttgttcgagcctaaaggccgcgtactgttacttgatggtcatcacttggcctatcggacgttccatgcactcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 141 - protein, 861 aa, Thermus aquaticus
MRGMLPLFEPKGRVLLLDGHHLAYRTFHALKGLTTSRGEPVQAVYGFAKSLLKALKEDGDDVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 142 - DNA, 2583 bp, Thermus aquaticus
  source 1..2583: mol_type = genomic DNA
atgcgtggtatgctgccgttgttcgagcctaaaggccgcgtactgttacttgatggtcatcacttggcctatcggacgttccatgcactcaaaggtctgacgaccagtcgtggcgaaccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctaccccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 143 - protein, 861 aa, Thermus aquaticus
MRGMLPLFEPKGRVLLLDGHRLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 144 - DNA, 2583 bp, Thermus aquaticus
  source 1..2583: mol_type = genomic DNA
atgcgtggtatgctgccgttgttcgagcctaaaggccgcgtactgttacttgatggtcatcgtttggcctatcggacgttccatgcactcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctaccccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 145 - protein, 861 aa, Thermus aquaticus
MRGMLPLFEPKGRVLLVDGHHLAYRTFHALKGLTTSRGEPVQAVYGFAKSLLKALKEDGDAVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLERWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTTKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 146 - DNA, 2583 bp, Thermus aquaticus
  source 1..2583: mol_type = genomic DNA
atgcgtggtatgctgccgttgttcgagcctaaaggccgcgtactgttagtcgatggtcatcacttggcctatcggacgttccatgcactcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaacgttggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccaccaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 147 - protein, 861 aa, Thermus aquaticus
MRGMLPLFEPKGRVLLVDGHHLAYRTFHALKGLTTSRGEPVQAVYGFAKSLLKALKEDGDDVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLERWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTTKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 148 - DNA, 2583 bp, Thermus aquaticus
  source 1..2583: mol_type = genomic DNA
atgcgtggtatgctgccgttgttcgagcctaaaggccgcgtactgttagtcgatggtcatcacttggcctatcggacgttccatgcactcaaaggtctgacgaccagtcgtggcgaaccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctaccccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaacgttggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccaccaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 149 - protein, 861 aa, Thermus aquaticus
MRGMLPLFEPKGRVLLVDGHHLAYRTFHALKGLTTSRGEPVQAVYGFAKSLLKALKEDGDDVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTTKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 150 - DNA, 2583 bp, Thermus aquaticus
  source 1..2583: mol_type = genomic DNA
atgcgtggtatgctgccgttgttcgagcctaaaggccgcgtactgttagtcgatggtcatcacttggcctatcggacgttccatgcactcaaaggtctgacgaccagtcgtggcgaaccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccaccaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 151 - protein, 861 aa, Thermus aquaticus
MRGMLPLFEPKGRVLLVDGHRLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLERWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTTKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 152 - DNA, 2583 bp, Thermus aquaticus
  source 1..2583: mol_type = genomic DNA
atgcgtggtatgctgccgttgttcgagcctaaaggccgcgtactgttagtcgatggtcatcgtttggcctatcggacgttccatgcactcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctaccccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaacgttggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccaccaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 153 - protein, 861 aa, Thermus aquaticus
MRGMLPLFEPKGRVLLVDGHRLAYRTFHALKGLTTSRGEPVQAVYGFAKSLLKALKEDGDAVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTTKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 154 - DNA, 2583 bp, Thermus aquaticus
  source 1..2583: mol_type = genomic DNA
atgcgtggtatgctgccgttgttcgagcctaaaggccgcgtactgttagtcgatggtcatcgtttggcctatcggacgttccatgcactcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccaccaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 155 - protein, 861 aa, Thermus aquaticus
MRGMLPLFEPKGRVLLVDGHRLAYRTFHALKGLTTSRGEPVQAVYGFAKSLLKALKEDGDDVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTTKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 156 - DNA, 2583 bp, Thermus aquaticus
  source 1..2583: mol_type = genomic DNA
atgcgtggtatgctgccgttgttcgagcctaaaggccgcgtactgttagtcgatggtcatcgtttggcctatcggacgttccatgcactcaaaggtctgacgaccagtcgtggcgaaccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctaccccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccaccaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 157 - protein, 861 aa, Thermus aquaticus
MRGMLPLFEPKGRVLLLDGHHLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLERWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTTKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 158 - DNA, 2583 bp, Thermus aquaticus
  source 1..2583: mol_type = genomic DNA
atgcgtggtatgctgccgttgttcgagcctaaaggccgcgtactgttacttgatggtcatcacttggcctatcggacgttccatgcactcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctaccccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaacgttggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccaccaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 159 - protein, 861 aa, Thermus aquaticus
MRGMLPLFEPKGRVLLLDGHHLAYRTFHALKGLTTSRGEPVQAVYGFAKSLLKALKEDGDAVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTTKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 160 - DNA, 2583 bp, Thermus aquaticus
  source 1..2583: mol_type = genomic DNA
atgcgtggtatgctgccgttgttcgagcctaaaggccgcgtactgttacttgatggtcatcacttggcctatcggacgttccatgcactcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccaccaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 161 - protein, 861 aa, Thermus aquaticus
MRGMLPLFEPKGRVLLLDGHHLAYRTFHALKGLTTSRGEPVQAVYGFAKSLLKALKEDGDDVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTTKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 162 - DNA, 2583 bp, Thermus aquaticus
  source 1..2583: mol_type = genomic DNA
atgcgtggtatgctgccgttgttcgagcctaaaggccgcgtactgttacttgatggtcatcacttggcctatcggacgttccatgcactcaaaggtctgacgaccagtcgtggcgaaccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctaccccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccaccaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 163 - protein, 861 aa, Thermus aquaticus
MRGMLPLFEPKGRVLLLDGHRLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTTKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 164 - DNA, 2583 bp, Thermus aquaticus
  source 1..2583: mol_type = genomic DNA
atgcgtggtatgctgccgttgttcgagcctaaaggccgcgtactgttacttgatggtcatcgtttggcctatcggacgttccatgcactcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctaccccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccaccaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 165 - protein, 861 aa, Thermus aquaticus
MRGMLPLFEPKGRVLLVDGHHLAYRTFHALKGLTTSRGEPVQAVYGFAKSLLKALKEDGDDVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLERWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 166 - DNA, 2583 bp, Thermus aquaticus
  source 1..2583: mol_type = genomic DNA
atgcgtggtatgctgccgttgttcgagcctaaaggccgcgtactgttagtcgatggtcatcacttggcctatcggacgttccatgcactcaaaggtctgacgaccagtcgtggcgaaccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaacgttggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 167 - protein, 861 aa, Thermus aquaticus
MRGMLPLFEPKGRVLLVDGHRLAYRTFHALKGLTTSRGEPVQAVYGFAKSLLKALKEDGDDVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLERWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 168 - DNA, 2583 bp, Thermus aquaticus
  source 1..2583: mol_type = genomic DNA
atgcgtggtatgctgccgttgttcgagcctaaaggccgcgtactgttagtcgatggtcatcgtttggcctatcggacgttccatgcactcaaaggtctgacgaccagtcgtggcgaaccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctaccccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaacgttggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 169 - protein, 861 aa, Thermus aquaticus
MRGMLPLFEPKGRVLLLDGHHLAYRTFHALKGLTTSRGEPVQAVYGFAKSLLKALKEDGDAVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLERWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 170 - DNA, 2583 bp, Thermus aquaticus
  source 1..2583: mol_type = genomic DNA
atgcgtggtatgctgccgttgttcgagcctaaaggccgcgtactgttacttgatggtcatcacttggcctatcggacgttccatgcactcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaacgttggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 171 - protein, 861 aa, Thermus aquaticus
MRGMLPLFEPKGRVLLLDGHHLAYRTFHALKGLTTSRGEPVQAVYGFAKSLLKALKEDGDDVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLERWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 172 - DNA, 2583 bp, Thermus aquaticus
  source 1..2583: mol_type = genomic DNA
atgcgtggtatgctgccgttgttcgagcctaaaggccgcgtactgttacttgatggtcatcacttggcctatcggacgttccatgcactcaaaggtctgacgaccagtcgtggcgaaccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctaccccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaacgttggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 173 - protein, 861 aa, Thermus aquaticus
MRGMLPLFEPKGRVLLLDGHHLAYRTFHALKGLTTSRGEPVQAVYGFAKSLLKALKEDGDDVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 174 - DNA, 2583 bp, Thermus aquaticus
  source 1..2583: mol_type = genomic DNA
atgcgtggtatgctgccgttgttcgagcctaaaggccgcgtactgttacttgatggtcatcacttggcctatcggacgttccatgcactcaaaggtctgacgaccagtcgtggcgaaccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 175 - protein, 861 aa, Thermus aquaticus
MRGMLPLFEPKGRVLLLDGHRLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLERWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 176 - DNA, 2583 bp, Thermus aquaticus
  source 1..2583: mol_type = genomic DNA
atgcgtggtatgctgccgttgttcgagcctaaaggccgcgtactgttacttgatggtcatcgtttggcctatcggacgttccatgcactcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctaccccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaacgttggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 177 - protein, 861 aa, Thermus aquaticus
MRGMLPLFEPKGRVLLLDGHRLAYRTFHALKGLTTSRGEPVQAVYGFAKSLLKALKEDGDDVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 178 - DNA, 2583 bp, Thermus aquaticus
  source 1..2583: mol_type = genomic DNA
atgcgtggtatgctgccgttgttcgagcctaaaggccgcgtactgttacttgatggtcatcgtttggcctatcggacgttccatgcactcaaaggtctgacgaccagtcgtggcgaaccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctaccccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 179 - protein, 861 aa, Thermus aquaticus
MRGMLPLFEPKGRVLLVDGHHLAYRTFHALKGLTTSRGEPVQAVYGFAKSLLKALKEDGDDVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLERWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTTKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 180 - DNA, 2583 bp, Thermus aquaticus
  source 1..2583: mol_type = genomic DNA
atgcgtggtatgctgccgttgttcgagcctaaaggccgcgtactgttagtcgatggtcatcacttggcctatcggacgttccatgcactcaaaggtctgacgaccagtcgtggcgaaccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaacgttggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccaccaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 181 - protein, 861 aa, Thermus aquaticus
MRGMLPLFEPKGRVLLVDGHRLAYRTFHALKGLTTSRGEPVQAVYGFAKSLLKALKEDGDAVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLERWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTTKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 182 - DNA, 2583 bp, Thermus aquaticus
  source 1..2583: mol_type = genomic DNA
atgcgtggtatgctgccgttgttcgagcctaaaggccgcgtactgttagtcgatggtcatcgtttggcctatcggacgttccatgcactcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaacgttggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccaccaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 183 - protein, 861 aa, Thermus aquaticus
MRGMLPLFEPKGRVLLVDGHRLAYRTFHALKGLTTSRGEPVQAVYGFAKSLLKALKEDGDDVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLERWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTTKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 184 - DNA, 2583 bp, Thermus aquaticus
  source 1..2583: mol_type = genomic DNA
atgcgtggtatgctgccgttgttcgagcctaaaggccgcgtactgttagtcgatggtcatcgtttggcctatcggacgttccatgcactcaaaggtctgacgaccagtcgtggcgaaccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctaccccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaacgttggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccaccaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 185 - protein, 861 aa, Thermus aquaticus
MRGMLPLFEPKGRVLLVDGHRLAYRTFHALKGLTTSRGEPVQAVYGFAKSLLKALKEDGDDVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTTKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 186 - DNA, 2583 bp, Thermus aquaticus
  source 1..2583: mol_type = genomic DNA
atgcgtggtatgctgccgttgttcgagcctaaaggccgcgtactgttagtcgatggtcatcgtttggcctatcggacgttccatgcactcaaaggtctgacgaccagtcgtggcgaaccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccaccaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 187 - protein, 861 aa, Thermus aquaticus
MRGMLPLFEPKGRVLLLDGHHLAYRTFHALKGLTTSRGEPVQAVYGFAKSLLKALKEDGDAVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLERWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTTKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 188 - DNA, 2583 bp, Thermus aquaticus
  source 1..2583: mol_type = genomic DNA
atgcgtggtatgctgccgttgttcgagcctaaaggccgcgtactgttacttgatggtcatcacttggcctatcggacgttccatgcactcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaacgttggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccaccaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 189 - protein, 861 aa, Thermus aquaticus
MRGMLPLFEPKGRVLLLDGHHLAYRTFHALKGLTTSRGEPVQAVYGFAKSLLKALKEDGDDVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLERWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTTKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 190 - DNA, 2583 bp, Thermus aquaticus
  source 1..2583: mol_type = genomic DNA
atgcgtggtatgctgccgttgttcgagcctaaaggccgcgtactgttacttgatggtcatcacttggcctatcggacgttccatgcactcaaaggtctgacgaccagtcgtggcgaaccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctaccccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaacgttggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccaccaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 191 - protein, 861 aa, Thermus aquaticus
MRGMLPLFEPKGRVLLLDGHHLAYRTFHALKGLTTSRGEPVQAVYGFAKSLLKALKEDGDDVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTTKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 192 - DNA, 2583 bp, Thermus aquaticus
  source 1..2583: mol_type = genomic DNA
atgcgtggtatgctgccgttgttcgagcctaaaggccgcgtactgttacttgatggtcatcacttggcctatcggacgttccatgcactcaaaggtctgacgaccagtcgtggcgaaccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccaccaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 193 - protein, 861 aa, Thermus aquaticus
MRGMLPLFEPKGRVLLLDGHRLAYRTFHALKGLTTSRGEPVQAVYGFAKSLLKALKEDGDAVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLERWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTTKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 194 - DNA, 2583 bp, Thermus aquaticus
  source 1..2583: mol_type = genomic DNA
atgcgtggtatgctgccgttgttcgagcctaaaggccgcgtactgttacttgatggtcatcgtttggcctatcggacgttccatgcactcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctaccccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaacgttggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccaccaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 195 - protein, 861 aa, Thermus aquaticus
MRGMLPLFEPKGRVLLLDGHRLAYRTFHALKGLTTSRGEPVQAVYGFAKSLLKALKEDGDAVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTTKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 196 - DNA, 2583 bp, Thermus aquaticus
  source 1..2583: mol_type = genomic DNA
atgcgtggtatgctgccgttgttcgagcctaaaggccgcgtactgttacttgatggtcatcgtttggcctatcggacgttccatgcactcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccaccaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 197 - protein, 861 aa, Thermus aquaticus
MRGMLPLFEPKGRVLLLDGHRLAYRTFHALKGLTTSRGEPVQAVYGFAKSLLKALKEDGDDVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTTKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 198 - DNA, 2583 bp, Thermus aquaticus
  source 1..2583: mol_type = genomic DNA
atgcgtggtatgctgccgttgttcgagcctaaaggccgcgtactgttacttgatggtcatcgtttggcctatcggacgttccatgcactcaaaggtctgacgaccagtcgtggcgaaccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctaccccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccaccaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 199 - protein, 861 aa, Thermus aquaticus
MRGMLPLFEPKGRVLLLDGHHLAYRTFHALKGLTTSRGEPVQAVYGFAKSLLKALKEDGDDVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLERWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 200 - DNA, 2583 bp, Thermus aquaticus
  source 1..2583: mol_type = genomic DNA
atgcgtggtatgctgccgttgttcgagcctaaaggccgcgtactgttacttgatggtcatcacttggcctatcggacgttccatgcactcaaaggtctgacgaccagtcgtggcgaaccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaacgttggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 201 - protein, 861 aa, Thermus aquaticus
MRGMLPLFEPKGRVLLLDGHRLAYRTFHALKGLTTSRGEPVQAVYGFAKSLLKALKEDGDDVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLERWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 202 - DNA, 2583 bp, Thermus aquaticus
  source 1..2583: mol_type = genomic DNA
atgcgtggtatgctgccgttgttcgagcctaaaggccgcgtactgttacttgatggtcatcgtttggcctatcggacgttccatgcactcaaaggtctgacgaccagtcgtggcgaaccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctaccccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaacgttggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 203 - protein, 861 aa, Thermus aquaticus
MRGMLPLFEPKGRVLLLDGHRLAYRTFHALKGLTTSRGEPVQAVYGFAKSLLKALKEDGDDVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 204 - DNA, 2583 bp, Thermus aquaticus
  source 1..2583: mol_type = genomic DNA
atgcgtggtatgctgccgttgttcgagcctaaaggccgcgtactgttacttgatggtcatcgtttggcctatcggacgttccatgcactcaaaggtctgacgaccagtcgtggcgaaccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccgaaaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 205 - protein, 861 aa, Thermus aquaticus
MRGMLPLFEPKGRVLLVDGHRLAYRTFHALKGLTTSRGEPVQAVYGFAKSLLKALKEDGDDVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLERWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTTKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 206 - DNA, 2583 bp, Thermus aquaticus
  source 1..2583: mol_type = genomic DNA
atgcgtggtatgctgccgttgttcgagcctaaaggccgcgtactgttagtcgatggtcatcgtttggcctatcggacgttccatgcactcaaaggtctgacgaccagtcgtggcgaaccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaacgttggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccaccaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 207 - protein, 861 aa, Thermus aquaticus
MRGMLPLFEPKGRVLLLDGHHLAYRTFHALKGLTTSRGEPVQAVYGFAKSLLKALKEDGDDVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLERWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTTKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 208 - DNA, 2583 bp, Thermus aquaticus
  source 1..2583: mol_type = genomic DNA
atgcgtggtatgctgccgttgttcgagcctaaaggccgcgtactgttacttgatggtcatcacttggcctatcggacgttccatgcactcaaaggtctgacgaccagtcgtggcgaaccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaacgttggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccaccaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 209 - protein, 861 aa, Thermus aquaticus
MRGMLPLFEPKGRVLLLDGHRLAYRTFHALKGLTTSRGEPVQAVYGFAKSLLKALKEDGDAVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLERWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTTKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 210 - DNA, 2583 bp, Thermus aquaticus
  source 1..2583: mol_type = genomic DNA
atgcgtggtatgctgccgttgttcgagcctaaaggccgcgtactgttacttgatggtcatcgtttggcctatcggacgttccatgcactcaaaggtctgacgaccagtcgtggcgaaccggtccaggctgtttatggtttcgctaagtctttgctcaaagcactgaaagaagacggggacgcggtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaacgttggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccaccaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 211 - protein, 861 aa, Thermus aquaticus
MRGMLPLFEPKGRVLLLDGHRLAYRTFHALKGLTTSRGEPVQAVYGFAKSLLKALKEDGDDVIVVFDAKAPSFRHEAYGGYKAGRAPTPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLERWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTTKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 212 - DNA, 2583 bp, Thermus aquaticus
  source 1..2583: mol_type = genomic DNA
atgcgtggtatgctgccgttgttcgagcctaaaggccgcgtactgttacttgatggtcatcgtttggcctatcggacgttccatgcactcaaaggtctgacgaccagtcgtggcgaaccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctaccccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaacgttggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccaccaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 213 - protein, 861 aa, Thermus aquaticus
MRGMLPLFEPKGRVLLLDGHRLAYRTFHALKGLTTSRGEPVQAVYGFAKSLLKALKEDGDDVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLEEWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTTKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 214 - DNA, 2583 bp, Thermus aquaticus
  source 1..2583: mol_type = genomic DNA
atgcgtggtatgctgccgttgttcgagcctaaaggccgcgtactgttacttgatggtcatcgtttggcctatcggacgttccatgcactcaaaggtctgacgaccagtcgtggcgaaccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaagagtggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccaccaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

SEQ ID NO: 215 - protein, 861 aa, Thermus aquaticus
MRGMLPLFEPKGRVLLLDGHRLAYRTFHALKGLTTSRGEPVQAVYGFAKSLLKALKEDGDDVIVVFDAKAPSFRHEAYGGYKAGRAPRPEDFPRQLALIKELVDLLGLARLEVPGYEADDVLASLAKKAEKEGYEVRILTADKDLYQLLSDRIHVLHPEGYLITPAWLWEKYGLRPDQWADYRALTGDESDNLPGVKGIGEKTARKLLERWGSLEALLKNLDRLKPAIREKILAHMDDLKLSWDLAKVRTDLPLEVDFAKRREPDRERLRAFLERLEFGSLLHEFGLLESPKALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTTKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKEAAALEIKRASQPELAPEDPEDVEHHHHHH

SEQ ID NO: 216 - DNA, 2583 bp, Thermus aquaticus
  source 1..2583: mol_type = genomic DNA
atgcgtggtatgctgccgttgttcgagcctaaaggccgcgtactgttacttgatggtcatcgtttggcctatcggacgttccatgcactcaaaggtctgacgaccagtcgtggcgaaccggtccaggctgtttatggtttcgctaagtctttgctcaaagcactgaaagaagacggggacgatgtaattgttgtatttgatgccaaagcaccgagcttccgccacgaagcttatggtggctacaaggcaggacgcgcccctcgtccagaagatttcccccgtcagctggcattaattaaggagttagtagaccttctcggcttagcgcgtctggaagttccgggttatgaggcggacgatgtccttgcatccttggctaaaaaggccgaaaaagagggctacgaagtccgcatcttgacggcagacaaagatctgtaccagcttctgtctgaccgtattcatgttttgcaccctgaaggctacttaatcactccggcctggctctgggaaaagtacggtctgcgtcccgatcagtgggcggattatcgggctttgacgggagatgagagcgacaacctgccaggagttaagggcattggtgaaaaaaccgcacgtaagctgcttgaacgttggggttccctggaagccttgttaaaaaatctggatcgtctcaagcccgcaattcgtgaaaagatcctggctcatatggacgatcttaaattaagttgggacctggccaaggtgcgcaccgatttaccgcttgaagtggattttgcaaaacgccgtgagccggaccgggaacgtttacgcgctttcttagagcgtctggaattcggttcactgcttcatgaattcggtctgttagagtctcctaaagcactcgaagaggcaccgtggccgcccccagaaggtgcttttgttggcttcgtactttcccgtaaggagcctatgtgggcagatcttctggctttagcggctgcacgcggtggccgtgttcaccgggcccctgagccatacaaagcgttacgtgatctgaaggaagcacgtggcttgctggcaaaagacctttctgttttggccctgcgcgagggtcttggactgccgccaggcgacgatcccatgttattggcctatctgttagaccctagcaataccacacctgaaggggtcgctcgtcggtatggcggtgaatggactgaggaagccggagagcgcgccgcattgtccgaacggctctttgcaaacttatggggtcgtctggaaggggaggaacgtctgttatggttgtatcgggaagtcgaacgtcctctttcggccgtattagcgcatatggaggcaacaggtgtgcgtttagatgtcgcgtaccttcgggccttatcactggaagttgcagaggaaatcgcccgtctcgaggctgaagtgttccggttggccggtcacccgtttaacctcaactcccgtgaccagctggaacgcgttttattcgatgagcttgggcttcccgcaattggcaaaaccaccaagactggcaaacgcagtacgagcgctgccgtccttgaggcactccgcgaggctcaccctattgtagaaaagatcctgcaataccgtgagttgacgaagcttaaaagcacttatattgatcctctcccggatctgatccatcctcgtaccggccgcttgcacacacgtttcaaccagacggcgactgcaaccggccgtctgtctagctcggatccaaatctccagaacattccggtccgtacacccttgggccaacgtatccgccgggcgtttatcgcggaagaaggatggttactggtcgcattggactactcgcagattgagctgcgcgtcctcgcacatctctctggtgacgaaaatttaatccgcgtgtttcaagaggggcgtgatattcacacagaaactgcctcatggatgttcggtgtcccacgtgaagcagtggatcctttgatgcgccgtgcagctaaaacaattaattttggagtgctgtacggaatgagcgctcatcgcttgagtcaggaactggcaatcccctacgaggaagcgcaggcattcatcgaacgttactttcaatcgtttccgaaagttcgcgcatggatcgagaagacgctcgaggaaggtcgtcgtcggggctatgtcgaaactctgtttggtcgccgtcggtacgtaccagatcttgaagcccgcgtcaaatcggtacgggaggctgcggagcgtatggcatttaatatgcctgtacagggtactgcagctgacctcatgaaactggcaatggtcaagcttttcccgcgcttggaggaaatgggcgcacgtatgcttctgcaggtccatgacgagctggtgttagaagcccctaaggagcgcgccgaagctgtcgcgcgcctcgctaaagaagtgatggagggcgtttacccattggccgtacccctcgaagtggaggtcggtattggagaagattggttatctgcaaaggaagcggccgcactcgagatcaaacgggctagccagccagaactcgccccggaagaccccgaggatgtcgagcaccaccaccaccaccac

(19) United States
(12) Patent Application Publication
(10) Pub. No.: US 2026/0022355 A1
(43) Pub. Date: Jan. 22, 2026

(54) TAQ DNA POLYMERASE STACKED MUTANTS

(71) Applicant: Integrated DNA Technologies, Inc., Coralville, IA (US)

(72) Inventors: Sarah Franz Beaudoin, Iowa City, IA (US); Christopher Anthony Vakulskas, North Liberty, IA (US)

(21) Appl. No.: 19/275,364

(22) Filed: Jul. 21, 2025

Related U.S. Application Data
(60) Provisional application No. 63/673,891, filed on Jul. 22, 2024.
(60) Provisional application No. 63/709,825, filed on Oct. 21, 2024.

(51) Int. Cl. C12N 9/12; C12N 15/10; C12P 19/34; C12Q 1/686

(52) U.S. Cl. CPC C12N 9/1252; C12N 15/1093; C12P 19/34; C12Q 1/686; C12Y 207/07007

(57) ABSTRACT

The present disclosure relates to Taq DNA Polymerase mutants comprising at least three mutations which are capable of high levels of DNA amplification in the presence of several PCR inhibitors and compositions comprising the same. Also disclosed are methods of performing mutagenesis on wild-type Taq DNA polymerase to obtain the disclosed Taq DNA polymerase mutants, DNA amplification using the same, and cDNA amplification using the same.

### RELATED APPLICATIONS

The present application claims priority to U.S. Provisional Patent Application No. 63/709,825, filed Oct. 21, 2024, and U.S. Provisional Application No. 63/673,891, filed on Jul. 22, 2024. The entire text of the aforementioned applications is incorporated herein by reference in its entirety.

### REFERENCE TO ELECTRONIC SEQUENCE LISTING

The contents of the electronic sequence listing (68467US02_SEQ_LIST_ST26.xml; Size: 558,982 bytes; and Date of Creation: Jul. 21, 2025) are herein incorporated by reference in its entirety.

### BACKGROUND

Polymerase-based assays, such as the polymerase chain reaction (PCR), have an important impact and widespread use in genetic diagnostics and molecular medicine. Polymerases synthesize DNA sequences by the addition of nucleotides to the 3′ end of a short oligonucleotide (abbreviated to “primer” in the following). The primer is hybridized to the single stranded sequence that is going to be amplified (“template”). DNA polymerases catalyze the formation of a phosphodiester bond between the 3′-oxygen at the 3′-terminus of the primer and the incoming deoxynucleoside triphosphate (“dNTP”). This chemical reaction (“primer extension”) adds a nucleotide to the primer (e.g., to the nascent growing DNA chain). Primer extension is base specific, in that the deoxynucleoside triphosphate that is complementary to the base in the template is added to the primer. The fidelity of DNA polymerase enzymes is very high and the rate of mutations introduced into the replicated DNA strand is low; however, the precise error rate varies between different DNA polymerase enzymes and these rates have been well characterized. The extension reaction can be repeated until the end of the template is reached.

DNA polymerase I is an enzyme that contributes to the process of prokaryotic DNA replication and repair. Taq DNA polymerase is a thermostable DNA polymerase I named after the thermophilic eubacterial microorganism, Thermus aquaticus, and was first isolated by Chen et al. in 1976. Due to the thermophilic characteristics of Taq DNA polymerase, it effectively and revolutionarily replaces the DNA polymerase I from Escherichia coli, the original variant that cannot withstand the protein denaturing temperatures used in PCR (Chen, A. 1976).

Mutations at various amino acid positions in the Taq DNA polymerase are known to improve various different properties, including resistance to PCR inhibitors (Ghadessy et al., 2001), increased DNA-binding affinity and blood resistance (Arezi, B. 2014), a more thermostable Phi29 DNA polymerase (Povilaitis, T. 2016) and a more thermostable strand-displacing polymerase (Milligan, J. N. 2018), among others. Some known mutations include K225E, E388V, K540R, D578G, N583S (Ghadessy F. J. 2001) and M747R and G59W, V155I, L245M, L375V, E507K, K508R, E734G and F749I (Arezi, B. 2014). These mutations may be at or near the binding pocket of the protein (Ghadessy F. J. 2001).

A need for mutant Taq DNA polymerase enzymes with novel and improved properties still exists in the art.

### BRIEF SUMMARY

The present technology relates to a Taq DNA polymerase mutant with at least one mutation. The present technology also relates to the ability and process to amplify DNA templates using a Taq DNA polymerase of the present technology. The present technology also involves the ability and process to encapsulate E. coli cells containing desired Taq DNA polymerase mutants in combination with compartmentalized self-replication (CSR), a form of in-vitro compartmentalization (IVC), to enrich for a desired/selected mutant(s) with desired/selected characteristics.

In certain aspects, the present technology relates to a Taq DNA polymerase mutant comprising a mutation at one or more amino acid positions, wherein the one or more mutations is selected from A141P, A227S, A227Y, A259T, A29P, A341K, A439M, A61D, A705S, A70M, A757S, A77S, A83H, D151S, D547R, E117R, E135A, E159G, E210G, E210R, E255Q, E263S, E296G, E388S, E39R, E404G, E423G, E423I, E465A, E507K, E507T, E524G, E724G, E742G, E742N, E774P, E774S, F27P, F27R, F632L, F73R, F73S, F749A, G115R, G12R, G160N, G160S, G195R, G286S, G395Y, G59F, G59T, G84A, H21R, H75R, K128R, K225R, K247A, K260G, K508R, K531R, K709L, K709R, K831E, L106V, L148P, L233D, L245H, L33P, L352P, L367V, L456P, L55R, L5P, L605P, L606V, L670P, L768P, L96P, M775Y, P194Y, P527Q, P589A, P89I, Q178R, Q582M, Q680R, Q690R, Q754R, Q782R, Q95T, R110M, R2F, R596S, R695S, R801Q, S290P, S383H, S383N, S383P, S543F, S679G, T386Q, T569A, T88R, V103A, V17L, V449A, V607N, V631A, V821D, W167E, W706F, Y16IN, Y16IR, Y161V, Y182S, Y24H, Y24T, Y339P, Y339V, Y455R, Y78R, and Y78S.

In a further aspect, the present technology relates to a Taq DNA polymerase mutant, wherein the Taq DNA polymerase mutant has a mutation at two or more amino acid positions.

In a still further aspect, the present technology relates to a Taq DNA polymerase mutant, wherein the Taq DNA polymerase mutant comprises a mutation at two or more amino acid positions, wherein the mutation at the first amino acid position is selected from A61D, E39R, H21R, L367V, R2F, Y182S, and T88R; and the mutation at the second amino acid position is selected from A61D, E39R, E210R, E507T, H21R, L367V, R2F, Y182S, and T88R; wherein the mutation at the first amino acid position and the mutation at the second amino acid position are different from one another.

In a still further aspect, the present technology relates to a Taq DNA polymerase mutant, wherein the Taq DNA polymerase mutant has a mutation at three or more amino acid positions. In an alternative aspect, the mutation at the first amino acid position is selected from H21R, R2F, T88R, E39R, A61D, L367V, and Y182S; the mutation at the second amino acid position is selected from H21R, R2F, T88R, E39R, A61D, L367V, and Y182S; and the mutation at the third amino acid position is selected from H21R, R2F, T88R, E39R, A61D, L367V, and Y182S, wherein the mutation at the first amino acid position, the mutation at the second amino acid position, and the mutation at the third amino acid position are different from one another. Alternatively, the mutation at the first amino acid position is H21R; the mutation at the second amino acid position is T88R, E39R or A61D; and the mutation at the third amino acid position is L367V or T88R.

In certain aspects, the present technology relates to a Taq DNA polymerase mutant, wherein the Taq DNA Polymerase comprises a mutation at three or more amino acid positions. In an alternative aspect, the mutation at the first amino acid position is selected from H21R, R2F, T88R, E39R, A61D, L367V, and Y182S; the mutation at the second amino acid position is selected from H21R, R2F, T88R, E39R, A61D, L367V, and Y182S; and the mutation at the third amino acid position is selected from H21R, R2F, T88R, E39R, A61D, L367V, and Y182S, wherein the mutation at the first amino acid position, the mutation at the second amino acid position, and the mutation at the third amino acid position are different from one another.

In certain aspects, the present technology relates to a Taq DNA polymerase mutant, wherein the Taq DNA Polymerase comprises a mutation at four or more amino acid positions. In an alternative aspect, the mutation at the first amino acid position is selected from H21R, R2F, and V17L; the mutation at the second amino acid position is selected from E39R, H21R, A29P, T88R, Y24H, F27R, and A61D; the mutation at the third amino acid position is selected from A61D, T88R, E210R, V631A, K709R, E724G, K831E, Y182S, and E39R, and the fourth amino acid position is selected from T88R, A61D, L367V, E507T, and E210R, wherein the mutation at the first amino acid position, the mutation at the second amino acid position, the mutation a the third amino acid and the mutation at the fourth amino acid position are different from one another. Alternatively, the mutation at the first amino acid position is H21R; the mutation at the second amino acid position is E39R or A61D; the mutation at the third amino acid position T88R; and the mutation at the fourth amino acid position is E210R.

In certain aspects, the present technology relates to a Taq DNA polymerase mutant, wherein the Taq DNA Polymerase comprises a mutation at five or more amino acid positions. In an alternative aspect, the mutation at the first amino acid position is H21R or V17L; the mutation at the second amino acid position is selected from the group consisting of A29P, E39R, H21R, Y24H, F27R and A61D; the mutation at the third amino acid position is selected from the group consisting of E39R, A61D, T88R; the mutation at the fourth amino acid position is T88R, E210R, and A61D; and the mutation at the fifth amino acid position is selected from T88R, E210R, E507T, K709R, V631A, E274G, E724G, and K831E, wherein the mutation at the first amino acid position, the mutation at the second amino acid position, the mutation at the third amino acid position, the mutation at the fourth amino acid position, and the mutation at the fifth amino acid position are different from one another. Alternatively, the mutation at the first amino acid position is V17L; the mutation at the second amino acid position is H21R; the mutation at the third amino acid position E39R or A61D; the mutation at the fourth amino acid position is T88R; and the mutation at the fifth amino acid position is E210R.

In certain aspects, the present technology relates to a Taq DNA polymerase mutant, wherein the Taq DNA Polymerase comprises a mutation at six or more amino acid positions. In this aspect, the mutation at the first amino acid position is V17L; the mutation at a second amino acid position is H21R; the mutation at a third amino acid position is A61D; the mutation at a fourth amino acid position is T88R; the mutation at the fifth amino acid position is selected from E210R; and the mutation at the sixth amino acid position is E507T.

In a still further aspect, the present technology relates to a Taq DNA polymerase mutant wherein the Taq DNA polymerase mutant exhibits, relative to wild-type DNA polymerase, increased DNA amplification, increased resistance to a PCR inhibitor, and/or increased tolerance to a PCR inhibitor.

In a still further aspect, the present technology relates to a Taq DNA polymerase mutant, wherein the Taq DNA polymerase mutant possesses an increased DNA amplification factor, which is at least a 20-fold increase relative to wild-type DNA polymerase. In an alternative aspect, the one or more mutations is selected from L106V, S383N, K260G, T386Q, T88R, E388S, E742N, A259T, W706F, F27P, E774P, E255Q, Y161R, A29P, F749A, E465A, F73R, S543F, G59F, G59T, E210R, A61D, Y78S, F73R, P589A, K709L, Y24H, L245H, F73S, E117R, P89I, R801Q, A705S, S383H, V17L, A141P, F27R, M775Y, E423I, R596S, G84A, E742G, E117R, Y24T, S290P, K531I, G286S. Y78R, Y339P, V631A, A77S, L233D, G12R, K831E, G195R, K709R, and L367V.

In a still further aspect, the present technology relates to a Taq DNA polymerase mutant, wherein the Taq DNA polymerase mutant possesses an increased DNA amplification factor, which is at least a 45-fold increase relative to wild-type DNA polymerase. In an alternative aspect, the one or more mutations is selected from L106V, S383N, K260G, T386Q, T88R, E388S, E742N, A259T, W706F, F27P, E774P, E255Q, Y161R, A29P, F749A, E465A, F73R, S543F, and G59F.

In a still further aspect, the present technology relates to a Taq DNA polymerase mutant, wherein the Taq DNA polymerase mutant possesses an increased DNA amplification factor which is at least a 60-fold increase relative to wild-type DNA polymerase. In an alternative aspect, the one or more mutations is selected from L106V, S383N, K260G, T386Q, T88R, and E388S.

In a still further aspect, the present technology relates to a Taq DNA polymerase mutant, wherein the PCR inhibitor is selected from the group consisting of humic acid, heparin, hematin, high-salt, ethanol, and guanidine thiocyanate.

In a still further aspect, the present technology relates to a Taq DNA polymerase mutant, wherein the PCR inhibitor is humic acid.

In a still further aspect, the present technology relates to a Taq DNA polymerase mutant, wherein the DNA amplification increases after a second round of amplification.

In certain aspects, the present technology relates to an isolated nucleic acid molecule comprising a nucleotide sequence encoding the Taq DNA polymerase mutant of any one of the previously described aspects.

In certain aspects, the present technology related to a composition comprising the Taq DNA polymerase mutant of the present technology as described herein.

In certain aspects, the present technology relates to a kit comprising the Taq DNA polymerase mutant of the present technology as described herein and one or more reagents utilized for a DNA synthesis reaction.

In certain aspects, the present technology relates to a method for conducting primer extension, comprising the step of contacting a Taq DNA polymerase mutant as described above with a primer, a polynucleotide template, and nucleoside triphosphates under conditions suitable for a primer extension method, thereby producing an extended primer.

In certain aspects, the present technology relates to a method for synthesizing complementary amplifying deoxyribonucleic acid (cDNA) comprising the steps of providing the Taq DNA polymerase mutant of any one of the previously described aspects of the present technology and contacting the Taq DNA polymerase mutant with a nucleic acid template to permit synthesis of cDNA.

In certain aspects, the present technology relates to a method for performing polymerase chain reaction (PCR) on a sample containing humic acid comprising the steps of providing the Taq DNA polymerase mutant of any of the previously described aspects of the present technology and contacting the Taq DNA polymerase mutant with a nucleic acid template to replicate and amplify the nucleic acid template.

In a further aspect, the present technology relates to the described methods, wherein the amplification of the nucleic acid template is at least a 5-fold increase relative to wild-type DNA polymerase.

In certain aspects, the present technology relates to a method for obtaining a Taq DNA polymerase mutant comprising the steps of (a) subjecting a polynucleotide encoding a Taq DNA polymerase to PCR to generate a library comprising an array of different polynucleotides; (b) screening the library for increased DNA amplification, increased resistance to a PCR inhibitor, and/or increased tolerance to a PCR inhibitor; (c) selecting one or more Taq DNA polymerases from screening step b; and (d) repeating steps a to c until a final Taq DNA polymerase is obtained, wherein the final Taq DNA polymerase comprises two or more amino acid mutations, wherein the two or more mutations are selected from A141P, A227S, A227Y, A259T, A29P, A341K, A439M, A61D, A705S, A70M, A757S, A77S, A83H, D151S, D547R, E117R, E135A, E159G, E210G, E210R, E255Q, E263S, E296G, E388S, E39R, E404G, E423G, E423I, E465A, E507K, E507T, E524G, E724G, E742G, E742N, E774P, E774S, F27P, F27R, F632L, F73R, F73S, F749A, G115R, G12R, G160N, G160S, G195R, G286S, G395Y, G59F, G59T, G84A, H21R, H75R, K128R, K225R, K247A, K260G, K508R, K531R, K709L, K709R, K831E, L106V, L148P, L233D, L245H, L33P, L352P, L367V, L456P, L55R, L5P, L605P, L606V, L670P, L768P, L96P, M775Y, P194Y, P527Q, P589A, P89I, Q178R, Q582M, Q680R, Q690R, Q754R, Q782R, Q95T, R110M, R2F, R596S, R695S, R801Q, S290P, S383H, S383N, S383P, S543F, S679G, T386Q, T569A, T88R, V103A, V17L, V449A, V607N, V631A, V821D, W167E, W706F, Y16IN, Y161R, Y161V, Y182S, Y24H, Y24T, Y339P, Y339V, Y455R, Y78R, and Y78S. Alternatively, the two or more mutations are selected from H21R, E39R, A61D, T88R, V17L, A29P, E210R, V631A, E724G, K831E, K709R, Y24H, and F27R.

In a still further aspect, the present technology relates to the previously described method, wherein the PCR inhibitor is selected from the group consisting of humic acid, heparin, hematin, high-salt, ethanol, and guanidine thiocyanate.

In a still further aspect, the present technology related to the previously described method, wherein the PCR inhibitor is humic acid.

### BRIEF DESCRIPTION OF THE DRAWINGS

Various aspects of the present disclosure will now be described, by way of example only, with reference to the attached Figures, wherein:

FIG. 1 is an exemplary Compartmentalized Self-Replication (CSR) diagram.

### DETAILED DESCRIPTION

The present technology relates to Taq DNA polymerase mutants. The present technology also relates to point mutations in the Taq DNA polymerase for mutagenesis and methods and kits for using the described Taq DNA polymerase mutants to amplify cDNA from DNA templates.

DNA polymerase is an enzyme that synthesizes long chains of, for example, nucleic acids, and is used to replicate a DNA template strand using base-pairing interactions. Taq DNA polymerase refers to an isolated DNA polymerase enzyme derived from the Thermus aquaticus microorganism. Some forms of Taq DNA polymerase have optimum temperatures for activity ranging from between about 75° C. to about 80° C.

The peptide and gene sequences of Taq DNA polymerase enzyme isolated from nature are well known in prior art and are listed in Table 1 (Lawyer, F. C., et al., J. Biol. Chem. 1989, 264:6427-6437; Genbank database ID J04639.1).

TABLE 1
DNA and amino acid sequence of native (wild type) Taq DNA polymerase.

| | | SEQ |
|---|---|---|
| | | ID |
| Type | Sequence | NO |
| Protein | MRGMLPLFEPKGRVLLVDGHHLAYRTFHALKGLTTSRGEPVQAVYGFAKSLLKAL | 1 |
| sequence | KEDGDAVIVVFDAKAPSFRHEAYGGYKAGRAPTPEDFPRQLALIKELVDLLGLAR | |
| (N to C | LEVPGYEADDVLASLAKKAEKEGYEVRILTADKDLYQLLSDRIHVLHPEGYLITP | |
| terminus) | AWLWEKYGLRPDQWADYRALTGDESDNLPGVKGIGEKTARKLLEEWGSLEALLKN | |
| | LDRLKPAIREKILAHMDDLKLSWDLAKVRTDLPLEVDFAKRREPDRERLRAFLER | |
| | LEFGSLLHEFGLLESPKALEEAPWPPPEGAFVGFVLSRKEPMWADLLALAAARGG | |
| | RVHRAPEPYKALRDLKEARGLLAKDLSVLALREGLGLPPGDDPMLLAYLLDPSNT | |
| | TPEGVARRYGGEWTEEAGERAALSERLFANLWGRLEGEERLLWLYREVERPLSAV | |
| | LAHMEATGVRLDVAYLRALSLEVAEEIARLEAEVFRLAGHPFNLNSRDQLERVLF | |
| | DELGLPAIGKTEKTGKRSTSAAVLEALREAHPIVEKILQYRELTKLKSTYIDPLP | |
| | DLIHPRTGRLHTRFNQTATATGRLSSSDPNLQNIPVRTPLGQRIRRAFIAEEGWL | |
| | LVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGR | |
| | RRGYVETLFGRRRYVPDLEARVKSVREAAERMAFNMPVQGTAADLMKLAMVKLFP | |
| | RLEEMGARMLLQVHDELVLEAPKERAEAVARLAKEVMEGVYPLAVPLEVEVGIGE | |
| | DWLSAKE | |
| DNA | aagctcagat ctacctgcct gagggcgtcc ggttccagct ggcccttccc | 2 |
| sequence | gagggggaga gggaggcgtt tctaaaagcc cttcaggacg ctacccgggg | |
| (5′ to 3′) | gcgggtggtg gaagggtaac atgaggggga tgctgcccct ctttgagccc | |
| | aagggccggg tcctcctggt ggacggccac cacctggcct accgcacctt | |
| | ccacgccctg aagggcctca ccaccagccg gggggagccg gtgcaggcgg | |
| | tctacggctt cgccaagagc ctcctcaagg ccctcaagga ggacggggac | |
| | gcggtgatcg tggtctttga cgccaaggcc ccctccttcc gccacgaggc | |
| | ctacgggggg tacaaggcgg gccgggcccc cacgccggag gactttcccc | |
| | ggcaactcgc cctcatcaag gagctggtgg acctcctggg gctggcgcgc | |
| | ctcgaggtcc cgggctacga ggcggacgac gtcctggcca gcctggccaa | |
| | gaaggcggaa aaggagggct acgaggtccg catcctcacc gccgacaaag | |
| | acctttacca gctcctttcc gaccgcatcc acgtcctcca ccccgagggg | |
| | tacctcatca ccccggcctg gctttgggaa aagtacggcc tgaggcccga | |
| | ccagtgggcc gactaccggg ccctgaccgg ggacgagtcc gacaaccttc | |
| | ccggggtcaa gggcatcggg gagaagacgg cgaggaagct tctggaggag | |
| | tgggggagcc tggaagccct cctcaagaac ctggaccggc tgaagcccgc | |
| | catccgggag aagatcctgg cccacatgga cgatctgaag ctctcctggg | |
| | acctggccaa ggtgcgcacc gacctgcccc tggaggtgga cttcgccaaa | |
| | aggcgggagc ccgaccggga gaggcttagg gcctttctgg agaggcttga | |
| | gtttggcagc ctcctccacg agttcggcct tctggaaagc cccaaggccc | |
| | tggaggaggc cccctggccc ccgccggaag cagggggggc cgggtccacc | |
| | gggcccccga gccttataaa gccctcaggg gggccttcgt gggctttgtg | |
| | ctttcccgca aggagcccat gtgggccgat cttctggccc tggccgccgc | |
| | acctgaagga ggcgcggggg cttctcgcca aagacctgag cgttctggcc | |
| | ctgagggaag gccttggcct cccgcccggc gacgacccca tgctcctcgc | |
| | ctacctcctg gacccttcca acaccacccc cgagggggtg gcccggcgct | |
| | acggcgggga gtggacggag gaggcggggg agcgggccgc cctttccgag | |
| | aggctcttcg ccaacctgtg ggggaggctt gagggggagg agaggctcct | |
| | ttggctttac cgggaggtgg agaggcccct ttccgctgtc ctggcccaca | |
| | tggaggccac gggggtgcgc ctggacgtgg cctatctcag ggccttgtcc | |
| | ctggaggtgg ccgaggagat cgcccgcctc gaggccgagg tcttccgcct | |
| | ggccggccac cccttcaacc tcaactcccg ggaccagctg gaaagggtcc | |
| | tctttgacga gctagggctt cccgccatcg gcaagacgga gaagaccggc | |
| | aagcgctcca ccagcgccgc cgtcctggag gccctccgcg aggcccaccc | |
| | catcgtggag aagatcctgc agtaccggga gctcaccaag ctgaagagca | |
| | cctacattga ccccttgccg gacctcatcc accccaggac gggccgcctc | |
| | cacacccgct tcaaccagac ggccacggcc acgggcaggc taagtagctc | |
| | cgatcccaac ctccagaaca tccccgtccg caccccgctt gggcagagga | |
| | tccgccgggc cttcatcgcc gaggaggggt ggctattggt ggccctggac | |
| | tatagccaga tagagctcag ggtgctggcc cacctctccg gcgacgagaa | |
| | cctgatccgg gtcttccagg aggggcggga catccacacg gagaccgcca | |
| | gctggatgtt cggcgtcccc cgggaggccg tggaccccct gatgcgccgg | |
| | gcggccaaga ccatcaactt cggggtcctc tacggcatgt cggcccaccg | |
| | cctctcccag gagctagcca tcccttacga ggaggcccag gccttcattg | |
| | agcgctactt tcagagcttc cccaaggtgc gggcctggat tgagaagacc | |
| | ctggaggagg gcaggaggcg ggggtacgtg gagaccctct tcggccgccg | |
| | ccgctacgtg ccagacctag aggcccgggt gaagagcgtg cgggaggcgg | |
| | ccgagcgcat ggccttcaac atgcccgtcc agggcaccgc cgccgacctc | |
| | atgaagctgg ctatggtgaa gctcttcccc aggctggagg aaatgggggc | |
| | caggatgctc cttcaggtcc acgacgagct ggtcctcgag gccccaaaag | |
| | agagggcgga ggccgtggcc cggctggcca aggaggtcat ggagggggtg | |
| | tatcccctgg ccgtgcccct ggaggtggag gtggggatag ggagggggtg | |
| | gctctccgcc aaggagtgat accacc | |

Taq DNA polymerase extends primers composed from deoxyribonucleotides, however, some chemical modifications of the primer are tolerated and do not decrease significantly the efficiency of such primer extension reactions. For example, when the nucleotide at the 3′ primer terminus is ribonucleotide instead of deoxyribonucleotide, Taq DNA polymerase can extend such primer with significant efficiency and speed.

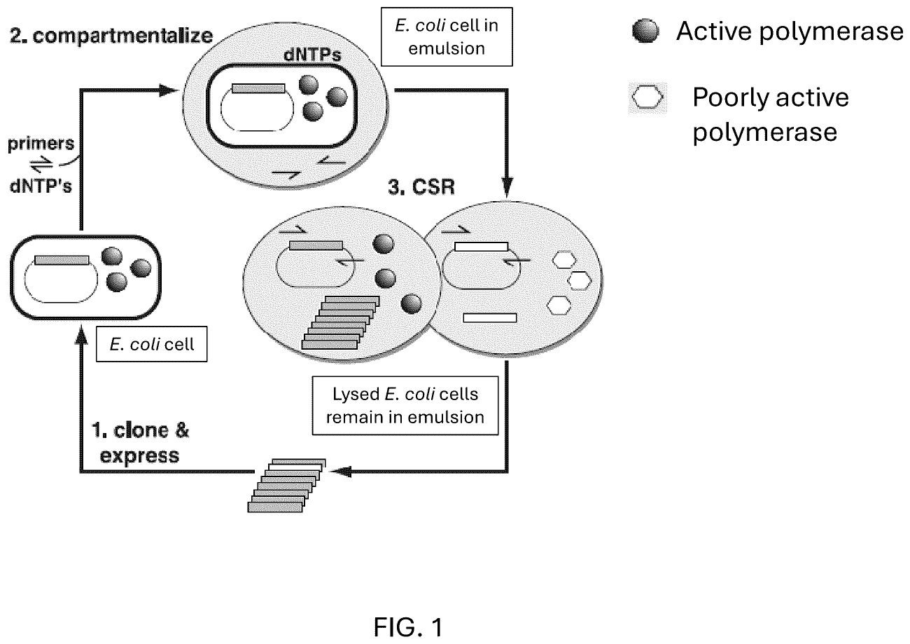

In-vitro compartmentalization (IVC) was first described in 1998 by Tawfik and Griffiths for the molecular evolution of methyltransferase. The technology occurs in isolated and noncommunicating compartments via a water-in-oil emulsion that allows for direct linkage of phenotype to genotype. Since its publication, this technology has been used in a wide variety of use cases. Compartmentalized self-replication (CSR) is a more focused subset of IVC that involves a polymerase self-replicating its own polymerase gene under a specific selection pressure, allowing for direct linkage to the polymerase gene that survived that selection. An exemplary CSR diagram is depicted in FIG. 1. Briefly, random mutagenized polymerase is cloned and expressed in a vector and the vector is resuspended in a polymerase buffer comprising primers matching a polymerase gene and dNTPs. The resuspended vector is then compartmentalized within a water-in-oil emulsion. The emulsion is placed into a thermocycler and polymerase cycling conditions are carried out with an initial heat lysis step to lyse the vector, releasing the expressed polymerase and plasmid containing the polymerase gene. The polymerase then amplifies its own gene.

CSR has been used to select for polymerase mutants that can endure and amplify DNA in the presence of known PCR inhibitors, including heparin, hematin or high salt, among others. These PCR inhibitors are known to carry over in DNA purifications that are ultimately used in standard PCR, qPCR and sequencing. Taq DNA polymerase is a frequently used polymerase in these applications and an inhibitor resistant variant is desired.

The Taq DNA polymerase mutants of the present technology, which have been isolated and identified, at least in part, through rational mutagenesis of a base Taq DNA polymerase, were found to amplify DNA produced through PCR at higher levels in the presence of PCR inhibitors compared to wild type Taq DNA polymerase.

Reference will now be made in detail to exemplary embodiments of the claimed invention. While the claimed invention will be described in conjunction with the exemplary embodiments, it will be understood that it is not intended to limit the claimed invention to those embodiments. To the contrary, it is intended to cover alternatives, modifications, and equivalents, as may be included within the spirit and scope of the claimed invention, as defined by the appended claims.

Those of ordinary skill in the art may make modifications and variations to the embodiments described herein without departing from the spirit or scope of the claimed invention. In addition, although certain methods and materials are described herein, other methods and materials that are similar or equivalent to those described herein can also be used to practice the claimed invention.

In addition, any of the compositions or methods provided, disclosed, or described herein can be combined with one or more of any of the other compositions and methods provided, disclosed, or described herein.

### I. Definitions

Unless otherwise defined, all technical and scientific terms used herein have the same meaning as commonly understood by one of ordinary skill in the art to which the methods described herein belong. Any reference to standard methods refers to the most recent available version of the method at the time of filing of this disclosure unless otherwise indicated.

For any method disclosed herein that includes discrete steps, the steps may be conducted in any feasible order. And, as appropriate, any combination of two or more steps may be conducted simultaneously.

All headings are for the convenience of the reader and should not be used to limit the meaning of the text that follows the heading, unless so specified.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the invention.

The term “comprises” and variations thereof do not have a limiting meaning where these terms appear in the description and claims. Such terms will be understood to imply the inclusion of a stated step or element or group of steps or elements but not the exclusion of any other step or element or group of steps or elements.

By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements. As used herein, the term “including” means, and is used interchangeably with, the phrase “including but not limited to.” As used herein, the term “such as” means, and is used interchangeably with, the phrase “such as, for example” or “such as but not limited.”

The singular form “a”, “an” and “the” include plural referents unless the context clearly dictates otherwise. These articles refer to one or to more than one (i.e., to at least one). As used herein, the term “or” is generally employed in its usual sense including “and/or” unless the content clearly dictates otherwise. The term “and/or” means any one or more of the items in the list joined by “and/or”. As an example, “x and/or y” means any element of the three-element set {(x), (y), (x, y)}. In other words, “x and/or y” means “one or both of x and y”. As another example, “x, y, and/or z” means any element of the seven-element set {(x), (y), (z), (x, y), (x, z), (y, z), (x, y, z)}. In other words, “x, y and/or z” means “one or more of x, y and z”.

Where ranges are given, endpoints include all numbers subsumed within that range (e.g., 1 to 5 includes 1, 1.5, 2, 2.75, 3, 3.80, 4, 5, etc.). Furthermore, unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise. Herein, “up to” a number (for example, up to 50) includes the number (for example, 50). The term “in the range” or “within a range” (and similar statements) includes the endpoints of the stated range.

Reference throughout this specification to “one aspect,” “an aspect,” “certain aspects,” or “some aspects,” etc., means that a particular feature, configuration, composition, or characteristic described in connection with the aspect is included in at least one aspect of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more aspects.

Unless otherwise indicated, all numbers expressing quantities of components, molecular weights, and so forth used in the specification and claims are to be understood as being modified in all instances by the term “about.” As used herein in connection with a measured quantity, the term “about” refers to that variation in the measured quantity as would be expected by the skilled artisan making the measurement and exercising a level of care commensurate with the objective of the measurement and the precision of the measuring equipment used. The term “about” as used in connection with a numerical value throughout the specification and the claims denotes an interval of accuracy, familiar and acceptable to a person skilled in the art. In general, such interval of accuracy is +/−10%. Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

The term “exemplary” means serving as a non-limiting example, instance, or illustration. As utilized herein, the terms “e.g.,” and “for example” set off lists of one or more non-limiting aspects, examples, instances, or illustrations.

As used herein, the term “substantially” refers to the qualitative condition of exhibiting total or near-total extent or degree of a characteristic or property of interest. Biological and chemical phenomena rarely, if ever, go to completion and/or proceed to completeness or achieve or avoid an absolute result. The term “substantially” is therefore used herein to capture the potential lack of completeness inherent in many biological and chemical phenomena. For example, “substantially” may refer to being within at least about 20%, alternatively at least about 10%, alternatively at least about 5% of a characteristic or property of interest.

As used herein, the terms “nucleic acid molecule” and “polynucleotide” refer to a polymer or large biomolecule comprised of nucleotides. The term “nucleic acid” includes deoxyribonucleic acid (DNA), ribonucleic acid (RNA), and analogs thereof. Non-limiting examples of nucleic acid molecules include DNA (e.g., genomic DNA, cDNA), RNA molecules (e.g., mRNA, rRNA, CRNA, tRNA), and chimeras thereof. A nucleic acid molecule can be obtained by cloning techniques or synthesized, using techniques that are known to those of skill in the art. DNA can be double-stranded or single-stranded (coding strand or non-coding strand, i.e., antisense). A nucleic acid backbone may comprise a variety of linkages known in the art, including one or more of sugar-phosphodiester linkages, peptide-nucleic acid bonds (referred to as “peptide nucleic acids” (PNA)), phosphorothioate linkages, methylphosphonate linkages, or combinations thereof. Sugar moieties of the nucleic acid may be ribose or deoxyribose, or similar compounds having known substitutions, for example, 2′ methoxy substitutions (containing a 2′-O-methylribofuranosyl moiety) and/or 2′ halide substitutions. Nitrogenous bases may be conventional bases (adenine (A), guanine (G), thymine (T), cytosine (C), and uracil (U)), known analogs thereof (e.g., inosine), known derivatives of purine or pyrimidine bases, or “abasic” residues in which the backbone includes no nitrogenous base for one or more residues. A nucleic acid may comprise only conventional sugars, bases, and linkages, as found in RNA and DNA, or may include both conventional components and substitutions (e.g., conventional bases linked via a methoxy backbone, or a nucleic acid including conventional bases and one or more base analogs). An “isolated nucleic acid molecule,” as is generally understood by those of skill in the art and as used herein, refers to a polymer of nucleotides, and includes but is not limited to DNA and RNA.

An “amino acid” refers to any monomer unit that can be incorporated into a peptide, polypeptide, or protein. As used herein, the term “amino acid” includes the following twenty natural or genetically encoded alpha-amino acids: alanine (Ala or A), arginine (Arg or R), asparagine (Asn or N), aspartic acid (Asp or D), cysteine (Cys or C), glutamine (Gln or Q), glutamic acid (Glu or E), glycine (Gly or G), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V). In cases where “X” residues are undefined, these should be defined as “any amino acid.” The structures of these twenty natural amino acids are shown in, e.g., Stryer et al., Biochemistry, 5.sup.th ed., Freeman and Company (2002), which is incorporated by reference. Additional amino acids, such as selenocysteine and pyrrolysine, can also be genetically coded for (Stadtman (1996) “Selenocysteine,” Annu Rev Biochem. 65:83-100 and Ibba et al. (2002) “Genetic code: introducing pyrrolysine,” Curr Biol. 12 (13): R464-R466, which are both incorporated by reference). The term “amino acid” also includes unnatural amino acids, modified amino acids (e.g., having modified side chains and/or backbones), and amino acid analogs. See, e.g., Zhang et al. (2004) “Selective incorporation of 5-hydroxytryptophan into proteins in mammalian cells,” Proc. Natl. Acad. Sci. U.S.A. 101 (24): 8882-8887, Anderson et al. (2004) “An expanded genetic code with a functional quadruplet codon” Proc. Natl. Acad. Sci. U.S.A. 101 (20): 7566-7571, Ikeda et al. (2003) “Synthesis of a novel histidine analogue and its efficient incorporation into a protein in vivo,” Protein Eng. Des. Sel. 16 (9): 699-706, Chin et al. (2003) “An Expanded Eukaryotic Genetic Code,” Science 301 (5635): 964-967, James et al. (2001) “Kinetic characterization of ribonuclease S mutants containing photoisomerizable phenylazophenylalanine residues,” Protein Eng. Des. Sel. 14 (12): 983-991, Kohrer et al. (2001) “Import of amber and ochre suppressor tRNAs into mammalian cells: A general approach to site-specific insertion of amino acid analogues into proteins,” Proc. Natl. Acad. Sci. U.S.A. 98 (25): 14310-14315, Bacher et al. (2001) “Selection and Characterization of Escherichia coli Variants Capable of Growth on an Otherwise Toxic Tryptophan Analogue,” J. Bacteriol. 183 (18): 5414-5425, Hamano-Takaku et al. (2000) “A Mutant Escherichia coli Tyrosyl-tRNA Synthetase Utilizes the Unnatural Amino Acid Azatyrosine More Efficiently than Tyrosine,” J. Biol. Chem. 275 (51): 40324-40328, and Budisa et al. (2001) “Proteins with {beta}-(thienopyrrolyl) alanines as alternative chromophores and pharmaceutically active amino acids,” Protein Sci. 10 (7): 1281-1292, which are each incorporated by reference.

As used herein, the term “probe” refers to a nucleic acid oligonucleotide that hybridizes specifically to a target sequence in a nucleic acid or its complement, under conditions that promote hybridization, thereby allowing detection of the target sequence or its amplified nucleic acid. Detection may either be direct (i.e., resulting from a probe hybridizing directly to the target or amplified sequence) or indirect (i.e., resulting from a probe hybridizing to an intermediate molecular structure that links the probe to the target or amplified sequence). A probe's “target” generally refers to a sequence within an amplified nucleic acid sequence (i.e., a subset of the amplified sequence) that hybridizes specifically to at least a portion of the probe sequence by standard hydrogen bonding or “base pairing.” Sequences that are “sufficiently complementary” allow stable hybridization of a probe sequence to a target sequence, even if the two sequences are not completely complementary. A probe may be labeled or unlabeled. A probe can be produced by molecular cloning of a specific DNA sequence, or it can be synthesized. Probes for use in the methods disclosed herein can be readily designed and used by those of skill in the art.

As used herein, the terms “accuracy” and “fidelity” are used interchangeably and refer to ability of a Taq DNA polymerase to accurately replicate a desired template, i.e., the ability of the Taq DNA polymerase to accurately perform cDNA synthesis in a polymerase reaction. The “fidelity” or “accuracy” of a polymerase can be assessed by determining the frequency of incorrect nucleotide incorporation into the synthesized DNA molecule, which may be referred to as the enzyme's error rate. As used herein, the term “increased fidelity” refers to Taq DNA polymerase mutants of the disclosure that exhibit an error rate lower than that of the base construct. For example, the Taq DNA polymerase mutants as disclosed herein can exhibit an error rate that is 10%, 15%, 20%, 25%, 30%, 35%, 40%, 45%, 50%, 55%, 60%, 65%, 70%, 75%, 80%, 85%, 90%, 95%, 100%, or 200% lower than, or at least 2-fold, 3-fold, 4-fold, 5-fold, or 10-fold, or more than 10-fold lower than the error rate of the wild-type Taq DNA polymerase.

As used herein, the term “mutation” refers to a change introduced into the nucleic acid sequence encoding a protein that changes the amino acid sequence of the protein, including but not limited to substitutions, insertions, deletions, point mutations, transpositions, inversions, frame shifts, nonsense mutations, truncations, or other forms of aberrations. A mutation may produce no discernible changes or result in a new property, function, or trait of the mutated protein. A Taq DNA polymerase mutant of the disclosure may have one or more mutations in the nucleic acid sequence encoding the Taq DNA polymerase mutant resulting in one or more mutations in the amino acid sequence of the Taq DNA polymerase mutant. A mutation can result in one or more amino acids being substituted for an alternate amino acid residue, including Ala, Arg, Asn, Asp, Cys, Gln, Glu, Gly, His, Ile, Leu, Lys, Met, Phe, Pro, Ser, Thr, Trp, Tyr, and/or Val. The resulting amino acid mutations may impart altered functional and biological properties to the Taq DNA polymerase mutant of the present technology including but not limited to increased resistance to PCR inhibitors, increased activity, enhanced DNA synthesis, enhanced stability or enhanced thermostability, reduced or eliminated Taq DNA polymerase activity, reduced terminal deoxynucleotidyl transferase activity, increased accuracy or increased fidelity, increased specificity, increased conversion rates, increased probe affinity, or altered half-life.

As used herein, the term “amplification” or “amplify” refers to any known in vitro procedure for obtaining multiple copies of a target nucleic acid sequence or its complement or fragments thereof. In vitro amplification refers to production of an amplified nucleic acid that may contain less than the complete target region sequence or its complement. Known in vitro amplification methods include, for example, transcription-mediated amplification, replicase-mediated amplification, polymerase chain reaction (PCR) amplification, ligase chain reaction (LCR) amplification, and strand-displacement amplification (SDA, including multiple strand-displacement amplification method (MSDA)). Replicase-mediated amplification uses self-replicating RNA molecules, and a replicase such as Q-β-replicase. PCR amplification uses DNA polymerase, primers, and thermal cycling to synthesize multiple copies of the two complementary strands of DNA or cDNA. PCR involves denaturation of a double-stranded DNA molecule, followed by annealing of DNA primers directed to the sequence of interest, and amplification/extension of the newly formed DNA strand. LCR amplification uses at least four separate oligonucleotides to amplify a target and its complementary strand by using multiple cycles of hybridization, ligation, and denaturation. SDA is a method in which a primer contains a recognition site for a restriction endonuclease that permits the endonuclease to nick one strand of a hemi-modified DNA duplex that includes the target sequence, followed by amplification in a series of primer extension and strand displacement steps. Other strand-displacement amplification methods known in the art (e.g., MSDA) do not require endonuclease nicking. Those of skill in the art will understand that the oligonucleotide primer sequences of the disclosure may be readily used in any in vitro amplification method based on primer extension by a polymerase. As commonly known in the art, oligonucleotides are designed to bind to a complementary sequence under selected conditions.

As used herein, the term “cDNA” (short for copy DNA; also called complementary DNA) refers to a synthetic DNA that has been transcribed from a specific mRNA through a reaction using the enzyme reverse transcriptase.

As used herein, the term “complementary” refers to the broad concept of sequence complementarity between regions of two nucleic acid strands or between two regions of the same nucleic acid strand. A first region of a nucleic acid is complementary to a second region of the same or a different nucleic acid if, when the two regions are arranged in an antiparallel fashion, at least one nucleotide of the first region is capable of base pairing with a nucleotide of the second region. In some embodiments, the first region comprises a first portion and the second region comprises a second portion, whereby, when the first and second portions are arranged in an antiparallel fashion, at least about 50%, 75%, 80%, 85%, 90%, 95%, 96%, 97%, 98%, or 99% of the nucleotides of the first portion are capable of base pairing with nucleotides in the second portion. In another embodiment, all nucleotides of the first portion are capable of base pairing with nucleotides in the second portion.

Polypeptide and polynucleotide sequences may be aligned, and percentages of identical amino acids or nucleotides in a specified region may be determined against another polypeptide or polynucleotide sequence, using computer algorithms that are publicly available. The percent identity of a polynucleotide or polypeptide sequence is determined by aligning polynucleotide and polypeptide sequences using appropriate algorithms, such as BLASTN or BLASTP, respectively, set to default parameters; identifying the number of identical nucleic or amino acids over the aligned portions; dividing the number of identical nucleic or amino acids by the total number of nucleic or amino acids of the polynucleotide or polypeptide of the disclosure; and then multiplying by 100 to determine the percent identity.

The term “decreased,” as used herein with regard to Taq DNA polymerase activity, refers to the level of Taq DNA polymerase activity of a Taq DNA polymerase mutant as compared to the Taq DNA polymerase base construct. An Taq DNA polymerase mutant has “decreased” Taq DNA polymerase activity if the level of its Taq DNA polymerase activity, as measured by the quantity of DNA synthesized or as measured by other methods known in the art is less than the Taq DNA polymerase base construct activity. For example, the Taq DNA polymerase activity of the Taq DNA polymerase mutant is decreased if the Taq DNA polymerase activity is at least 95%, 90%, 85%, 80%, 75%, 70%, 65%, 60%, 55%, 50%, 45%, 40%, 35%, 30%, 25%, 20%, 15%, 10%, or 5% less than, or at least 2-fold, 3-fold, 4-fold, 5-fold, 10-fold, or more than 10-fold less than the Taq DNA polymerase base construct activity.

The term “increased,” as used herein with regard to Taq DNA polymerase activity, refers to the level of Taq DNA polymerase activity of an Taq DNA polymerase mutant as compared to the Taq DNA polymerase base construct. An Taq DNA polymerase mutant has “increased” Taq DNA polymerase activity if the level of its Taq DNA polymerase activity, as measured by the quantity of DNA synthesized or as measured by other methods known in the art, is more than the Taq DNA polymerase base construct activity. For example, the Taq DNA polymerase activity of the Taq DNA polymerase mutant is increased if the Taq DNA polymerase activity is at least 10%, 15%, 20%, 25%, 30%, 35%, 40%, 45%, 50%, 55%, 60%, 65%, 70%, 75%, 80%, 85%, 90%, 95%, or 100% more than, or at least 2-fold, 3-fold, 4-fold, 5-fold, or 10-fold, or more than 10-fold more than the Taq DNA polymerase base construct activity.

As used herein, the terms “stacked” or “stacked mutation” refers to a Taq DNA polymerase mutant with at least two point mutations.

The invention is defined in the claims. However, below is a non-exhaustive listing of non-limiting exemplary aspects. Any one or more of the features of these aspects may be combined with any one or more features of another example, embodiment, or aspect described herein.

For any method disclosed herein that includes discrete steps, the steps may be conducted in any feasible order. And, as appropriate, any combination of two or more steps may be conducted simultaneously.

All headings are for the convenience of the reader and should not be used to limit the meaning of the text that follows the heading, unless so specified.

### II. Taq DNA Polymerase Mutants

In general, Taq DNA polymerase mutants are known to show beneficial features as compared to wild-type Taq DNA polymerase. This may include increased DNA amplification, increased resistance to a PCR inhibitor, and/or increased tolerance to a PCR inhibitor.

PCR inhibitors are commonly encountered in a variety of materials analyzed in clinical, food, and environmental testing. Clinical samples such as blood, serum or plasma, urine, and stool may contain several substances that can impede PCR amplification. For instance, native immunoglobulin (IgG) in blood is a potent PCR inhibitor due to its strong affinity for single-stranded DNA. In food samples, certain components can compromise PCR efficiency. For example, milk contains the enzyme plasmin, which degrades Taq polymerase, and a high calcium concentrations that might inhibit PCR. Plant samples often contain various inhibitory substances, for example, polysaccharides like dextran sulfate, xylan, dextran sulfate, and gutti gum can affect PCR efficiency. Additionally, phenols in berries and tomatoes are known to particularly inhibit RT-PCR assays.

Environmental samples, such as dead biomass and soil, may contain humic and fulvic acids, which can inhibit PCR even at low concentrations. For example, wild-type Taq DNA polymerase is typically inhibited in the presence of less than 1 ng of humic acid in a PCR reaction. In sewage sludge and wastewater, inhibitors include fats, proteins, polyphenols, heavy metals, polysaccharides, metal ions, and RNases. Other potential PCR inhibitors include components of bacterial cells, non-target DNA, contaminants, and laboratory items such as pollen, glove powder, laboratory plasticware, and cellulose. In some embodiments, the PCR inhibitor is humic acid. In another embodiment, the PCR is humic acid. In another embodiment, the PCR inhibitor is heparin. In another embodiment, the PCR inhibitor is hematin. In another embodiment, the PCR inhibitor is high-salt. In another embodiment, the PCR inhibitor is ethanol. In another embodiment, the PCR inhibitor is guanidine thiocyanate. In another embodiment, the PCR inhibitor is a combination of these inhibitors.

In some embodiments, the disclosed Taq DNA polymerase mutants possess an increased DNA amplification capability which is at least a 20-fold increase relative to wild-type DNA polymerase, alternatively at least a 45-fold increase relative to wild-type DNA polymerase, or alternatively at least a 60-fold increase relative to wild-type DNA polymerase.

In certain embodiments, the fold-increase of a disclosed Taq DNA polymerase mutant is determined by quantifying the self-replication of the Taq DNA polymerase produced through Compartmentalized Self-Replication (CSR) using gene specific primers to amplify DNA encoding the disclosed Taq DNA polymerase mutant. The fold-increase is calculated by taking the ratio of Taq DNA polymerase mutant DNA replicated to Taq DNA Polymerase wild-type DNA replicated.

In some embodiments the increased DNA amplification is determined after a first round of enrichment, alternatively after a second round of enrichment wherein extracted DNA from an emulsion is cloned back into an expression vector before the second round of enrichment.

In some embodiments, the disclosed Taq DNA polymerase mutants are resistant to polymerase inhibition by humic acid. In other embodiments, the PCR is humic acid. In other embodiments, the PCR inhibitor is heparin. In other embodiments, the PCR inhibitor is hematin. In other embodiments, the PCR inhibitor is high-salt. In other embodiments, the PCR inhibitor is ethanol. In other embodiments, the PCR inhibitor is guanidine thiocyanate. In other embodiments, the PCR inhibitor is a combination of these inhibitors.

In another embodiment, the disclosed Taq DNA polymerase mutants are resistant to polymerase inhibition by humic acid at concentrations up to 11.7 ng/mL.

In another embodiment, the disclosed Taq DNA polymerase mutants are resistant to polymerase inhibition by heparin at concentrations up to 1.53 U/mL

In another embodiment, the disclosed Taq DNA polymerase mutants are resistant to polymerase inhibition by hematin at concentrations up to 16 μM.

In another embodiment, the disclosed Taq DNA polymerase mutants are resistant to polymerase inhibition by salts at concentrations up to 114 mM.

In another embodiment, the disclosed Taq DNA polymerase mutants are resistant to polymerase inhibition by ethanol at concentrations up to 3.9% ethanol by volume.

In another embodiment, the disclosed Taq DNA polymerase mutants are resistant to guanidine thiocyanate found in buffer solutions at concentrations up to 1.60% by weight of the buffer solution.

In some embodiment, suitable Taq DNA polymerase mutants may be generated by modifying wild type Taq DNA polymerase. In a non-limiting example, a wild type Taq DNA polymerase may be modified, wherein the modification results in at least one amino acid mutation. In other aspects, the modification results in at least three amino acid mutations, alternatively at least four amino acid mutations, alternatively at least five amino acid mutations, or alternatively at least six amino acid mutations.

In one embodiment, the Taq DNA Polymerase mutant comprises the amino acid sequence of SEQ ID NO: 1, with at least one or more mutation at an amino acid selected from A141P, A227S, A227Y, A259T, A29P, A341K, A439M, A61D, A705S, A70M, A757S, A77S, A83H, D151S, D547R, E117R, E135A, E159G, E210G, E210R, E255Q, E263S, E296G, E388S, E39R, E39R, E39R, E404G, E423G, E423I, E465A, E507K, E507T, E524G, E724G, E742G, E742N, E774P, E774S, F27P, F27R, F632L, F73R, F73S, F749A, G115R, G12R, G160N, G160S, G195R, G286S, G395Y, G59F, G59T, G84A, H21R, H75R, K128R, K225R, K247A, K260G, K508R, K531R, K709L, K709R, K831E, L106V, L148P, L233D, L245H, L33P, L352P, L367V, L456P, L55R, L5P, L605P, L606V, L670P, L768P, L96P, M775Y, P194Y, P527Q, P589A, P89I, Q178R, Q582M, Q680R, Q690R, Q754R, Q782R, Q95T, R110M, R2F, R596S, R695S, R801Q, S290P, S383H, S383N, S383P, S543F, S679G, T386Q, T569A, T88R, V103A, V17L, V449A, V607N, V631A, V821D, W167E, W706F, Y16IN, Y161R, Y161V, Y182S, Y24H, Y24T, Y339P, Y339V, Y455R, Y78R, and Y78S. By way of example, in the aforementioned L106V mutation, the lysine amino acid at position 106 is changed to a valine amino acid.

In a further embodiment, the Taq DNA polymerase mutant has a mutation at two or more amino acid positions.

In a still further embodiment, the Taq DNA polymerase mutant comprises a mutation at two amino acid positions. In this embodiment, the mutation at the first amino acid position is selected from A61D, E39R, H21R, L367V, R2F, Y182S, and T88R; and mutation at the second amino acid position is selected from A61D, E39R, E210R, E507T, H21R, L367V, R2F, Y182S, and T88R. In this embodiment, the mutation at the first amino acid position and the mutation at the second amino acid position are different from one another.

In a further embodiment, the Taq DNA polymerase mutant has a mutation at three or more amino acid positions.

In an alternative embodiment, the Taq DNA polymerase mutant comprises a mutation at three amino acid positions. In this embodiment, the mutation at the first amino acid position is selected from H21R, R2F, T88R, E39R, A61D, L367V, and Y182S; the mutation at the second amino acid position is selected from H21R, R2F, T88R, E39R, A61D, L367V, and Y182S; and the mutation at the third amino acid position is selected from H21R, R2F, T88R, E39R, A61D, L367V, and Y182S. In this embodiment, the mutation at the first amino acid position, the mutation at the second amino acid position, and the mutation at the third amino acid position are different from one another. “Different from one another,” as used herein, means that a mutation cannot be repeated, i.e. if a mutation is selected as the first mutation, that same mutation cannot also be selected as a subsequent mutation. By way of a non-limiting example, if the H21R mutation is selected as the first mutation, the H21R mutation cannot then be selected as the second or third mutation.

In a still further embodiment, the mutation at the first amino acid position is H21R; the mutation at the second amino acid position is T88R, E39R or A61D; and the mutation at the third amino acid position is L367V or T88R.

In a still further embodiment, the Taq DNA Polymerase comprises a mutation at three or more amino acid positions. In this embodiment, the mutation is selected from H21R, E39R, A61D, T88R, V17L, A29P, E210R, V631A, E724G, K831E, K709R, Y24H, and F27R.

In an alternative embodiment, the Taq DNA Polymerase comprises a mutation at three amino acid positions. In this embodiment, the mutation at the first amino acid position is H21R or V17L; the mutation at the second amino acid position is selected from \H21R, A29P, T88R, Y24H, and F27R; the mutation at the third amino acid position is selected from T88R, E210R, V631A, K709R, E724G, and K831E. In this embodiment, the mutations at all three amino acid positions are different from one another.

In a further embodiment, the Taq DNA polymerase mutant has a mutation at four or more amino acid positions. In this embodiment, the mutation at the first amino acid position is selected from H21R, R2F, and V17L; the mutation at the second amino acid position is selected from E39R, H21R, A29P, T88R, Y24H, F27R, and A61D; the mutation at the third amino acid position is selected from A61D, T88R, E210R, V631A, K709R, E724G, K831E, Y182S, and E39R, and the mutation at the fourth amino acid position is selected from T88R, A61D, L367V, E507T, and E210R. In this embodiment, the mutations at all four amino acid positions are different from one another.

In a further embodiment, the mutation at the first amino acid position is H21R; the mutation at the second amino acid position is E39R or A61D; the mutation at the third amino acid position is T88R; and the mutation at the fourth amino acid position is E210R.

In a further embodiment, the Taq DNA polymerase mutant has a mutation at five or more amino acid positions. In this embodiment, the mutation at the first amino acid position is H21R or V17L; the second amino acid position is selected from the group consisting A29P, E39R, H21R, Y24H, F27R, and A61D; the third amino acid position is selected from the group consisting of E39R, A61D, and T88R; the fourth amino acid position is selected from the group consisting of T88R, E210R, and A61D; and the fifth amino acid position is selected from the group consisting of T88R, E210R, E507T, K709R, V631A, E274G, E724G, and K831E. In this embodiment, all five mutations are different.

In a further embodiment, the mutation at the first amino acid position is V17L; the mutation at the second amino acid position is H21R; the mutation at the third amino acid position is E39R or A61D; the mutation at the fourth amino acid position is T88R; and the mutation at the fifth amino acid position is E210R.

In a further embodiment, the Taq DNA polymerase mutant has a mutation at six or more amino acid positions. In this embodiment, the mutation at the first amino acid position is V17L; the mutation at a second amino acid position is H21R; the mutation at a third amino acid position is A61D; the mutation at a fourth amino acid position is T88R; the mutation at the fifth amino acid position is E210R; and the mutation at the sixth amino acid position is E507T.

In some aspects, to generate the Taq DNA polymerase mutants of the present disclosure, the wild-type Taq DNA Polymerase was modified via nicking mutagenesis. The term “nicking,” as used herein, refers to the cleavage of only one strand of the double-stranded portion of a fully or partially double-stranded nucleic acid. The position where the nucleic acid is nicked is referred to as the “nicking site” (NS). A “nicking agent” (NA) is an agent that nicks a partially or fully double-stranded nucleic acid. It may be an enzyme or any other chemical compound or composition. In certain embodiments, a nicking agent may recognize a particular nucleotide sequence of a fully or partially double-stranded nucleic acid and cleave only one strand of the fully or partially double-stranded nucleic acid at a specific position (i.e., the NS) relative to the location of the recognition sequence. Such nicking agents (referred to as “sequence specific nicking agents”) include, but are not limited to, nicking endonucleases (e.g., Nt.BstNBI).

A “nicking endonuclease” (NE), as used herein, thus refers to an endonuclease that recognizes a nucleotide sequence of a completely or partially double-stranded nucleic acid molecule and cleaves only one strand of the nucleic acid molecule at a specific location relative to the recognition sequence.

In a non-limiting example, the present disclosure provides methods for obtaining a Taq DNA polymerase mutant. In this embodiment, the method includes (a) subjecting a polynucleotide encoding a Taq DNA polymerase to PCR to generate a library comprising an array of different polynucleotides; (b) screening the library for increased DNA amplification, increased resistance to a PCR inhibitor, and/or increased tolerance to a PCR inhibitor; (c) selecting one or more Taq DNA polymerases from screening step b; and (d) repeating steps a to c until a final Taq DNA polymerase is obtained, wherein the final Taq DNA polymerase comprises at least a mutation at one or more amino acid positions, wherein the one or more mutations is selected from A141P, A227S, A227Y, A259T, A29P, A341K, A439M, A61D, A705S, A70M, A757S, A77S, A83H, D151S, D547R, E117R, E135A, E159G, E210G, E210R, E255Q, E263S, E296G, E388S, E39R, E404G, E423G, E423I, E465A, E507K, E507T, E524G, E724G, E742G, E742N, E774P, E774S, F27P, F27R, F632L, F73R, F73S, F749A, G115R, G12R, G160N, G160S, G195R, G286S, G395Y, G59F, G59T, G84A, H21R, H75R, K128R, K225R, K247A, K260G, K508R, K531R, K709L, K709R, K831E, L106V, L148P, L233D, L245H, L33P, L352P, L367V, L456P, L55R, L5P, L605P, L606V, L670P, L768P, L96P, M775Y, P194Y, P527Q, P589A, P89I, Q178R, Q582M, Q680R, Q690R, Q754R, Q782R, Q95T, R110M, R2F, R596S, R695S, R801Q, S290P, S383H, S383N, S383P, S543F, S679G, T386Q, T569A, T88R, V103A, V17L, V449A, V607N, V631A, V821D, W167E, W706F, Y16IN, Y161R, Y161V, Y182S, Y24H, Y24T, Y339P, Y339V, Y455R, Y78R, and Y78S. In some embodiments, the final Taq DNA polymerase comprises mutations at three or more amino acid positions, wherein the three or more mutations are selected from A141P, A227S, A227Y, A259T, A29P, A341K, A439M, A61D, A705S, A70M, A757S, A77S, A83H, D151S, D547R, E117R, E135A, E159G, E210G, E210R, E255Q, E263S, E296G, E388S, E39R, E404G, E423G, E423I, E465A, E507K, E507T, E524G, E724G, E742G, E742N, E774P, E774S, F27P, F27R, F632L, F73R, F73S, F749A, G115R, G12R, G160N, G160S, G195R, G286S, G395Y, G59F, G59T, G84A, H21R, H75R, K128R, K225R, K247A, K260G, K508R, K531R, K709L, K709R, K831E, L106V, L148P, L233D, L245H, L33P, L352P, L367V, L456P, L55R, L5P, L605P, L606V, L670P, L768P, L96P, M775Y, P194Y, P527Q, P589A, P89I, Q178R, Q582M, Q680R, Q690R, Q754R, Q782R, Q95T, R110M, R2F, R596S, R695S, R801Q, S290P, S383H, S383N, S383P, S543F, S679G, T386Q, T569A, T88R, V103A, V17L, V449A, V607N, V631A, V821D, W167E, W706F, Y16IN, Y16IR, Y161V, Y182S, Y24H, Y24T, Y339P, Y339V, Y455R, Y78R, and Y78S. In some embodiments, the final Taq DNA polymerase comprises six or more mutations selected from the group consisting of H21R, E39R, A61D, T88R, V17L, A29P, E210R, V631A, E724G, K831E, K709R, Y24H, E507T, and F27R.

In yet another embodiment, the present disclosure provides a method of obtaining a Taq DNA polymerase mutant, wherein the Taq DNA polymerase mutant has a mutation at two or more amino acid positions. In an embodiment, the first amino acid position is selected from A61D, E39R, H21R, L367V, R2F, Y182S, and T88R; and the second amino acid position is selected from A61D, E39R, E210R, E507T, H21R, L367V, R2F, Y182S, and T88R, wherein the mutation at the first amino acid position and the mutation at the second amino acid position are different from one another.

In yet another embodiment, the present disclosure provides a method of obtaining a Taq DNA polymerase mutant, wherein the Taq DNA polymerase mutant has a mutation at three or more amino acid positions. In an alternative embodiment, the mutation at the first amino acid position is selected from H21R, R2F, T88R, E39R, A61D, L367V, and Y182S; the mutation at the second amino acid position is selected from H21R, R2F, T88R, E39R, A61D, L367V, and Y182S; and the mutation at the third amino acid position is selected from H21R, R2F, T88R, E39R, A61D, L367V, and Y182S, wherein the mutation at the first amino acid position, the mutation at the second amino acid position, and mutation at the third amino acid position are different from one another. Alternatively, the mutation at the first amino acid position is H21R; the mutation at the second amino acid position is selected from T88R, A61D, E39R; and mutation at the third amino acid position is selected from L367V or T88R.

In yet another embodiment, the present disclosure provides a method of obtaining a Taq DNA polymerase mutant, wherein the Taq DNA polymerase mutant comprises a mutation at three or more amino acid positions, wherein the mutation is selected from the group consisting of H21R, E39R, A61D, T88R, V17L, A29P, E210R, V631A, E724G, K831E, K709R, Y24H, and F27R. In an alternative embodiment, the mutation at the first amino acid position is selected from H21R or V17L; the mutation at the second amino acid position is selected from H21R, A29P, T88R, Y24H, and F27R; and the mutation at the third amino acid position is selected from T88R, E210R, V631A, K709R, E724G, and K831E, wherein the mutation at the first amino acid position, the mutation at the second amino acid position, and the mutation at the third amino acid position are different from one another. Alternatively, the mutation at the first amino acid position is H21R or V17L; the mutation at the second amino acid position is H21R or T88R; and the mutation at the third amino acid position is T88R or E210R.

In yet another embodiment, the present disclosure provides a method of obtaining a Taq DNA polymerase mutant, wherein the Taq DNA polymerase mutant comprises a mutation at four or more amino acid positions. In an alternative embodiment, the mutation at the first amino acid position is selected from H21R, R2F, and V17L; the mutation at the second amino acid position is selected from E39R, H21R, A29P, T88R, Y24H, F27R, and A61D; the mutation at the third amino acid position is selected from A61D, T88R, E210R, V631A, K709R, E724G, K831E, Y182S and E39R; and the mutation at the fourth amino acid position is selected from T88R, A61D, L367V, E507T, and E210R, wherein the mutation at the first amino acid position, the mutation at the second amino acid position, the mutation at the third amino acid position, and the mutation at the fourth amino acid position are different from one another. Alternatively, the mutation at the first amino acid position is H21R; the mutation at the second amino acid position is E39R or A61D; the mutation at the third amino acid position is T88R; and the mutation at the fourth amino acid position is E210R.

In yet another embodiment, the present disclosure provides a method of obtaining a Taq DNA polymerase mutant, wherein the Taq DNA polymerase has mutation at five or more amino acid positions. In an alternative embodiment, the Taq DNA Polymerase comprises a mutation at five amino acid positions, wherein, the mutation at the first amino acid position is H21R or V17L; the mutation at the second amino acid position is selected from A29P, E39R, H21R, Y24H, F27R and A61D; the mutation at the third amino acid position is selected from E39R, A61D, and T88R; the mutation at the fourth amino acid position is selected from T88R, E210R, and A61D; and the mutation at a fifth amino acid position is selected from T88R, E210R, E507T, K709R, V631A, E274G, E724G, and K831E, wherein the mutation at the first amino acid position, the mutation at the second amino acid position, the mutation at the third amino acid position, the mutation at the fourth amino acid position, and the mutation at the fifth amino acid position are different from one another. Alternatively, the mutation at the first amino acid position is V17L; the mutation at the second amino acid position is H21R; the mutation at the third amino acid position is E39R or A61D; the mutation at the fourth amino acid position is T88R; and the mutation at the fifth amino acid position is E210R.

In yet another embodiment, the present disclosure provides a method of obtaining a Taq DNA polymerase mutant, wherein the Taq DNA polymerase mutant comprises a mutation at six or more amino acid positions. In an alternative embodiment, the mutation at a first amino acid position is V17L; the mutation at a second amino acid position is H21R; the mutation at a third amino acid position is A61D; the mutation at a fourth amino acid position is T88R; the mutation at the fifth amino acid position is E210R; and the mutation at the sixth amino acid position is E507T.

In some embodiments, the Taq DNA polymerase mutants of the present technology are encoded into a nucleic acid molecule. As used herein, the nucleic acid molecule refers to any polynucleotide that encodes a polypeptide of interest and is capable of being translated to produce the encoded polypeptide in vitro, in vivo, in situ, or ex vivo. In some aspects, the nucleic acid molecule is mRNA, DNA, RNA, FFPE DNA, or cDNA, and fragments of the same.

In addition to messenger RNA, several non-coding types of RNA exist which may be involved in regulation of transcription and/or translation, or immunostimulation. Within the present disclosure the term “RNA” further encompasses any type of single stranded (ssRNA) or double stranded RNA (dsRNA) molecule known in the art, such as viral RNA, retroviral RNA and replicon RNA, small interfering RNA (siRNA), antisense RNA (asRNA), circular RNA (circRNA), ribozymes, aptamers, riboswitches, immunostimulating/immunostimulatory RNA, transfer RNA (tRNA), ribosomal RNA (rRNA), small nuclear RNA (snRNA), small nucleolar RNA (snoRNA), microRNA (miRNA), and Piwi-interacting RNA (piRNA).

In another embodiment, the Taq DNA polymerase mutants of the present technology are present in a composition. In some embodiments, a composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to; antimicrobials; antioxidants (such as ascorbic acid, sodium sulfite or sodium hydrogen-sulfite); buffers (such as acetate, borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, sucrose, trehalose, mannose or dextrins); coloring, flavoring and diluting agents; emulsifying agents; salt-forming counterions (such as sodium or potassium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); or tonicity enhancing agents (such as alkali metal halides, preferably sodium or potassium chloride, mannitol sorbitol).

The disclosure also provides kits for use in reverse transcription or related technologies. These kits include one or more of the following: a Taq DNA Polymerase mutant enzyme, reagents and buffers for conducting a PCR reaction, a box, vial tubes, ampules, and the like. Kits can also include instructions for use of the kit for practicing any of the methods disclosed herein or other methods known to those of skill in the art.

### III. Methods of Use

This disclosure also relates to the activity of Taq DNA Polymerase mutants, as measured by the quantity of DNA produced by the Taq DNA Polymerase mutants disclosed herein.

cDNA can be prepared using one-step or two-step procedures and can be obtained from a variety of template molecules. As used herein, the term “template molecule” refers to a biological molecule that carries the genetic code for use in making a new nucleic acid strand. For example, in DNA replication, the unwound double helix and each single-stranded DNA molecule is used as a template to synthesize a complementary strand. Reverse transcription generates cDNA from RNA. One of skill in the art will understand that cDNA molecules may be prepared from a variety of nucleic acid template molecules. In one embodiment, the nucleic acid template can be single-stranded or double-stranded DNA. In one embodiment, RNA can be used in cDNA synthesis. In certain embodiments, the Taq DNA Polymerase mutants of the disclosure exhibit increased or enhanced thermostability and/or Taq DNA Polymerase activity as compared to a Taq DNA Polymerase base construct. In other embodiments, the Taq DNA Polymerase mutants of the disclosure exhibit altered half-life, reduced or eliminated Taq DNA Polymerase H activity, reduced terminal deoxynucleotidyl transferase activity, increased accuracy or fidelity, or increased specificity.

The disclosure of the present technology also provides methods for synthesizing cDNA using the Taq DNA Polymerase mutants of the disclosure that have single or double amino acid mutations. The Taq DNA Polymerase mutants of the disclosure may be used in methods that produce a first strand cDNA or a first and second strand cDNA. One of skill in the art will understand that first and second strand cDNA may form a double-stranded DNA molecule, which may include a full-length cDNA sequence and cDNA libraries. The cDNA molecules that have been reverse transcribed by the Taq DNA Polymerase mutants of the disclosure may be isolated, or the reaction mixture containing the cDNA molecules may be directly used in downstream applications or for further analysis or manipulation. Amplification methods that may be used to practice the methods of the disclosure are described herein and are well known in the art. Reverse transcription reactions may be carried out using non-specific primers, such as an anchored oligo-dT primer, or random sequence primers, or using a target-specific primer complementary to the RNA for each genetic probe being monitored, or using thermostable DNA polymerases (such as AMV RTase or MMLV RTase).

Amplification methods utilize pairs of primers that selectively hybridize to nucleic acids corresponding to a specific nucleotide sequence of interest that are contacted with the isolated nucleic acid under conditions that permit selective hybridization. Once hybridized, the nucleic acid: primer complex is contacted with one or more enzymes that facilitate template-dependent nucleic acid synthesis. Multiple rounds of amplification, also referred to as “cycles,” are conducted until a sufficient amount of amplification product is produced. Next, the amplification product is detected. In certain methods, the detection may be performed by visual means. Alternatively, the detection may involve indirect identification of the product via chemiluminescence, radioactive scintigraphy of incorporated radiolabel or fluorescent label, or even via a system using electrical or thermal impulse signals.

Methods based on ligation of two (or more) oligonucleotides in the presence of a nucleic acid having the sequence of the resulting “di-oligonucleotide,” thereby amplifying the di-oligonucleotide, also may be used in the amplification step of the present disclosure.

In some embodiments of the disclosure, the detection process can utilize a hybridization technique, for example, wherein a specific primer or probe is selected to anneal to a target biomarker of interest, and thereafter detection of selective hybridization is made. As commonly known in the art, the oligonucleotide probes and primers can be designed by taking into consideration the melting point of hybridization thereof with its targeted sequence.

One of skill in the art will recognize that cDNA molecules made using the Taq DNA Polymerase mutants of the disclosure can be used in a variety of additional downstream applications. For example, amplification methods may include one-step PCR, two-step PCR, real-time or quantitative PCR, hot-start PCR, nested PCR, touch down PCR, differential display PCR (DDRT-PCR), microarray technologies, inverse PCR, Rapid amplification of PCR ends (RACE or anchored PCR), multiplex PCR, and site directed PCR mutagenesis. Synthesized cDNA and cDNA libraries created with the Taq DNA Polymerase mutants of the disclosure can be used in cloning and/or sequencing for further characterization. One of skill in the art will recognize that nucleic acid amplification using cDNA prepared with the Taq DNA Polymerase mutants of the disclosure may include additional techniques not listed herein.

To enable hybridization to occur under the methods presented above, oligonucleotide primers and probes should comprise an oligonucleotide sequence that has at least 70%, 75%, 80%, 85%, 90%, 95%, 96%, 97%, 98%, 99%, or 100% identity to a portion of the sequence of interest.

A. DNA Amplification from Soil Samples

Humic acid is a known polymerase inhibitor frequently found in soil samples. Humic acids can bind to DNA polymerases, directly inhibiting their ability to catalyze the amplification reaction. Humic acids can also bind to the DNA template itself, preventing the polymerase from accessing the DNA and reducing amplification efficiency. Humic acids may alter the chemical environment of the PCR reaction, such as pH or ion concentration, further impeding polymerase activity. Due to its inhibitory effects, humic acid can cause false negatives or poor amplification in PCR-based assays. To mitigate this, various strategies like additional purification steps, dilution of the sample, or the use of polymerases engineered to be more tolerant of inhibitors are employed when working with soil samples.

The disclosed Taq DNA polymerase mutants have increased resistance to humic acid inhibition of the polymerase reaction. The disclosed Taq DNA polymerase mutants allow DNA amplification without first purifying the soil sample to remove humic acid.

In one embodiment, the disclosed Taq DNA polymerase mutants can be used to amplify DNA obtained from soil samples containing humic acid. Alternatively, the disclosed Taq DNA polymerase mutants can be used to amplify DNA from a soil sample where the humic acid content is unknown.

The specific makeup of the soil samples is not particularly limited.

B. DNA Amplification from Blood Samples

Hematin, a breakdown product of hemoglobin, is commonly found in blood and can inhibit PCR by interfering with the activity of DNA polymerases. It can bind to DNA or the polymerase enzyme itself, reducing the efficiency of amplification. Heparin is an anticoagulant used in blood collection and also inhibits polymerase activity, interfering with the interaction between the polymerase and the DNA template, thus hindering the amplification process.

Both substances are problematic for diagnostic tests that rely on PCR, so strategies such as dilution, special reagents, or sample preparation techniques (like DNA purification) are often employed to reduce or eliminate their inhibitory effects.

The disclosed Taq DNA polymerase mutants have increased resistance to hematin and/or heparin inhibition of the polymerase reaction. The disclosed Taq DNA polymerase mutants allow DNA amplification without first treating the blood sample to reduce or eliminate the inhibitory effects of hematin and/or heparin.

In one embodiment, the disclosed Taq DNA polymerase mutants can be used to amplify DNA obtained from blood samples containing hematin and/or heparin. Alternatively, the disclosed Taq DNA polymerase mutants can be used to amplify DNA from a blood sample where the hematin and/or heparin content is unknown.

C. DNA Amplification from Other Solutions

Ethanol is often used in nucleic acid purification steps, particularly during washing. Residual ethanol left in the sample after purification can inhibit the activity of DNA polymerases in PCR, interfering with amplification. In most methods, the ethanol must be fully evaporated before resuspending DNA in buffer or water for PCR.

Salts, like sodium chloride or potassium chloride, can inhibit PCR if present in high concentrations. They affect the enzyme's performance by altering the ionic environment, interfering with polymerase binding to the DNA template, or affecting the melting temperature of the DNA. This can lead to inefficient or failed amplification.

Guanidine thiocyanate, a chaotropic agent used to lyse cells and denature proteins during nucleic acid extraction, is included in some buffer solutions of commercial kits. It disrupts hydrogen bonds, which aids in nucleic acid isolation but, if not properly removed during purification, can inhibit polymerase activity in PCR. Its presence in the reaction mix can disrupt the function of polymerases by destabilizing proteins and altering the conditions required for successful amplification.

To avoid these inhibitory effects, careful sample preparation and buffer washing steps are necessary. For example, in commercial PCR kits, these substances are typically washed out or neutralized before the actual PCR reaction takes place.

The disclosed Taq DNA polymerase mutants have increased resistance to ethanol, high concentrations of salts, and guanidine thiocyanate. The disclosed Taq DNA polymerase mutants allow DNA amplification without first evaporating, neutralizing, or washing the sample to remove these inhibitors.

In one embodiment, the disclosed Taq DNA polymerase mutants can be used to amplify DNA obtained from samples containing ethanol, salts, and/or guanidine thiocyanate. Alternatively, the disclosed Taq DNA polymerase mutants can be used to amplify DNA from a sample where the ethanol, salt, and/or guanidine thiocyanate content is unknown.

In certain embodiments of the disclosure, after extraction, DNA is amplified, and transcribed into cDNA, which can then serve as template for multiple rounds of amplification by the Taq DNA Polymerase mutants of the present technology. Amplification methods that may be used to practice the methods of the disclosure are described herein and are well known in the art. Reverse transcription reactions may be carried out using non-specific primers, such as an anchored oligo-dT primer, or random sequence primers, or using a target-specific primer complementary to the RNA for each genetic probe being monitored, or using thermostable DNA polymerases, such as Taq DNA Polymerase or the Taq DNA Polymerase mutants of the disclosure.

In certain embodiments, the DNA isolated from a sample (e.g., after amplification and/or conversion to cDNA) is labeled with a detectable agent before being analyzed. The role of a detectable agent is to facilitate detection of DNA or to allow visualization of hybridized nucleic acid fragments (e.g., nucleic acid fragments hybridized to genetic probes in an array-based assay). In some embodiments, the detectable agent is selected such that it generates a signal which can be measured and whose intensity is related to the amount of labeled nucleic acids present in the sample being analyzed.

### EXAMPLES

The presently described technology and its advantages will be better understood by reference to the following examples. These examples are provided to describe specific embodiments of the present technology. By providing these specific examples, it is not intended limit the scope and spirit of the present technology. It will be understood by those skilled in the art that the full scope of the presently described technology encompasses the subject matter defined by the claims appending this specification, and any alterations, modifications, or equivalents of those claims.

### Example 1: Generation of Comprehensive Taq DNA Polymerase Mutant Library

Eight saturation mutagenesis libraries were generated by nicking mutagenesis as described by Wrenbeck et. al. NNK primers were ordered as Integrated DNA Technologies oPools™ DNA Oligo Pools and used in the first strand synthesis of the technique. Each generated Taq DNA polymerase library was transformed into E. coli cells, colonies collected, and DNA extracted and sequenced to confirm >99% of all possible amino acid substitutions were available.

### Example 2: DNA Amplification by PCR

Each sequence confirmed Taq DNA polymerase mutant library was transformed into BL21-CodonPlus E. coli Cells (Agilent), colonies collected, used to inoculate Luria Broth media with kanamycin (0.05 mg/mL) and grown at 37° C. until an OD of approximately 0.6 was reached. The addition of IPTG (0.5 mM final) was used to induce protein expression, followed by growth at 37° C. for 3 hours. Cells were harvested at 17,000×g for 1 minute.

The cell pellets were re-suspended in a Taq polymerase buffer containing the following: 10 mM Tris HCl (pH 9.0), 50 mM KCl, 0.1% Triton X-100, 1.5 mM MgCl₂. The eight Taq DNA polymerase libraries were combined and added to a Taq polymerase reaction mixture containing the following: 10 mM Tris HCl (pH 9.0), 50 mM KCl, 0.1% Triton X-100, 1.5 mM MgCl₂, 500 μM dNTPs, 125 nM of forward and reverse primers and 6.67 ng/μL humic acid. An emulsion was introduced using a Dolomite u Encapsulator System. The resulting emulsion was evenly distributed into a PCR plate and the following cycling conditions were carried out: 95° C. for 5 minutes, followed by 30 cycles of 95° C. for 30 seconds, 64° C. for 1 minute, 68° C. for 2 minutes, 45 seconds, and a final extension at 68° C. for 5 minutes.

After the PCR cycling, the emulsion was broken using 5 volumes of diethyl ether, the amplified DNA purified by QIAquick PCR purification kit (Qiagen) and subsequently treated with DpnI at 37° C. for 1 hour, followed by an addition purification by QIAquick PCR purification kit (Qiagen). An additional amplification was performed with KOD polymerase using recommended manufacturing conditions at a low cycle number to not bias the libraries, followed by a purification of the amplified DNA.

### Example 3: Second Round of Enrichment

In order to accomplish a second round of enrichment, a restriction enzyme digest with XbaI and NotI was performed on both the original plasmid vector and the amplified Taq polymerase library, followed by a gel cut purification to result in purified DNA fragments with sticky ends. Phosphorylation of the plasmid backbone was accomplished with antartic phosphatase, followed by a ligation of the phosphorylated plasmid backbone and the Taq polymerase library with T4 DNA ligase. The resulting Taq polymerase library was transformed into E. coli cells, colonies collected, DNA extracted and transformed into BL21-CodonPlus E. coli Cells for protein expression of the Taq polymerase library, as described above.

The presently described technology is now described in such full, clear, concise and exact terms as to enable any person skilled in the art to which it pertains, to practice the same. It is to be understood that the foregoing describes preferred embodiments of the technology and that modifications may be made therein without departing from the spirit or scope of the invention as set forth in the appended claims.

### Example 4: DNA Amplification

The resulting cells containing expressed Taq polymerase library were re-suspended in a Taq polymerase buffer and an emulsion was introduced as described above. The resulting DNA from both rounds 1 and 2 of selection were sequenced by amplicon sequencing using gene specific primers. Pre-selected Taq DNA polymerase libraries were also sequenced, and a fold-change was used as the enriched sequencing output.

From the entire Taq DNA polymerase gene and after two rounds of enrichment, 116 amino acid substitutions showed an enrichment >15-fold over the wild-type Taq DNA polymerase and 354 amino acid substitutions having >10 enrichment over the wild-type Taq DNA polymerase.

All amino acid substitutions that showed an enrichment with >20-fold over the wild-type Taq DNA were cloned and evaluated as single mutants for humic acid resistance. These amino acid substitutions are summarized in Table 2.

TABLE 2
Humic acid resistance of single mutation Taq DNA Polymerase variants.

| | | FA | Humic Acid Concentration | | | | | | | |
|---|---|---|---|---|---|---|---|---|---|---|
| # | Mutant | Plate # | 0 ng/μL | 1.7 ng/μL | 3.3 ng/μL | 5 ng/μL | 6.7 ng/μL | 8.3 ng/μL | 10 ng/μL | 11.7 ng/μL |
| 1 | WT | 1 | 0.095 | 0.0156 | 0.0004 | 0 | 0 | 0 | 0 | 0 |
| 2 | E507K | 1 | 0.0086 | 0.0002 | 0 | 0 | 0 | 0 | 0 | 0 |
| 3 | A227S | 1 | 0.016 | 0.0003 | 0 | 0 | 0 | 0 | 0 | 0 |
| 4 | W706F | 1 | 0.1324 | 0.1054 | 0.012 | 0.0006 | 0 | 0 | 0 | 0 |
| 5 | K709L | 1 | 0.033 | 0.0004 | 0 | 0 | 0 | 0 | 0 | 0 |
| 6 | P527Q | 1 | 0.0964 | 0.0011 | 0 | 0 | 0 | 0 | 0 | 0 |
| 7 | G395Y | 1 | 0.0299 | 0.0008 | 0 | 0 | 0 | 0 | 0 | 0 |
| 8 | E135A | 1 | 0.0318 | 0.0021 | 0 | 0 | 0 | 0 | 0 | 0 |
| 9 | H75R | 1 | 0.0932 | 0.0676 | 0.0352 | 0.0015 | 0 | 0 | 0 | 0 |
| 10 | E159G | 1 | 0.0461 | 0.0168 | 0 | 0 | 0 | 0 | 0 | 0 |
| 11 | R695S | 1 | 0.0136 | 0 | 0 | 0 | 0 | 0 | 0 | 0 |
| 12 | E39R | 1 | 0.0867 | 0.0725 | 0.0653 | 0.034 | 0.001 | 0 | 0 | 0 |
| 1 | WT | 2 | 0.1185 | 0.0299 | 0.0001 | 0 | 0 | 0 | 0 | 0 |
| 2 | E507K | 2 | 0.0086 | 0 | 0 | 0 | 0 | 0 | 0 | 0 |
| 13 | L55R | 2 | 0.0191 | 0.0004 | 0 | 0 | 0 | 0 | 0 | 0 |
| 14 | V821D | 2 | 0 | 0 | 0 | 0 | 0 | 0 | 0 | 0 |
| 15 | Y161V | 2 | 0 | 0 | 0 | 0 | 0 | 0 | 0 | 0 |
| 16 | K247A | 2 | 0.0306 | 0 | 0 | 0 | 0 | 0 | 0 | 0 |
| 17 | Y339V | 2 | 0.0785 | 0.0085 | 0 | 0 | 0 | 0 | 0 | 0 |
| 18 | F632L | 2 | 0.0095 | 0.0001 | 0 | 0 | 0 | 0 | 0 | 0 |
| 19 | A439M | 2 | 0.1288 | 0.0874 | 0.0679 | 0.0002 | 0 | 0 | 0 | 0 |
| 20 | A757S | 2 | 0.0007 | 0 | 0 | 0 | 0 | 0 | 0 | 0 |
| 21 | R801Q | 2 | 0.0695 | 0.0003 | 0 | 0 | 0 | 0 | 0 | 0 |
| 22 | A341K | 2 | 0.0008 | 0 | 0 | 0 | 0 | 0 | 0 | 0 |
| 1 | WT | 3 | 0.1242 | 0.0159 | 0.0001 | 0 | 0 | 0 | 0 | 0 |
| 2 | E507K | 3 | 0.0129 | 0 | 0 | 0 | 0 | 0 | 0 | 0 |
| 23 | A705S | 3 | 0.0105 | 0 | 0 | 0 | 0 | 0 | 0 | 0 |
| 24 | A227Y | 3 | 0.0109 | 0.0005 | 0 | 0 | 0 | 0 | 0 | 0 |
| 1 | WT | 4 | 0.2494 | 0.1618 | 0.0043 | 0 | 0 | 0 | 0 | 0 |
| 12 | E39R | 4 | 0.1496 | 0.1411 | 0.1497 | 0.0969 | 0.0108 | 0 | 0 | 0 |
| 25 | E255Q | 4 | 0.1605 | 0.0681 | 0.0008 | 0 | 0 | 0 | 0 | 0 |
| 26 | E423I | 4 | 0.0362 | 0.0124 | 0.0004 | 0 | 0 | 0 | 0 | 0 |
| 27 | S383H | 4 | 0.1497 | 0.0426 | 0.0004 | 0 | 0 | 0 | 0 | 0 |
| 28 | G160N | 4 | 0.1844 | 0.1189 | 0.0165 | 0 | 0 | 0 | 0 | 0 |
| 29 | W167E | 1 | 0.2777 | 0.2088 | 0.0117 | 0 | 0 | 0 | 0 | 0 |
| 30 | Y161R | 4 | 0.2198 | 0.1317 | 0.0449 | 0.0007 | 0 | 0 | 0 | 0 |
| 31 | L606V | 4 | 0.2054 | 0.1635 | 0.0229 | 0.0003 | 0 | 0 | 0 | 0 |
| 32 | Y339P | 1 | 0.0965 | 0.0675 | 0.0003 | 0 | 0 | 0 | 0 | 0 |
| 33 | H21R | 4 | 0.2089 | 0.1908 | 0.0609 | 0.0021 | 0 | 0 | 0 | 0 |
| 34 | E263S | 4 | 0.2417 | 0.1836 | 0.0324 | 0.0005 | 0 | 0 | 0 | 0 |
| 1 | WT | 5 | 0.2556 | 0.1587 | 0.0045 | 0 | 0 | 0 | 0 | 0 |
| 12 | E39R | 5 | 0.1939 | 0.1259 | 0.1675 | 0.0724 | 0.0048 | 0 | 0 | 0 |
| 35 | T569A | 5 | 0.001 | 0 | 0 | 0 | 0 | 0 | 0 | 0 |
| 36 | R110M | 5 | 0.2272 | 0.1446 | 0.0014 | 0 | 0 | 0 | 0 | 0 |
| 37 | L367V | 5 | 0.2717 | 0.1851 | 0.0903 | 0.0017 | 0 | 0 | 0 | 0 |
| 38 | P194Y | 5 | 0.2311 | 0.1709 | 0.0041 | 0 | 0 | 0 | 0 | 0 |
| 39 | G160S | 5 | 0.1271 | 0.0007 | 0 | 0 | 0 | 0 | 0 | 0 |
| 1 | WT | 6 | 0.3024 | 0.1972 | 0.0338 | 0 | 0 | 0 | 0 | 0 |
| 12 | E39R | 6 | 0.24 | 0.148 | 0.1602 | 0.009 | 0 | 0 | 0 | 0 |
| 40 | V607N | 6 | 0 | 0 | 0 | 0 | 0 | 0 | 0 | 0 |
| 41 | G59T | 6 | 0.3105 | 0.2997 | 0.1917 | 0.0252 | 0 | 0 | 0 | 0 |
| 42 | R2F | 6 | 0.3244 | 0.2985 | 0.3256 | 0.1907 | 0.0042 | 0 | 0 | 0 |
| 43 | L605P | 6 | 0 | 0 | 0 | 0 | 0 | 0 | 0 | 0 |
| 44 | Y161N | 6 | 0.2828 | 0.2709 | 0.1185 | 0.0007 | 0 | 0 | 0 | 0 |
| 45 | Q95T | 6 | 0.3323 | 0.2388 | 0.0252 | 0 | 0 | 0 | 0 | 0 |
| 46 | Q754R | 6 | 0 | 0 | 0 | 0 | 0 | 0 | 0 | 0 |
| 47 | Q582M | 6 | 0.17 | 0.1857 | 0.0122 | 0.0003 | 0 | 0 | 0 | 0 |
| 48 | T88R | 6 | 0.2046 | 0.1789 | 0.1494 | 0.0068 | 0 | 0 | 0 | 0 |
| 49 | E465A | 6 | 0.3242 | 0.2989 | 0.2174 | 0.0521 | 0 | 0 | 0 | 0 |
| 1 | WT | 7 | 0.2163 | 0.1085 | 0.018 | 0 | 0 | 0 | 0 | 0 |
| 12 | E39R | 7 | 0.1835 | 0.1811 | 0.091 | 0.0145 | 0 | 0 | 0 | 0 |
| 50 | E507T | 7 | 0.2522 | 0.3468 | 0.3229 | 0.2241 | 0.184 | 0.1192 | 0.0029 | 0 |
| 51 | E507L | 7 | 0.2165 | 0.2102 | 0.2484 | 0.2131 | 0.0751 | 0 | 0 | 0 |
| 52 | Y455R | 7 | 0.0238 | 0.0115 | 0 | 0 | 0 | 0 | 0 | 0 |
| 53 | L33P | 7 | 0.325 | 0.2894 | 0.0541 | 0 | 0 | 0 | 0 | 0 |
| 54 | L5P | 7 | 0.2587 | 0.1896 | 0.0804 | 0.0007 | 0 | 0 | 0 | 0 |
| 55 | K128R | 7 | 0.3212 | 0.3327 | 0.2818 | 0.185 | 0.0383 | 0 | 0 | 0 |
| 56 | L148P | 7 | 0.1168 | 0.006 | 0 | 0 | 0 | 0 | 0 | 0 |
| 57 | K225R | 7 | 0.1792 | 0.1269 | 0.0016 | 0 | 0 | 0 | 0 | 0 |
| 1 | WT | 8 | 0.2512 | 0.1131 | 0.0032 | 0 | 0 | 0 | 0 | 0 |
| 12 | E39R | 8 | 0.1465 | 0.132 | 0.1001 | 0.0365 | 0 | 0 | 0 | 0 |
| 58 | Y78S | 8 | 0.3507 | 0.2281 | 0.0597 | 0 | 0 | 0 | 0 | 0 |
| 59 | D547R | 8 | 0 | 0 | 0 | 0 | 0 | 0 | 0 | 0 |
| 60 | T386Q | 8 | 0.1039 | 0.0238 | 0 | 0 | 0 | 0 | 0 | 0 |
| 61 | S383P | 8 | 0 | 0 | 0 | 0 | 0 | 0 | 0 | 0 |
| 62 | Y78R | 8 | 0.1399 | 0.0592 | 0.0021 | 0 | 0 | 0 | 0 | 0 |
| 63 | Y182S | 8 | 0.2081 | 0.1363 | 0.0851 | 0.013 | 0.0005 | 0 | 0 | 0 |
| 64 | A83H | 8 | 0.2205 | 0.1354 | 0.0197 | 0 | 0 | 0 | 0 | 0 |
| 65 | L96P | 8 | 0.1848 | 0.0047 | 0 | 0 | 0 | 0 | 0 | 0 |
| 66 | Q178R | 8 | 0.2031 | 0.1349 | 0.0383 | 0.0007 | 0 | 0 | 0 | 0 |
| 67 | L352P | 8 | 0 | 0 | 0 | 0 | 0 | 0 | 0 | 0 |
| 1 | WT | 9 | 0.2423 | 0.1207 | 0.0018 | 0 | 0 | 0 | 0 | 0 |
| 12 | E39R | 9 | 0.1753 | 0.1457 | 0.0913 | 0.0257 | 0 | 0 | 0 | 0 |
| 68 | E296G | 9 | 0.1099 | 0.003 | 0 | 0 | 0 | 0 | 0 | 0 |
| 69 | E423G | 9 | 0 | 0 | 0 | 0 | 0 | 0 | 0 | 0 |
| 70 | E404G | 9 | 0.0565 | 0.0076 | 0 | 0 | 0 | 0 | 0 | 0 |
| 71 | K508R | 9 | 0.189 | 0.0429 | 0 | 0 | 0 | 0 | 0 | 0 |
| 72 | V449A | 9 | 0.0007 | 0 | 0 | 0 | 0 | 0 | 0 | 0 |
| 73 | K531R | 9 | 0.0925 | 0.0095 | 0 | 0 | 0 | 0 | 0 | 0 |
| 74 | E524G | 9 | 0.105 | 0.013 | 0 | 0 | 0 | 0 | 0 | 0 |
| 75 | Q680R | 9 | 0.0911 | 0.0345 | 0 | 0 | 0 | 0 | 0 | 0 |
| 76 | L670P | 9 | 0 | 0 | 0 | 0 | 0 | 0 | 0 | 0 |
| 77 | S679G | 9 | 0.0196 | 0 | 0 | 0 | 0 | 0 | 0 | 0 |
| 1 | WT | 10 | 0.2115 | 0.127 | 0.0031 | 0 | 0 | 0 | 0 | 0 |
| 12 | E39R | 10 | 0.2069 | 0.1511 | 0.1318 | 0.0293 | 0 | 0 | 0 | 0 |
| 78 | Q690R | 10 | 0.1327 | 0.0654 | 0.0112 | 0.0006 | 0 | 0 | 0 | 0 |
| 79 | L768P | 10 | 0 | 0 | 0 | 0 | 0 | 0 | 0 | 0 |
| 80 | Q782R | 10 | 0 | 0 | 0 | 0 | 0 | 0 | 0 | 0 |
| 1 | WT | 11 | 0.1844 | 0.0136 | 0 | 0 | 0 | 0 | 0 | 0 |
| 12 | E39R | 11 | 0.1374 | 0.1428 | 0.0663 | 0 | 0 | 0 | 0 | 0 |
| 81 | A61D | 11 | 0.2997 | 0.2942 | 0.1723 | 0.012 | 0 | 0 | 0 | 0 |
| 82 | E774S | 11 | 0.2192 | 0.1957 | 0.0982 | 0 | 0 | 0 | 0 | 0 |
| 83 | E388S | 11 | 0.1687 | 0.0821 | 0.0667 | 0.0015 | 0 | 0 | 0 | 0 |
| 84 | F749A | 11 | 0.2015 | 0.1352 | 0.0188 | 0 | 0 | 0 | 0 | 0 |
| 85 | G115R | 11 | 0.118 | 0.0837 | 0.0031 | 0 | 0 | 0 | 0 | 0 |
| 86 | A70M | 11 | 0.2437 | 0.1638 | 0.0177 | 0 | 0 | 0 | 0 | 0 |
| 87 | D151S | 11 | 0.0801 | 0.0043 | 0 | 0 | 0 | 0 | 0 | 0 |
| 88 | P589A | 11 | 0.3164 | 0.2865 | 0.1916 | 0.02 | 0 | 0 | 0 | 0 |
| 89 | V103A | 11 | 0.2229 | 0.1111 | 0.0014 | 0 | 0 | 0 | 0 | 0 |
| 90 | E210G | 11 | 0.0378 | 0 | 0 | 0 | 0 | 0 | 0 | 0 |
| 1 | WT | 12 | 0.2172 | 0.0191 | 0 | 0 | 0 | 0 | 0 | 0 |
| 12 | E39R | 12 | 0.1705 | 0.1581 | 0.093 | 0 | 0 | 0 | 0 | 0 |
| 91 | LA56P | 12 | 0 | 0 | 0 | 0 | 0 | 0 | 0 | 0 |

Among the 91 single mutations in Taq DNA polymerase tested for humic acid tolerance, 33 had increased resistance over the WT Taq DNA polymerase. Fourteen of the 33 substitutions performed amplification at a higher dose of humic acid, over the WT Taq DNA polymerase. These 14 substitutions are the following: R2F, H2101R, E39R, G59T, A61D, H75R, T88R, K128R, Y182S, L367V, E465A, E507T/L and P589A. Among the 34 single mutations in Taq DNA polymerase tested for humic acid tolerance, 33 had increased resistance over the WT Taq DNA polymerase. All of the 33 substitutions performed amplification at a higher dose of humic acid, over the WT Taq DNA polymerase. These 33 substitutions are the following: V17L, Y24T/H, F27P/R, A29P, G59F, F73S/R, A77S, G84A, P89I, L106V, E117R, A141P, G195R, E210R, L233D, L245H, K260G, A259T, G286S, S290P, S383N, S543F, R596S, V631A, K709R, E742N/G, E774P, M775Y and K831E. Among these 33 positions, 9 showed a substantial increase in humic acid resistance over the WT Taq DNA polymerase. These 9 positions are the following: V17L, Y24H, F27R, A29P, E210R, V631A, K709R, E742G and K831E.

### Example 5: Evaluation of Stacked Mutants for Resistance to Humic Acid

Taq DNA Polymerase mutants were evaluated for resistance to humic acid by exposing the Taq DNA Polymerase mutants to increasing concentrations of humic acid ranging from 0 ng/μL to 117 ng/μL. The concentration of DNA amplified and the fold-change are listed in Table 3:

TABLE 3
Humic acid tolerance of Taq DNA polymerase stacked mutants.

| | | | | | | | FA | | Humic Acid Titration | | | | | | |
|---|---|---|---|---|---|---|---|---|---|---|---|---|---|---|---|
| Mutant | Mutant | Mutant | Mutant | Mutant | Mutant | Mutant | Plate | 0 | 1.7 | 3.3 | 5 | 6.7 | 8.3 | 10 | 11.7 |
| ID | 1 | 2 | 3 | 4 | 5 | 6 | # | ng/μL | ng/μL | ng/μL | ng/μL | ng/μL | ng/μL | ng/μL | ng/μL |
| WT | — | — | — | — | | | 1 | 0.0879 | 0.0125 | 0.0002 | 0 | 0 | 0 | 0 | 0 |
| M1 | E39R | | | | | | 1 | 0.0731 | 0.0282 | 0.0123 | 0.0001 | 0 | 0 | 0 | 0 |
| M8 | E39R | A61D | | | | | 1 | 0.0317 | 0.0027 | 0 | 0 | 0 | 0 | 0 | 0 |
| M9 | H21R | A61D | | | | | 1 | 0.0602 | 0.0058 | 0 | 0 | 0 | 0 | 0 | 0 |
| M10 | A61D | L367V | | | | | 1 | 0.1235 | 0.021 | 0.0002 | 0 | 0 | 0 | 0 | 0 |
| M11 | R2F | A61D | | | | | 1 | 0.1266 | 0.0219 | 0.0037 | 0 | 0 | 0 | 0 | 0 |
| M12 | A61D | T88R | | | | | 1 | 0.0193 | 0.0005 | 0 | 0 | 0 | 0 | 0 | 0 |
| M13 | A61D | Y182S | | | | | 1 | 0.0387 | 0.014 | 0.001 | 0 | 0 | 0 | 0 | 0 |
| M14 | H21R | E39R | | | | | 1 | 0.0442 | 0.0351 | 0.0126 | 0.0003 | 0 | 0 | 0 | 0 |
| M15 | E39R | L367V | | | | | 1 | 0.0988 | 0.0207 | 0.0135 | 0.0002 | 0 | 0 | 0 | 0 |
| M16 | R2F | E39R | | | | | 1 | 0.0826 | 0.0493 | 0.0115 | 0.0002 | 0 | 0 | 0 | 0 |
| M17 | E39R | T88R | | | | | 1 | 0.0238 | 0.0136 | 0.0016 | 0 | 0 | 0 | 0 | 0 |
| WT | — | — | — | — | | | 2 | 0.0403 | 0.0076 | 0.0002 | 0 | 0 | 0 | 0 | 0 |
| M1 | E39R | | | | | | 2 | 0.051 | 0.0123 | 0.0094 | 0.0002 | 0 | 0 | 0 | 0 |
| M18 | E39R | Y182S | | | | | 2 | 0.017 | 0.0077 | 0.0042 | 0.0002 | 0 | 0 | 0 | 0 |
| M19 | H21R | L367V | | | | | 2 | 0.0279 | 0.0016 | 0 | 0 | 0 | 0 | 0 | 0 |
| M20 | R2F | H21R | | | | | 2 | 0.0281 | 0.0059 | 0 | 0 | 0 | 0 | 0 | 0 |
| M21 | H21R | T88R | | | | | 2 | 0.0385 | 0.0336 | 0.0185 | 0.0065 | 0.0003 | 0 | 0 | 0 |
| M22 | H21R | Y182S | | | | | 2 | 0 | 0 | 0 | 0 | 0 | 0 | 0 | 0 |
| M23 | R2F | L367V | | | | | 2 | 0.0346 | 0.0053 | 0 | 0 | 0 | 0 | 0 | 0 |
| M24 | T88R | L367V | | | | | 2 | 0.0003 | 0 | 0 | 0 | 0 | 0 | 0 | 0 |
| M25 | Y182S | L367V | | | | | 2 | 0.0062 | 0.0015 | 0 | 0 | 0 | 0 | 0 | 0 |
| M26 | R2F | T88R | | | | | 2 | 0.0276 | 0.0068 | 0.0001 | 0 | 0 | 0 | 0 | 0 |
| M27 | R2F | Y182S | | | | | 2 | 0.0087 | 0.0001 | 0 | 0 | 0 | 0 | 0 | 0 |
| WT | — | — | — | — | | | 3 | 0.0451 | 0.0073 | 0.0002 | 0 | 0 | 0 | 0 | 0 |
| M1 | E39R | | | | | | 3 | 0.0386 | 0.0177 | 0.0081 | 0.0001 | 0 | 0 | 0 | 0 |
| M28 | T88R | Y182S | | | | | 3 | 0.016 | 0.006 | 0.0019 | 0.0002 | 0 | 0 | 0 | 0 |
| WT | — | — | — | — | | | 4 | 0.1289 | 0.0041 | 0 | 0 | 0 | 0 | 0 | 0 |
| M21 | H21R | T88R | | | | | 4 | 0.1293 | 0.1284 | 0.1063 | 0.0797 | 0.0204 | 0 | 0 | 0 |
| M29 | H21R | E39R | T88R | | | | 4 | 0.0984 | 0.0683 | 0.0323 | 0.0019 | 0 | 0 | 0 | 0 |
| M30 | H21R | T88R | L367V | | | | 4 | 0.1974 | 0.1207 | 0.0935 | 0.057 | 0.0033 | 0 | 0 | 0 |
| M31 | R2F | H21R | T88R | | | | 4 | 0.1862 | 0.1448 | 0.0879 | 0.0508 | 0 | 0 | 0 | 0 |
| M32 | H21R | T88R | Y182S | | | | 4 | 0.0164 | 0.0061 | 0 | 0 | 0 | 0 | 0 | 0 |
| M33 | H21R | A61D | T88R | | | | 4 | 0.1858 | 0.1493 | 0.113 | 0.0607 | 0.0003 | 0 | 0 | 0 |
| M36 | H21R | E39R | T88R | Y182S | | | 4 | 0.0508 | 0.0617 | 0.037 | 0.0143 | 0.001 | 0 | 0 | 0 |
| M37 | H21R | E39R | A61D | T88R | | | 4 | 0.1808 | 0.1864 | 0.1814 | 0.1327 | 0.0994 | 0.0108 | 0 | 0 |
| M38 | R2F | H21R | T88R | L367V | | | 4 | 0.1281 | 0.1073 | 0.0508 | 0.0369 | 0.0003 | 0 | 0 | 0 |
| WT | — | — | — | — | | | 5 | 0.0749 | 0.0037 | 0 | 0 | 0 | 0 | 0 | 0 |
| M21 | | | | | | | 5 | 0.0945 | 0.0565 | 0.0576 | 0.0261 | 0.0186 | 0 | 0 | 0 |
| M39 | H21R | T88R | Y182S | L367V | | | 5 | 0.0399 | 0.0181 | 0.0088 | 0.0006 | 0 | 0 | 0 | 0 |
| M40 | H21R | A61D | T88R | L367V | | | 5 | 0.1156 | 0.0671 | 0.0484 | 0.0011 | 0 | 0 | 0 | 0 |
| M41 | R2F | H21R | T88R | Y182S | | | 5 | 0.0775 | 0.0494 | 0.0428 | 0.0152 | 0.0056 | 0 | 0 | 0 |
| M42 | R2F | H21R | A61D | T88R | | | 5 | 0.0635 | 0.065 | 0.0526 | 0.0352 | 0.0198 | 0 | 0 | 0 |
| M43 | H21R | A61D | T88R | Y182S | | | 5 | 0.0856 | 0.0655 | 0.0472 | 0.0437 | 0.0243 | 0 | 0 | 0 |
| WT | — | — | — | — | — | | 6 | 0.1104 | 0.0375 | 0.0002 | 0 | 0 | 0 | 0 | 0 |
| M37 | H21R | E39R | A61D | T88R | — | | 6 | 0.1857 | 0.2286 | 0.1498 | 0.118 | 0.0634 | 0.0029 | 0 | 0 |
| M44 | V17L | H21R | T88R | — | — | | 6 | 0.2418 | 0.1781 | 0.1847 | 0.1561 | 0.0863 | 0.0626 | 0.0113 | 0 |
| M47 | H21R | A29P | T88R | — | — | | 6 | 0.2756 | 0.1728 | 0.2328 | 0.1274 | 0.0417 | 0 | 0 | 0 |
| M48 | H21R | T88R | E210R | — | — | | 6 | 0.2018 | 0.2728 | 0.213 | 0.2261 | 0.1466 | 0.1043 | 0.0771 | 0.0239 |
| M49 | H21R | T88R | V631A | — | — | | 6 | 0.1176 | 0.0609 | 0.0156 | 0 | 0 | 0 | 0 | 0 |
| M50 | H21R | T88R | K709R | — | — | | 6 | 0.0305 | 0.0077 | 0.0002 | 0 | 0 | 0 | 0 | 0 |
| M51 | H21R | T88R | E724G | — | — | | 6 | 0.0234 | 0.0411 | 0.0183 | 0.0079 | 0 | 0 | 0 | 0 |
| M52 | H21R | T88R | K831E | — | — | | 6 | 0.0723 | 0.0338 | 0.0282 | 0.0146 | 0.0019 | 0 | 0 | 0 |
| M56 | H21R | A29P | E39R | A61D | T88R | | 6 | 0.2706 | 0.2829 | 0.2767 | 0.1341 | 0.069 | 0.0001 | 0 | 0 |
| M57 | H21R | E39R | A61D | T88R | E210R | | 6 | 0.235 | 0.1561 | 0.1829 | 0.1341 | 0.0689 | 0.025 | 0.0001 | 0 |
| M59 | H21R | E39R | A61D | T88R | K709R | | 6 | 0.1795 | 0.1468 | 0.1481 | 0.108 | 0.0281 | 0.0002 | 0 | 0 |
| WT | — | — | — | — | — | | 7 | 0.0938 | 0.0156 | 0 | 0 | 0 | 0 | 0 | 0 |
| M37 | H21R | E39R | A61D | T88R | — | | 7 | 0.2977 | 0.1785 | 0.2185 | 0.2284 | 0.1447 | 0.0629 | 0.0397 | 0 |
| M45 | H21R | Y24H | T88R | — | — | | 7 | 0.19 | 0.1059 | 0.0657 | 0.0098 | 0 | 0 | 0 | 0 |
| M46 | H21R | F27R | T88R | — | — | | 7 | 0.1058 | 0.058 | 0.0581 | 0.0282 | 0.0016 | 0 | 0 | 0 |
| M53 | V17L | H21R | E39R | A61D | T88R | | 7 | 0.136 | 0.0995 | 0.0464 | 0.0071 | 0 | 0 | 0 | 0 |
| M54 | H21R | Y24H | E39R | A61D | T88R | | 7 | 0.1875 | 0.1304 | 0.0763 | 0.0132 | 0.0002 | 0 | 0 | 0 |
| M55 | H21R | F27R | E39R | A61D | T88R | | 7 | 0.2345 | 0.1425 | 0.1394 | 0.1278 | 0.0594 | 0.0118 | 0 | 0 |
| M58 | H21R | E39R | A61D | T88R | V631A | | 7 | 0.1085 | 0.0822 | 0.0159 | 0.0008 | 0 | 0 | 0 | 0 |
| M60 | H21R | E39R | A61D | T88R | E724G | | 7 | 0.0647 | 0.0559 | 0.0234 | 0 | 0 | 0 | 0 | 0 |
| M61 | H21R | E39R | A61D | T88R | K831E | | 7 | 0.013 | 0.007 | 0.0053 | 0.001 | 0 | 0 | 0 | 0 |
| WT | — | — | — | — | — | | 8 | 0.0595 | 0.0096 | 0 | 0 | 0 | 0 | 0 | 0 |
| M37 | H21R | E39R | A61D | T88R | — | | 8 | 0.1329 | 0.1358 | 0.1101 | 0.0871 | 0.0516 | 0.0043 | 0 | 0 |
| M44 | V17L | H21R | T88R | — | — | | 8 | 0.1176 | 0.0852 | 0.1235 | 0.0922 | 0.0552 | 0.0188 | 0.0001 | 0 |
| M48 | H21R | T88R | E210R | — | — | | 8 | 0.1329 | 0.1339 | 0.1156 | 0.1194 | 0.1096 | 0.0679 | 0.0409 | 0.0275 |
| M62 | V17L | H21R | T88R | E210R | — | | 8 | 0.1031 | 0.0841 | 0.0834 | 0.066 | 0.033 | 0.0093 | 0 | 0 |
| M63 | H21R | E39R | T88R | E210R | — | | 8 | 0.1136 | 0.1085 | 0.0896 | 0.0754 | 0.0517 | 0.0244 | 0.0015 | 0 |
| M64 | H21R | A61D | T88R | E210R | — | | 8 | 0.1337 | 0.1499 | 0.1027 | 0.1326 | 0.0778 | 0.0349 | 0.0002 | 0 |
| M65 | V17L | H21R | E39R | T88R | E210R | | 8 | 0.076 | 0.0456 | 0.0284 | 0.0059 | 0 | 0 | 0 | 0 |
| M66 | V17L | H21R | A61D | T88R | E210R | | 8 | 0.1202 | 0.1052 | 0.0751 | 0.063 | 0.0471 | 0.0051 | 0 | 0 |
| WT | — | — | — | — | — | — | 15 | 0.1259 | 0.0219 | 0 | 0 | 0 | 0 | 0 | 0 |
| M67 | E210R | E507T | — | — | — | — | 15 | 0.1077 | 0.0668 | 0.0466 | 0.0044 | 0 | 0 | 0 | 0 |
| M68 | T88R | E507T | — | — | — | — | 15 | 0.0646 | 0.0394 | 0.0254 | 0.012 | 0 | 0 | 0 | 0 |
| M69 | T88R | E210R | — | — | — | — | 15 | 0.0845 | 0.0584 | 0.0368 | 0.0253 | 0.0076 | 0 | 0 | 0 |
| M70 | A61D | E507T | — | — | — | — | 15 | 0.1155 | 0.1458 | 0.0767 | 0.0657 | 0.0315 | 0 | 0 | 0 |
| M71 | A61D | E210R | — | — | — | — | 15 | 0.1421 | 0.0828 | 0.0312 | 0.0002 | 0 | 0 | 0 | 0 |
| M72 | H21R | E507T | — | — | — | — | 15 | 0.1858 | 0.1843 | 0.1888 | 0.1329 | 0.1222 | 0.0619 | 0.0121 | 0.0026 |
| M73 | H21R | E210R | — | — | — | — | 15 | 0.1173 | 0.0702 | 0.0365 | 0.0053 | 0 | 0 | 0 | 0 |
| M74 | V17L | E507T | — | — | — | — | 15 | 0.1103 | 0.064 | 0.0504 | 0.045 | 0.005 | 0 | 0 | 0 |
| M75 | V17L | E210R | — | — | — | — | 15 | 0.0422 | 0.0121 | 0 | 0 | 0 | 0 | 0 | 0 |
| M76 | V17L | T88R | — | — | — | — | 15 | 0.0341 | 0 | 0 | 0 | 0 | 0 | 0 | 0 |
| WT | — | — | — | — | — | — | 16 | 0.0897 | 0.0243 | 0 | 0 | 0 | 0 | 0 | 0 |
| M77 | V17L | A61D | — | — | — | — | 16 | 0.1114 | 0.0004 | 0 | 0 | 0 | 0 | 0 | 0 |
| M78 | V17L | H21R | — | — | — | — | 16 | 0.0518 | 0.0167 | 0.0005 | 0.0003 | 0 | 0 | 0 | 0 |
| M79 | T88R | E210R | E507T | — | — | — | 16 | 0.0692 | 0.0419 | 0.0344 | 0.0256 | 0.0071 | 0 | 0 | 0 |
| M80 | A61D | E210R | E507T | — | — | — | 16 | 0.1063 | 0.0712 | 0.0522 | 0.0371 | 0.0008 | 0 | 0 | 0 |
| M81 | A61D | T88R | E507T | — | — | — | 16 | 0.1565 | 0.0961 | 0.0787 | 0.0362 | 0.0003 | 0 | 0 | 0 |
| M82 | H21R | E210R | E507T | — | — | — | 16 | 0.1012 | 0.08 | 0.035 | 0.003 | 0 | 0 | 0 | 0 |
| M83 | H21R | T88R | E507T | — | — | — | 16 | 0.1329 | 0.0968 | 0.0561 | 0.0349 | 0.0148 | 0 | 0 | 0 |
| M84 | H21R | A61D | E507T | — | — | — | 16 | 0.1007 | 0.0607 | 0.0447 | 0.0147 | 0.0002 | 0 | 0 | 0 |
| M85 | V17L | E210R | E507T | — | — | — | 16 | 0.1061 | 0.0612 | 0.0378 | 0.0193 | 0.0004 | 0 | 0 | 0 |
| M86 | V17L | T88R | E507T | — | — | — | 16 | 0.0315 | 0.0268 | 0.0209 | 0.0121 | 0.0009 | 0 | 0 | 0 |
| WT | — | — | — | — | — | — | 17 | 0.9542 | 0.0878 | 0 | 0 | 0 | 0 | 0 | 0 |
| M87 | V17L | A61D | E507T | — | — | — | 17 | 0.2544 | 0.2096 | 0.1811 | 0.0666 | 0.022 | 0 | 0 | 0 |
| M88 | V17L | H21R | E507T | — | — | — | 17 | 0.0574 | 0.0227 | 0.0149 | 0 | 0 | 0 | 0 | 0 |
| M89 | A61D | T88R | E210R | — | — | — | 17 | 0.2353 | 0.0349 | 0.0927 | 0.033 | 0 | 0 | 0 | 0 |
| M90 | H21R | A61D | E210R | — | — | — | 17 | 0.1828 | 0.2222 | 0.1845 | 0.2065 | 0.0662 | 0.0006 | 0.0137 | 0 |
| M91 | V17L | T88R | E210R | — | — | — | 17 | 0.098 | 0.041 | 0.0317 | 0.0135 | 0 | 0 | 0 | 0 |
| M92 | V17L | A61D | E210R | — | — | — | 17 | 0.2727 | 0.129 | 0.0034 | 0 | 0 | 0 | 0 | 0 |
| M93 | V17L | A61D | T88R | — | — | — | 17 | 0.0657 | 0.0087 | 0 | 0 | 0 | 0 | 0 | 0 |
| M94 | V17L | H21R | E210R | — | — | — | 17 | 0.0606 | 0.0482 | 0.0006 | 0 | 0 | 0 | 0 | 0 |
| M95 | V17L | H21R | A61D | — | — | — | 17 | 0.2376 | 0.0732 | 0.0149 | 0 | 0 | 0 | 0 | 0 |
| M96 | A61D | T88R | E210R | E507T | — | — | 17 | 0.2583 | 0.2317 | 0.1215 | 0.1629 | 0.0566 | 0.0425 | 0 | 0 |
| WT | — | — | — | — | — | — | 18 | 0.2665 | 0.0704 | 0 | 0 | 0 | 0 | 0 | 0 |
| M97 | H21R | T88R | E210R | E507T | — | — | 18 | 0.2522 | 0.1654 | 0.1272 | 0.1032 | 0.0362 | 0.0007 | 0 | 0 |
| M98 | H21R | A61D | E210R | E507T | — | — | 18 | 0.1478 | 0.1693 | 0.0568 | 0.0078 | 0 | 0 | 0 | 0 |
| M99 | H21R | A61D | T88R | E507T | — | — | 18 | 0.044 | 0.0104 | 0.0027 | 0 | 0 | 0 | 0 | 0 |
| M100 | V17L | T88R | E210R | E507T | — | — | 18 | 0.0739 | 0.0651 | 0.0349 | 0.0118 | 0 | 0 | 0 | 0 |
| M101 | V17L | A61D | E210R | E507T | — | — | 18 | 0.1859 | 0.231 | 0.2 | 0.1946 | 0.0439 | 0 | 0 | 0 |
| M102 | V17L | A61D | T88R | E507T | — | — | 18 | 0.2443 | 0.1427 | 0.092 | 0.0425 | 0.0277 | 0 | 0 | 0 |
| M103 | V17L | H21R | E210R | E507T | — | — | 18 | 0.2509 | 0.2154 | 0.1411 | 0.0506 | 0.0208 | 0 | 0 | 0 |
| M104 | V17L | H21R | T88R | E507T | — | — | 18 | 0.1899 | 0.0814 | 0.0987 | 0.0362 | 0.0078 | 0 | 0 | 0 |
| M105 | V17L | H21R | A61D | E507T | — | — | 18 | 0.2002 | 0.0083 | 0.2002 | 0.1429 | 0.0025 | 0 | 0 | 0 |
| M106 | V17L | A61D | T88R | E210R | — | — | 18 | 0.2219 | 0.1652 | 0.0608 | 0.0587 | 0.0093 | 0 | 0 | 0 |
| WT | — | — | — | — | — | — | 19 | 0.0875 | 0.0409 | 0.0001 | 0 | 0 | 0 | 0 | 0 |
| M107 | V17L | H21R | A61D | E210R | — | — | 19 | 0.0897 | 0.047 | 0.0192 | 0.0008 | 0 | 0 | 0 | 0 |
| M108 | V17L | H21R | A61D | T88R | — | — | 19 | 0.0466 | 0.0537 | 0.0269 | 0.008 | 0 | 0 | 0 | 0 |
| M109 | H21R | A61D | T88R | E210R | E507T | — | 19 | 0.0871 | 0.0306 | 0.0458 | 0.0286 | 0.0176 | 0.0011 | 0 | 0 |
| M110 | V17L | A61D | T88R | E210R | E507T | — | 19 | 0.1101 | 0.0443 | 0.0314 | 0.0125 | 0 | 0 | 0 | 0 |
| M111 | V17L | H21R | T88R | E210R | E507T | — | 19 | 0.074 | 0.0443 | 0.0309 | 0.0211 | 0.0005 | 0 | 0 | 0 |
| M112 | V17L | H21R | A61D | E210R | E507T | — | 19 | 0.1081 | 0.0633 | 0.0488 | 0.0345 | 0.0075 | 0 | 0 | 0 |
| M113 | V17L | H21R | A61D | T88R | E507T | — | 19 | 0.0792 | 0.0408 | 0.0324 | 0.0187 | 0.0004 | 0 | 0 | 0 |
| M114 | V17L | H21R | A61D | T88R | E210R | E507T | 19 | 0.0845 | 0.0736 | 0.046 | 0.0317 | 0.0136 | 0.0004 | 0 | 0 |

Among the 106 stacked variants of Taq DNA polymerase tested for humic acid tolerance, 104 had increased resistance over the WT Taq DNA polymerase. Of these 104 variants, 63 performed amplification at a higher dose of humic acid, over the WT Taq DNA polymerase. Of these 63 variants, 32 variants could amplify at a dose 4× higher than WT Taq DNA polymerase. These 32 variants are the following: V17L/H21R/T88R (M44) (SEQ ID NO: 75), H21R/F27R/T88R (M46) (SEQ ID NO: 79), H21R/A29P/T88R (M47) (SEQ ID NO: 81), H21R/T88R/E210R (M48) (SEQ ID NO: 83), H21R/T88R/K831E (M52) (SEQ ID NO: 91), H21R/F27R/E39R/A61D/T88R (M55) (SEQ ID NO: 97), H21R/A29P/E39R/A61D/T88R (M56) (SEQ ID NO: 99), H21R/E39R/A61D/T88R/E210R (M57) (SEQ ID NO: 101), H21R/E39R/A61D/T88R/K709R (M59) (SEQ ID NO: 105), V17L/H21R/T88R/E210R (M62) (SEQ ID NO: 111), H21R/E39R/T88R/E210R (M63) (SEQ ID NO: 113), H21R/A61D/T88R/E210R (M64) (SEQ ID NO: 115), V17L/H21R/A61D/T88R/E210R (M66) (SEQ ID NO: 119), T88R/E210R (M69) (SEQ ID NO: 125), A61D/E507T (M70) (SEQ ID NO: 127), H21R/E507T (M72) (SEQ ID NO: 131), V17L/E507T (M74) (SEQ ID NO: 135), T88R/E210R/E507T (M79) (SEQ ID NO: 145), H21R/T88R/E507T (M83) (SEQ ID NO: 153), V17L/A61D/E507T (M87) (SEQ ID NO: 161), H21R/A61D/E210R (M90) (SEQ ID NO: 167), A61D/T88R/E210R/E507T (M96) (SEQ ID NO: 179), H21R/T88R/E210R/E507T (M97) (SEQ ID NO: 181), V17L/A61D/E210R/E507T (M101) (SEQ ID NO: 189), V17L/A61D/T88R/E507T (M102) (SEQ ID NO: 191), V17L/H21R/E210R/E507T (M103) (SEQ ID NO: 193), V17L/H21R/T88R/E507T (M104) (SEQ ID NO: 195), V17L/H21R/A61D/E507T (M105) (SEQ ID NO: 197), V17L/A61D/T88R/E210R (M106) ID (SEQ NO: 199), H21R/A61D/T88R/E210R/E507T (M109) (SEQ ID NO: 205), V17L/H21R/A61D/E210R/E507T (M112) (SEQ ID NO: 211), and V17L/H21R/A61D/T88R/E210R/E507T (M114) (SEQ ID NO: 215). Of these 32 variants, 5 variants could amplify at a dose 6× higher than WT Taq DNA polymerase. These 5 variants are the following: V17L/H21R/T88R (M44), H21R/T88R/E210R (M48), H21R/E39R/T88R/E210R (M63), H21R/E507T (M72), and H21R/A61D/E210R (M90). Of these 5 variants, 2 variants could amplify at a dose 7× higher than WT Taq DNA polymerase. These variants are H21R/T88R/E210R (M48) and H21R/E507T (M72).

### Example 6: Evaluating Taq DNA Polymerase Stacked Mutants for Resistance to Heparin

Taq DNA Polymerase mutants were evaluated for resistance to heparin by exposing the Taq DNA Polymerase mutants to increasing concentrations of humic acid ranging from 0 ng/μL to 14.6 ng/μL. The concentration of DNA amplified and the fold-change are listed in Table 4:

TABLE 4
Heparin tolerance of Taq DNA polymerase stacked mutants

| | | | | | | | Heparin Titration | | | | | | | | |
|---|---|---|---|---|---|---|---|---|---|---|---|---|---|---|---|
| Mutant | Mutant | Mutant | Mutant | Mutant | Mutant | Mutant | FA | 0 | 0.5 | 0.88 | 1.53 | 2.68 | 4.69 | 8.21 | 14.36 |
| ID | 1 | 2 | 3 | 4 | 5 | 6 | Plate # | U/mL | U/mL | U/mL | U/mL | U/mL | U/mL | U/mL | U/mL |
| WT | — | — | — | — | — | | 9 | 0.0829 | 0.0191 | 0.0001 | 0 | 0 | 0 | 0 | 0 |
| M44 | V17L | H21R | T88R | — | — | | 9 | 0.1191 | 0.152 | 0.0652 | 0.0001 | 0 | 0 | 0 | 0 |
| M48 | H21R | T88R | E210R | — | — | | 9 | 0.1317 | 0.0071 | 0 | 0 | 0 | 0 | 0 | 0 |
| M62 | V17L | H21R | T88R | E210R | — | | 9 | 0.0329 | 0.0002 | 0 | 0 | 0 | 0 | 0 | 0 |
| M63 | H21R | E39R | T88R | E210R | — | | 9 | 0.0831 | 0 | 0 | 0 | 0 | 0 | 0 | 0 |
| M64 | H21R | A61D | T88R | E210R | — | | 9 | 0.15 | 0.0009 | 0 | 0 | 0 | 0 | 0 | 0 |
| M65 | V17L | H21R | E39R | T88R | E210R | | 9 | 0.0317 | 0.0001 | 0 | 0 | 0 | 0 | 0 | 0 |
| M66 | V17L | H21R | A61D | T88R | E210R | | 9 | 0.0643 | 0.0001 | 0 | 0 | 0 | 0 | 0 | 0 |
| WT | — | — | — | — | — | | — | 20 | 0.1363 | 0.1045 | 0.0739 | 0.0025 | 0 | 0 | 0 |
| M69 | T88R | E210R | — | — | — | | — | 20 | 0.1131 | 0 | 0 | 0 | 0 | 0 | 0 |
| M70 | A61D | E507T | — | — | — | | — | 20 | 0.2159 | 0.1969 | 0.139 | 0.0696 | 0 | 0 | 0 |
| M72 | H21R | E507T | — | — | — | | — | 20 | 0.2746 | 0.2297 | 0.2473 | 0.2245 | 0.1126 | 0.001 | 0 |
| M74 | V17L | E507T | — | — | — | | — | 20 | 0.1733 | 0.1 | 0.0813 | 0.0148 | 0 | 0 | 0 |
| M79 | T88R | E210R | E507T | — | — | | — | 20 | 0.0963 | 0 | 0 | 0 | 0 | 0 | 0 |
| M83 | H21R | T88R | E507T | — | — | | — | 20 | 0.147 | 0.1151 | 0.0748 | 0.0034 | 0 | 0 | 0 |
| M87 | V17L | A61D | E507T | — | — | | — | 20 | 0.2529 | 0.0015 | 0.2139 | 0.1698 | 0 | 0 | 0 |
| M90 | H21R | A61D | E210R | — | — | | — | 20 | 0.282 | 0.0364 | 0 | 0 | 0 | 0 | 0 |
| M96 | A61D | T88R | E210R | E507T | — | | — | 20 | 0.2126 | 0.0003 | 0 | 0 | 0 | 0 | 0 |
| M97 | H21R | T88R | E210R | E507T | — | | — | 20 | 0.1873 | 0.0072 | 0 | 0 | 0 | 0 | 0 |
| WT | — | — | — | — | — | | — | 21 | 0.1452 | 0.0917 | 0.0682 | 0.0022 | 0 | 0 | 0 |
| M101 | V17L | A61D | E210R | E507T | — | | — | 21 | 0.0046 | 0.0182 | 0 | 0 | 0 | 0 | 0 |
| M102 | V17L | A61D | T88R | E507T | — | | — | 21 | 0.115 | 0.0588 | 0.0328 | 0.0007 | 0 | 0 | 0 |
| M103 | V17L | H21R | E210R | E507T | — | | — | 21 | 0.1683 | 0.0002 | 0 | 0 | 0 | 0 | 0 |
| M104 | V17L | H21R | T88R | E507T | — | | — | 21 | 0.1184 | 0.0686 | 0.0289 | 0.0004 | 0 | 0 | 0 |
| M109 | H21R | A61D | T88R | E210R | E507T | | — | 21 | 0.1156 | 0 | 0 | 0 | 0 | 0 | 0 |
| M112 | V17L | H21R | A61D | E210R | E507T | | — | 21 | 0.1191 | 0 | 0 | 0 | 0 | 0 | 0 |
| M114 | V17L | H21R | A61D | T88R | E210R | E507T | | 21 | 0.1297 | 0 | 0 | 0 | 0 | 0 | 0 |

### Example 7: Evaluating Taq DNA Polymerase Stacked Mutants for Resistance to Hematin

Taq DNA Polymerase mutants were evaluated for resistance to humic acid by exposing the Taq DNA Polymerase mutants to increasing concentrations of hematin ranging from 0 μM to 64 μM. The concentration of DNA amplified and the fold-change are listed in Table 5:

TABLE 5
Hematin tolerance of Taq DNA polymerase stacked mutants

| | | | | | | | FA | | | | | | | | |
|---|---|---|---|---|---|---|---|---|---|---|---|---|---|---|---|
| Mutant | Mutant | Mutant | Mutant | Mutant | Mutant | Mutant | Plate | Hematin Titration | | | | | | | |
| ID | 1 | 2 | 3 | 4 | 5 | 6 | # | 0 μM | 1 μM | 2 μM | 4 μM | 8 μM | 16 μM | 32 μM | 64 μM |
| WT | — | — | — | — | — | | 10 | 0.0244 | 0.0084 | 0.0038 | 0 | 0 | 0 | 0 | 0 |
| M44 | V17L | H21R | T88R | — | — | | 10 | 0.0512 | 0.0388 | 0.0262 | 0.0161 | 0.006 | 0 | 0 | 0 |
| M48 | H21R | T88R | E210R | — | — | | 10 | 0.0791 | 0.0516 | 0.0276 | 0.013 | 0.009 | 0.0058 | 0 | 0 |
| M62 | V17L | H21R | T88R | E210R | — | | 10 | 0.0279 | 0.0151 | 0.0119 | 0.0094 | 0.0031 | 0 | 0 | 0 |
| M63 | H21R | E39R | T88R | E210R | — | | 10 | 0.0506 | 0.0313 | 0.0309 | 0.0167 | 0.0106 | 0.0027 | 0 | 0 |
| M64 | H21R | A61D | T88R | E210R | — | | 10 | 0.0766 | 0.0407 | 0.0249 | 0.0139 | 0.0076 | 0.0021 | 0 | 0 |
| M65 | V17L | H21R | E39R | T88R | E210R | | 10 | 0.0223 | 0.0129 | 0.0135 | 0.0065 | 0.0007 | 0 | 0 | 0 |
| M66 | V17L | H21R | A61D | T88R | E210R | | 10 | 0.0502 | 0.0259 | 0.0205 | 0.0116 | 0.0063 | 0 | 0 | 0 |
| WT | — | — | — | — | — | — | 22 | 0.0822 | 0.0435 | 0.013 | 0.0004 | 0 | 0 | 0 | 0 |
| M69 | T88R | E210R | — | — | — | — | 22 | 0.0361 | 0.0237 | 0.0177 | 0.0133 | 0.0067 | 0 | 0 | 0 |
| M70 | A61D | E507T | — | — | — | — | 22 | 0.1211 | 0.0973 | 0.0752 | 0.0428 | 0.0282 | 0.0001 | 0 | 0 |
| M72 | H21R | E507T | — | — | — | — | 22 | 0.1401 | 0.1122 | 0.091 | 0.0528 | 0.0288 | 0.0013 | 0 | 0 |
| M74 | V17L | E507T | — | — | — | — | 22 | 0.0835 | 0.0559 | 0.0275 | 0.019 | 0.0061 | 0 | 0 | 0 |
| M79 | T88R | E210R | E507T | — | — | — | 22 | 0.047 | 0.0351 | 0.0262 | 0.022 | 0.0098 | 0.0015 | 0 | 0 |
| M83 | H21R | T88R | E507T | — | — | — | 22 | 0.0767 | 0.0458 | 0.0397 | 0.0283 | 0.0171 | 0.0031 | 0 | 0 |
| M87 | V17L | A61D | E507T | — | — | — | 22 | 0.0977 | 0.0949 | 0.0441 | 0.023 | 0.0073 | 0 | 0 | 0 |
| M90 | H21R | A61D | E210R | — | — | — | 22 | 0.1207 | 0.1078 | 0.0879 | 0.0369 | 0.0153 | 0.0005 | 0 | 0 |
| M96 | A61D | T88R | E210R | E507T | — | — | 22 | 0.0935 | 0.0785 | 0.0462 | 0.03 | 0.0146 | 0.004 | 0 | 0 |
| M97 | H21R | T88R | E210R | E507T | — | — | 22 | 0.0891 | 0.076 | 0.0616 | 0.0324 | 0.0154 | 0.0079 | 0 | 0 |
| WT | — | — | — | — | — | — | 23 | 0.0908 | 0.0411 | 0.013 | 0.0004 | 0 | 0 | 0 | 0 |
| M101 | M101 | V17L | A61D | E210R | E507T | — | 23 | 0.1112 | 0.1072 | 0.0632 | 0.0396 | 0.0186 | 0.0053 | 0 | 0 |
| M102 | M102 | V17L | A61D | T88R | E507T | — | 23 | 0.0693 | 0.0367 | 0.028 | 0.0152 | 0.0068 | 0 | 0 | 0 |
| M103 | M103 | V17L | H21R | E210R | E507T | — | 23 | 0.0937 | 0.0517 | 0.0422 | 0.025 | 0.0113 | 0.0015 | 0 | 0 |
| M104 | M104 | V17L | H21R | T88R | E507T | — | 23 | 0.07 | 0.0359 | 0.031 | 0.0176 | 0.008 | 0 | 0 | 0 |
| M109 | H21R | A61D | T88R | E210R | E507T | — | 23 | 0.0584 | 0.0341 | 0.0282 | 0.0232 | 0.0104 | 0.0032 | 0 | 0 |
| M112 | V17L | H21R | A61D | E210R | E507T | — | 23 | 0.0669 | 0.047 | 0.0422 | 0.0213 | 0.0103 | 0.0008 | 0 | 0 |
| M114 | V17L | H21R | A61D | T88R | E210R | E507T | 23 | 0.0323 | 0.0288 | 0.0193 | 0.0081 | 0.0022 | 0 | 0 | 0 |
| M103 | V17L | H21R | E210R | E507T | — | — | 23 | 0.0937 | 0.0517 | 0.0422 | 0.025 | 0.0113 | 0.0015 | 0 | 0 |
| M104 | V17L | H21R | T88R | E507T | — | — | 23 | 0.07 | 0.0359 | 0.031 | 0.0176 | 0.008 | 0 | 0 | 0 |
| M109 | H21R | A61D | T88R | E210R | E507T | — | 23 | 0.0584 | 0.0341 | 0.0282 | 0.0232 | 0.0104 | 0.0032 | 0 | 0 |
| M112 | V17L | H21R | A61D | E210R | E507T | — | 23 | 0.0669 | 0.047 | 0.0422 | 0.0213 | 0.0103 | 0.0008 | 0 | 0 |
| M114 | V17L | H21R | A61D | T88R | E210R | E507T | 23 | 0.0323 | 0.0288 | 0.0193 | 0.0081 | 0.0022 | 0 | 0 | 0 |

### Example 8: Evaluating Taq DNA Polymerase Stacked Mutants for Resistance to NaCl

Taq DNA Polymerase mutants were evaluated for resistance to heparin by exposing the Taq DNA Polymerase mutants to increasing concentrations of humic acid ranging from 0 mM to 171 mM. The concentration of DNA amplified and the fold-change are listed in Table 6:

TABLE 6
NaCl tolerance of Taq DNA polymerase stacked mutants.

| Mutant | Mutant | Mutant | Mutant | Mutant | Mutant | FA | NaCl Titration | | | | | | | |
|---|---|---|---|---|---|---|---|---|---|---|---|---|---|---|
| ID | 1 | 2 | 3 | 4 | 5 | Plate # | 0 mM | 15 mM | 23 mM | 34 mM | 51 mM | 76 mM | 114 mM | 171 mM |
| WT | — | — | — | — | — | 11 | 0.0529 | 0.0289 | 0.0054 | 0.0003 | 0 | 0 | 0 | 0 |
| M44 | V17L | H21R | T88R | — | — | 11 | 0.075 | 0.0912 | 0.0558 | 0.0463 | 0.0354 | 0.0056 | 0 | 0 |
| M48 | H21R | T88R | E210R | — | — | 11 | 0.0957 | 0.0701 | 0.0821 | 0.0619 | 0.058 | 0.0437 | 0.0028 | 0 |
| M62 | V17L | H21R | T88R | E210R | — | 11 | 0.031 | 0.0405 | 0.0329 | 0.0305 | 0.0174 | 0.0074 | 0 | 0 |
| M63 | H21R | E39R | T88R | E210R | — | 11 | 0.0712 | 0.026 | 0.0384 | 0.0295 | 0.0271 | 0.0066 | 0.0001 | 0 |
| M64 | H21R | A61D | T88R | E210R | — | 11 | 0.1135 | 0.0687 | 0.08 | 0.0725 | 0.0626 | 0.0347 | 0.0009 | 0 |
| M65 | V17L | H21R | E39R | T88R | E210R | 11 | 0.0227 | 0.0129 | 0.0098 | 0.0059 | 0.0014 | 0 | 0 | 0 |
| M66 | V17L | H21R | A61D | T88R | E210R | 11 | 0.0631 | 0.0556 | 0.0452 | 0.053 | 0.0312 | 0.0135 | 0 | 0 |

### Example 9: Evaluating Taq DNA Polymerase Stacked Mutants for Resistance to Alcohol

Taq DNA Polymerase mutants were evaluated for resistance to ethanol by exposing the Taq DNA Polymerase mutants to increasing concentrations of ethanol ranging from 0% to 19.9%. The concentration of DNA amplified and the fold-change are listed in Table 7:

TABLE 7
Ethanol tolerance of Taq DNA polymerase stacked mutants.

| Mutant | Mutant | Mutant | Mutant | Mutant | Mutant | FA | Ethanol Titration | | | | | | | |
|---|---|---|---|---|---|---|---|---|---|---|---|---|---|---|
| ID | 1 | 2 | 3 | 4 | 5 | Plate # | 0.0% | 1.8% | 2.6% | 3.9% | 5.9% | 8.9% | 13.3% | 19.9% |
| WT | — | — | — | — | — | 12 | 0.0195 | 0.0102 | 0.0048 | 0 | 0 | 0 | 0 | 0 |
| M44 | V17L | H21R | T88R | — | — | 12 | 0.0519 | 0.0277 | 0.014 | 0.0018 | 0 | 0 | 0 | 0 |
| M48 | H21R | T88R | E210R | — | — | 12 | 0.0709 | 0.0267 | 0.0194 | 0.001 | 0 | 0 | 0 | 0 |
| M62 | V17L | H21R | T88R | E210R | — | 12 | 0.0252 | 0.0223 | 0.0064 | 0 | 0 | 0 | 0 | 0 |
| M63 | H21R | E39R | T88R | E210R | — | 12 | 0.0518 | 0.0255 | 0.0064 | 0 | 0 | 0 | 0 | 0 |
| M64 | H21R | A61D | T88R | E210R | — | 12 | 0.0675 | 0.0578 | 0.0206 | 0.0023 | 0 | 0 | 0 | 0 |
| M65 | V17L | H21R | E39R | T88R | E210R | 12 | 0.0253 | 0.0134 | 0.0006 | 0 | 0 | 0 | 0 | 0 |
| M66 | V17L | H21R | A61D | T88R | E210R | 12 | 0.0505 | 0.048 | 0.0139 | 0 | 0 | 0 | 0 | 0 |

### Example 10: Evaluation of Taq DNA Polymerase Stacked Mutants for Resistance to Guanidine Thiocyanate

Taq DNA Polymerase mutants were evaluated for resistance to humic acid by exposing the Taq DNA Polymerase mutants to increasing concentrations of hematin ranging from 0% to 3.2%. The concentration of DNA amplified and the fold-change are listed in Table 8:

TABLE 8
Extraction Wash Buffer tolerance of Taq DNA polymerase stacked mutants. Wash Buffer from the MagMax ™ Viral/Pathogen Kit.

| | | | | | | | FA | | | | | | | | |
|---|---|---|---|---|---|---|---|---|---|---|---|---|---|---|---|
| Mutant | Mutant | Mutant | Mutant | Mutant | Mutant | Mutant | Plate | Extraction Wash Buffer Titration | | | | | | | |
| ID | 1 | 2 | 3 | 4 | 5 | 6 | # | 0% | 0.05% | 0.10% | 0.20% | 0.40% | 0.80% | 1.60% | 3.20% |
| WT | — | — | — | — | — | | 14 | 0.0137 | 0.0109 | 0.008 | 0.0147 | 0.0049 | 0.0002 | 0 | 0 |
| M44 | V17L | H21R | T88R | — | — | | 14 | 0.0562 | 0.0413 | 0.0374 | 0.0511 | 0.0379 | 0.0149 | 0.0045 | 0 |
| M48 | H21R | T88R | E210R | — | — | | 14 | 0.0383 | 0.0311 | 0.032 | 0.0656 | 0.0299 | 0.0184 | 0.0176 | 0 |
| M62 | V17L | H21R | T88R | E210R | — | | 14 | 0.0176 | 0.021 | 0.0188 | 0.0276 | 0.0245 | 0.0047 | 0.002 | 0 |
| M63 | H21R | E39R | T88R | E210R | — | | 14 | 0.0453 | 0.0417 | 0.0407 | 0.0353 | 0.0277 | 0.0114 | 0.0024 | 0 |
| M64 | H21R | A61D | T88R | E210R | — | | 14 | 0.0543 | 0.0829 | 0.074 | 0.068 | 0.0455 | 0.0265 | 0.0191 | 0 |
| M65 | V17L | H21R | E39R | T88R | E210R | | 14 | 0.0178 | 0.0172 | 0.0166 | 0.0181 | 0.0112 | 0.0026 | 0 | 0 |
| M66 | V17L | H21R | A61D | T88R | E210R | | 14 | 0.0309 | 0.042 | 0.0471 | 0.0487 | 0.0274 | 0.0174 | 0.0032 | 0 |
| WT | — | — | — | — | — | — | 24 | 0.0875 | 0.0916 | 0.0991 | 0.085 | 0.0732 | 0.0205 | 0 | 0 |
| M69 | T88R | E210R | — | — | — | — | 24 | 0.0463 | 0.0658 | 0.0693 | 0.0797 | 0.0692 | 0.0412 | 0.0112 | 0 |
| M70 | A61D | E507T | — | — | — | — | 24 | 0.0956 | 0.1402 | 0.1461 | 0.1173 | 0.1247 | 0.0862 | 0.0367 | 0 |
| M72 | H21R | E507T | — | — | — | — | 24 | 0.1446 | 0.1445 | 0.1689 | 0.1709 | 0.1552 | 0.118 | 0.0845 | 0 |
| M74 | V17L | E507T | — | — | — | — | 24 | 0.0893 | 0.1059 | 0.1061 | 0.1163 | 0.1053 | 0.0663 | 0.0198 | 0 |
| M79 | T88R | E210R | E507T | — | — | — | 24 | 0.0695 | 0.0654 | 0.0821 | 0.0906 | 0.0681 | 0.0496 | 0.0168 | 0 |
| M83 | H21R | T88R | E507T | — | — | — | 24 | 0.0811 | 0.0853 | 0.0921 | 0.0992 | 0.0896 | 0.065 | 0.0208 | 0 |
| M87 | V17L | A61D | E507T | — | — | — | 24 | 0.1207 | 0.1298 | 0.1475 | 0.1548 | 0.148 | 0.1202 | 0.0439 | 0 |
| M90 | H21R | A61D | E210R | — | — | — | 24 | 0.134 | 0.1444 | 0.1755 | 0.1923 | 0.1789 | 0.1316 | 0.0552 | 0 |
| M96 | A61D | T88R | E210R | E507T | — | — | 24 | 0.0956 | 0.1217 | 0.141 | 0.1225 | 0.1294 | 0.1024 | 0.0474 | 0 |
| M97 | H21R | T88R | E210R | E507T | — | — | 24 | 0.1031 | 0.1108 | 0.1154 | 0.1339 | 0.1455 | 0.1103 | 0.0758 | 0.0119 |
| WT | — | — | — | — | — | — | 25 | 0.0844 | 0.086 | 0.0909 | 0.0941 | 0.0821 | 0.0209 | 0.0001 | 0 |
| M101 | V17L | A61D | E210R | E507T | — | — | 25 | 0.1118 | 0.1198 | 0.1305 | 0.1543 | 0.1275 | 0.0982 | 0.0469 | 0 |
| M102 | V17L | A61D | T88R | E507T | — | — | 25 | 0.0582 | 0.0673 | 0.0703 | 0.0696 | 0.0537 | 0.0303 | 0.0054 | 0 |
| M103 | V17L | H21R | E210R | E507T | — | — | 25 | 0.0936 | 0.0842 | 0.0958 | 0.0898 | 0.0802 | 0.044 | 0.011 | 0 |
| M104 | V17L | H21R | T88R | E507T | — | — | 25 | 0.0686 | 0.0656 | 0.0646 | 0.0733 | 0.0538 | 0.0298 | 0.0049 | 0 |
| M109 | H21R | A61D | T88R | E210R | E507T | — | 25 | 0.0539 | 0.0659 | 0.0615 | 0.0761 | 0.0538 | 0.0272 | 0.0055 | 0 |
| M112 | V17L | H21R | A61D | E210R | E507T | — | 25 | 0.0794 | 0.0787 | 0.0822 | 0.0829 | 0.0658 | 0.0276 | 0.0016 | 0 |
| M114 | V17L | H21R | A61D | T88R | E210R | E507T | 25 | 0.0486 | 0.0621 | 0.0634 | 0.0599 | 0.0535 | 0.0312 | 0.0015 | 0 |

TABLE 9
Extraction Binding Buffer tolerance of Taq DNA polymerase stacked mutants - binding Buffer from the MagMax ™ Viral/Pathogen Kit.

| Mutant | Mutant | Mutant | Mutant | Mutant | Mutant | FA | Extraction Bind Buffer Titration | | | | | | | |
|---|---|---|---|---|---|---|---|---|---|---|---|---|---|---|
| ID | 1 | 2 | 3 | 4 | 5 | Plate # | 0% | 0.05% | 0.10% | 0.20% | 0.40% | 0.80% | 1.60% | 3.20% |
| WT | — | — | — | — | — | 13 | 0.0158 | 0.0085 | 0.0033 | 0.0034 | 0 | 0 | 0 | 0 |
| M44 | V17L | H21R | T88R | — | — | 13 | 0.0399 | 0.0247 | 0.0253 | 0.0253 | 0.0089 | 0.0008 | 0 | 0 |
| M48 | H21R | T88R | E210R | — | — | 13 | 0.0383 | 0.0342 | 0.0344 | 0.02 | 0.0127 | 0.0042 | 0 | 0 |
| M62 | V17L | H21R | T88R | E210R | — | 13 | 0.0117 | 0.0144 | 0.016 | 0.0086 | 0.0053 | 0.0006 | 0 | 0 |
| M63 | H21R | E39R | T88R | E210R | — | 13 | 0.0311 | 0.0219 | 0.0245 | 0.0148 | 0.0075 | 0.002 | 0 | 0 |
| M64 | H21R | A61D | T88R | E210R | — | 13 | 0.052 | 0.0366 | 0.0412 | 0.0417 | 0.028 | 0.0024 | 0 | 0 |
| M65 | V17L | H21R | E39R | T88R | E210R | 13 | 0.0111 | 0.0113 | 0.006 | 0.0036 | 0.0014 | 0 | 0 | 0 |
| M66 | V17L | H21R | A61D | T88R | E210R | 13 | 0.03 | 0.034 | 0.0307 | 0.0189 | 0.0096 | 0.0004 | 0 | 0 |

Among the 24 variants tested with the inhibitors of Examples 6-10, 3 outperformed the others for specific inhibitors. These 7 variants are the following: V17L/H21R/T88R (M44) (SEQ ID NO: 75) and H21R/E507T (M72) (SEQ ID NO: 131) for heparin resistance; H21R/T88R/E210R (M48) (SEQ ID NO: 83), H21R/T88R/E507T (M83) (SEQ ID NO: 153), H21R/T88R/E210R/E507T (M97) (SEQ ID NO: 181) and H21R/A61D/T88R/E210R/E507T (M109) (SEQ ID NO: 205) for humic acid and hematin resistance; and H21R/A61D/T88R/E210R (M64) (SEQ ID NO: 115) and H21R/T88R/E210R/E507T (M97) (SEQ ID NO: 181) for NaCl, ethanol and guanidine thiocyanate (contained specifically in extraction kit components) resistance.

TABLE 10
Taq DNA Polymerase mutants

| | | SEQ |
|---|---|---|
| Mutant | | ID |
| Type | Sequence | NO |
| M8 | MRGMLPLFEPKGRVLLVDGHHLAYRTFHALKGLTTSRGRPVQAVYGFAKSLLKALKEDGD | 3 |
| (E39R/A | DVIVVFDAKAPSFRHEAYGGYKAGRAPTPEDFPRQLALIKELVDLLGLARLEVPGYEADD | |
| 61D); | VLASLAKKAEKEGYEVRILTADKDLYQLLSDRIHVLHPEGYLITPAWLWEKYGLRPDQWA | |
| Protein | DYRALTGDESDNLPGVKGIGEKTARKLLEEWGSLEALLKNLDRLKPAIREKILAHMDDLK | |
| sequence | LSWDLAKVRTDLPLEVDFAKRREPDRERLRAFLERLEFGSLLHEFGLLESPKALEEAPWP | |
| (N to C | PPEGAFVGFVLSRKEPMWADLLALAAARGGRVHRAPEPYKALRDLKEARGLLAKDLSVLA | |
| terminus) | LREGLGLPPGDDPMLLAYLLDPSNTTPEGVARRYGGEWTEEAGERAALSERLFANLWGRL | |
| | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M8 | ATGCGTGGTATGCTGCCGTTGTTCGAGCCTAAAGGCCGCGTACTGTTAGTCGATGGTCAT | 4 |
| (E39R/A | CACTTGGCCTATCGGACGTTCCATGCACTCAAAGGTCTGACGACCAGTCGTGGCCGTCCG | |
| 61D); | GTCCAGGCTGTTTATGGTTTCGCTAAGTCTTTGCTCAAAGCACTGAAAGAAGACGGGGAC | |
| DNA | GATGTAATTGTTGTATTTGATGCCAAAGCACCGAGCTTCCGCCACGAAGCTTATGGTGGC | |
| sequence | TACAAGGCAGGACGCGCCCCTACCCCAGAAGATTTCCCCCGTCAGCTGGCATTAATTAAG | |
| (5′ to 3′) | GAGTTAGTAGACCTTCTCGGCTTAGCGCGTCTGGAAGTTCCGGGTTATGAGGCGGACGAT | |
| | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M9 | MRGMLPLFEPKGRVLLVDGHRLAYRTFHALKGLTTSRGEPVQAVYGFAKSLLKALKEDGD | 5 |
| (H21R/A | DVIVVFDAKAPSFRHEAYGGYKAGRAPTPEDFPRQLALIKELVDLLGLARLEVPGYEADD | |
| 61D); | VLASLAKKAEKEGYEVRILTADKDLYQLLSDRIHVLHPEGYLITPAWLWEKYGLRPDQWA | |
| Protein | DYRALTGDESDNLPGVKGIGEKTARKLLEEWGSLEALLKNLDRLKPAIREKILAHMDDLK | |
| sequence | LSWDLAKVRTDLPLEVDFAKRREPDRERLRAFLERLEFGSLLHEFGLLESPKALEEAPWP | |
| (N to C | PPEGAFVGFVLSRKEPMWADLLALAAARGGRVHRAPEPYKALRDLKEARGLLAKDLSVLA | |
| terminus) | LREGLGLPPGDDPMLLAYLLDPSNTTPEGVARRYGGEWTEEAGERAALSERLFANLWGRL | |
| | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M9 | ATGCGTGGTATGCTGCCGTTGTTCGAGCCTAAAGGCCGCGTACTGTTAGTCGATGGTCAT | 6 |
| (H21R/A | CGTTTGGCCTATCGGACGTTCCATGCACTCAAAGGTCTGACGACCAGTCGTGGCGAACCG | |
| 61D); | GTCCAGGCTGTTTATGGTTTCGCTAAGTCTTTGCTCAAAGCACTGAAAGAAGACGGGGAC | |
| DNA | GATGTAATTGTTGTATTTGATGCCAAAGCACCGAGCTTCCGCCACGAAGCTTATGGTGGC | |
| sequence | TACAAGGCAGGACGCGCCCCTACCCCAGAAGATTTCCCCCGTCAGCTGGCATTAATTAAG | |
| (5′ to 3′) | GAGTTAGTAGACCTTCTCGGCTTAGCGCGTCTGGAAGTTCCGGGTTATGAGGCGGACGAT | |
| | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M10 | MRGMLPLFEPKGRVLLVDGHHLAYRTFHALKGLTTSRGEPVQAVYGFAKSLLKALKEDGD | 7 |
| (A61D/L | DVIVVFDAKAPSFRHEAYGGYKAGRAPTPEDFPRQLALIKELVDLLGLARLEVPGYEADD | |
| 367V); | VLASLAKKAEKEGYEVRILTADKDLYQLLSDRIHVLHPEGYLITPAWLWEKYGLRPDQWA | |
| Protein | DYRALTGDESDNLPGVKGIGEKTARKLLEEWGSLEALLKNLDRLKPAIREKILAHMDDLK | |
| sequence | LSWDLAKVRTDLPLEVDFAKRREPDRERLRAFLERLEFGSLLHEFGLLESPKALEEAPWP | |
| (N to C | PPEGAFVGFVLSRKEPMWADLLALAAARGGRVHRAPEPYKALRDLKEARGLLAKDLSVLA | |
| terminus) | LREGLGVPPGDDPMLLAYLLDPSNTTPEGVARRYGGEWTEEAGERAALSERLFANLWGRL | |
| | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M10 | ATGCGTGGTATGCTGCCGTTGTTCGAGCCTAAAGGCCGCGTACTGTTAGTCGATGGTCAT | 8 |
| (A61D/L | CACTTGGCCTATCGGACGTTCCATGCACTCAAAGGTCTGACGACCAGTCGTGGCGAACCG | |
| 367V); | GTCCAGGCTGTTTATGGTTTCGCTAAGTCTTTGCTCAAAGCACTGAAAGAAGACGGGGAC | |
| DNA | GATGTAATTGTTGTATTTGATGCCAAAGCACCGAGCTTCCGCCACGAAGCTTATGGTGGC | |
| sequence | TACAAGGCAGGACGCGCCCCTACCCCAGAAGATTTCCCCCGTCAGCTGGCATTAATTAAG | |
| (5′ to 3′) | GAGTTAGTAGACCTTCTCGGCTTAGCGCGTCTGGAAGTTCCGGGTTATGAGGCGGACGAT | |
| | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGAGTCCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M11 | MFGMLPLFEPKGRVLLVDGHHLAYRTFHALKGLTTSRGEPVQAVYGFAKSLLKALKEDGD | 9 |
| (R2F/A6 | DVIVVFDAKAPSFRHEAYGGYKAGRAPTPEDFPRQLALIKELVDLLGLARLEVPGYEADD | |
| 1D); | VLASLAKKAEKEGYEVRILTADKDLYQLLSDRIHVLHPEGYLITPAWLWEKYGLRPDQWA | |
| Protein | DYRALTGDESDNLPGVKGIGEKTARKLLEEWGSLEALLKNLDRLKPAIREKILAHMDDLK | |
| sequence | LSWDLAKVRTDLPLEVDFAKRREPDRERLRAFLERLEFGSLLHEFGLLESPKALEEAPWP | |
| (N to C | PPEGAFVGFVLSRKEPMWADLLALAAARGGRVHRAPEPYKALRDLKEARGLLAKDLSVLA | |
| terminus) | LREGLGLPPGDDPMLLAYLLDPSNTTPEGVARRYGGEWTEEAGERAALSERLFANLWGRL | |
| | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M11 | ATGTTTGGTATGCTGCCGTTGTTCGAGCCTAAAGGCCGCGTACTGTTAGTCGATGGTCAT | 10 |
| (R2F/A6 | CACTTGGCCTATCGGACGTTCCATGCACTCAAAGGTCTGACGACCAGTCGTGGCGAACCG | |
| 1D); | GTCCAGGCTGTTTATGGTTTCGCTAAGTCTTTGCTCAAAGCACTGAAAGAAGACGGGGAC | |
| DNA | GATGTAATTGTTGTATTTGATGCCAAAGCACCGAGCTTCCGCCACGAAGCTTATGGTGGC | |
| sequence | TACAAGGCAGGACGCGCCCCTACCCCAGAAGATTTCCCCCGTCAGCTGGCATTAATTAAG | |
| (5′ to 3′) | GAGTTAGTAGACCTTCTCGGCTTAGCGCGTCTGGAAGTTCCGGGTTATGAGGCGGACGAT | |
| | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M12 | MRGMLPLFEPKGRVLLVDGHHLAYRTFHALKGLTTSRGEPVQAVYGFAKSLLKALKEDGD | 11 |
| (A61D/T | DVIVVFDAKAPSFRHEAYGGYKAGRAPRPEDFPRQLALIKELVDLLGLARLEVPGYEADD | |
| 88R); | VLASLAKKAEKEGYEVRILTADKDLYQLLSDRIHVLHPEGYLITPAWLWEKYGLRPDQWA | |
| Protein | DYRALTGDESDNLPGVKGIGEKTARKLLEEWGSLEALLKNLDRLKPAIREKILAHMDDLK | |
| sequence | LSWDLAKVRTDLPLEVDFAKRREPDRERLRAFLERLEFGSLLHEFGLLESPKALEEAPWP | |
| (N to C | PPEGAFVGFVLSRKEPMWADLLALAAARGGRVHRAPEPYKALRDLKEARGLLAKDLSVLA | |
| terminus) | LREGLGLPPGDDPMLLAYLLDPSNTTPEGVARRYGGEWTEEAGERAALSERLFANLWGRL | |
| | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M12 | ATGCGTGGTATGCTGCCGTTGTTCGAGCCTAAAGGCCGCGTACTGTTAGTCGATGGTCAT | 12 |
| (A61D/T | CACTTGGCCTATCGGACGTTCCATGCACTCAAAGGTCTGACGACCAGTCGTGGCGAACCG | |
| 88R); | GTCCAGGCTGTTTATGGTTTCGCTAAGTCTTTGCTCAAAGCACTGAAAGAAGACGGGGAC | |
| DNA | GATGTAATTGTTGTATTTGATGCCAAAGCACCGAGCTTCCGCCACGAAGCTTATGGTGGC | |
| sequence | TACAAGGCAGGACGCGCCCCTCGTCCAGAAGATTTCCCCCGTCAGCTGGCATTAATTAAG | |
| (5′ to 3′) | GAGTTAGTAGACCTTCTCGGCTTAGCGCGTCTGGAAGTTCCGGGTTATGAGGCGGACGAT | |
| | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M13 | MRGMLPLFEPKGRVLLVDGHHLAYRTFHALKGLTTSRGEPVQAVYGFAKSLLKALKEDGD | 13 |
| (A61D/Y | DVIVVFDAKAPSFRHEAYGGYKAGRAPTPEDFPRQLALIKELVDLLGLARLEVPGYEADD | |
| 182S); | VLASLAKKAEKEGYEVRILTADKDLYQLLSDRIHVLHPEGYLITPAWLWEKYGLRPDQWA | |
| Protein | DSRALTGDESDNLPGVKGIGEKTARKLLEEWGSLEALLKNLDRLKPAIREKILAHMDDLK | |
| sequence | LSWDLAKVRTDLPLEVDFAKRREPDRERLRAFLERLEFGSLLHEFGLLESPKALEEAPWP | |
| (N to C | PPEGAFVGFVLSRKEPMWADLLALAAARGGRVHRAPEPYKALRDLKEARGLLAKDLSVLA | |
| terminus) | LREGLGLPPGDDPMLLAYLLDPSNTTPEGVARRYGGEWTEEAGERAALSERLFANLWGRL | |
| | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M13 | ATGCGTGGTATGCTGCCGTTGTTCGAGCCTAAAGGCCGCGTACTGTTAGTCGATGGTCAT | 14 |
| (A61D/Y | CACTTGGCCTATCGGACGTTCCATGCACTCAAAGGTCTGACGACCAGTCGTGGCGAACCG | |
| 182S); | GTCCAGGCTGTTTATGGTTTCGCTAAGTCTTTGCTCAAAGCACTGAAAGAAGACGGGGAC | |
| DNA | GATGTAATTGTTGTATTTGATGCCAAAGCACCGAGCTTCCGCCACGAAGCTTATGGTGGC | |
| sequence | TACAAGGCAGGACGCGCCCCTACCCCAGAAGATTTCCCCCGTCAGCTGGCATTAATTAAG | |
| (5′ to 3′) | GAGTTAGTAGACCTTCTCGGCTTAGCGCGTCTGGAAGTTCCGGGTTATGAGGCGGACGAT | |
| | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATAGCCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M14 | MRGMLPLFEPKGRVLLVDGHRLAYRTFHALKGLTTSRGRPVQAVYGFAKSLLKALKEDGD | 15 |
| (H21R/E | AVIVVFDAKAPSFRHEAYGGYKAGRAPTPEDFPRQLALIKELVDLLGLARLEVPGYEADD | |
| 39R); | VLASLAKKAEKEGYEVRILTADKDLYQLLSDRIHVLHPEGYLITPAWLWEKYGLRPDQWA | |
| Protein | DYRALTGDESDNLPGVKGIGEKTARKLLEEWGSLEALLKNLDRLKPAIREKILAHMDDLK | |
| sequence | LSWDLAKVRTDLPLEVDFAKRREPDRERLRAFLERLEFGSLLHEFGLLESPKALEEAPWP | |
| (N to C | PPEGAFVGFVLSRKEPMWADLLALAAARGGRVHRAPEPYKALRDLKEARGLLAKDLSVLA | |
| terminus) | LREGLGLPPGDDPMLLAYLLDPSNTTPEGVARRYGGEWTEEAGERAALSERLFANLWGRL | |
| | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M14 | ATGCGTGGTATGCTGCCGTTGTTCGAGCCTAAAGGCCGCGTACTGTTAGTCGATGGTCAT | 16 |
| (H21R/E | CGTTTGGCCTATCGGACGTTCCATGCACTCAAAGGTCTGACGACCAGTCGTGGCCGTCCG | |
| 39R); | GTCCAGGCTGTTTATGGTTTCGCTAAGTCTTTGCTCAAAGCACTGAAAGAAGACGGGGAC | |
| DNA | GCGGTAATTGTTGTATTTGATGCCAAAGCACCGAGCTTCCGCCACGAAGCTTATGGTGGC | |
| sequence | TACAAGGCAGGACGCGCCCCTACCCCAGAAGATTTCCCCCGTCAGCTGGCATTAATTAAG | |
| (5′ to 3′) | GAGTTAGTAGACCTTCTCGGCTTAGCGCGTCTGGAAGTTCCGGGTTATGAGGCGGACGAT | |
| | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M15 | MRGMLPLFEPKGRVLLVDGHHLAYRTFHALKGLTTSRGRPVQAVYGFAKSLLKALKEDGD | 17 |
| (E39R/L | AVIVVFDAKAPSFRHEAYGGYKAGRAPTPEDFPRQLALIKELVDLLGLARLEVPGYEADD | |
| 367V); | VLASLAKKAEKEGYEVRILTADKDLYQLLSDRIHVLHPEGYLITPAWLWEKYGLRPDQWA | |
| Protein | DYRALTGDESDNLPGVKGIGEKTARKLLEEWGSLEALLKNLDRLKPAIREKILAHMDDLK | |
| sequence | LSWDLAKVRTDLPLEVDFAKRREPDRERLRAFLERLEFGSLLHEFGLLESPKALEEAPWP | |
| (N to C | PPEGAFVGFVLSRKEPMWADLLALAAARGGRVHRAPEPYKALRDLKEARGLLAKDLSVLA | |
| terminus) | LREGLGVPPGDDPMLLAYLLDPSNTTPEGVARRYGGEWTEEAGERAALSERLFANLWGRL | |
| | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M15 | ATGCGTGGTATGCTGCCGTTGTTCGAGCCTAAAGGCCGCGTACTGTTAGTCGATGGTCAT | 18 |
| (E39R/L | CACTTGGCCTATCGGACGTTCCATGCACTCAAAGGTCTGACGACCAGTCGTGGCCGTCCG | |
| 367V); | GTCCAGGCTGTTTATGGTTTCGCTAAGTCTTTGCTCAAAGCACTGAAAGAAGACGGGGAC | |
| DNA | GCGGTAATTGTTGTATTTGATGCCAAAGCACCGAGCTTCCGCCACGAAGCTTATGGTGGC | |
| sequence | TACAAGGCAGGACGCGCCCCTACCCCAGAAGATTTCCCCCGTCAGCTGGCATTAATTAAG | |
| (5′ to 3′) | GAGTTAGTAGACCTTCTCGGCTTAGCGCGTCTGGAAGTTCCGGGTTATGAGGCGGACGAT | |
| | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGAGTCCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M16 | MFGMLPLFEPKGRVLLVDGHHLAYRTFHALKGLTTSRGRPVQAVYGFAKSLLKALKEDGD | 19 |
| (R2F/E39 | AVIVVFDAKAPSFRHEAYGGYKAGRAPTPEDFPRQLALIKELVDLLGLARLEVPGYEADD | |
| R); | VLASLAKKAEKEGYEVRILTADKDLYQLLSDRIHVLHPEGYLITPAWLWEKYGLRPDQWA | |
| Protein | DYRALTGDESDNLPGVKGIGEKTARKLLEEWGSLEALLKNLDRLKPAIREKILAHMDDLK | |
| sequence | LSWDLAKVRTDLPLEVDFAKRREPDRERLRAFLERLEFGSLLHEFGLLESPKALEEAPWP | |
| (N to C | PPEGAFVGFVLSRKEPMWADLLALAAARGGRVHRAPEPYKALRDLKEARGLLAKDLSVLA | |
| terminus) | LREGLGLPPGDDPMLLAYLLDPSNTTPEGVARRYGGEWTEEAGERAALSERLFANLWGRL | |
| | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M16 | ATGTTTGGTATGCTGCCGTTGTTCGAGCCTAAAGGCCGCGTACTGTTAGTCGATGGTCAT | 20 |
| (R2F/E39 | CACTTGGCCTATCGGACGTTCCATGCACTCAAAGGTCTGACGACCAGTCGTGGCCGTCCG | |
| R); DNA | GTCCAGGCTGTTTATGGTTTCGCTAAGTCTTTGCTCAAAGCACTGAAAGAAGACGGGGAC | |
| sequence | GCGGTAATTGTTGTATTTGATGCCAAAGCACCGAGCTTCCGCCACGAAGCTTATGGTGGC | |
| (5′ to 3′) | TACAAGGCAGGACGCGCCCCTACCCCAGAAGATTTCCCCCGTCAGCTGGCATTAATTAAG | |
| | GAGTTAGTAGACCTTCTCGGCTTAGCGCGTCTGGAAGTTCCGGGTTATGAGGCGGACGAT | |
| | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M17 | MRGMLPLFEPKGRVLLVDGHHLAYRTFHALKGLTTSRGRPVQAVYGFAKSLLKALKEDGD | 21 |
| (E39R/T | AVIVVFDAKAPSFRHEAYGGYKAGRAPRPEDFPRQLALIKELVDLLGLARLEVPGYEADD | |
| 88R); | VLASLAKKAEKEGYEVRILTADKDLYQLLSDRIHVLHPEGYLITPAWLWEKYGLRPDQWA | |
| Protein | DYRALTGDESDNLPGVKGIGEKTARKLLEEWGSLEALLKNLDRLKPAIREKILAHMDDLK | |
| sequence | LSWDLAKVRTDLPLEVDFAKRREPDRERLRAFLERLEFGSLLHEFGLLESPKALEEAPWP | |
| (N to C | PPEGAFVGFVLSRKEPMWADLLALAAARGGRVHRAPEPYKALRDLKEARGLLAKDLSVLA | |
| terminus) | LREGLGLPPGDDPMLLAYLLDPSNTTPEGVARRYGGEWTEEAGERAALSERLFANLWGRL | |
| | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M17 | ATGCGTGGTATGCTGCCGTTGTTCGAGCCTAAAGGCCGCGTACTGTTAGTCGATGGTCAT | 22 |
| (E39R/T | CACTTGGCCTATCGGACGTTCCATGCACTCAAAGGTCTGACGACCAGTCGTGGCCGTCCG | |
| 88R); | GTCCAGGCTGTTTATGGTTTCGCTAAGTCTTTGCTCAAAGCACTGAAAGAAGACGGGGAC | |
| DNA | GCGGTAATTGTTGTATTTGATGCCAAAGCACCGAGCTTCCGCCACGAAGCTTATGGTGGC | |
| sequence | TACAAGGCAGGACGCGCCCCTCGTCCAGAAGATTTCCCCCGTCAGCTGGCATTAATTAAG | |
| (5′ to 3′) | GAGTTAGTAGACCTTCTCGGCTTAGCGCGTCTGGAAGTTCCGGGTTATGAGGCGGACGAT | |
| | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M18 | MRGMLPLFEPKGRVLLVDGHHLAYRTFHALKGLTTSRGRPVQAVYGFAKSLLKALKEDGD | 23 |
| (E39R/Y | AVIVVFDAKAPSFRHEAYGGYKAGRAPTPEDFPRQLALIKELVDLLGLARLEVPGYEADD | |
| 182S); | VLASLAKKAEKEGYEVRILTADKDLYQLLSDRIHVLHPEGYLITPAWLWEKYGLRPDQWA | |
| Protein | DSRALTGDESDNLPGVKGIGEKTARKLLEEWGSLEALLKNLDRLKPAIREKILAHMDDLK | |
| sequence | LSWDLAKVRTDLPLEVDFAKRREPDRERLRAFLERLEFGSLLHEFGLLESPKALEEAPWP | |
| (N to C | PPEGAFVGFVLSRKEPMWADLLALAAARGGRVHRAPEPYKALRDLKEARGLLAKDLSVLA | |
| terminus) | LREGLGLPPGDDPMLLAYLLDPSNTTPEGVARRYGGEWTEEAGERAALSERLFANLWGRL | |
| | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M18 | ATGCGTGGTATGCTGCCGTTGTTCGAGCCTAAAGGCCGCGTACTGTTAGTCGATGGTCAT | 24 |
| (E39R/Y | CACTTGGCCTATCGGACGTTCCATGCACTCAAAGGTCTGACGACCAGTCGTGGCCGTCCG | |
| 182S); | GTCCAGGCTGTTTATGGTTTCGCTAAGTCTTTGCTCAAAGCACTGAAAGAAGACGGGGAC | |
| DNA | GCGGTAATTGTTGTATTTGATGCCAAAGCACCGAGCTTCCGCCACGAAGCTTATGGTGGC | |
| sequence | TACAAGGCAGGACGCGCCCCTACCCCAGAAGATTTCCCCCGTCAGCTGGCATTAATTAAG | |
| (5′ to 3′) | GAGTTAGTAGACCTTCTCGGCTTAGCGCGTCTGGAAGTTCCGGGTTATGAGGCGGACGAT | |
| | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATAGCCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M19 | MRGMLPLFEPKGRVLLVDGHRLAYRTFHALKGLTTSRGEPVQAVYGFAKSLLKALKEDGD | 25 |
| (H21R/L | AVIVVFDAKAPSFRHEAYGGYKAGRAPTPEDFPRQLALIKELVDLLGLARLEVPGYEADD | |
| 367V); | VLASLAKKAEKEGYEVRILTADKDLYQLLSDRIHVLHPEGYLITPAWLWEKYGLRPDQWA | |
| Protein | DYRALTGDESDNLPGVKGIGEKTARKLLEEWGSLEALLKNLDRLKPAIREKILAHMDDLK | |
| sequence | LSWDLAKVRTDLPLEVDFAKRREPDRERLRAFLERLEFGSLLHEFGLLESPKALEEAPWP | |
| (N to C | PPEGAFVGFVLSRKEPMWADLLALAAARGGRVHRAPEPYKALRDLKEARGLLAKDLSVLA | |
| terminus) | LREGLGVPPGDDPMLLAYLLDPSNTTPEGVARRYGGEWTEEAGERAALSERLFANLWGRL | |
| | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M19 | ATGCGTGGTATGCTGCCGTTGTTCGAGCCTAAAGGCCGCGTACTGTTAGTCGATGGTCAT | 26 |
| (H21R/L | CGTTTGGCCTATCGGACGTTCCATGCACTCAAAGGTCTGACGACCAGTCGTGGCGAACCG | |
| 367V); | GTCCAGGCTGTTTATGGTTTCGCTAAGTCTTTGCTCAAAGCACTGAAAGAAGACGGGGAC | |
| DNA | GCGGTAATTGTTGTATTTGATGCCAAAGCACCGAGCTTCCGCCACGAAGCTTATGGTGGC | |
| sequence | TACAAGGCAGGACGCGCCCCTACCCCAGAAGATTTCCCCCGTCAGCTGGCATTAATTAAG | |
| (5′ to 3′) | GAGTTAGTAGACCTTCTCGGCTTAGCGCGTCTGGAAGTTCCGGGTTATGAGGCGGACGAT | |
| | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGAGTCCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M20 | MFGMLPLFEPKGRVLLVDGHRLAYRTFHALKGLTTSRGEPVQAVYGFAKSLLKALKEDGD | 27 |
| (R2F/H2 | AVIVVFDAKAPSFRHEAYGGYKAGRAPTPEDFPRQLALIKELVDLLGLARLEVPGYEADD | |
| 1R); | VLASLAKKAEKEGYEVRILTADKDLYQLLSDRIHVLHPEGYLITPAWLWEKYGLRPDQWA | |
| Protein | DYRALTGDESDNLPGVKGIGEKTARKLLEEWGSLEALLKNLDRLKPAIREKILAHMDDLK | |
| sequence | LSWDLAKVRTDLPLEVDFAKRREPDRERLRAFLERLEFGSLLHEFGLLESPKALEEAPWP | |
| (N to C | PPEGAFVGFVLSRKEPMWADLLALAAARGGRVHRAPEPYKALRDLKEARGLLAKDLSVLA | |
| terminus) | LREGLGLPPGDDPMLLAYLLDPSNTTPEGVARRYGGEWTEEAGERAALSERLFANLWGRL | |
| | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M20 | ATGTTTGGTATGCTGCCGTTGTTCGAGCCTAAAGGCCGCGTACTGTTAGTCGATGGTCAT | 28 |
| (R2F/H2 | CGTTTGGCCTATCGGACGTTCCATGCACTCAAAGGTCTGACGACCAGTCGTGGCGAACCG | |
| 1R); | GTCCAGGCTGTTTATGGTTTCGCTAAGTCTTTGCTCAAAGCACTGAAAGAAGACGGGGAC | |
| DNA | GCGGTAATTGTTGTATTTGATGCCAAAGCACCGAGCTTCCGCCACGAAGCTTATGGTGGC | |
| sequence | TACAAGGCAGGACGCGCCCCTACCCCAGAAGATTTCCCCCGTCAGCTGGCATTAATTAAG | |
| (5′ to 3′) | GAGTTAGTAGACCTTCTCGGCTTAGCGCGTCTGGAAGTTCCGGGTTATGAGGCGGACGAT | |
| | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M21 | MRGMLPLFEPKGRVLLVDGHRLAYRTFHALKGLTTSRGEPVQAVYGFAKSLLKALKEDGD | 29 |
| (H21R/T | AVIVVFDAKAPSFRHEAYGGYKAGRAPRPEDFPRQLALIKELVDLLGLARLEVPGYEADD | |
| 88R); | VLASLAKKAEKEGYEVRILTADKDLYQLLSDRIHVLHPEGYLITPAWLWEKYGLRPDQWA | |
| Protein | DYRALTGDESDNLPGVKGIGEKTARKLLEEWGSLEALLKNLDRLKPAIREKILAHMDDLK | |
| sequence | LSWDLAKVRTDLPLEVDFAKRREPDRERLRAFLERLEFGSLLHEFGLLESPKALEEAPWP | |
| (N to C | PPEGAFVGFVLSRKEPMWADLLALAAARGGRVHRAPEPYKALRDLKEARGLLAKDLSVLA | |
| terminus) | LREGLGLPPGDDPMLLAYLLDPSNTTPEGVARRYGGEWTEEAGERAALSERLFANLWGRL | |
| | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M21 | ATGCGTGGTATGCTGCCGTTGTTCGAGCCTAAAGGCCGCGTACTGTTAGTCGATGGTCAT | 30 |
| (H21R/T | CGTTTGGCCTATCGGACGTTCCATGCACTCAAAGGTCTGACGACCAGTCGTGGCGAACCG | |
| 88R); | GTCCAGGCTGTTTATGGTTTCGCTAAGTCTTTGCTCAAAGCACTGAAAGAAGACGGGGAC | |
| DNA | GCGGTAATTGTTGTATTTGATGCCAAAGCACCGAGCTTCCGCCACGAAGCTTATGGTGGC | |
| sequence | TACAAGGCAGGACGCGCCCCTCGTCCAGAAGATTTCCCCCGTCAGCTGGCATTAATTAAG | |
| (5′ to 3′) | GAGTTAGTAGACCTTCTCGGCTTAGCGCGTCTGGAAGTTCCGGGTTATGAGGCGGACGAT | |
| | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M22 | MRGMLPLFEPKGRVLLVDGHRLAYRTFHALKGLTTSRGEPVQAVYGFAKSLLKALKEDGD | 31 |
| (H21R/Y | AVIVVFDAKAPSFRHEAYGGYKAGRAPTPEDFPRQLALIKELVDLLGLARLEVPGYEADD | |
| 182S); | VLASLAKKAEKEGYEVRILTADKDLYQLLSDRIHVLHPEGYLITPAWLWEKYGLRPDQWA | |
| Protein | DSRALTGDESDNLPGVKGIGEKTARKLLEEWGSLEALLKNLDRLKPAIREKILAHMDDLK | |
| sequence | LSWDLAKVRTDLPLEVDFAKRREPDRERLRAFLERLEFGSLLHEFGLLESPKALEEAPWP | |
| (N to C | PPEGAFVGFVLSRKEPMWADLLALAAARGGRVHRAPEPYKALRDLKEARGLLAKDLSVLA | |
| terminus) | LREGLGLPPGDDPMLLAYLLDPSNTTPEGVARRYGGEWTEEAGERAALSERLFANLWGRL | |
| | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M22 | ATGCGTGGTATGCTGCCGTTGTTCGAGCCTAAAGGCCGCGTACTGTTAGTCGATGGTCAT | 32 |
| (H21R/Y | CGTTTGGCCTATCGGACGTTCCATGCACTCAAAGGTCTGACGACCAGTCGTGGCGAACCG | |
| 182S); | GTCCAGGCTGTTTATGGTTTCGCTAAGTCTTTGCTCAAAGCACTGAAAGAAGACGGGGAC | |
| DNA | GCGGTAATTGTTGTATTTGATGCCAAAGCACCGAGCTTCCGCCACGAAGCTTATGGTGGC | |
| sequence | TACAAGGCAGGACGCGCCCCTACCCCAGAAGATTTCCCCCGTCAGCTGGCATTAATTAAG | |
| (5′ to 3′) | GAGTTAGTAGACCTTCTCGGCTTAGCGCGTCTGGAAGTTCCGGGTTATGAGGCGGACGAT | |
| | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATAGCCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M23 | MFGMLPLFEPKGRVLLVDGHHLAYRTFHALKGLTTSRGEPVQAVYGFAKSLLKALKEDGD | 33 |
| (R2F/L36 | AVIVVFDAKAPSFRHEAYGGYKAGRAPTPEDFPRQLALIKELVDLLGLARLEVPGYEADD | |
| 7V); | VLASLAKKAEKEGYEVRILTADKDLYQLLSDRIHVLHPEGYLITPAWLWEKYGLRPDQWA | |
| Protein | DYRALTGDESDNLPGVKGIGEKTARKLLEEWGSLEALLKNLDRLKPAIREKILAHMDDLK | |
| sequence | LSWDLAKVRTDLPLEVDFAKRREPDRERLRAFLERLEFGSLLHEFGLLESPKALEEAPWP | |
| (N to C | PPEGAFVGFVLSRKEPMWADLLALAAARGGRVHRAPEPYKALRDLKEARGLLAKDLSVLA | |
| terminus) | LREGLGVPPGDDPMLLAYLLDPSNTTPEGVARRYGGEWTEEAGERAALSERLFANLWGRL | |
| | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M23 | ATGTTTGGTATGCTGCCGTTGTTCGAGCCTAAAGGCCGCGTACTGTTAGTCGATGGTCAT | 34 |
| (R2F/L36 | CACTTGGCCTATCGGACGTTCCATGCACTCAAAGGTCTGACGACCAGTCGTGGCGAACCG | |
| 7V); | GTCCAGGCTGTTTATGGTTTCGCTAAGTCTTTGCTCAAAGCACTGAAAGAAGACGGGGAC | |
| DNA | GCGGTAATTGTTGTATTTGATGCCAAAGCACCGAGCTTCCGCCACGAAGCTTATGGTGGC | |
| sequence | TACAAGGCAGGACGCGCCCCTACCCCAGAAGATTTCCCCCGTCAGCTGGCATTAATTAAG | |
| (5′ to 3′) | GAGTTAGTAGACCTTCTCGGCTTAGCGCGTCTGGAAGTTCCGGGTTATGAGGCGGACGAT | |
| | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGAGTCCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M24 | MRGMLPLFEPKGRVLLVDGHHLAYRTFHALKGLTTSRGEPVQAVYGFAKSLLKALKEDGD | 35 |
| (T88R/L | AVIVVFDAKAPSFRHEAYGGYKAGRAPRPEDFPRQLALIKELVDLLGLARLEVPGYEADD | |
| 367V); | VLASLAKKAEKEGYEVRILTADKDLYQLLSDRIHVLHPEGYLITPAWLWEKYGLRPDQWA | |
| Protein | DYRALTGDESDNLPGVKGIGEKTARKLLEEWGSLEALLKNLDRLKPAIREKILAHMDDLK | |
| sequence | LSWDLAKVRTDLPLEVDFAKRREPDRERLRAFLERLEFGSLLHEFGLLESPKALEEAPWP | |
| (N to C | PPEGAFVGFVLSRKEPMWADLLALAAARGGRVHRAPEPYKALRDLKEARGLLAKDLSVLA | |
| terminus) | LREGLGVPPGDDPMLLAYLLDPSNTTPEGVARRYGGEWTEEAGERAALSERLFANLWGRL | |
| | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M24 | ATGCGTGGTATGCTGCCGTTGTTCGAGCCTAAAGGCCGCGTACTGTTAGTCGATGGTCAT | 36 |
| (T88R/L | CACTTGGCCTATCGGACGTTCCATGCACTCAAAGGTCTGACGACCAGTCGTGGCGAACCG | |
| 367V); | GTCCAGGCTGTTTATGGTTTCGCTAAGTCTTTGCTCAAAGCACTGAAAGAAGACGGGGAC | |
| DNA | GCGGTAATTGTTGTATTTGATGCCAAAGCACCGAGCTTCCGCCACGAAGCTTATGGTGGC | |
| sequence | TACAAGGCAGGACGCGCCCCTCGTCCAGAAGATTTCCCCCGTCAGCTGGCATTAATTAAG | |
| (5′ to 3′) | GAGTTAGTAGACCTTCTCGGCTTAGCGCGTCTGGAAGTTCCGGGTTATGAGGCGGACGAT | |
| | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGAGTCCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M25 | MRGMLPLFEPKGRVLLVDGHHLAYRTFHALKGLTTSRGEPVQAVYGFAKSLLKALKEDGD | 37 |
| (Y182S/ | AVIVVFDAKAPSFRHEAYGGYKAGRAPTPEDFPRQLALIKELVDLLGLARLEVPGYEADD | |
| L367V); | VLASLAKKAEKEGYEVRILTADKDLYQLLSDRIHVLHPEGYLITPAWLWEKYGLRPDQWA | |
| Protein | DSRALTGDESDNLPGVKGIGEKTARKLLEEWGSLEALLKNLDRLKPAIREKILAHMDDLK | |
| sequence | LSWDLAKVRTDLPLEVDFAKRREPDRERLRAFLERLEFGSLLHEFGLLESPKALEEAPWP | |
| (N to C | PPEGAFVGFVLSRKEPMWADLLALAAARGGRVHRAPEPYKALRDLKEARGLLAKDLSVLA | |
| terminus) | LREGLGVPPGDDPMLLAYLLDPSNTTPEGVARRYGGEWTEEAGERAALSERLFANLWGRL | |
| | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M25 | ATGCGTGGTATGCTGCCGTTGTTCGAGCCTAAAGGCCGCGTACTGTTAGTCGATGGTCAT | 38 |
| (Y182S/ | CACTTGGCCTATCGGACGTTCCATGCACTCAAAGGTCTGACGACCAGTCGTGGCGAACCG | |
| L367V); | GTCCAGGCTGTTTATGGTTTCGCTAAGTCTTTGCTCAAAGCACTGAAAGAAGACGGGGAC | |
| DNA | GCGGTAATTGTTGTATTTGATGCCAAAGCACCGAGCTTCCGCCACGAAGCTTATGGTGGC | |
| sequence | TACAAGGCAGGACGCGCCCCTACCCCAGAAGATTTCCCCCGTCAGCTGGCATTAATTAAG | |
| (5′ to 3′) | GAGTTAGTAGACCTTCTCGGCTTAGCGCGTCTGGAAGTTCCGGGTTATGAGGCGGACGAT | |
| | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATAGCCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGAGTCCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M26 | MFGMLPLFEPKGRVLLVDGHHLAYRTFHALKGLTTSRGEPVQAVYGFAKSLLKALKEDGD | 39 |
| (R2F/T88 | AVIVVFDAKAPSFRHEAYGGYKAGRAPRPEDFPRQLALIKELVDLLGLARLEVPGYEADD | |
| R); | VLASLAKKAEKEGYEVRILTADKDLYQLLSDRIHVLHPEGYLITPAWLWEKYGLRPDQWA | |
| Protein | DYRALTGDESDNLPGVKGIGEKTARKLLEEWGSLEALLKNLDRLKPAIREKILAHMDDLK | |
| sequence | LSWDLAKVRTDLPLEVDFAKRREPDRERLRAFLERLEFGSLLHEFGLLESPKALEEAPWP | |
| (N to C | PPEGAFVGFVLSRKEPMWADLLALAAARGGRVHRAPEPYKALRDLKEARGLLAKDLSVLA | |
| terminus) | LREGLGLPPGDDPMLLAYLLDPSNTTPEGVARRYGGEWTEEAGERAALSERLFANLWGRL | |
| | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M26 | ATGTTTGGTATGCTGCCGTTGTTCGAGCCTAAAGGCCGCGTACTGTTAGTCGATGGTCAT | 40 |
| (R2F/T88 | CACTTGGCCTATCGGACGTTCCATGCACTCAAAGGTCTGACGACCAGTCGTGGCGAACCG | |
| R); DNA | GTCCAGGCTGTTTATGGTTTCGCTAAGTCTTTGCTCAAAGCACTGAAAGAAGACGGGGAC | |
| sequence | GCGGTAATTGTTGTATTTGATGCCAAAGCACCGAGCTTCCGCCACGAAGCTTATGGTGGC | |
| (5′ to 3′) | TACAAGGCAGGACGCGCCCCTCGTCCAGAAGATTTCCCCCGTCAGCTGGCATTAATTAAG | |
| | GAGTTAGTAGACCTTCTCGGCTTAGCGCGTCTGGAAGTTCCGGGTTATGAGGCGGACGAT | |
| | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M27 | MFGMLPLFEPKGRVLLVDGHHLAYRTFHALKGLTTSRGEPVQAVYGFAKSLLKALKEDGD | 41 |
| (R2F/Y1 | AVIVVFDAKAPSFRHEAYGGYKAGRAPTPEDFPRQLALIKELVDLLGLARLEVPGYEADD | |
| 82S); | VLASLAKKAEKEGYEVRILTADKDLYQLLSDRIHVLHPEGYLITPAWLWEKYGLRPDQWA | |
| Protein | DSRALTGDESDNLPGVKGIGEKTARKLLEEWGSLEALLKNLDRLKPAIREKILAHMDDLK | |
| sequence | LSWDLAKVRTDLPLEVDFAKRREPDRERLRAFLERLEFGSLLHEFGLLESPKALEEAPWP | |
| (N to C | PPEGAFVGFVLSRKEPMWADLLALAAARGGRVHRAPEPYKALRDLKEARGLLAKDLSVLA | |
| terminus) | LREGLGLPPGDDPMLLAYLLDPSNTTPEGVARRYGGEWTEEAGERAALSERLFANLWGRL | |
| | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M27 | ATGTTTGGTATGCTGCCGTTGTTCGAGCCTAAAGGCCGCGTACTGTTAGTCGATGGTCAT | 42 |
| (R2F/Y1 | CACTTGGCCTATCGGACGTTCCATGCACTCAAAGGTCTGACGACCAGTCGTGGCGAACCG | |
| 82S); | GTCCAGGCTGTTTATGGTTTCGCTAAGTCTTTGCTCAAAGCACTGAAAGAAGACGGGGAC | |
| DNA | GCGGTAATTGTTGTATTTGATGCCAAAGCACCGAGCTTCCGCCACGAAGCTTATGGTGGC | |
| sequence | TACAAGGCAGGACGCGCCCCTACCCCAGAAGATTTCCCCCGTCAGCTGGCATTAATTAAG | |
| (5′ to 3′) | GAGTTAGTAGACCTTCTCGGCTTAGCGCGTCTGGAAGTTCCGGGTTATGAGGCGGACGAT | |
| | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATAGCCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M28 | MRGMLPLFEPKGRVLLVDGHHLAYRTFHALKGLTTSRGEPVQAVYGFAKSLLKALKEDGD | 43 |
| (T88R/Y | AVIVVFDAKAPSFRHEAYGGYKAGRAPRPEDFPRQLALIKELVDLLGLARLEVPGYEADD | |
| 182S); | VLASLAKKAEKEGYEVRILTADKDLYQLLSDRIHVLHPEGYLITPAWLWEKYGLRPDQWA | |
| Protein | DSRALTGDESDNLPGVKGIGEKTARKLLEEWGSLEALLKNLDRLKPAIREKILAHMDDLK | |
| sequence | LSWDLAKVRTDLPLEVDFAKRREPDRERLRAFLERLEFGSLLHEFGLLESPKALEEAPWP | |
| (N to C | PPEGAFVGFVLSRKEPMWADLLALAAARGGRVHRAPEPYKALRDLKEARGLLAKDLSVLA | |
| terminus) | LREGLGLPPGDDPMLLAYLLDPSNTTPEGVARRYGGEWTEEAGERAALSERLFANLWGRL | |
| | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M28 | ATGCGTGGTATGCTGCCGTTGTTCGAGCCTAAAGGCCGCGTACTGTTAGTCGATGGTCAT | 44 |
| (T88R/Y | CACTTGGCCTATCGGACGTTCCATGCACTCAAAGGTCTGACGACCAGTCGTGGCGAACCG | |
| 182S); | GTCCAGGCTGTTTATGGTTTCGCTAAGTCTTTGCTCAAAGCACTGAAAGAAGACGGGGAC | |
| DNA | GCGGTAATTGTTGTATTTGATGCCAAAGCACCGAGCTTCCGCCACGAAGCTTATGGTGGC | |
| sequence | TACAAGGCAGGACGCGCCCCTCGTCCAGAAGATTTCCCCCGTCAGCTGGCATTAATTAAG | |
| (5′ to 3′) | GAGTTAGTAGACCTTCTCGGCTTAGCGCGTCTGGAAGTTCCGGGTTATGAGGCGGACGAT | |
| | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATAGCCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M29 | MRGMLPLFEPKGRVLLVDGHRLAYRTFHALKGLTTSRGRPVQAVYGFAKSLLKALKEDGD | 45 |
| (H21R/E | AVIVVFDAKAPSFRHEAYGGYKAGRAPRPEDFPRQLALIKELVDLLGLARLEVPGYEADD | |
| 39R/T88 | VLASLAKKAEKEGYEVRILTADKDLYQLLSDRIHVLHPEGYLITPAWLWEKYGLRPDQWA | |
| R); | DYRALTGDESDNLPGVKGIGEKTARKLLEEWGSLEALLKNLDRLKPAIREKILAHMDDLK | |
| Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| (N to C | LREGLGLPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M29 | ATGCGTGGTATGCTGCCGTTGTTCGAGCCTAAAGGCCGCGTACTGTTAGTCGATGGTCAT | 46 |
| (H21R/E | CGTTTGGCCTATCGGACGTTCCATGCACTCAAAGGTCTGACGACCAGTCGTGGCCGTCCG | |
| 39R/T88 | GTCCAGGCTGTTTATGGTTTCGCTAAGTCTTTGCTCAAAGCACTGAAAGAAGACGGGGAC | |
| R); DNA | GCGGTAATTGTTGTATTTGATGCCAAAGCACCGAGCTTCCGCCACGAAGCTTATGGTGGC | |
| sequence | TACAAGGCAGGACGCGCCCCTCGTCCAGAAGATTTCCCCCGTCAGCTGGCATTAATTAAG | |
| (5′ to 3′) | GAGTTAGTAGACCTTCTCGGCTTAGCGCGTCTGGAAGTTCCGGGTTATGAGGCGGACGAT | |
| | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M30 | MRGMLPLFEPKGRVLLVDGHRLAYRTFHALKGLTTSRGEPVQAVYGFAKSLLKALKEDGD | 47 |
| (H21R/T | AVIVVFDAKAPSFRHEAYGGYKAGRAPRPEDFPRQLALIKELVDLLGLARLEVPGYEADD | |
| 88R/L36 | VLASLAKKAEKEGYEVRILTADKDLYQLLSDRIHVLHPEGYLITPAWLWEKYGLRPDQWA | |
| 7V); | DYRALTGDESDNLPGVKGIGEKTARKLLEEWGSLEALLKNLDRLKPAIREKILAHMDDLK | |
| Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| (N to C | LREGLGVPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M30 | ATGCGTGGTATGCTGCCGTTGTTCGAGCCTAAAGGCCGCGTACTGTTAGTCGATGGTCAT | 48 |
| (H21R/T | CGTTTGGCCTATCGGACGTTCCATGCACTCAAAGGTCTGACGACCAGTCGTGGCGAACCG | |
| 88R/L36 | GTCCAGGCTGTTTATGGTTTCGCTAAGTCTTTGCTCAAAGCACTGAAAGAAGACGGGGAC | |
| 7V); | GCGGTAATTGTTGTATTTGATGCCAAAGCACCGAGCTTCCGCCACGAAGCTTATGGTGGC | |
| DNA | TACAAGGCAGGACGCGCCCCTCGTCCAGAAGATTTCCCCCGTCAGCTGGCATTAATTAAG | |
| sequence | GAGTTAGTAGACCTTCTCGGCTTAGCGCGTCTGGAAGTTCCGGGTTATGAGGCGGACGAT | |
| (5′ to 3′) | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGAGTCCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M31 | MFGMLPLFEPKGRVLLVDGHRLAYRTFHALKGLTTSRGEPVQAVYGFAKSLLKALKEDGD | 49 |
| (R2F/H2 | AVIVVFDAKAPSFRHEAYGGYKAGRAPRPEDFPRQLALIKELVDLLGLARLEVPGYEADD | |
| 1R/T88R | VLASLAKKAEKEGYEVRILTADKDLYQLLSDRIHVLHPEGYLITPAWLWEKYGLRPDQWA | |
| ); Protein | DYRALTGDESDNLPGVKGIGEKTARKLLEEWGSLEALLKNLDRLKPAIREKILAHMDDLK | |
| sequence | LSWDLAKVRTDLPLEVDFAKRREPDRERLRAFLERLEFGSLLHEFGLLESPKALEEAPWP | |
| (N to C | PPEGAFVGFVLSRKEPMWADLLALAAARGGRVHRAPEPYKALRDLKEARGLLAKDLSVLA | |
| terminus) | LREGLGLPPGDDPMLLAYLLDPSNTTPEGVARRYGGEWTEEAGERAALSERLFANLWGRL | |
| | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M31 | ATGTTTGGTATGCTGCCGTTGTTCGAGCCTAAAGGCCGCGTACTGTTAGTCGATGGTCAT | 50 |
| (R2F/H2 | CGTTTGGCCTATCGGACGTTCCATGCACTCAAAGGTCTGACGACCAGTCGTGGCGAACCG | |
| 1R/T88R | GTCCAGGCTGTTTATGGTTTCGCTAAGTCTTTGCTCAAAGCACTGAAAGAAGACGGGGAC | |
| ); DNA | GCGGTAATTGTTGTATTTGATGCCAAAGCACCGAGCTTCCGCCACGAAGCTTATGGTGGC | |
| sequence | TACAAGGCAGGACGCGCCCCTCGTCCAGAAGATTTCCCCCGTCAGCTGGCATTAATTAAG | |
| (5′ to 3′) | GAGTTAGTAGACCTTCTCGGCTTAGCGCGTCTGGAAGTTCCGGGTTATGAGGCGGACGAT | |
| | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M32 | MRGMLPLFEPKGRVLLVDGHRLAYRTFHALKGLTTSRGEPVQAVYGFAKSLLKALKEDGD | 51 |
| (H21R/ | AVIVVFDAKAPSFRHEAYGGYKAGRAPRPEDFPRQLALIKELVDLLGLARLEVPGYEADD | |
| T88R/ | VLASLAKKAEKEGYEVRILTADKDLYQLLSDRIHVLHPEGYLITPAWLWEKYGLRPDQWA | |
| Y182S); | DSRALTGDESDNLPGVKGIGEKTARKLLEEWGSLEALLKNLDRLKPAIREKILAHMDDLK | |
| Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| (N to C | LREGLGLPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M32 | ATGCGTGGTATGCTGCCGTTGTTCGAGCCTAAAGGCCGCGTACTGTTAGTCGATGGTCAT | 52 |
| (H21R/ | CGTTTGGCCTATCGGACGTTCCATGCACTCAAAGGTCTGACGACCAGTCGTGGCGAACCG | |
| T88R/ | GTCCAGGCTGTTTATGGTTTCGCTAAGTCTTTGCTCAAAGCACTGAAAGAAGACGGGGAC | |
| Y182S); | GCGGTAATTGTTGTATTTGATGCCAAAGCACCGAGCTTCCGCCACGAAGCTTATGGTGGC | |
| DNA | TACAAGGCAGGACGCGCCCCTCGTCCAGAAGATTTCCCCCGTCAGCTGGCATTAATTAAG | |
| sequence | GAGTTAGTAGACCTTCTCGGCTTAGCGCGTCTGGAAGTTCCGGGTTATGAGGCGGACGAT | |
| (5′ to 3′) | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATAGCCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M33 | MRGMLPLFEPKGRVLLVDGHRLAYRTFHALKGLTTSRGEPVQAVYGFAKSLLKALKEDGD | 53 |
| (H21R/ | DVIVVFDAKAPSFRHEAYGGYKAGRAPRPEDFPRQLALIKELVDLLGLARLEVPGYEADD | |
| A61D/ | VLASLAKKAEKEGYEVRILTADKDLYQLLSDRIHV | |
| T88R); | LHPEGYLITPAWLWEKYGLRPDQWADYRALTGDESDNLPGVKGIGEKTARKLLEEWGSLE | |
| Protein | ALLKNLDRLKPAIREKILAHMDDLKLSWDLAKVRTDLPLEVDFAKRREPDRERLRAFLER | |
| sequence | LEFGSLLHEFGLLESPKALEEAPWPPPEGAFVGFVLSRKEPMWADLLALAAARGGRVHRA | |
| (N to C | PEPYKALRDLKEARGLLAKDLSVLALREGLGLPPGDDPMLLAYLLDPSNTTPEGVARRYG | |
| terminus) | GEWTEEAGERAALSERLFANLWGRLEGEERLLWLYREVERPLSAVLAHMEATGVRLDVAY | |
| | LRALSLEVAEEIARLEAEVFRLAGHPFNLNSRDQLERVLFDELGLPAIGKTEKTGKRSTS | |
| | AAVLEALREAHPIVEKILQYRELTKLKSTYIDPLPDLIHPRTGRLHTRFNQTATATGRLS | |
| | SSDPNLQNIPVRTPLGQRIRRAFIAEEGWLLVALDYSQIELRVLAHLSGDENLIRVFQEG | |
| | RDIHTETASWMFGVPREAVDPLMRRAAKTINFGVLYGMSAHRLSQELAIPYEEAQAFIER | |
| | YFQSFPKVRAWIEKTLEEGRRRGYVETLFGRRRYVPDLEARVKSVREAAERMAFNMPVQG | |
| | TAADLMKLAMVKLFPRLEEMGARMLLQVHDELVLEAPKERAEAVARLAKEVMEGVYPLAV | |
| | PLEVEVGIGEDWLSAKEAAALEIKRASQPELAPEDPEDVEHHHHHH | |
| M33 | ATGCGTGGTATGCTGCCGTTGTTCGAGCCTAAAGGCCGCGTACTGTTAGTCGATGGTCAT | 54 |
| (H21R/A | CGTTTGGCCTATCGGACGTTCCATGCACTCAAAGGTCTGACGACCAGTCGTGGCGAACCG | |
| 61D/T88 | GTCCAGGCTGTTTATGGTTTCGCTAAGTCTTTGCTCAAAGCACTGAAAGAAGACGGGGAC | |
| R); DNA | GATGTAATTGTTGTATTTGATGCCAAAGCACCGAGCTTCCGCCACGAAGCTTATGGTGGC | |
| sequence | TACAAGGCAGGACGCGCCCCTCGTCCAGAAGATTTCCCCCGTCAGCTGGCATTAATTAAG | |
| (5′ to 3′) | GAGTTAGTAGACCTTCTCGGCTTAGCGCGTCTGGAAGTTCCGGGTTATGAGGCGGACGAT | |
| | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M34 | MRGMLPLFEPKGRVLLVDGHRLAYRTFHALKGLTTSRGRPVQAVYGFAKSLLKALKEDGD | 55 |
| (H21R/E | AVIVVFDAKAPSFRHEAYGGYKAGRAPRPEDFPRQLALIKELVDLLGLARLEVPGYEADD | |
| 39R/T88 | VLASLAKKAEKEGYEVRILTADKDLYQLLSDRIHVLHPEGYLITPAWLWEKYGLRPDQWA | |
| R/L367V | DYRALTGDESDNLPGVKGIGEKTARKLLEEWGSLEALLKNLDRLKPAIREKILAHMDDLK | |
| ); Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| (N to C | LREGLGVPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M34 | ATGCGTGGTATGCTGCCGTTGTTCGAGCCTAAAGGCCGCGTACTGTTAGTCGATGGTCAT | 56 |
| (H21R/E | CGTTTGGCCTATCGGACGTTCCATGCACTCAAAGGTCTGACGACCAGTCGTGGCCGTCCG | |
| 39R/T88 | GTCCAGGCTGTTTATGGTTTCGCTAAGTCTTTGCTCAAAGCACTGAAAGAAGACGGGGAC | |
| R/L367V | GCGGTAATTGTTGTATTTGATGCCAAAGCACCGAGCTTCCGCCACGAAGCTTATGGTGGC | |
| ); DNA | TACAAGGCAGGACGCGCCCCTCGTCCAGAAGATTTCCCCCGTCAGCTGGCATTAATTAAG | |
| sequence | GAGTTAGTAGACCTTCTCGGCTTAGCGCGTCTGGAAGTTCCGGGTTATGAGGCGGACGAT | |
| (5′ to 3′) | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGAGTCCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M35 | MFGMLPLFEPKGRVLLVDGHRLAYRTFHALKGLTTSRGRPVQAVYGFAKSLLKALKEDGD | 57 |
| (R2F/H2 | AVIVVFDAKAPSFRHEAYGGYKAGRAPRPEDFPRQLALIKELVDLLGLARLEVPGYEADD | |
| 1R/E39R | VLASLAKKAEKEGYEVRILTADKDLYQLLSDRIHVLHPEGYLITPAWLWEKYGLRPDQWA | |
| /T88R); | DYRALTGDESDNLPGVKGIGEKTARKLLEEWGSLEALLKNLDRLKPAIREKILAHMDDLK | |
| Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| (N to C | LREGLGLPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M35 | ATGTTTGGTATGCTGCCGTTGTTCGAGCCTAAAGGCCGCGTACTGTTAGTCGATGGTCAT | 58 |
| (R2F/H2 | CGTTTGGCCTATCGGACGTTCCATGCACTCAAAGGTCTGACGACCAGTCGTGGCCGTCCG | |
| 1R/E39R | GTCCAGGCTGTTTATGGTTTCGCTAAGTCTTTGCTCAAAGCACTGAAAGAAGACGGGGAC | |
| /T88R); | GCGGTAATTGTTGTATTTGATGCCAAAGCACCGAGCTTCCGCCACGAAGCTTATGGTGGC | |
| DNA | TACAAGGCAGGACGCGCCCCTCGTCCAGAAGATTTCCCCCGTCAGCTGGCATTAATTAAG | |
| sequence | GAGTTAGTAGACCTTCTCGGCTTAGCGCGTCTGGAAGTTCCGGGTTATGAGGCGGACGAT | |
| (5′ to 3′) | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M36 | MRGMLPLFEPKGRVLLVDGHRLAYRTFHALKGLTTSRGRPVQAVYGFAKSLLKALKEDGD | 59 |
| (H21R/E | AVIVVFDAKAPSFRHEAYGGYKAGRAPRPEDFPRQLALIKELVDLLGLARLEVPGYEADD | |
| 39R/T88 | VLASLAKKAEKEGYEVRILTADKDLYQLLSDRIHVLHPEGYLITPAWLWEKYGLRPDQWA | |
| R/Y182S | DSRALTGDESDNLPGVKGIGEKTARKLLEEWGSLEALLKNLDRLKPAIREKILAHMDDLK | |
| ); Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| (N to C | LREGLGLPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M36 | ATGCGTGGTATGCTGCCGTTGTTCGAGCCTAAAGGCCGCGTACTGTTAGTCGATGGTCAT | 60 |
| (H21R/E | CGTTTGGCCTATCGGACGTTCCATGCACTCAAAGGTCTGACGACCAGTCGTGGCCGTCCG | |
| 39R/T88 | GTCCAGGCTGTTTATGGTTTCGCTAAGTCTTTGCTCAAAGCACTGAAAGAAGACGGGGAC | |
| R/Y182S | GCGGTAATTGTTGTATTTGATGCCAAAGCACCGAGCTTCCGCCACGAAGCTTATGGTGGC | |
| ); DNA | TACAAGGCAGGACGCGCCCCTCGTCCAGAAGATTTCCCCCGTCAGCTGGCATTAATTAAG | |
| sequence | GAGTTAGTAGACCTTCTCGGCTTAGCGCGTCTGGAAGTTCCGGGTTATGAGGCGGACGAT | |
| (5′ to 3′) | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATAGCCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M37 | MRGMLPLFEPKGRVLLVDGHRLAYRTFHALKGLTTSRGRPVQAVYGFAKSLLKALKEDGD | 61 |
| (H21R/E | DVIVVFDAKAPSFRHEAYGGYKAGRAPRPEDFPRQLALIKELVDLLGLARLEVPGYEADD | |
| 39R/A61 | VLASLAKKAEKEGYEVRILTADKDLYQLLSDRIHVLHPEGYLITPAWLWEKYGLRPDQWA | |
| D/T88R); | DYRALTGDESDNLPGVKGIGEKTARKLLEEWGSLEALLKNLDRLKPAIREKILAHMDDLK | |
| Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| (N to C | LREGLGLPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M37 | ATGCGTGGTATGCTGCCGTTGTTCGAGCCTAAAGGCCGCGTACTGTTAGTCGATGGTCAT | 62 |
| (H21R/E | CGTTTGGCCTATCGGACGTTCCATGCACTCAAAGGTCTGACGACCAGTCGTGGCCGTCCG | |
| 39R/A61 | GTCCAGGCTGTTTATGGTTTCGCTAAGTCTTTGCTCAAAGCACTGAAAGAAGACGGGGAC | |
| D/T88R); | GATGTAATTGTTGTATTTGATGCCAAAGCACCGAGCTTCCGCCACGAAGCTTATGGTGGC | |
| DNA | TACAAGGCAGGACGCGCCCCTCGTCCAGAAGATTTCCCCCGTCAGCTGGCATTAATTAAG | |
| sequence | GAGTTAGTAGACCTTCTCGGCTTAGCGCGTCTGGAAGTTCCGGGTTATGAGGCGGACGAT | |
| (5′ to 3′) | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M38 | MFGMLPLFEPKGRVLLVDGHRLAYRTFHALKGLTTSRGEPVQAVYGFAKSLLKALKEDGD | 63 |
| (R2F/H2 | AVIVVFDAKAPSFRHEAYGGYKAGRAPRPEDFPRQLALIKELVDLLGLARLEVPGYEADD | |
| 1R/T88R | VLASLAKKAEKEGYEVRILTADKDLYQLLSDRIHVLHPEGYLITPAWLWEKYGLRPDQWA | |
| /L367V); | DYRALTGDESDNLPGVKGIGEKTARKLLEEWGSLEALLKNLDRLKPAIREKILAHMDDLK | |
| Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| (N to C | LREGLGVPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M38 | ATGTTTGGTATGCTGCCGTTGTTCGAGCCTAAAGGCCGCGTACTGTTAGTCGATGGTCAT | 64 |
| (R2F/H2 | CGTTTGGCCTATCGGACGTTCCATGCACTCAAAGGTCTGACGACCAGTCGTGGCGAACCG | |
| 1R/T88R | GTCCAGGCTGTTTATGGTTTCGCTAAGTCTTTGCTCAAAGCACTGAAAGAAGACGGGGAC | |
| /L367V); | GCGGTAATTGTTGTATTTGATGCCAAAGCACCGAGCTTCCGCCACGAAGCTTATGGTGGC | |
| DNA | TACAAGGCAGGACGCGCCCCTCGTCCAGAAGATTTCCCCCGTCAGCTGGCATTAATTAAG | |
| sequence | GAGTTAGTAGACCTTCTCGGCTTAGCGCGTCTGGAAGTTCCGGGTTATGAGGCGGACGAT | |
| (5′ to 3′) | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGAGTCCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M39 | MRGMLPLFEPKGRVLLVDGHRLAYRTFHALKGLTTSRGEPVQAVYGFAKSLLKALKEDGD | 65 |
| (H21R/T | AVIVVFDAKAPSFRHEAYGGYKAGRAPRPEDFPRQLALIKELVDLLGLARLEVPGYEADD | |
| 88R/Y18 | VLASLAKKAEKEGYEVRILTADKDLYQLLSDRIHVLHPEGYLITPAWLWEKYGLRPDQWA | |
| 2S/L367 | DSRALTGDESDNLPGVKGIGEKTARKLLEEWGSLEALLKNLDRLKPAIREKILAHMDDLK | |
| V) | LSWDLAKVRTDLPLEVDFAKRREPDRERLRAFLERLEFGSLLHEFGLLESPKALEEAPWP | |
| Protein | PPEGAFVGFVLSRKEPMWADLLALAAARGGRVHRAPEPYKALRDLKEARGLLAKDLSVLA | |
| sequence | LREGLGVPPGDDPMLLAYLLDPSNTTPEGVARRYGGEWTEEAGERAALSERLFANLWGRL | |
| (N to C | EGEERLLWLYREVERPLSAVLAHMEATGVRLDVAYLRALSLEVAEEIARLEAEVFRLAGH | |
| terminus) | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M39 | ATGCGTGGTATGCTGCCGTTGTTCGAGCCTAAAGGCCGCGTACTGTTAGTCGATGGTCAT | 66 |
| (H21R/T | CGTTTGGCCTATCGGACGTTCCATGCACTCAAAGGTCTGACGACCAGTCGTGGCGAACCG | |
| 88R/Y18 | GTCCAGGCTGTTTATGGTTTCGCTAAGTCTTTGCTCAAAGCACTGAAAGAAGACGGGGAC | |
| 2S/L367 | GCGGTAATTGTTGTATTTGATGCCAAAGCACCGAGCTTCCGCCACGAAGCTTATGGTGGC | |
| V); DNA | TACAAGGCAGGACGCGCCCCTCGTCCAGAAGATTTCCCCCGTCAGCTGGCATTAATTAAG | |
| sequence | GAGTTAGTAGACCTTCTCGGCTTAGCGCGTCTGGAAGTTCCGGGTTATGAGGCGGACGAT | |
| (5′ to 3′) | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATAGCCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGAGTCCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M40 | MRGMLPLFEPKGRVLLVDGHRLAYRTFHALKGLTTSRGEPVQAVYGFAKSLLKALKEDGD | 67 |
| (H21R/A | DVIVVFDAKAPSFRHEAYGGYKAGRAPRPEDFPRQLALIKELVDLLGLARLEVPGYEADD | |
| 61D/T88 | VLASLAKKAEKEGYEVRILTADKDLYQLLSDRIHVLHPEGYLITPAWLWEKYGLRPDQWA | |
| R/L367V | DYRALTGDESDNLPGVKGIGEKTARKLLEEWGSLEALLKNLDRLKPAIREKILAHMDDLK | |
| ); Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| (N to C | LREGLGVPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M40 | ATGCGTGGTATGCTGCCGTTGTTCGAGCCTAAAGGCCGCGTACTGTTAGTCGATGGTCAT | 68 |
| (H21R/A | CGTTTGGCCTATCGGACGTTCCATGCACTCAAAGGTCTGACGACCAGTCGTGGCGAACCG | |
| 61D/T88R | GTCCAGGCTGTTTATGGTTTCGCTAAGTCTTTGCTCAAAGCACTGAAAGAAGACGGGGAC | |
| /L367V); | GATGTAATTGTTGTATTTGATGCCAAAGCACCGAGCTTCCGCCACGAAGCTTATGGTGGC | |
| DNA | TACAAGGCAGGACGCGCCCCTCGTCCAGAAGATTTCCCCCGTCAGCTGGCATTAATTAAG | |
| sequence | GAGTTAGTAGACCTTCTCGGCTTAGCGCGTCTGGAAGTTCCGGGTTATGAGGCGGACGAT | |
| (5′ to 3′) | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGAGTCCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M41 | MFGMLPLFEPKGRVLLVDGHRLAYRTFHALKGLTTSRGEPVQAVYGFAKSLLKALKEDGD | 69 |
| (R2F/H2 | AVIVVFDAKAPSFRHEAYGGYKAGRAPRPEDFPRQLALIKELVDLLGLARLEVPGYEADD | |
| 1R/T88R | VLASLAKKAEKEGYEVRILTADKDLYQLLSDRIHVLHPEGYLITPAWLWEKYGLRPDQWA | |
| /Y182S); | DSRALTGDESDNLPGVKGIGEKTARKLLEEWGSLEALLKNLDRLKPAIREKILAHMDDLK | |
| Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| (N to C | LREGLGLPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M41 | ATGTTTGGTATGCTGCCGTTGTTCGAGCCTAAAGGCCGCGTACTGTTAGTCGATGGTCAT | 70 |
| (R2F/H2 | CGTTTGGCCTATCGGACGTTCCATGCACTCAAAGGTCTGACGACCAGTCGTGGCGAACCG | |
| 1R/T88R | GTCCAGGCTGTTTATGGTTTCGCTAAGTCTTTGCTCAAAGCACTGAAAGAAGACGGGGAC | |
| /Y182S); | GCGGTAATTGTTGTATTTGATGCCAAAGCACCGAGCTTCCGCCACGAAGCTTATGGTGGC | |
| DNA | TACAAGGCAGGACGCGCCCCTCGTCCAGAAGATTTCCCCCGTCAGCTGGCATTAATTAAG | |
| sequence | GAGTTAGTAGACCTTCTCGGCTTAGCGCGTCTGGAAGTTCCGGGTTATGAGGCGGACGAT | |
| (5′ to 3′) | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATAGCCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M42 | MFGMLPLFEPKGRVLLVDGHRLAYRTFHALKGLTTSRGEPVQAVYGFAKSLLKALKEDGD | 71 |
| (R2F/H2 | DVIVVFDAKAPSFRHEAYGGYKAGRAPRPEDFPRQLALIKELVDLLGLARLEVPGYEADD | |
| 1R/A61D | VLASLAKKAEKEGYEVRILTADKDLYQLLSDRIHVLHPEGYLITPAWLWEKYGLRPDQWA | |
| /T88R); | DYRALTGDESDNLPGVKGIGEKTARKLLEEWGSLEALLKNLDRLKPAIREKILAHMDDLK | |
| Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| (N to C | LREGLGLPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M42 | ATGTTTGGTATGCTGCCGTTGTTCGAGCCTAAAGGCCGCGTACTGTTAGTCGATGGTCAT | 72 |
| (R2F/H2 | CGTTTGGCCTATCGGACGTTCCATGCACTCAAAGGTCTGACGACCAGTCGTGGCGAACCG | |
| 1R/A61D | GTCCAGGCTGTTTATGGTTTCGCTAAGTCTTTGCTCAAAGCACTGAAAGAAGACGGGGAC | |
| /T88R); | GATGTAATTGTTGTATTTGATGCCAAAGCACCGAGCTTCCGCCACGAAGCTTATGGTGGC | |
| DNA | TACAAGGCAGGACGCGCCCCTCGTCCAGAAGATTTCCCCCGTCAGCTGGCATTAATTAAG | |
| sequence | GAGTTAGTAGACCTTCTCGGCTTAGCGCGTCTGGAAGTTCCGGGTTATGAGGCGGACGAT | |
| (5′ to 3′) | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M43 | MRGMLPLFEPKGRVLLVDGHRLAYRTFHALKGLTTSRGEPVQAVYGFAKSLLKALKEDGD | 73 |
| (H21R/A | DVIVVFDAKAPSFRHEAYGGYKAGRAPRPEDFPRQLALIKELVDLLGLARLEVPGYEADD | |
| 61D/T88 | VLASLAKKAEKEGYEVRILTADKDLYQLLSDRIHVLHPEGYLITPAWLWEKYGLRPDQWA | |
| R/Y182S | DSRALTGDESDNLPGVKGIGEKTARKLLEEWGSLEALLKNLDRLKPAIREKILAHMDDLK | |
| ); Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| (N to C | LREGLGLPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M43 | ATGCGTGGTATGCTGCCGTTGTTCGAGCCTAAAGGCCGCGTACTGTTAGTCGATGGTCAT | 74 |
| (H21R/A | CGTTTGGCCTATCGGACGTTCCATGCACTCAAAGGTCTGACGACCAGTCGTGGCGAACCG | |
| 61D/T88 | GTCCAGGCTGTTTATGGTTTCGCTAAGTCTTTGCTCAAAGCACTGAAAGAAGACGGGGAC | |
| R/Y182S | GATGTAATTGTTGTATTTGATGCCAAAGCACCGAGCTTCCGCCACGAAGCTTATGGTGGC | |
| ); DNA | TACAAGGCAGGACGCGCCCCTCGTCCAGAAGATTTCCCCCGTCAGCTGGCATTAATTAAG | |
| sequence | GAGTTAGTAGACCTTCTCGGCTTAGCGCGTCTGGAAGTTCCGGGTTATGAGGCGGACGAT | |
| (5′ to 3′) | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATAGCCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M44 | MRGMLPLFEPKGRVLLLDGHRLAYRTFHALKGLTTSRGEPVQAVYGFAKSLLKALKEDGD | 75 |
| (V17L/H | AVIVVFDAKAPSFRHEAYGGYKAGRAPRPEDFPRQLALIKELVDLLGLARLEVPGYEADD | |
| 21R/T88 | VLASLAKKAEKEGYEVRILTADKDLYQLLSDRIHVLHPEGYLITPAWLWEKYGLRPDQWA | |
| R); | DYRALTGDESDNLPGVKGIGEKTARKLLEEWGSLEALLKNLDRLKPAIREKILAHMDDLK | |
| Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| (N to C | LREGLGLPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M44 | ATGCGTGGTATGCTGCCGTTGTTCGAGCCTAAAGGCCGCGTACTGTTACTTGATGGTCAT | 76 |
| (V17L/H | CGTTTGGCCTATCGGACGTTCCATGCACTCAAAGGTCTGACGACCAGTCGTGGCGAACCG | |
| 21R/T88 | GTCCAGGCTGTTTATGGTTTCGCTAAGTCTTTGCTCAAAGCACTGAAAGAAGACGGGGAC | |
| R); DNA | GCGGTAATTGTTGTATTTGATGCCAAAGCACCGAGCTTCCGCCACGAAGCTTATGGTGGC | |
| sequence | TACAAGGCAGGACGCGCCCCTCGTCCAGAAGATTTCCCCCGTCAGCTGGCATTAATTAAG | |
| (5′ to 3′) | GAGTTAGTAGACCTTCTCGGCTTAGCGCGTCTGGAAGTTCCGGGTTATGAGGCGGACGAT | |
| | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M45 | MRGMLPLFEPKGRVLLVDGHRLAHRTFHALKGLTTSRGEPVQAVYGFAKSLLKALKEDGD | 77 |
| (H21R/Y | AVIVVFDAKAPSFRHEAYGGYKAGRAPRPEDFPRQLALIKELVDLLGLARLEVPGYEADD | |
| 24H/T88 | VLASLAKKAEKEGYEVRILTADKDLYQLLSDRIHVLHPEGYLITPAWLWEKYGLRPDQWA | |
| R); | DYRALTGDESDNLPGVKGIGEKTARKLLEEWGSLEALLKNLDRLKPAIREKILAHMDDLK | |
| Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| (N to C | LREGLGLPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M45 | ATGCGTGGTATGCTGCCGTTGTTCGAGCCTAAAGGCCGCGTACTGTTAGTCGATGGTCAT | 78 |
| (H21R/Y | CGTTTGGCCCATCGGACGTTCCATGCACTCAAAGGTCTGACGACCAGTCGTGGCGAACCG | |
| 24H/T88 | GTCCAGGCTGTTTATGGTTTCGCTAAGTCTTTGCTCAAAGCACTGAAAGAAGACGGGGAC | |
| R); DNA | GCGGTAATTGTTGTATTTGATGCCAAAGCACCGAGCTTCCGCCACGAAGCTTATGGTGGC | |
| sequence | TACAAGGCAGGACGCGCCCCTCGTCCAGAAGATTTCCCCCGTCAGCTGGCATTAATTAAG | |
| (5′ to 3′) | GAGTTAGTAGACCTTCTCGGCTTAGCGCGTCTGGAAGTTCCGGGTTATGAGGCGGACGAT | |
| | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M46 | MRGMLPLFEPKGRVLLVDGHRLAYRTRHALKGLTTSRGEPVQAVYGFAKSLLKALKEDGD | 79 |
| (H21R/F | AVIVVFDAKAPSFRHEAYGGYKAGRAPRPEDFPRQLALIKELVDLLGLARLEVPGYEADD | |
| 27R/T88 | VLASLAKKAEKEGYEVRILTADKDLYQLLSDRIHVLHPEGYLITPAWLWEKYGLRPDQWA | |
| R); | DYRALTGDESDNLPGVKGIGEKTARKLLEEWGSLEALLKNLDRLKPAIREKILAHMDDLK | |
| Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| (N to C | LREGLGLPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M46 | ATGCGTGGTATGCTGCCGTTGTTCGAGCCTAAAGGCCGCGTACTGTTAGTCGATGGTCAT | 80 |
| (H21R/F | CGTTTGGCCTATCGGACGCGTCATGCACTCAAAGGTCTGACGACCAGTCGTGGCGAACCG | |
| 27R/T88 | GTCCAGGCTGTTTATGGTTTCGCTAAGTCTTTGCTCAAAGCACTGAAAGAAGACGGGGAC | |
| R); DNA | GCGGTAATTGTTGTATTTGATGCCAAAGCACCGAGCTTCCGCCACGAAGCTTATGGTGGC | |
| sequence | TACAAGGCAGGACGCGCCCCTCGTCCAGAAGATTTCCCCCGTCAGCTGGCATTAATTAAG | |
| (5′ to 3′) | GAGTTAGTAGACCTTCTCGGCTTAGCGCGTCTGGAAGTTCCGGGTTATGAGGCGGACGAT | |
| | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M47 | MRGMLPLFEPKGRVLLVDGHRLAYRTFHPLKGLTTSRGEPVQAVYGFAKSLLKALKEDGD | 81 |
| (H21R/A | AVIVVFDAKAPSFRHEAYGGYKAGRAPRPEDFPRQLALIKELVDLLGLARLEVPGYEADD | |
| 29P/T88 | VLASLAKKAEKEGYEVRILTADKDLYQLLSDRIHVLHPEGYLITPAWLWEKYGLRPDQWA | |
| R); | DYRALTGDESDNLPGVKGIGEKTARKLLEEWGSLEALLKNLDRLKPAIREKILAHMDDLK | |
| Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| (N to C | LREGLGLPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M47 | ATGCGTGGTATGCTGCCGTTGTTCGAGCCTAAAGGCCGCGTACTGTTAGTCGATGGTCAT | 82 |
| (H21R/A | CGTTTGGCCTATCGGACGTTCCATCCGCTCAAAGGTCTGACGACCAGTCGTGGCGAACCG | |
| 29P/T88 | GTCCAGGCTGTTTATGGTTTCGCTAAGTCTTTGCTCAAAGCACTGAAAGAAGACGGGGAC | |
| R); DNA | GCGGTAATTGTTGTATTTGATGCCAAAGCACCGAGCTTCCGCCACGAAGCTTATGGTGGC | |
| sequence | TACAAGGCAGGACGCGCCCCTCGTCCAGAAGATTTCCCCCGTCAGCTGGCATTAATTAAG | |
| (5′ to 3′) | GAGTTAGTAGACCTTCTCGGCTTAGCGCGTCTGGAAGTTCCGGGTTATGAGGCGGACGAT | |
| | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M48 | MRGMLPLFEPKGRVLLVDGHRLAYRTFHALKGLTTSRGEPVQAVYGFAKSLLKALKEDGD | 83 |
| (H21R/T | AVIVVFDAKAPSFRHEAYGGYKAGRAPRPEDFPRQLALIKELVDLLGLARLEVPGYEADD | |
| 88R/E21 | VLASLAKKAEKEGYEVRILTADKDLYQLLSDRIHVLHPEGYLITPAWLWEKYGLRPDQWA | |
| OR); | DYRALTGDESDNLPGVKGIGEKTARKLLERWGSLEALLKNLDRLKPAIREKILAHMDDLK | |
| Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| (N to C | LREGLGLPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M48 | ATGCGTGGTATGCTGCCGTTGTTCGAGCCTAAAGGCCGCGTACTGTTAGTCGATGGTCAT | 84 |
| (H21R/T | CGTTTGGCCTATCGGACGTTCCATGCACTCAAAGGTCTGACGACCAGTCGTGGCGAACCG | |
| 88R/E21 | GTCCAGGCTGTTTATGGTTTCGCTAAGTCTTTGCTCAAAGCACTGAAAGAAGACGGGGAC | |
| OR); | GCGGTAATTGTTGTATTTGATGCCAAAGCACCGAGCTTCCGCCACGAAGCTTATGGTGGC | |
| DNA | TACAAGGCAGGACGCGCCCCTCGTCCAGAAGATTTCCCCCGTCAGCTGGCATTAATTAAG | |
| sequence | GAGTTAGTAGACCTTCTCGGCTTAGCGCGTCTGGAAGTTCCGGGTTATGAGGCGGACGAT | |
| (5′ to 3′) | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAACGTTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M49 | MRGMLPLFEPKGRVLLVDGHRLAYRTFHALKGLTTSRGEPVQAVYGFAKSLLKALKEDGD | 85 |
| (H21R/T | AVIVVFDAKAPSFRHEAYGGYKAGRAPRPEDFPRQLALIKELVDLLGLARLEVPGYEADD | |
| 88R/V63 | VLASLAKKAEKEGYEVRILTADKDLYQLLSDRIHVLHPEGYLITPAWLWEKYGLRPDQWA | |
| 1A); | DYRALTGDESDNLPGVKGIGEKTARKLLEEWGSLEALLKNLDRLKPAIREKILAHMDDLK | |
| Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| (N to C | LREGLGLPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRAFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M49 | ATGCGTGGTATGCTGCCGTTGTTCGAGCCTAAAGGCCGCGTACTGTTAGTCGATGGTCAT | 86 |
| (H21R/T | CGTTTGGCCTATCGGACGTTCCATGCACTCAAAGGTCTGACGACCAGTCGTGGCGAACCG | |
| 88R/V63 | GTCCAGGCTGTTTATGGTTTCGCTAAGTCTTTGCTCAAAGCACTGAAAGAAGACGGGGAC | |
| 1A); | GCGGTAATTGTTGTATTTGATGCCAAAGCACCGAGCTTCCGCCACGAAGCTTATGGTGGC | |
| DNA | TACAAGGCAGGACGCGCCCCTCGTCCAGAAGATTTCCCCCGTCAGCTGGCATTAATTAAG | |
| sequence | GAGTTAGTAGACCTTCTCGGCTTAGCGCGTCTGGAAGTTCCGGGTTATGAGGCGGACGAT | |
| (5′ to 3′) | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGCGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M50 | MRGMLPLFEPKGRVLLVDGHRLAYRTFHALKGLTTSRGEPVQAVYGFAKSLLKALKEDGD | 87 |
| (H21R/T | AVIVVFDAKAPSFRHEAYGGYKAGRAPRPEDFPRQLALIKELVDLLGLARLEVPGYEADD | |
| 88R/K70 | VLASLAKKAEKEGYEVRILTADKDLYQLLSDRIHVLHPEGYLITPAWLWEKYGLRPDQWA | |
| 9R); | DYRALTGDESDNLPGVKGIGEKTARKLLEEWGSLEALLKNLDRLKPAIREKILAHMDDLK | |
| Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| (N to C | LREGLGLPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIERTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M50 | ATGCGTGGTATGCTGCCGTTGTTCGAGCCTAAAGGCCGCGTACTGTTAGTCGATGGTCAT | 88 |
| (H21R/T | CGTTTGGCCTATCGGACGTTCCATGCACTCAAAGGTCTGACGACCAGTCGTGGCGAACCG | |
| 88R/K70 | GTCCAGGCTGTTTATGGTTTCGCTAAGTCTTTGCTCAAAGCACTGAAAGAAGACGGGGAC | |
| 9R); | GCGGTAATTGTTGTATTTGATGCCAAAGCACCGAGCTTCCGCCACGAAGCTTATGGTGGC | |
| DNA | TACAAGGCAGGACGCGCCCCTCGTCCAGAAGATTTCCCCCGTCAGCTGGCATTAATTAAG | |
| sequence | GAGTTAGTAGACCTTCTCGGCTTAGCGCGTCTGGAAGTTCCGGGTTATGAGGCGGACGAT | |
| (5′ to 3′) | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGCGTACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M51 | MRGMLPLFEPKGRVLLVDGHRLAYRTFHALKGLTTSRGEPVQAVYGFAKSLLKALKEDGD | 89 |
| (H21R/T | AVIVVFDAKAPSFRHEAYGGYKAGRAPRPEDFPRQLALIKELVDLLGLARLEVPGYEADD | |
| 88R/E72 | VLASLAKKAEKEGYEVRILTADKDLYQLLSDRIHVLHPEGYLITPAWLWEKYGLRPDQWA | |
| 4G); | DYRALTGDESDNLPGVKGIGEKTARKLLEEWGSLEALLKNLDRLKPAIREKILAHMDDLK | |
| Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| (N to C | LREGLGLPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVRGAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M51 | ATGCGTGGTATGCTGCCGTTGTTCGAGCCTAAAGGCCGCGTACTGTTAGTCGATGGTCAT | 90 |
| (H21R/T | CGTTTGGCCTATCGGACGTTCCATGCACTCAAAGGTCTGACGACCAGTCGTGGCGAACCG | |
| 88R/E72 | GTCCAGGCTGTTTATGGTTTCGCTAAGTCTTTGCTCAAAGCACTGAAAGAAGACGGGGAC | |
| 4G); | GCGGTAATTGTTGTATTTGATGCCAAAGCACCGAGCTTCCGCCACGAAGCTTATGGTGGC | |
| DNA | TACAAGGCAGGACGCGCCCCTCGTCCAGAAGATTTCCCCCGTCAGCTGGCATTAATTAAG | |
| sequence | GAGTTAGTAGACCTTCTCGGCTTAGCGCGTCTGGAAGTTCCGGGTTATGAGGCGGACGAT | |
| (5′ to 3′) | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGGCGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M52 | MRGMLPLFEPKGRVLLVDGHRLAYRTFHALKGLTTSRGEPVQAVYGFAKSLLKALKEDGD | 91 |
| (H21R/T | AVIVVFDAKAPSFRHEAYGGYKAGRAPRPEDFPRQLALIKELVDLLGLARLEVPGYEADD | |
| 88R/K83 | VLASLAKKAEKEGYEVRILTADKDLYQLLSDRIHVLHPEGYLITPAWLWEKYGLRPDQWA | |
| 1E); | DYRALTGDESDNLPGVKGIGEKTARKLLEEWGSLEALLKNLDRLKPAIREKILAHMDDLK | |
| Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| (N to C | LREGLGLPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAEEAAALEIKR | |
| | ASQPELAPEDPEDVEHHHHHH | |
| M52 | ATGCGTGGTATGCTGCCGTTGTTCGAGCCTAAAGGCCGCGTACTGTTAGTCGATGGTCAT | 92 |
| (H21R/T | CGTTTGGCCTATCGGACGTTCCATGCACTCAAAGGTCTGACGACCAGTCGTGGCGAACCG | |
| 88R/K83 | GTCCAGGCTGTTTATGGTTTCGCTAAGTCTTTGCTCAAAGCACTGAAAGAAGACGGGGAC | |
| 1E); | GCGGTAATTGTTGTATTTGATGCCAAAGCACCGAGCTTCCGCCACGAAGCTTATGGTGGC | |
| DNA | TACAAGGCAGGACGCGCCCCTCGTCCAGAAGATTTCCCCCGTCAGCTGGCATTAATTAAG | |
| sequence | GAGTTAGTAGACCTTCTCGGCTTAGCGCGTCTGGAAGTTCCGGGTTATGAGGCGGACGAT | |
| (5′ to 3′) | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAGAGGAAgcggccgcactcgagatcaaacgg | |
| | gctagccagccagaactcgccccggaagaccccgaggatgtcgagcaccaccaccaccac | |
| | cac | |
| M53 | MRGMLPLFEPKGRVLLLDGHRLAYRTFHALKGLTTSRGRPVQAVYGFAKSLLKALKEDGD | 93 |
| (V17L/H | DVIVVFDAKAPSFRHEAYGGYKAGRAPRPEDFPRQLALIKELVDLLGLARLEVPGYEADD | |
| 21R/E39 | VLASLAKKAEKEGYEVRILTADKDLYQLLSDRIHVLHPEGYLITPAWLWEKYGLRPDQWA | |
| R/A61D/ | DYRALTGDESDNLPGVKGIGEKTARKLLEEWGSLEALLKNLDRLKPAIREKILAHMDDLK | |
| T88R); | LSWDLAKVRTDLPLEVDFAKRREPDRERLRAFLERLEFGSLLHEFGLLESPKALEEAPWP | |
| Protein | PPEGAFVGFVLSRKEPMWADLLALAAARGGRVHRAPEPYKALRDLKEARGLLAKDLSVLA | |
| sequence | LREGLGLPPGDDPMLLAYLLDPSNTTPEGVARRYGGEWTEEAGERAALSERLFANLWGRL | |
| (N to C | EGEERLLWLYREVERPLSAVLAHMEATGVRLDVAYLRALSLEVAEEIARLEAEVFRLAGH | |
| terminus) | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M53 | ATGCGTGGTATGCTGCCGTTGTTCGAGCCTAAAGGCCGCGTACTGTTACTTGATGGTCAT | 94 |
| (V17L/H | CGTTTGGCCTATCGGACGTTCCATGCACTCAAAGGTCTGACGACCAGTCGTGGCCGTCCG | |
| 21R/E39 | GTCCAGGCTGTTTATGGTTTCGCTAAGTCTTTGCTCAAAGCACTGAAAGAAGACGGGGAC | |
| R/A61D/ | GATGTAATTGTTGTATTTGATGCCAAAGCACCGAGCTTCCGCCACGAAGCTTATGGTGGC | |
| T88R); | TACAAGGCAGGACGCGCCCCTCGTCCAGAAGATTTCCCCCGTCAGCTGGCATTAATTAAG | |
| DNA | GAGTTAGTAGACCTTCTCGGCTTAGCGCGTCTGGAAGTTCCGGGTTATGAGGCGGACGAT | |
| sequence | GTCCTTGCATCCTTGGCTAAAAAGGCCGAAAAAGAGGGCTACGAAGTCCGCATCTTGACG | |
| (5′ to 3′) | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M54 | MRGMLPLFEPKGRVLLVDGHRLAHRTFHALKGLTTSRGRPVQAVYGFAKSLLKALKEDGD | 95 |
| (H21R/Y | DVIVVFDAKAPSFRHEAYGGYKAGRAPRPEDFPRQLALIKELVDLLGLARLEVPGYEADD | |
| 24H/E39 | VLASLAKKAEKEGYEVRILTADKDLYQLLSDRIHVLHPEGYLITPAWLWEKYGLRPDQWA | |
| R/A61D/ | DYRALTGDESDNLPGVKGIGEKTARKLLEEWGSLEALLKNLDRLKPAIREKILAHMDDLK | |
| T88R); | LSWDLAKVRTDLPLEVDFAKRREPDRERLRAFLERLEFGSLLHEFGLLESPKALEEAPWP | |
| Protein | PPEGAFVGFVLSRKEPMWADLLALAAARGGRVHRAPEPYKALRDLKEARGLLAKDLSVLA | |
| sequence | LREGLGLPPGDDPMLLAYLLDPSNTTPEGVARRYGGEWTEEAGERAALSERLFANLWGRL | |
| (N to C | EGEERLLWLYREVERPLSAVLAHMEATGVRLDVAYLRALSLEVAEEIARLEAEVFRLAGH | |
| terminus) | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M54 | ATGCGTGGTATGCTGCCGTTGTTCGAGCCTAAAGGCCGCGTACTGTTAGTCGATGGTCAT | 96 |
| (H21R/Y | CGTTTGGCCCATCGGACGTTCCATGCACTCAAAGGTCTGACGACCAGTCGTGGCCGTCCG | |
| 24H/E39 | GTCCAGGCTGTTTATGGTTTCGCTAAGTCTTTGCTCAAAGCACTGAAAGAAGACGGGGAC | |
| R/A61D/ | GATGTAATTGTTGTATTTGATGCCAAAGCACCGAGCTTCCGCCACGAAGCTTATGGTGGC | |
| T88R); | TACAAGGCAGGACGCGCCCCTCGTCCAGAAGATTTCCCCCGTCAGCTGGCATTAATTAAG | |
| DNA | GAGTTAGTAGACCTTCTCGGCTTAGCGCGTCTGGAAGTTCCGGGTTATGAGGCGGACGAT | |
| sequence | GTCCTTGCATCCTTGGCTAAAAAGGCCGAAAAAGAGGGCTACGAAGTCCGCATCTTGACG | |
| (5′ to 3′) | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M55 | MRGMLPLFEPKGRVLLVDGHRLAYRTRHALKGLTTSRGRPVQAVYGFAKSLLKALKEDGD | 97 |
| (H21R/F | DVIVVFDAKAPSFRHEAYGGYKAGRAPRPEDFPRQLALIKELVDLLGLARLEVPGYEADD | |
| 27R/E39 | VLASLAKKAEKEGYEVRILTADKDLYQLLSDRIHVLHPEGYLITPAWLWEKYGLRPDQWA | |
| R/A61D/ | DYRALTGDESDNLPGVKGIGEKTARKLLEEWGSLEALLKNLDRLKPAIREKILAHMDDLK | |
| T88R); | LSWDLAKVRTDLPLEVDFAKRREPDRERLRAFLERLEFGSLLHEFGLLESPKALEEAPWP | |
| Protein | PPEGAFVGFVLSRKEPMWADLLALAAARGGRVHRAPEPYKALRDLKEARGLLAKDLSVLA | |
| sequence | LREGLGLPPGDDPMLLAYLLDPSNTTPEGVARRYGGEWTEEAGERAALSERLFANLWGRL | |
| (N to C | EGEERLLWLYREVERPLSAVLAHMEATGVRLDVAYLRALSLEVAEEIARLEAEVFRLAGH | |
| terminus) | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M55 | ATGCGTGGTATGCTGCCGTTGTTCGAGCCTAAAGGCCGCGTACTGTTAGTCGATGGTCAT | 98 |
| (H21R/F | CGTTTGGCCTATCGGACGCGTCATGCACTCAAAGGTCTGACGACCAGTCGTGGCCGTCCG | |
| 27R/E39 | GTCCAGGCTGTTTATGGTTTCGCTAAGTCTTTGCTCAAAGCACTGAAAGAAGACGGGGAC | |
| R/A61D/ | GATGTAATTGTTGTATTTGATGCCAAAGCACCGAGCTTCCGCCACGAAGCTTATGGTGGC | |
| T88R); | TACAAGGCAGGACGCGCCCCTCGTCCAGAAGATTTCCCCCGTCAGCTGGCATTAATTAAG | |
| DNA | GAGTTAGTAGACCTTCTCGGCTTAGCGCGTCTGGAAGTTCCGGGTTATGAGGCGGACGAT | |
| sequence | GTCCTTGCATCCTTGGCTAAAAAGGCCGAAAAAGAGGGCTACGAAGTCCGCATCTTGACG | |
| (5′ to 3′) | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M56 | MRGMLPLFEPKGRVLLVDGHRLAYRTFHPLKGLTTSRGRPVQAVYGFAKSLLKALKEDGD | 99 |
| (H21R/A | DVIVVFDAKAPSFRHEAYGGYKAGRAPRPEDFPRQLALIKELVDLLGLARLEVPGYEADD | |
| 29P/E39 | VLASLAKKAEKEGYEVRILTADKDLYQLLSDRIHVLHPEGYLITPAWLWEKYGLRPDQWA | |
| R/A61D/ | DYRALTGDESDNLPGVKGIGEKTARKLLEEWGSLEALLKNLDRLKPAIREKILAHMDDLK | |
| T88R); | LSWDLAKVRTDLPLEVDFAKRREPDRERLRAFLERLEFGSLLHEFGLLESPKALEEAPWP | |
| Protein | PPEGAFVGFVLSRKEPMWADLLALAAARGGRVHRAPEPYKALRDLKEARGLLAKDLSVLA | |
| sequence | LREGLGLPPGDDPMLLAYLLDPSNTTPEGVARRYGGEWTEEAGERAALSERLFANLWGRL | |
| (N to C | EGEERLLWLYREVERPLSAVLAHMEATGVRLDVAYLRALSLEVAEEIARLEAEVFRLAGH | |
| terminus) | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M56 | ATGCGTGGTATGCTGCCGTTGTTCGAGCCTAAAGGCCGCGTACTGTTAGTCGATGGTCAT | 100 |
| (H21R/A | CGTTTGGCCTATCGGACGTTCCATCCGCTCAAAGGTCTGACGACCAGTCGTGGCCGTCCG | |
| 29P/E39 | GTCCAGGCTGTTTATGGTTTCGCTAAGTCTTTGCTCAAAGCACTGAAAGAAGACGGGGAC | |
| R/A61D/ | GATGTAATTGTTGTATTTGATGCCAAAGCACCGAGCTTCCGCCACGAAGCTTATGGTGGC | |
| T88R); | TACAAGGCAGGACGCGCCCCTCGTCCAGAAGATTTCCCCCGTCAGCTGGCATTAATTAAG | |
| DNA | GAGTTAGTAGACCTTCTCGGCTTAGCGCGTCTGGAAGTTCCGGGTTATGAGGCGGACGAT | |
| sequence | GTCCTTGCATCCTTGGCTAAAAAGGCCGAAAAAGAGGGCTACGAAGTCCGCATCTTGACG | |
| (5′ to 3′) | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M57 | MRGMLPLFEPKGRVLLVDGHRLAYRTFHALKGLTTSRGRPVQAVYGFAKSLLKALKEDGD | 101 |
| (H21R/E | DVIVVFDAKAPSFRHEAYGGYKAGRAPRPEDFPRQLALIKELVDLLGLARLEVPGYEADD | |
| 39R/A61 | VLASLAKKAEKEGYEVRILTADKDLYQLLSDRIHVLHPEGYLITPAWLWEKYGLRPDQWA | |
| D/T88R/ | DYRALTGDESDNLPGVKGIGEKTARKLLERWGSLEALLKNLDRLKPAIREKILAHMDDLK | |
| E210R); | LSWDLAKVRTDLPLEVDFAKRREPDRERLRAFLERLEFGSLLHEFGLLESPKALEEAPWP | |
| Protein | PPEGAFVGFVLSRKEPMWADLLALAAARGGRVHRAPEPYKALRDLKEARGLLAKDLSVLA | |
| sequence | LREGLGLPPGDDPMLLAYLLDPSNTTPEGVARRYGGEWTEEAGERAALSERLFANLWGRL | |
| (N to C | EGEERLLWLYREVERPLSAVLAHMEATGVRLDVAYLRALSLEVAEEIARLEAEVFRLAGH | |
| terminus) | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M57 | ATGCGTGGTATGCTGCCGTTGTTCGAGCCTAAAGGCCGCGTACTGTTAGTCGATGGTCAT | 102 |
| (H21R/E | CGTTTGGCCTATCGGACGTTCCATGCACTCAAAGGTCTGACGACCAGTCGTGGCCGTCCG | |
| 39R/A61 | GTCCAGGCTGTTTATGGTTTCGCTAAGTCTTTGCTCAAAGCACTGAAAGAAGACGGGGAC | |
| D/T88R/ | GATGTAATTGTTGTATTTGATGCCAAAGCACCGAGCTTCCGCCACGAAGCTTATGGTGGC | |
| E210R); | TACAAGGCAGGACGCGCCCCTCGTCCAGAAGATTTCCCCCGTCAGCTGGCATTAATTAAG | |
| DNA | GAGTTAGTAGACCTTCTCGGCTTAGCGCGTCTGGAAGTTCCGGGTTATGAGGCGGACGAT | |
| sequence | GTCCTTGCATCCTTGGCTAAAAAGGCCGAAAAAGAGGGCTACGAAGTCCGCATCTTGACG | |
| (5′ to 3′) | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAACGTTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M58 | MRGMLPLFEPKGRVLLVDGHRLAYRTFHALKGLTTSRGRPVQAVYGFAKSLLKALKEDGD | 103 |
| (H21R/E | DVIVVFDAKAPSFRHEAYGGYKAGRAPRPEDFPRQLALIKELVDLLGLARLEVPGYEADD | |
| 39R/A61 | VLASLAKKAEKEGYEVRILTADKDLYQLLSDRIHVLHPEGYLITPAWLWEKYGLRPDQWA | |
| D/T88R/ | DYRALTGDESDNLPGVKGIGEKTARKLLEEWGSLEALLKNLDRLKPAIREKILAHMDDLK | |
| V631A); | LSWDLAKVRTDLPLEVDFAKRREPDRERLRAFLERLEFGSLLHEFGLLESPKALEEAPWP | |
| Protein | PPEGAFVGFVLSRKEPMWADLLALAAARGGRVHRAPEPYKALRDLKEARGLLAKDLSVLA | |
| sequence | LREGLGLPPGDDPMLLAYLLDPSNTTPEGVARRYGGEWTEEAGERAALSERLFANLWGRL | |
| (N to C | EGEERLLWLYREVERPLSAVLAHMEATGVRLDVAYLRALSLEVAEEIARLEAEVFRLAGH | |
| terminus) | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRAFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M58 | ATGCGTGGTATGCTGCCGTTGTTCGAGCCTAAAGGCCGCGTACTGTTAGTCGATGGTCAT | 104 |
| (H21R/E | CGTTTGGCCTATCGGACGTTCCATGCACTCAAAGGTCTGACGACCAGTCGTGGCCGTCCG | |
| 39R/A61 | GTCCAGGCTGTTTATGGTTTCGCTAAGTCTTTGCTCAAAGCACTGAAAGAAGACGGGGAC | |
| D/T88R/ | GATGTAATTGTTGTATTTGATGCCAAAGCACCGAGCTTCCGCCACGAAGCTTATGGTGGC | |
| V631A); | TACAAGGCAGGACGCGCCCCTCGTCCAGAAGATTTCCCCCGTCAGCTGGCATTAATTAAG | |
| DNA | GAGTTAGTAGACCTTCTCGGCTTAGCGCGTCTGGAAGTTCCGGGTTATGAGGCGGACGAT | |
| sequence | GTCCTTGCATCCTTGGCTAAAAAGGCCGAAAAAGAGGGCTACGAAGTCCGCATCTTGACG | |
| (5′ to 3′) | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGCGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M59 | MRGMLPLFEPKGRVLLVDGHRLAYRTFHALKGLTTSRGRPVQAVYGFAKSLLKALKEDGD | 105 |
| (H21R/E | DVIVVFDAKAPSFRHEAYGGYKAGRAPRPEDFPRQLALIKELVDLLGLARLEVPGYEADD | |
| 39R/A61 | VLASLAKKAEKEGYEVRILTADKDLYQLLSDRIHVLHPEGYLITPAWLWEKYGLRPDQWA | |
| D/T88R/ | DYRALTGDESDNLPGVKGIGEKTARKLLEEWGSLEALLKNLDRLKPAIREKILAHMDDLK | |
| K709R); | LSWDLAKVRTDLPLEVDFAKRREPDRERLRAFLERLEFGSLLHEFGLLESPKALEEAPWP | |
| Protein | PPEGAFVGFVLSRKEPMWADLLALAAARGGRVHRAPEPYKALRDLKEARGLLAKDLSVLA | |
| sequence | LREGLGLPPGDDPMLLAYLLDPSNTTPEGVARRYGGEWTEEAGERAALSERLFANLWGRL | |
| (N to C | EGEERLLWLYREVERPLSAVLAHMEATGVRLDVAYLRALSLEVAEEIARLEAEVFRLAGH | |
| terminus) | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIERTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M59 | ATGCGTGGTATGCTGCCGTTGTTCGAGCCTAAAGGCCGCGTACTGTTAGTCGATGGTCAT | 106 |
| (H21R/E | CGTTTGGCCTATCGGACGTTCCATGCACTCAAAGGTCTGACGACCAGTCGTGGCCGTCCG | |
| 39R/A61 | GTCCAGGCTGTTTATGGTTTCGCTAAGTCTTTGCTCAAAGCACTGAAAGAAGACGGGGAC | |
| D/T88R/ | GATGTAATTGTTGTATTTGATGCCAAAGCACCGAGCTTCCGCCACGAAGCTTATGGTGGC | |
| K709R); | TACAAGGCAGGACGCGCCCCTCGTCCAGAAGATTTCCCCCGTCAGCTGGCATTAATTAAG | |
| DNA | GAGTTAGTAGACCTTCTCGGCTTAGCGCGTCTGGAAGTTCCGGGTTATGAGGCGGACGAT | |
| sequence | GTCCTTGCATCCTTGGCTAAAAAGGCCGAAAAAGAGGGCTACGAAGTCCGCATCTTGACG | |
| (5′ to 3′) | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGCGTACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M60 | MRGMLPLFEPKGRVLLVDGHRLAYRTFHALKGLTTSRGRPVQAVYGFAKSLLKALKEDGD | 107 |
| (H21R/E | DVIVVFDAKAPSFRHEAYGGYKAGRAPRPEDFPRQLALIKELVDLLGLARLEVPGYEADD | |
| 39R/A61 | VLASLAKKAEKEGYEVRILTADKDLYQLLSDRIHVLHPEGYLITPAWLWEKYGLRPDQWA | |
| D/T88R/ | DYRALTGDESDNLPGVKGIGEKTARKLLEEWGSLEALLKNLDRLKPAIREKILAHMDDLK | |
| E724G); | LSWDLAKVRTDLPLEVDFAKRREPDRERLRAFLERLEFGSLLHEFGLLESPKALEEAPWP | |
| Protein | PPEGAFVGFVLSRKEPMWADLLALAAARGGRVHRAPEPYKALRDLKEARGLLAKDLSVLA | |
| sequence | LREGLGLPPGDDPMLLAYLLDPSNTTPEGVARRYGGEWTEEAGERAALSERLFANLWGRL | |
| (N to C | EGEERLLWLYREVERPLSAVLAHMEATGVRLDVAYLRALSLEVAEEIARLEAEVFRLAGH | |
| terminus) | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVRGAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M60 | ATGCGTGGTATGCTGCCGTTGTTCGAGCCTAAAGGCCGCGTACTGTTAGTCGATGGTCAT | 108 |
| (H21R/E | CGTTTGGCCTATCGGACGTTCCATGCACTCAAAGGTCTGACGACCAGTCGTGGCCGTCCG | |
| 39R/A61 | GTCCAGGCTGTTTATGGTTTCGCTAAGTCTTTGCTCAAAGCACTGAAAGAAGACGGGGAC | |
| D/T88R/ | GATGTAATTGTTGTATTTGATGCCAAAGCACCGAGCTTCCGCCACGAAGCTTATGGTGGC | |
| E724G); | TACAAGGCAGGACGCGCCCCTCGTCCAGAAGATTTCCCCCGTCAGCTGGCATTAATTAAG | |
| DNA | GAGTTAGTAGACCTTCTCGGCTTAGCGCGTCTGGAAGTTCCGGGTTATGAGGCGGACGAT | |
| sequence | GTCCTTGCATCCTTGGCTAAAAAGGCCGAAAAAGAGGGCTACGAAGTCCGCATCTTGACG | |
| (5′ to 3′) | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGGCGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M61 | MRGMLPLFEPKGRVLLVDGHRLAYRTFHALKGLTTSRGRPVQAVYGFAKSLLKALKEDGD | 109 |
| (H21R/E | DVIVVFDAKAPSFRHEAYGGYKAGRAPRPEDFPRQLALIKELVDLLGLARLEVPGYEADD | |
| 39R/A61 | VLASLAKKAEKEGYEVRILTADKDLYQLLSDRIHVLHPEGYLITPAWLWEKYGLRPDQWA | |
| D/T88R/ | DYRALTGDESDNLPGVKGIGEKTARKLLEEWGSLEALLKNLDRLKPAIREKILAHMDDLK | |
| K831E); | LSWDLAKVRTDLPLEVDFAKRREPDRERLRAFLERLEFGSLLHEFGLLESPKALEEAPWP | |
| Protein | PPEGAFVGFVLSRKEPMWADLLALAAARGGRVHRAPEPYKALRDLKEARGLLAKDLSVLA | |
| sequence | LREGLGLPPGDDPMLLAYLLDPSNTTPEGVARRYGGEWTEEAGERAALSERLFANLWGRL | |
| (N to C | EGEERLLWLYREVERPLSAVLAHMEATGVRLDVAYLRALSLEVAEEIARLEAEVFRLAGH | |
| terminus) | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAEEAAALEIKR | |
| | ASQPELAPEDPEDVEHHHHHH | |
| M61 | ATGCGTGGTATGCTGCCGTTGTTCGAGCCTAAAGGCCGCGTACTGTTAGTCGATGGTCAT | 110 |
| (H21R/E | CGTTTGGCCTATCGGACGTTCCATGCACTCAAAGGTCTGACGACCAGTCGTGGCCGTCCG | |
| 39R/A61 | GTCCAGGCTGTTTATGGTTTCGCTAAGTCTTTGCTCAAAGCACTGAAAGAAGACGGGGAC | |
| D/T88R/ | GATGTAATTGTTGTATTTGATGCCAAAGCACCGAGCTTCCGCCACGAAGCTTATGGTGGC | |
| K831E); | TACAAGGCAGGACGCGCCCCTCGTCCAGAAGATTTCCCCCGTCAGCTGGCATTAATTAAG | |
| DNA | GAGTTAGTAGACCTTCTCGGCTTAGCGCGTCTGGAAGTTCCGGGTTATGAGGCGGACGAT | |
| sequence | GTCCTTGCATCCTTGGCTAAAAAGGCCGAAAAAGAGGGCTACGAAGTCCGCATCTTGACG | |
| (5′ to 3′) | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAGAGGAAgcggccgcactcgagatcaaacgg | |
| | gctagccagccagaactcgccccggaagaccccgaggatgtcgagcaccaccaccaccac | |
| | cac | |
| M62 | MRGMLPLFEPKGRVLLLDGHRLAYRTFHALKGLTTSRGEPVQAVYGFAKSLLKALKEDGD | 111 |
| (V17L/H | AVIVVFDAKAPSFRHEAYGGYKAGRAPRPEDFPRQLALIKELVDLLGLARLEVPGYEADD | |
| 21R/T88 | VLASLAKKAEKEGYEVRILTADKDLYQLLSDRIHVLHPEGYLITPAWLWEKYGLRPDQWA | |
| R/E210R | DYRALTGDESDNLPGVKGIGEKTARKLLERWGSLEALLKNLDRLKPAIREKILAHMDDLK | |
| ); Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| (N to C | LREGLGLPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M62 | ATGCGTGGTATGCTGCCGTTGTTCGAGCCTAAAGGCCGCGTACTGTTACTTGATGGTCAT | 112 |
| (V17L/H | CGTTTGGCCTATCGGACGTTCCATGCACTCAAAGGTCTGACGACCAGTCGTGGCGAACCG | |
| 21R/T88 | GTCCAGGCTGTTTATGGTTTCGCTAAGTCTTTGCTCAAAGCACTGAAAGAAGACGGGGAC | |
| R/E210R | GCGGTAATTGTTGTATTTGATGCCAAAGCACCGAGCTTCCGCCACGAAGCTTATGGTGGC | |
| ); DNA | TACAAGGCAGGACGCGCCCCTCGTCCAGAAGATTTCCCCCGTCAGCTGGCATTAATTAAG | |
| sequence | GAGTTAGTAGACCTTCTCGGCTTAGCGCGTCTGGAAGTTCCGGGTTATGAGGCGGACGAT | |
| (5′ to 3′) | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAACGTTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M63 | MRGMLPLFEPKGRVLLVDGHRLAYRTFHALKGLTTSRGRPVQAVYGFAKSLLKALKEDGD | 113 |
| (H21R/E | AVIVVFDAKAPSFRHEAYGGYKAGRAPRPEDFPRQLALIKELVDLLGLARLEVPGYEADD | |
| 39R/T88 | VLASLAKKAEKEGYEVRILTADKDLYQLLSDRIHVLHPEGYLITPAWLWEKYGLRPDQWA | |
| R/E210R | DYRALTGDESDNLPGVKGIGEKTARKLLERWGSLEALLKNLDRLKPAIREKILAHMDDLK | |
| ); Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| (N to C | LREGLGLPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M63 | ATGCGTGGTATGCTGCCGTTGTTCGAGCCTAAAGGCCGCGTACTGTTAGTCGATGGTCAT | 114 |
| (H21R/E | CGTTTGGCCTATCGGACGTTCCATGCACTCAAAGGTCTGACGACCAGTCGTGGCCGTCCG | |
| 39R/T88 | GTCCAGGCTGTTTATGGTTTCGCTAAGTCTTTGCTCAAAGCACTGAAAGAAGACGGGGAC | |
| R/E210R | GCGGTAATTGTTGTATTTGATGCCAAAGCACCGAGCTTCCGCCACGAAGCTTATGGTGGC | |
| ); DNA | TACAAGGCAGGACGCGCCCCTCGTCCAGAAGATTTCCCCCGTCAGCTGGCATTAATTAAG | |
| sequence | GAGTTAGTAGACCTTCTCGGCTTAGCGCGTCTGGAAGTTCCGGGTTATGAGGCGGACGAT | |
| (5′ to 3′) | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAACGTTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M64 | MRGMLPLFEPKGRVLLVDGHRLAYRTFHALKGLTTSRGEPVQAVYGFAKSLLKALKEDGD | 115 |
| (H21R/A | DVIVVFDAKAPSFRHEAYGGYKAGRAPRPEDFPRQLALIKELVDLLGLARLEVPGYEADD | |
| 61D/T88 | VLASLAKKAEKEGYEVRILTADKDLYQLLSDRIHVLHPEGYLITPAWLWEKYGLRPDQWA | |
| R/E210R | DYRALTGDESDNLPGVKGIGEKTARKLLERWGSLEALLKNLDRLKPAIREKILAHMDDLK | |
| ); Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| (N to C | LREGLGLPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M64 | ATGCGTGGTATGCTGCCGTTGTTCGAGCCTAAAGGCCGCGTACTGTTAGTCGATGGTCAT | 116 |
| (H21R/A | CGTTTGGCCTATCGGACGTTCCATGCACTCAAAGGTCTGACGACCAGTCGTGGCGAACCG | |
| 61D/T88 | GTCCAGGCTGTTTATGGTTTCGCTAAGTCTTTGCTCAAAGCACTGAAAGAAGACGGGGAC | |
| R/E210R | GATGTAATTGTTGTATTTGATGCCAAAGCACCGAGCTTCCGCCACGAAGCTTATGGTGGC | |
| ); DNA | TACAAGGCAGGACGCGCCCCTCGTCCAGAAGATTTCCCCCGTCAGCTGGCATTAATTAAG | |
| sequence | GAGTTAGTAGACCTTCTCGGCTTAGCGCGTCTGGAAGTTCCGGGTTATGAGGCGGACGAT | |
| (5′ to 3′) | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAACGTTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M65 | MRGMLPLFEPKGRVLLLDGHRLAYRTFHALKGLTTSRGRPVQAVYGFAKSLLKALKEDGD | 117 |
| (V17L/H | AVIVVFDAKAPSFRHEAYGGYKAGRAPRPEDFPRQLALIKELVDLLGLARLEVPGYEADD | |
| 21R/E39 | VLASLAKKAEKEGYEVRILTADKDLYQLLSDRIHVLHPEGYLITPAWLWEKYGLRPDQWA | |
| R/T88R/ | DYRALTGDESDNLPGVKGIGEKTARKLLERWGSLEALLKNLDRLKPAIREKILAHMDDLK | |
| E210R); | LSWDLAKVRTDLPLEVDFAKRREPDRERLRAFLERLEFGSLLHEFGLLESPKALEEAPWP | |
| Protein | PPEGAFVGFVLSRKEPMWADLLALAAARGGRVHRAPEPYKALRDLKEARGLLAKDLSVLA | |
| sequence | LREGLGLPPGDDPMLLAYLLDPSNTTPEGVARRYGGEWTEEAGERAALSERLFANLWGRL | |
| (N to C | EGEERLLWLYREVERPLSAVLAHMEATGVRLDVAYLRALSLEVAEEIARLEAEVFRLAGH | |
| terminus) | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M65 | ATGCGTGGTATGCTGCCGTTGTTCGAGCCTAAAGGCCGCGTACTGTTACTTGATGGTCAT | 118 |
| (V17L/H | CGTTTGGCCTATCGGACGTTCCATGCACTCAAAGGTCTGACGACCAGTCGTGGCCGTCCG | |
| 21R/E39 | GTCCAGGCTGTTTATGGTTTCGCTAAGTCTTTGCTCAAAGCACTGAAAGAAGACGGGGAC | |
| R/T88R/ | GCGGTAATTGTTGTATTTGATGCCAAAGCACCGAGCTTCCGCCACGAAGCTTATGGTGGC | |
| E210R); | TACAAGGCAGGACGCGCCCCTCGTCCAGAAGATTTCCCCCGTCAGCTGGCATTAATTAAG | |
| DNA | GAGTTAGTAGACCTTCTCGGCTTAGCGCGTCTGGAAGTTCCGGGTTATGAGGCGGACGAT | |
| sequence | GTCCTTGCATCCTTGGCTAAAAAGGCCGAAAAAGAGGGCTACGAAGTCCGCATCTTGACG | |
| (5′ to 3′) | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAACGTTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M66 | MRGMLPLFEPKGRVLLLDGHRLAYRTFHALKGLTTSRGEPVQAVYGFAKSLLKALKEDGD | 119 |
| (V17L/H | DVIVVFDAKAPSFRHEAYGGYKAGRAPRPEDFPRQLALIKELVDLLGLARLEVPGYEADD | |
| 21R/A61 | VLASLAKKAEKEGYEVRILTADKDLYQLLSDRIHVLHPEGYLITPAWLWEKYGLRPDQWA | |
| D/T88R/ | DYRALTGDESDNLPGVKGIGEKTARKLLERWGSLEALLKNLDRLKPAIREKILAHMDDLK | |
| E210R); | LSWDLAKVRTDLPLEVDFAKRREPDRERLRAFLERLEFGSLLHEFGLLESPKALEEAPWP | |
| Protein | PPEGAFVGFVLSRKEPMWADLLALAAARGGRVHRAPEPYKALRDLKEARGLLAKDLSVLA | |
| sequence | LREGLGLPPGDDPMLLAYLLDPSNTTPEGVARRYGGEWTEEAGERAALSERLFANLWGRL | |
| (N to C | EGEERLLWLYREVERPLSAVLAHMEATGVRLDVAYLRALSLEVAEEIARLEAEVFRLAGH | |
| terminus) | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M66 | ATGCGTGGTATGCTGCCGTTGTTCGAGCCTAAAGGCCGCGTACTGTTACTTGATGGTCAT | 120 |
| (V17L/H | CGTTTGGCCTATCGGACGTTCCATGCACTCAAAGGTCTGACGACCAGTCGTGGCGAACCG | |
| 21R/A61 | GTCCAGGCTGTTTATGGTTTCGCTAAGTCTTTGCTCAAAGCACTGAAAGAAGACGGGGAC | |
| D/T88R/ | GATGTAATTGTTGTATTTGATGCCAAAGCACCGAGCTTCCGCCACGAAGCTTATGGTGGC | |
| E210R); | TACAAGGCAGGACGCGCCCCTCGTCCAGAAGATTTCCCCCGTCAGCTGGCATTAATTAAG | |
| DNA | GAGTTAGTAGACCTTCTCGGCTTAGCGCGTCTGGAAGTTCCGGGTTATGAGGCGGACGAT | |
| sequence | GTCCTTGCATCCTTGGCTAAAAAGGCCGAAAAAGAGGGCTACGAAGTCCGCATCTTGACG | |
| (5′ to 3′) | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAACGTTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M67 | MRGMLPLFEPKGRVLLVDGHHLAYRTFHALKGLTTSRGEPVQAVYGFAKSLLKALKEDGD | 121 |
| (E507T/E | AVIVVFDAKAPSFRHEAYGGYKAGRAPTPEDFPRQLALIKELVDLLGLARLEVPGYEADD | |
| 210R); | VLASLAKKAEKEGYEVRILTADKDLYQLLSDRIHVLHPEGYLITPAWLWEKYGLRPDQWA | |
| Protein | DYRALTGDESDNLPGVKGIGEKTARKLLERWGSLEALLKNLDRLKPAIREKILAHMDDLK | |
| sequence | LSWDLAKVRTDLPLEVDFAKRREPDRERLRAFLERLEFGSLLHEFGLLESPKALEEAPWP | |
| (N to C | PPEGAFVGFVLSRKEPMWADLLALAAARGGRVHRAPEPYKALRDLKEARGLLAKDLSVLA | |
| terminus) | LREGLGLPPGDDPMLLAYLLDPSNTTPEGVARRYGGEWTEEAGERAALSERLFANLWGRL | |
| | EGEERLLWLYREVERPLSAVLAHMEATGVRLDVAYLRALSLEVAEEIARLEAEVFRLAGH | |
| | PFNLNSRDQLERVLFDELGLPAIGKTTKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M67 | ATGCGTGGTATGCTGCCGTTGTTCGAGCCTAAAGGCCGCGTACTGTTAGTCGATGGTCAT | 122 |
| (E507T/E | CACTTGGCCTATCGGACGTTCCATGCACTCAAAGGTCTGACGACCAGTCGTGGCGAACCG | |
| 210R); | GTCCAGGCTGTTTATGGTTTCGCTAAGTCTTTGCTCAAAGCACTGAAAGAAGACGGGGAC | |
| DNA | GCGGTAATTGTTGTATTTGATGCCAAAGCACCGAGCTTCCGCCACGAAGCTTATGGTGGC | |
| sequence | TACAAGGCAGGACGCGCCCCTACCCCAGAAGATTTCCCCCGTCAGCTGGCATTAATTAAG | |
| (5′ to 3′) | GAGTTAGTAGACCTTCTCGGCTTAGCGCGTCTGGAAGTTCCGGGTTATGAGGCGGACGAT | |
| | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAACGTTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCACCAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M68 | MRGMLPLFEPKGRVLLVDGHHLAYRTFHALKGLTTSRGEPVQAVYGFAKSLLKALKEDGD | 123 |
| (T88R/E | AVIVVFDAKAPSFRHEAYGGYKAGRAPRPEDFPRQLALIKELVDLLGLARLEVPGYEADD | |
| 507T); | VLASLAKKAEKEGYEVRILTADKDLYQLLSDRIHVLHPEGYLITPAWLWEKYGLRPDQWA | |
| Protein | DYRALTGDESDNLPGVKGIGEKTARKLLEEWGSLEALLKNLDRLKPAIREKILAHMDDLK | |
| sequence | LSWDLAKVRTDLPLEVDFAKRREPDRERLRAFLERLEFGSLLHEFGLLESPKALEEAPWP | |
| (N to C | PPEGAFVGFVLSRKEPMWADLLALAAARGGRVHRAPEPYKALRDLKEARGLLAKDLSVLA | |
| terminus) | LREGLGLPPGDDPMLLAYLLDPSNTTPEGVARRYGGEWTEEAGERAALSERLFANLWGRL | |
| | EGEERLLWLYREVERPLSAVLAHMEATGVRLDVAYLRALSLEVAEEIARLEAEVFRLAGH | |
| | PFNLNSRDOLERVLFDELGLPAIGKTTKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M68 | ATGCGTGGTATGCTGCCGTTGTTCGAGCCTAAAGGCCGCGTACTGTTAGTCGATGGTCAT | 124 |
| (T88R/E | CACTTGGCCTATCGGACGTTCCATGCACTCAAAGGTCTGACGACCAGTCGTGGCGAACCG | |
| 507T); | GTCCAGGCTGTTTATGGTTTCGCTAAGTCTTTGCTCAAAGCACTGAAAGAAGACGGGGAC | |
| DNA | GCGGTAATTGTTGTATTTGATGCCAAAGCACCGAGCTTCCGCCACGAAGCTTATGGTGGC | |
| sequence | TACAAGGCAGGACGCGCCCCTCGTCCAGAAGATTTCCCCCGTCAGCTGGCATTAATTAAG | |
| (5′ to 3′) | GAGTTAGTAGACCTTCTCGGCTTAGCGCGTCTGGAAGTTCCGGGTTATGAGGCGGACGAT | |
| | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCACCAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M69 | MRGMLPLFEPKGRVLLVDGHHLAYRTFHALKGLTTSRGEPVQAVYGFAKSLLKALKEDGD | 125 |
| (T88R/E | AVIVVFDAKAPSFRHEAYGGYKAGRAPRPEDFPRQLALIKELVDLLGLARLEVPGYEADD | |
| 210R); | VLASLAKKAEKEGYEVRILTADKDLYQLLSDRIHVLHPEGYLITPAWLWEKYGLRPDQWA | |
| Protein | DYRALTGDESDNLPGVKGIGEKTARKLLERWGSLEALLKNLDRLKPAIREKILAHMDDLK | |
| sequence | LSWDLAKVRTDLPLEVDFAKRREPDRERLRAFLERLEFGSLLHEFGLLESPKALEEAPWP | |
| (N to C | PPEGAFVGFVLSRKEPMWADLLALAAARGGRVHRAPEPYKALRDLKEARGLLAKDLSVLA | |
| terminus) | LREGLGLPPGDDPMLLAYLLDPSNTTPEGVARRYGGEWTEEAGERAALSERLFANLWGRL | |
| | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M69 | ATGCGTGGTATGCTGCCGTTGTTCGAGCCTAAAGGCCGCGTACTGTTAGTCGATGGTCAT | 126 |
| (T88R/E | CACTTGGCCTATCGGACGTTCCATGCACTCAAAGGTCTGACGACCAGTCGTGGCGAACCG | |
| 210R); | GTCCAGGCTGTTTATGGTTTCGCTAAGTCTTTGCTCAAAGCACTGAAAGAAGACGGGGAC | |
| DNA | GCGGTAATTGTTGTATTTGATGCCAAAGCACCGAGCTTCCGCCACGAAGCTTATGGTGGC | |
| sequence | TACAAGGCAGGACGCGCCCCTCGTCCAGAAGATTTCCCCCGTCAGCTGGCATTAATTAAG | |
| (5′ to 3′) | GAGTTAGTAGACCTTCTCGGCTTAGCGCGTCTGGAAGTTCCGGGTTATGAGGCGGACGAT | |
| | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAACGTTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M70 | MRGMLPLFEPKGRVLLVDGHHLAYRTFHALKGLTTSRGEPVQAVYGFAKSLLKALKEDGD | 127 |
| (A61D/E | DVIVVFDAKAPSFRHEAYGGYKAGRAPTPEDFPRQLALIKELVDLLGLARLEVPGYEADD | |
| 507T); | VLASLAKKAEKEGYEVRILTADKDLYQLLSDRIHVLHPEGYLITPAWLWEKYGLRPDQWA | |
| Protein | DYRALTGDESDNLPGVKGIGEKTARKLLEEWGSLEALLKNLDRLKPAIREKILAHMDDLK | |
| sequence | LSWDLAKVRTDLPLEVDFAKRREPDRERLRAFLERLEFGSLLHEFGLLESPKALEEAPWP | |
| (N to C | PPEGAFVGFVLSRKEPMWADLLALAAARGGRVHRAPEPYKALRDLKEARGLLAKDLSVLA | |
| terminus) | LREGLGLPPGDDPMLLAYLLDPSNTTPEGVARRYGGEWTEEAGERAALSERLFANLWGRL | |
| | EGEERLLWLYREVERPLSAVLAHMEATGVRLDVAYLRALSLEVAEEIARLEAEVFRLAGH | |
| | PFNLNSRDQLERVLFDELGLPAIGKTTKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M70 | ATGCGTGGTATGCTGCCGTTGTTCGAGCCTAAAGGCCGCGTACTGTTAGTCGATGGTCAT | 128 |
| (A61D/E | CACTTGGCCTATCGGACGTTCCATGCACTCAAAGGTCTGACGACCAGTCGTGGCGAACCG | |
| 507T); | GTCCAGGCTGTTTATGGTTTCGCTAAGTCTTTGCTCAAAGCACTGAAAGAAGACGGGGAC | |
| DNA | GATGTAATTGTTGTATTTGATGCCAAAGCACCGAGCTTCCGCCACGAAGCTTATGGTGGC | |
| sequence | TACAAGGCAGGACGCGCCCCTACCCCAGAAGATTTCCCCCGTCAGCTGGCATTAATTAAG | |
| (5′ to 3′) | GAGTTAGTAGACCTTCTCGGCTTAGCGCGTCTGGAAGTTCCGGGTTATGAGGCGGACGAT | |
| | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTT | |
| | ACCGCTTGAAGTGGATTTTGCAAAACGCCGTGAGCCGGACCGGGAACGTTTACGCGCTTT | |
| | CTTAGAGCGTCTGGAATTCGGTTCACTGCTTCATGAATTCGGTCTGTTAGAGTCTCCTAA | |
| | AGCACTCGAAGAGGCACCGTGGCCGCCCCCAGAAGGTGCTTTTGTTGGCTTCGTACTTTC | |
| | CCGTAAGGAGCCTATGTGGGCAGATCTTCTGGCTTTAGCGGCTGCACGCGGTGGCCGTGT | |
| | TCACCGGGCCCCTGAGCCATACAAAGCGTTACGTGATCTGAAGGAAGCACGTGGCTTGCT | |
| | GGCAAAAGACCTTTCTGTTTTGGCCCTGCGCGAGGGTCTTGGACTGCCGCCAGGCGACGA | |
| | TCCCATGTTATTGGCCTATCTGTTAGACCCTAGCAATACCACACCTGAAGGGGTCGCTCG | |
| | TCGGTATGGCGGTGAATGGACTGAGGAAGCCGGAGAGCGCGCCGCATTGTCCGAACGGCT | |
| | CTTTGCAAACTTATGGGGTCGTCTGGAAGGGGAGGAACGTCTGTTATGGTTGTATCGGGA | |
| | AGTCGAACGTCCTCTTTCGGCCGTATTAGCGCATATGGAGGCAACAGGTGTGCGTTTAGA | |
| | TGTCGCGTACCTTCGGGCCTTATCACTGGAAGTTGCAGAGGAAATCGCCCGTCTCGAGGC | |
| | TGAAGTGTTCCGGTTGGCCGGTCACCCGTTTAACCTCAACTCCCGTGACCAGCTGGAACG | |
| | CGTTTTATTCGATGAGCTTGGGCTTCCCGCAATTGGCAAAACCACCAAGACTGGCAAACG | |
| | CAGTACGAGCGCTGCCGTCCTTGAGGCACTCCGCGAGGCTCACCCTATTGTAGAAAAGAT | |
| | CCTGCAATACCGTGAGTTGACGAAGCTTAAAAGCACTTATATTGATCCTCTCCCGGATCT | |
| | GATCCATCCTCGTACCGGCCGCTTGCACACACGTTTCAACCAGACGGCGACTGCAACCGG | |
| | CCGTCTGTCTAGCTCGGATCCAAATCTCCAGAACATTCCGGTCCGTACACCCTTGGGCCA | |
| | ACGTATCCGCCGGGCGTTTATCGCGGAAGAAGGATGGTTACTGGTCGCATTGGACTACTC | |
| | GCAGATTGAGCTGCGCGTCCTCGCACATCTCTCTGGTGACGAAAATTTAATCCGCGTGTT | |
| | TCAAGAGGGGCGTGATATTCACACAGAAACTGCCTCATGGATGTTCGGTGTCCCACGTGA | |
| | AGCAGTGGATCCTTTGATGCGCCGTGCAGCTAAAACAATTAATTTTGGAGTGCTGTACGG | |
| | AATGAGCGCTCATCGCTTGAGTCAGGAACTGGCAATCCCCTACGAGGAAGCGCAGGCATT | |
| | CATCGAACGTTACTTTCAATCGTTTCCGAAAGTTCGCGCATGGATCGAGAAGACGCTCGA | |
| | GGAAGGTCGTCGTCGGGGCTATGTCGAAACTCTGTTTGGTCGCCGTCGGTACGTACCAGA | |
| | TCTTGAAGCCCGCGTCAAATCGGTACGGGAGGCTGCGGAGCGTATGGCATTTAATATGCC | |
| | TGTACAGGGTACTGCAGCTGACCTCATGAAACTGGCAATGGTCAAGCTTTTCCCGCGCTT | |
| | GGAGGAAATGGGCGCACGTATGCTTCTGCAGGTCCATGACGAGCTGGTGTTAGAAGCCCC | |
| | TAAGGAGCGCGCCGAAGCTGTCGCGCGCCTCGCTAAAGAAGTGATGGAGGGCGTTTACCC | |
| | ATTGGCCGTACCCCTCGAAGTGGAGGTCGGTATTGGAGAAGATTGGTTATCTGCAAAGGA | |
| | Agcggccgcactcgagatcaaacgggctagccagccagaactcgccccggaagaccccga | |
| | ggatgtcgagcaccaccaccaccaccac | |
| M71 | MRGMLPLFEPKGRVLLVDGHHLAYRTFHALKGLTTSRGEPVQAVYGFAKSLLKALKEDGD | 129 |
| (A61D/E | DVIVVFDAKAPSFRHEAYGGYKAGRAPTPEDFPRQLALIKELVDLLGLARLEVPGYEADD | |
| 210R); | VLASLAKKAEKEGYEVRILTADKDLYQLLSDRIHVLHPEGYLITPAWLWEKYGLRPDQWA | |
| Protein | DYRALTGDESDNLPGVKGIGEKTARKLLERWGSLEALLKNLDRLKPAIREKILAHMDDLK | |
| sequence | LSWDLAKVRTDLPLEVDFAKRREPDRERLRAFLERLEFGSLLHEFGLLESPKALEEAPWP | |
| (N to C | PPEGAFVGFVLSRKEPMWADLLALAAARGGRVHRAPEPYKALRDLKEARGLLAKDLSVLA | |
| terminus) | LREGLGLPPGDDPMLLAYLLDPSNTTPEGVARRYGGEWTEEAGERAALSERLFANLWGRL | |
| | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M71 | ATGCGTGGTATGCTGCCGTTGTTCGAGCCTAAAGGCCGCGTACTGTTAGTCGATGGTCAT | 130 |
| (A61D/E | CACTTGGCCTATCGGACGTTCCATGCACTCAAAGGTCTGACGACCAGTCGTGGCGAACCG | |
| 210R); | GTCCAGGCTGTTTATGGTTTCGCTAAGTCTTTGCTCAAAGCACTGAAAGAAGACGGGGAC | |
| DNA | GATGTAATTGTTGTATTTGATGCCAAAGCACCGAGCTTCCGCCACGAAGCTTATGGTGGC | |
| sequence | TACAAGGCAGGACGCGCCCCTACCCCAGAAGATTTCCCCCGTCAGCTGGCATTAATTAAG | |
| (5′ to 3′) | GAGTTAGTAGACCTTCTCGGCTTAGCGCGTCTGGAAGTTCCGGGTTATGAGGCGGACGAT | |
| | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAACGTTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M72 | MRGMLPLFEPKGRVLLVDGHRLAYRTFHALKGLTTSRGEPVQAVYGFAKSLLKALKEDGD | 131 |
| (H21R/E | AVIVVFDAKAPSFRHEAYGGYKAGRAPTPEDFPRQLALIKELVDLLGLARLEVPGYEADD | |
| 507T); | VLASLAKKAEKEGYEVRILTADKDLYQLLSDRIHVLHPEGYLITPAWLWEKYGLRPDQWA | |
| Protein | DYRALTGDESDNLPGVKGIGEKTARKLLEEWGSLEALLKNLDRLKPAIREKILAHMDDLK | |
| sequence | LSWDLAKVRTDLPLEVDFAKRREPDRERLRAFLERLEFGSLLHEFGLLESPKALEEAPWP | |
| (N to C | PPEGAFVGFVLSRKEPMWADLLALAAARGGRVHRAPEPYKALRDLKEARGLLAKDLSVLA | |
| terminus) | LREGLGLPPGDDPMLLAYLLDPSNTTPEGVARRYGGEWTEEAGERAALSERLFANLWGRL | |
| | EGEERLLWLYREVERPLSAVLAHMEATGVRLDVAYLRALSLEVAEEIARLEAEVFRLAGH | |
| | PFNLNSRDQLERVLFDELGLPAIGKTTKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M72 | ATGCGTGGTATGCTGCCGTTGTTCGAGCCTAAAGGCCGCGTACTGTTAGTCGATGGTCAT | 132 |
| (H21R/E | CGTTTGGCCTATCGGACGTTCCATGCACTCAAAGGTCTGACGACCAGTCGTGGCGAACCG | |
| 507T); | GTCCAGGCTGTTTATGGTTTCGCTAAGTCTTTGCTCAAAGCACTGAAAGAAGACGGGGAC | |
| DNA | GCGGTAATTGTTGTATTTGATGCCAAAGCACCGAGCTTCCGCCACGAAGCTTATGGTGGC | |
| sequence | TACAAGGCAGGACGCGCCCCTACCCCAGAAGATTTCCCCCGTCAGCTGGCATTAATTAAG | |
| (5′ to 3′) | GAGTTAGTAGACCTTCTCGGCTTAGCGCGTCTGGAAGTTCCGGGTTATGAGGCGGACGAT | |
| | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCG | |
| | ATCAGTGGGCGGATTATCGGGCTTTGACGGGAGATGAGAGCGACAACCTGCCAGGAGTTA | |
| | AGGGCATTGGTGAAAAAACCGCACGTAAGCTGCTTGAAGAGTGGGGTTCCCTGGAAGCCT | |
| | TGTTAAAAAATCTGGATCGTCTCAAGCCCGCAATTCGTGAAAAGATCCTGGCTCATATGG | |
| | ACGATCTTAAATTAAGTTGGGACCTGGCCAAGGTGCGCACCGATTTACCGCTTGAAGTGG | |
| | ATTTTGCAAAACGCCGTGAGCCGGACCGGGAACGTTTACGCGCTTTCTTAGAGCGTCTGG | |
| | AATTCGGTTCACTGCTTCATGAATTCGGTCTGTTAGAGTCTCCTAAAGCACTCGAAGAGG | |
| | CACCGTGGCCGCCCCCAGAAGGTGCTTTTGTTGGCTTCGTACTTTCCCGTAAGGAGCCTA | |
| | TGTGGGCAGATCTTCTGGCTTTAGCGGCTGCACGCGGTGGCCGTGTTCACCGGGCCCCTG | |
| | AGCCATACAAAGCGTTACGTGATCTGAAGGAAGCACGTGGCTTGCTGGCAAAAGACCTTT | |
| | CTGTTTTGGCCCTGCGCGAGGGTCTTGGACTGCCGCCAGGCGACGATCCCATGTTATTGG | |
| | CCTATCTGTTAGACCCTAGCAATACCACACCTGAAGGGGTCGCTCGTCGGTATGGCGGTG | |
| | AATGGACTGAGGAAGCCGGAGAGCGCGCCGCATTGTCCGAACGGCTCTTTGCAAACTTAT | |
| | GGGGTCGTCTGGAAGGGGAGGAACGTCTGTTATGGTTGTATCGGGAAGTCGAACGTCCTC | |
| | TTTCGGCCGTATTAGCGCATATGGAGGCAACAGGTGTGCGTTTAGATGTCGCGTACCTTC | |
| | GGGCCTTATCACTGGAAGTTGCAGAGGAAATCGCCCGTCTCGAGGCTGAAGTGTTCCGGT | |
| | TGGCCGGTCACCCGTTTAACCTCAACTCCCGTGACCAGCTGGAACGCGTTTTATTCGATG | |
| | AGCTTGGGCTTCCCGCAATTGGCAAAACCACCAAGACTGGCAAACGCAGTACGAGCGCTG | |
| | CCGTCCTTGAGGCACTCCGCGAGGCTCACCCTATTGTAGAAAAGATCCTGCAATACCGTG | |
| | AGTTGACGAAGCTTAAAAGCACTTATATTGATCCTCTCCCGGATCTGATCCATCCTCGTA | |
| | CCGGCCGCTTGCACACACGTTTCAACCAGACGGCGACTGCAACCGGCCGTCTGTCTAGCT | |
| | CGGATCCAAATCTCCAGAACATTCCGGTCCGTACACCCTTGGGCCAACGTATCCGCCGGG | |
| | CGTTTATCGCGGAAGAAGGATGGTTACTGGTCGCATTGGACTACTCGCAGATTGAGCTGC | |
| | GCGTCCTCGCACATCTCTCTGGTGACGAAAATTTAATCCGCGTGTTTCAAGAGGGGCGTG | |
| | ATATTCACACAGAAACTGCCTCATGGATGTTCGGTGTCCCACGTGAAGCAGTGGATCCTT | |
| | TGATGCGCCGTGCAGCTAAAACAATTAATTTTGGAGTGCTGTACGGAATGAGCGCTCATC | |
| | GCTTGAGTCAGGAACTGGCAATCCCCTACGAGGAAGCGCAGGCATTCATCGAACGTTACT | |
| | TTCAATCGTTTCCGAAAGTTCGCGCATGGATCGAGAAGACGCTCGAGGAAGGTCGTCGTC | |
| | GGGGCTATGTCGAAACTCTGTTTGGTCGCCGTCGGTACGTACCAGATCTTGAAGCCCGCG | |
| | TCAAATCGGTACGGGAGGCTGCGGAGCGTATGGCATTTAATATGCCTGTACAGGGTACTG | |
| | CAGCTGACCTCATGAAACTGGCAATGGTCAAGCTTTTCCCGCGCTTGGAGGAAATGGGCG | |
| | CACGTATGCTTCTGCAGGTCCATGACGAGCTGGTGTTAGAAGCCCCTAAGGAGCGCGCCG | |
| | AAGCTGTCGCGCGCCTCGCTAAAGAAGTGATGGAGGGCGTTTACCCATTGGCCGTACCCC | |
| | TCGAAGTGGAGGTCGGTATTGGAGAAGATTGGTTATCTGCAAAGGAAgcggccgcactcg | |
| | agatcaaacgggctagccagccagaactcgccccggaagaccccgaggatgtcgagcacc | |
| | accaccaccaccac | |
| M73 | MRGMLPLFEPKGRVLLVDGHRLAYRTFHALKGLTTSRGEPVQAVYGFAKSLLKALKEDGD | 133 |
| (H21R/E | AVIVVFDAKAPSFRHEAYGGYKAGRAPTPEDFPRQLALIKELVDLLGLARLEVPGYEADD | |
| 210R); | VLASLAKKAEKEGYEVRILTADKDLYQLLSDRIHVLHPEGYLITPAWLWEKYGLRPDQWA | |
| Protein | DYRALTGDESDNLPGVKGIGEKTARKLLERWGSLEALLKNLDRLKPAIREKILAHMDDLK | |
| sequence | LSWDLAKVRTDLPLEVDFAKRREPDRERLRAFLERLEFGSLLHEFGLLESPKALEEAPWP | |
| (N to C | PPEGAFVGFVLSRKEPMWADLLALAAARGGRVHRAPEPYKALRDLKEARGLLAKDLSVLA | |
| terminus) | LREGLGLPPGDDPMLLAYLLDPSNTTPEGVARRYGGEWTEEAGERAALSERLFANLWGRL | |
| | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M73 | ATGCGTGGTATGCTGCCGTTGTTCGAGCCTAAAGGCCGCGTACTGTTAGTCGATGGTCAT | 134 |
| (H21R/E | CGTTTGGCCTATCGGACGTTCCATGCACTCAAAGGTCTGACGACCAGTCGTGGCGAACCG | |
| 210R); | GTCCAGGCTGTTTATGGTTTCGCTAAGTCTTTGCTCAAAGCACTGAAAGAAGACGGGGAC | |
| DNA | GCGGTAATTGTTGTATTTGATGCCAAAGCACCGAGCTTCCGCCACGAAGCTTATGGTGGC | |
| sequence | TACAAGGCAGGACGCGCCCCTACCCCAGAAGATTTCCCCCGTCAGCTGGCATTAATTAAG | |
| (5′ to 3′) | GAGTTAGTAGACCTTCTCGGCTTAGCGCGTCTGGAAGTTCCGGGTTATGAGGCGGACGAT | |
| | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAACGTTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M74 | MRGMLPLFEPKGRVLLLDGHHLAYRTFHALKGLTTSRGEPVQAVYGFAKSLLKALKEDGD | 135 |
| (V17L/E | AVIVVFDAKAPSFRHEAYGGYKAGRAPTPEDFPRQLALIKELVDLLGLARLEVPGYEADD | |
| 507T); | VLASLAKKAEKEGYEVRILTADKDLYQLLSDRIHVLHPEGYLITPAWLWEKYGLRPDQWA | |
| Protein | DYRALTGDESDNLPGVKGIGEKTARKLLEEWGSLEALLKNLDRLKPAIREKILAHMDDLK | |
| sequence | LSWDLAKVRTDLPLEVDFAKRREPDRERLRAFLERLEFGSLLHEFGLLESPKALEEAPWP | |
| (N to C | PPEGAFVGFVLSRKEPMWADLLALAAARGGRVHRAPEPYKALRDLKEARGLLAKDLSVLA | |
| terminus) | LREGLGLPPGDDPMLLAYLLDPSNTTPEGVARRYGGEWTEEAGERAALSERLFANLWGRL | |
| | EGEERLLWLYREVERPLSAVLAHMEATGVRLDVAYLRALSLEVAEEIARLEAEVFRLAGH | |
| | PFNLNSRDQLERVLFDELGLPAIGKTTKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M74 | ATGCGTGGTATGCTGCCGTTGTTCGAGCCTAAAGGCCGCGTACTGTTACTTGATGGTCAT | 136 |
| (V17L/E | CACTTGGCCTATCGGACGTTCCATGCACTCAAAGGTCTGACGACCAGTCGTGGCGAACCG | |
| 507T); | GTCCAGGCTGTTTATGGTTTCGCTAAGTCTTTGCTCAAAGCACTGAAAGAAGACGGGGAC | |
| DNA | GCGGTAATTGTTGTATTTGATGCCAAAGCACCGAGCTTCCGCCACGAAGCTTATGGTGGC | |
| sequence | TACAAGGCAGGACGCGCCCCTACCCCAGAAGATTTCCCCCGTCAGCTGGCATTAATTAAG | |
| (5′ to 3′) | GAGTTAGTAGACCTTCTCGGCTTAGCGCGTCTGGAAGTTCCGGGTTATGAGGCGGACGAT | |
| | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCACCAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M75 | MRGMLPLFEPKGRVLLLDGHHLAYRTFHALKGLTTSRGEPVQAVYGFAKSLLKALKEDGD | 137 |
| (V17L/E | AVIVVFDAKAPSFRHEAYGGYKAGRAPTPEDFPRQLALIKELVDLLGLARLEVPGYEADD | |
| 210R); | VLASLAKKAEKEGYEVRILTADKDLYQLLSDRIHVLHPEGYLITPAWLWEKYGLRPDQWA | |
| Protein | DYRALTGDESDNLPGVKGIGEKTARKLLERWGSLEALLKNLDRLKPAIREKILAHMDDLK | |
| sequence | LSWDLAKVRTDLPLEVDFAKRREPDRERLRAFLERLEFGSLLHEFGLLESPKALEEAPWP | |
| (N to C | PPEGAFVGFVLSRKEPMWADLLALAAARGGRVHRAPEPYKALRDLKEARGLLAKDLSVLA | |
| terminus) | LREGLGLPPGDDPMLLAYLLDPSNTTPEGVARRYGGEWTEEAGERAALSERLFANLWGRL | |
| | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M75 | ATGCGTGGTATGCTGCCGTTGTTCGAGCCTAAAGGCCGCGTACTGTTACTTGATGGTCAT | 138 |
| (V17L/E | CACTTGGCCTATCGGACGTTCCATGCACTCAAAGGTCTGACGACCAGTCGTGGCGAACCG | |
| 210R); | GTCCAGGCTGTTTATGGTTTCGCTAAGTCTTTGCTCAAAGCACTGAAAGAAGACGGGGAC | |
| DNA | GCGGTAATTGTTGTATTTGATGCCAAAGCACCGAGCTTCCGCCACGAAGCTTATGGTGGC | |
| sequence | TACAAGGCAGGACGCGCCCCTACCCCAGAAGATTTCCCCCGTCAGCTGGCATTAATTAAG | |
| (5′ to 3′) | GAGTTAGTAGACCTTCTCGGCTTAGCGCGTCTGGAAGTTCCGGGTTATGAGGCGGACGAT | |
| | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAACGTTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M76 | MRGMLPLFEPKGRVLLLDGHHLAYRTFHALKGLTTSRGEPVQAVYGFAKSLLKALKEDGD | 139 |
| (V17L/T | AVIVVFDAKAPSFRHEAYGGYKAGRAPRPEDFPRQLALIKELVDLLGLARLEVPGYEADD | |
| 88R); | VLASLAKKAEKEGYEVRILTADKDLYQLLSDRIHVLHPEGYLITPAWLWEKYGLRPDQWA | |
| Protein | DYRALTGDESDNLPGVKGIGEKTARKLLEEWGSLEALLKNLDRLKPAIREKILAHMDDLK | |
| sequence | LSWDLAKVRTDLPLEVDFAKRREPDRERLRAFLERLEFGSLLHEFGLLESPKALEEAPWP | |
| (N to C | PPEGAFVGFVLSRKEPMWADLLALAAARGGRVHRAPEPYKALRDLKEARGLLAKDLSVLA | |
| terminus) | LREGLGLPPGDDPMLLAYLLDPSNTTPEGVARRYGGEWTEEAGERAALSERLFANLWGRL | |
| | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M76 | ATGCGTGGTATGCTGCCGTTGTTCGAGCCTAAAGGCCGCGTACTGTTACTTGATGGTCAT | 140 |
| (V17L/T | CACTTGGCCTATCGGACGTTCCATGCACTCAAAGGTCTGACGACCAGTCGTGGCGAACCG | |
| 88R); | GTCCAGGCTGTTTATGGTTTCGCTAAGTCTTTGCTCAAAGCACTGAAAGAAGACGGGGAC | |
| DNA | GCGGTAATTGTTGTATTTGATGCCAAAGCACCGAGCTTCCGCCACGAAGCTTATGGTGGC | |
| sequence | TACAAGGCAGGACGCGCCCCTCGTCCAGAAGATTTCCCCCGTCAGCTGGCATTAATTAAG | |
| (5′ to 3′) | GAGTTAGTAGACCTTCTCGGCTTAGCGCGTCTGGAAGTTCCGGGTTATGAGGCGGACGAT | |
| | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M77 | MRGMLPLFEPKGRVLLLDGHHLAYRTFHALKGLTTSRGEPVQAVYGFAKSLLKALKEDGD | 141 |
| (V17L/A | DVIVVFDAKAPSFRHEAYGGYKAGRAPTPEDFPRQLALIKELVDLLGLARLEVPGYEADD | |
| 61D); | VLASLAKKAEKEGYEVRILTADKDLYQLLSDRIHVLHPEGYLITPAWLWEKYGLRPDQWA | |
| Protein | DYRALTGDESDNLPGVKGIGEKTARKLLEEWGSLEALLKNLDRLKPAIREKILAHMDDLK | |
| sequence | LSWDLAKVRTDLPLEVDFAKRREPDRERLRAFLERLEFGSLLHEFGLLESPKALEEAPWP | |
| (N to C | PPEGAFVGFVLSRKEPMWADLLALAAARGGRVHRAPEPYKALRDLKEARGLLAKDLSVLA | |
| terminus) | LREGLGLPPGDDPMLLAYLLDPSNTTPEGVARRYGGEWTEEAGERAALSERLFANLWGRL | |
| | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M77 | ATGCGTGGTATGCTGCCGTTGTTCGAGCCTAAAGGCCGCGTACTGTTACTTGATGGTCAT | 142 |
| (V17L/A | CACTTGGCCTATCGGACGTTCCATGCACTCAAAGGTCTGACGACCAGTCGTGGCGAACCG | |
| 61D); | GTCCAGGCTGTTTATGGTTTCGCTAAGTCTTTGCTCAAAGCACTGAAAGAAGACGGGGAC | |
| DNA | GATGTAATTGTTGTATTTGATGCCAAAGCACCGAGCTTCCGCCACGAAGCTTATGGTGGC | |
| sequence | TACAAGGCAGGACGCGCCCCTACCCCAGAAGATTTCCCCCGTCAGCTGGCATTAATTAAG | |
| (5′ to 3′) | GAGTTAGTAGACCTTCTCGGCTTAGCGCGTCTGGAAGTTCCGGGTTATGAGGCGGACGAT | |
| | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M78 | MRGMLPLFEPKGRVLLLDGHRLAYRTFHALKGLTTSRGEPVQAVYGFAKSLLKALKEDGD | 143 |
| (V17L/H | AVIVVFDAKAPSFRHEAYGGYKAGRAPTPEDFPRQLALIKELVDLLGLARLEVPGYEADD | |
| 21R); | VLASLAKKAEKEGYEVRILTADKDLYQLLSDRIHVLHPEGYLITPAWLWEKYGLRPDQWA | |
| Protein | DYRALTGDESDNLPGVKGIGEKTARKLLEEWGSLEALLKNLDRLKPAIREKILAHMDDLK | |
| sequence | LSWDLAKVRTDLPLEVDFAKRREPDRERLRAFLERLEFGSLLHEFGLLESPKALEEAPWP | |
| (N to C | PPEGAFVGFVLSRKEPMWADLLALAAARGGRVHRAPEPYKALRDLKEARGLLAKDLSVLA | |
| terminus) | LREGLGLPPGDDPMLLAYLLDPSNTTPEGVARRYGGEWTEEAGERAALSERLFANLWGRL | |
| | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M78 | ATGCGTGGTATGCTGCCGTTGTTCGAGCCTAAAGGCCGCGTACTGTTACTTGATGGTCAT | 144 |
| (V17L/H | CGTTTGGCCTATCGGACGTTCCATGCACTCAAAGGTCTGACGACCAGTCGTGGCGAACCG | |
| 21R); | GTCCAGGCTGTTTATGGTTTCGCTAAGTCTTTGCTCAAAGCACTGAAAGAAGACGGGGAC | |
| DNA | GCGGTAATTGTTGTATTTGATGCCAAAGCACCGAGCTTCCGCCACGAAGCTTATGGTGGC | |
| sequence | TACAAGGCAGGACGCGCCCCTACCCCAGAAGATTTCCCCCGTCAGCTGGCATTAATTAAG | |
| (5′ to 3′) | GAGTTAGTAGACCTTCTCGGCTTAGCGCGTCTGGAAGTTCCGGGTTATGAGGCGGACGAT | |
| | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M79 | MRGMLPLFEPKGRVLLVDGHHLAYRTFHALKGLTTSRGEPVQAVYGFAKSLLKALKEDGD | 145 |
| (T88R/E | AVIVVFDAKAPSFRHEAYGGYKAGRAPRPEDFPRQLALIKELVDLLGLARLEVPGYEADD | |
| 210R/E5 | VLASLAKKAEKEGYEVRILTADKDLYQLLSDRIHVLHPEGYLITPAWLWEKYGLRPDQWA | |
| 07T); | DYRALTGDESDNLPGVKGIGEKTARKLLERWGSLEALLKNLDRLKPAIREKILAHMDDLK | |
| Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| (N to C | LREGLGLPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTTKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M79 | ATGCGTGGTATGCTGCCGTTGTTCGAGCCTAAAGGCCGCGTACTGTTAGTCGATGGTCAT | 146 |
| (T88R/E | CACTTGGCCTATCGGACGTTCCATGCACTCAAAGGTCTGACGACCAGTCGTGGCGAACCG | |
| 210R/E5 | GTCCAGGCTGTTTATGGTTTCGCTAAGTCTTTGCTCAAAGCACTGAAAGAAGACGGGGAC | |
| 07T); | GCGGTAATTGTTGTATTTGATGCCAAAGCACCGAGCTTCCGCCACGAAGCTTATGGTGGC | |
| DNA | TACAAGGCAGGACGCGCCCCTCGTCCAGAAGATTTCCCCCGTCAGCTGGCATTAATTAAG | |
| sequence | GAGTTAGTAGACCTTCTCGGCTTAGCGCGTCTGGAAGTTCCGGGTTATGAGGCGGACGAT | |
| (5′ to 3′) | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAACGTTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCACCAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M80 | MRGMLPLFEPKGRVLLVDGHHLAYRTFHALKGLTTSRGEPVQAVYGFAKSLLKALKEDGD | 147 |
| (A61D/E | DVIVVFDAKAPSFRHEAYGGYKAGRAPTPEDFPRQLALIKELVDLLGLARLEVPGYEADD | |
| 210R/E5 | VLASLAKKAEKEGYEVRILTADKDLYQLLSDRIHVLHPEGYLITPAWLWEKYGLRPDQWA | |
| 07T); | DYRALTGDESDNLPGVKGIGEKTARKLLERWGSLEALLKNLDRLKPAIREKILAHMDDLK | |
| Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| (N to C | LREGLGLPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTTKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M80 | ATGCGTGGTATGCTGCCGTTGTTCGAGCCTAAAGGCCGCGTACTGTTAGTCGATGGTCAT | 148 |
| (A61D/E | CACTTGGCCTATCGGACGTTCCATGCACTCAAAGGTCTGACGACCAGTCGTGGCGAACCG | |
| 210R/E5 | GTCCAGGCTGTTTATGGTTTCGCTAAGTCTTTGCTCAAAGCACTGAAAGAAGACGGGGAC | |
| 07T); DNA | GATGTAATTGTTGTATTTGATGCCAAAGCACCGAGCTTCCGCCACGAAGCTTATGGTGGC | |
| sequence | TACAAGGCAGGACGCGCCCCTACCCCAGAAGATTTCCCCCGTCAGCTGGCATTAATTAAG | |
| (5′ to 3′) | GAGTTAGTAGACCTTCTCGGCTTAGCGCGTCTGGAAGTTCCGGGTTATGAGGCGGACGAT | |
| | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAACGTTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCACCAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M81 | MRGMLPLFEPKGRVLLVDGHHLAYRTFHALKGLTTSRGEPVQAVYGFAKSLLKALKEDGD | 149 |
| (A61D/T | DVIVVFDAKAPSFRHEAYGGYKAGRAPRPEDFPRQLALIKELVDLLGLARLEVPGYEADD | |
| 88R/E50 | VLASLAKKAEKEGYEVRILTADKDLYQLLSDRIHVLHPEGYLITPAWLWEKYGLRPDQWA | |
| 7T); | DYRALTGDESDNLPGVKGIGEKTARKLLEEWGSLEALLKNLDRLKPAIREKILAHMDDLK | |
| Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| (N to C | LREGLGLPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTTKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M81 | ATGCGTGGTATGCTGCCGTTGTTCGAGCCTAAAGGCCGCGTACTGTTAGTCGATGGTCAT | 150 |
| (A61D/T | CACTTGGCCTATCGGACGTTCCATGCACTCAAAGGTCTGACGACCAGTCGTGGCGAACCG | |
| 88R/E50 | GTCCAGGCTGTTTATGGTTTCGCTAAGTCTTTGCTCAAAGCACTGAAAGAAGACGGGGAC | |
| 7T); | GATGTAATTGTTGTATTTGATGCCAAAGCACCGAGCTTCCGCCACGAAGCTTATGGTGGC | |
| DNA | TACAAGGCAGGACGCGCCCCTCGTCCAGAAGATTTCCCCCGTCAGCTGGCATTAATTAAG | |
| sequence | GAGTTAGTAGACCTTCTCGGCTTAGCGCGTCTGGAAGTTCCGGGTTATGAGGCGGACGAT | |
| (5′ to 3′) | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCACCAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M82 | MRGMLPLFEPKGRVLLVDGHRLAYRTFHALKGLTTSRGEPVQAVYGFAKSLLKALKEDGD | 151 |
| (H21R/E | AVIVVFDAKAPSFRHEAYGGYKAGRAPTPEDFPRQLALIKELVDLLGLARLEVPGYEADD | |
| 210R/E5 | VLASLAKKAEKEGYEVRILTADKDLYQLLSDRIHVLHPEGYLITPAWLWEKYGLRPDQWA | |
| 07T); | DYRALTGDESDNLPGVKGIGEKTARKLLERWGSLEALLKNLDRLKPAIREKILAHMDDLK | |
| Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| (N to C | LREGLGLPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTTKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M82 | ATGCGTGGTATGCTGCCGTTGTTCGAGCCTAAAGGCCGCGTACTGTTAGTCGATGGTCAT | 152 |
| (H21R/E | CGTTTGGCCTATCGGACGTTCCATGCACTCAAAGGTCTGACGACCAGTCGTGGCGAACCG | |
| 210R/E5 | GTCCAGGCTGTTTATGGTTTCGCTAAGTCTTTGCTCAAAGCACTGAAAGAAGACGGGGAC | |
| 07T); | GCGGTAATTGTTGTATTTGATGCCAAAGCACCGAGCTTCCGCCACGAAGCTTATGGTGGC | |
| DNA | TACAAGGCAGGACGCGCCCCTACCCCAGAAGATTTCCCCCGTCAGCTGGCATTAATTAAG | |
| sequence | GAGTTAGTAGACCTTCTCGGCTTAGCGCGTCTGGAAGTTCCGGGTTATGAGGCGGACGAT | |
| (5′ to 3′) | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAACGTTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCACCAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M83 | MRGMLPLFEPKGRVLLVDGHRLAYRTFHALKGLTTSRGEPVQAVYGFAKSLLKALKEDGD | 153 |
| (H21R/T | AVIVVFDAKAPSFRHEAYGGYKAGRAPRPEDFPRQLALIKELVDLLGLARLEVPGYEADD | |
| 88R/E50 | VLASLAKKAEKEGYEVRILTADKDLYQLLSDRIHVLHPEGYLITPAWLWEKYGLRPDQWA | |
| 7T); | DYRALTGDESDNLPGVKGIGEKTARKLLEEWGSLEALLKNLDRLKPAIREKILAHMDDLK | |
| Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| (N to C | LREGLGLPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTTKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M83 | ATGCGTGGTATGCTGCCGTTGTTCGAGCCTAAAGGCCGCGTACTGTTAGTCGATGGTCAT | 154 |
| (H21R/T | CGTTTGGCCTATCGGACGTTCCATGCACTCAAAGGTCTGACGACCAGTCGTGGCGAACCG | |
| 88R/E50 | GTCCAGGCTGTTTATGGTTTCGCTAAGTCTTTGCTCAAAGCACTGAAAGAAGACGGGGAC | |
| 7T); | GCGGTAATTGTTGTATTTGATGCCAAAGCACCGAGCTTCCGCCACGAAGCTTATGGTGGC | |
| DNA | TACAAGGCAGGACGCGCCCCTCGTCCAGAAGATTTCCCCCGTCAGCTGGCATTAATTAAG | |
| sequence | GAGTTAGTAGACCTTCTCGGCTTAGCGCGTCTGGAAGTTCCGGGTTATGAGGCGGACGAT | |
| (5′ to 3′) | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCACCAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M84 | MRGMLPLFEPKGRVLLVDGHRLAYRTFHALKGLTTSRGEPVQAVYGFAKSLLKALKEDGD | 155 |
| (H21R/A | DVIVVFDAKAPSFRHEAYGGYKAGRAPTPEDFPRQLALIKELVDLLGLARLEVPGYEADD | |
| 61D/E50 | VLASLAKKAEKEGYEVRILTADKDLYQLLSDRIHVLHPEGYLITPAWLWEKYGLRPDQWA | |
| 7T); | DYRALTGDESDNLPGVKGIGEKTARKLLEEWGSLEALLKNLDRLKPAIREKILAHMDDLK | |
| Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| (N to C | LREGLGLPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTTKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M84 | ATGCGTGGTATGCTGCCGTTGTTCGAGCCTAAAGGCCGCGTACTGTTAGTCGATGGTCAT | 156 |
| (H21R/A | CGTTTGGCCTATCGGACGTTCCATGCACTCAAAGGTCTGACGACCAGTCGTGGCGAACCG | |
| 61D/E50 | GTCCAGGCTGTTTATGGTTTCGCTAAGTCTTTGCTCAAAGCACTGAAAGAAGACGGGGAC | |
| 7T); | GATGTAATTGTTGTATTTGATGCCAAAGCACCGAGCTTCCGCCACGAAGCTTATGGTGGC | |
| DNA | TACAAGGCAGGACGCGCCCCTACCCCAGAAGATTTCCCCCGTCAGCTGGCATTAATTAAG | |
| sequence | GAGTTAGTAGACCTTCTCGGCTTAGCGCGTCTGGAAGTTCCGGGTTATGAGGCGGACGAT | |
| (5′ to 3′) | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCACCAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M85 | MRGMLPLFEPKGRVLLLDGHHLAYRTFHALKGLTTSRGEPVQAVYGFAKSLLKALKEDGD | 157 |
| (V17L/E | AVIVVFDAKAPSFRHEAYGGYKAGRAPTPEDFPRQLALIKELVDLLGLARLEVPGYEADD | |
| 210R/E5 | VLASLAKKAEKEGYEVRILTADKDLYQLLSDRIHVLHPEGYLITPAWLWEKYGLRPDQWA | |
| 07T); | DYRALTGDESDNLPGVKGIGEKTARKLLERWGSLEALLKNLDRLKPAIREKILAHMDDLK | |
| Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| (N to C | LREGLGLPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTTKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M85 | ATGCGTGGTATGCTGCCGTTGTTCGAGCCTAAAGGCCGCGTACTGTTACTTGATGGTCAT | 158 |
| (V17L/E | CACTTGGCCTATCGGACGTTCCATGCACTCAAAGGTCTGACGACCAGTCGTGGCGAACCG | |
| 210R/E5 | GTCCAGGCTGTTTATGGTTTCGCTAAGTCTTTGCTCAAAGCACTGAAAGAAGACGGGGAC | |
| 07T); | GCGGTAATTGTTGTATTTGATGCCAAAGCACCGAGCTTCCGCCACGAAGCTTATGGTGGC | |
| DNA | TACAAGGCAGGACGCGCCCCTACCCCAGAAGATTTCCCCCGTCAGCTGGCATTAATTAAG | |
| sequence | GAGTTAGTAGACCTTCTCGGCTTAGCGCGTCTGGAAGTTCCGGGTTATGAGGCGGACGAT | |
| (5′ to 3′) | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAACGTTGGGGTTCCCTGGAAGCCTTGTTAAAAAAT | |
| | CTGGATCGTCTCAAGCCCGC | |
| | AATTCGTGAAAAGATCCTGGCTCATATGGACGATCTTAAATTAAGTTGGGACCTGGCCAA | |
| | GGTGCGCACCGATTTACCGCTTGAAGTGGATTTTGCAAAACGCCGTGAGCCGGACCGGGA | |
| | ACGTTTACGCGCTTTCTTAGAGCGTCTGGAATTCGGTTCACTGCTTCATGAATTCGGTCT | |
| | GTTAGAGTCTCCTAAAGCACTCGAAGAGGCACCGTGGCCGCCCCCAGAAGGTGCTTTTGT | |
| | TGGCTTCGTACTTTCCCGTAAGGAGCCTATGTGGGCAGATCTTCTGGCTTTAGCGGCTGC | |
| | ACGCGGTGGCCGTGTTCACCGGGCCCCTGAGCCATACAAAGCGTTACGTGATCTGAAGGA | |
| | AGCACGTGGCTTGCTGGCAAAAGACCTTTCTGTTTTGGCCCTGCGCGAGGGTCTTGGACT | |
| | GCCGCCAGGCGACGATCCCATGTTATTGGCCTATCTGTTAGACCCTAGCAATACCACACC | |
| | TGAAGGGGTCGCTCGTCGGTATGGCGGTGAATGGACTGAGGAAGCCGGAGAGCGCGCCGC | |
| | ATTGTCCGAACGGCTCTTTGCAAACTTATGGGGTCGTCTGGAAGGGGAGGAACGTCTGTT | |
| | ATGGTTGTATCGGGAAGTCGAACGTCCTCTTTCGGCCGTATTAGCGCATATGGAGGCAAC | |
| | AGGTGTGCGTTTAGATGTCGCGTACCTTCGGGCCTTATCACTGGAAGTTGCAGAGGAAAT | |
| | CGCCCGTCTCGAGGCTGAAGTGTTCCGGTTGGCCGGTCACCCGTTTAACCTCAACTCCCG | |
| | TGACCAGCTGGAACGCGTTTTATTCGATGAGCTTGGGCTTCCCGCAATTGGCAAAACCAC | |
| | CAAGACTGGCAAACGCAGTACGAGCGCTGCCGTCCTTGAGGCACTCCGCGAGGCTCACCC | |
| | TATTGTAGAAAAGATCCTGCAATACCGTGAGTTGACGAAGCTTAAAAGCACTTATATTGA | |
| | TCCTCTCCCGGATCTGATCCATCCTCGTACCGGCCGCTTGCACACACGTTTCAACCAGAC | |
| | GGCGACTGCAACCGGCCGTCTGTCTAGCTCGGATCCAAATCTCCAGAACATTCCGGTCCG | |
| | TACACCCTTGGGCCAACGTATCCGCCGGGCGTTTATCGCGGAAGAAGGATGGTTACTGGT | |
| | CGCATTGGACTACTCGCAGATTGAGCTGCGCGTCCTCGCACATCTCTCTGGTGACGAAAA | |
| | TTTAATCCGCGTGTTTCAAGAGGGGCGTGATATTCACACAGAAACTGCCTCATGGATGTT | |
| | CGGTGTCCCACGTGAAGCAGTGGATCCTTTGATGCGCCGTGCAGCTAAAACAATTAATTT | |
| | TGGAGTGCTGTACGGAATGAGCGCTCATCGCTTGAGTCAGGAACTGGCAATCCCCTACGA | |
| | GGAAGCGCAGGCATTCATCGAACGTTACTTTCAATCGTTTCCGAAAGTTCGCGCATGGAT | |
| | CGAGAAGACGCTCGAGGAAGGTCGTCGTCGGGGCTATGTCGAAACTCTGTTTGGTCGCCG | |
| | TCGGTACGTACCAGATCTTGAAGCCCGCGTCAAATCGGTACGGGAGGCTGCGGAGCGTAT | |
| | GGCATTTAATATGCCTGTACAGGGTACTGCAGCTGACCTCATGAAACTGGCAATGGTCAA | |
| | GCTTTTCCCGCGCTTGGAGGAAATGGGCGCACGTATGCTTCTGCAGGTCCATGACGAGCT | |
| | GGTGTTAGAAGCCCCTAAGGAGCGCGCCGAAGCTGTCGCGCGCCTCGCTAAAGAAGTGAT | |
| | GGAGGGCGTTTACCCATTGGCCGTACCCCTCGAAGTGGAGGTCGGTATTGGAGAAGATTG | |
| | GTTATCTGCAAAGGAAgcggccgcactcgagatcaaacgggctagccagccagaactcgc | |
| | cccggaagaccccgaggatgtcgagcaccaccaccaccaccac | |
| M86 | MRGMLPLFEPKGRVLLLDGHHLAYRTFHALKGLTTSRGEPVQAVYGFAKSLLKALKEDGD | 159 |
| (V17L/T | AVIVVFDAKAPSFRHEAYGGYKAGRAPRPEDFPRQLALIKELVDLLGLARLEVPGYEADD | |
| 88R/E50 | VLASLAKKAEKEGYEVRILTADKDLYQLLSDRIHVLHPEGYLITPAWLWEKYGLRPDQWA | |
| 7T); | DYRALTGDESDNLPGVKGIGEKTARKLLEEWGSLEALLKNLDRLKPAIREKILAHMDDLK | |
| Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| (N to C | LREGLGLPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTTKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M86 | ATGCGTGGTATGCTGCCGTTGTTCGAGCCTAAAGGCCGCGTACTGTTACTTGATGGTCAT | 160 |
| (V17L/T | CACTTGGCCTATCGGACGTTCCATGCACTCAAAGGTCTGACGACCAGTCGTGGCGAACCG | |
| 88R/E50 | GTCCAGGCTGTTTATGGTTTCGCTAAGTCTTTGCTCAAAGCACTGAAAGAAGACGGGGAC | |
| 7T); | GCGGTAATTGTTGTATTTGATGCCAAAGCACCGAGCTTCCGCCACGAAGCTTATGGTGGC | |
| DNA | TACAAGGCAGGACGCGCCCCTCGTCCAGAAGATTTCCCCCGTCAGCTGGCATTAATTAAG | |
| sequence | GAGTTAGTAGACCTTCTCGGCTTAGCGCGTCTGGAAGTTCCGGGTTATGAGGCGGACGAT | |
| (5′ to 3′) | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCACCAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M87 | MRGMLPLFEPKGRVLLLDGHHLAYRTFHALKGLTTSRGEPVQAVYGFAKSLLKALKEDGD | 161 |
| (V17L/A | DVIVVFDAKAPSFRHEAYGGYKAGRAPTPEDFPRQLALIKELVDLLGLARLEVPGYEADD | |
| 61D/E50 | VLASLAKKAEKEGYEVRILTADKDLYQLLSDRIHVLHPEGYLITPAWLWEKYGLRPDQWA | |
| 7T); | DYRALTGDESDNLPGVKGIGEKTARKLLEEWGSLEALLKNLDRLKPAIREKILAHMDDLK | |
| Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| (N to C | LREGLGLPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTTKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M87 | ATGCGTGGTATGCTGCCGTTGTTCGAGCCTAAAGGCCGCGTACTGTTACTTGATGGTCAT | 162 |
| (V17L/A | CACTTGGCCTATCGGACGTTCCATGCACTCAAAGGTCTGACGACCAGTCGTGGCGAACCG | |
| 61D/E50 | GTCCAGGCTGTTTATGGTTTCGCTAAGTCTTTGCTCAAAGCACTGAAAGAAGACGGGGAC | |
| 7T); | GATGTAATTGTTGTATTTGATGCCAAAGCACCGAGCTTCCGCCACGAAGCTTATGGTGGC | |
| DNA | TACAAGGCAGGACGCGCCCCTACCCCAGAAGATTTCCCCCGTCAGCTGGCATTAATTAAG | |
| sequence | GAGTTAGTAGACCTTCTCGGCTTAGCGCGTCTGGAAGTTCCGGGTTATGAGGCGGACGAT | |
| (5′ to 3′) | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCACCAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M88 | MRGMLPLFEPKGRVLLLDGHRLAYRTFHALKGLTTSRGEPVQAVYGFAKSLLKALKEDGD | 163 |
| (V17L/H | AVIVVFDAKAPSFRHEAYGGYKAGRAPTPEDFPRQLALIKELVDLLGLARLEVPGYEADD | |
| 21R/E50 | VLASLAKKAEKEGYEVRILTADKDLYQLLSDRIHVLHPEGYLITPAWLWEKYGLRPDQWA | |
| 7T); | DYRALTGDESDNLPGVKGIGEKTARKLLEEWGSLEALLKNLDRLKPAIREKILAHMDDLK | |
| Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| (N to C | LREGLGLPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTTKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M88 | ATGCGTGGTATGCTGCCGTTGTTCGAGCCTAAAGGCCGCGTACTGTTACTTGATGGTCAT | 164 |
| (V17L/H | CGTTTGGCCTATCGGACGTTCCATGCACTCAAAGGTCTGACGACCAGTCGTGGCGAACCG | |
| 21R/E50 | GTCCAGGCTGTTTATGGTTTCGCTAAGTCTTTGCTCAAAGCACTGAAAGAAGACGGGGAC | |
| 7T); | GCGGTAATTGTTGTATTTGATGCCAAAGCACCGAGCTTCCGCCACGAAGCTTATGGTGGC | |
| DNA | TACAAGGCAGGACGCGCCCCTACCCCAGAAGATTTCCCCCGTCAGCTGGCATTAATTAAG | |
| sequence | GAGTTAGTAGACCTTCTCGGCTTAGCGCGTCTGGAAGTTCCGGGTTATGAGGCGGACGAT | |
| (5′ to 3′) | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCACCAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M89 | MRGMLPLFEPKGRVLLVDGHHLAYRTFHALKGLTTSRGEPVQAVYGFAKSLLKALKEDGD | 165 |
| (A61D/T | DVIVVFDAKAPSFRHEAYGGYKAGRAPRPEDFPRQLALIKELVDLLGLARLEVPGYEADD | |
| 88R/E21 | VLASLAKKAEKEGYEVRILTADKDLYQLLSDRIHVLHPEGYLITPAWLWEKYGLRPDQWA | |
| 0R); | DYRALTGDESDNLPGVKGIGEKTARKLLERWGSLEALLKNLDRLKPAIREKILAHMDDLK | |
| Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| (N to C | LREGLGLPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M89 | ATGCGTGGTATGCTGCCGTTGTTCGAGCCTAAAGGCCGCGTACTGTTAGTCGATGGTCAT | 166 |
| (A61D/T | CACTTGGCCTATCGGACGTTCCATGCACTCAAAGGTCTGACGACCAGTCGTGGCGAACCG | |
| 88R/E21 | GTCCAGGCTGTTTATGGTTTCGCTAAGTCTTTGCTCAAAGCACTGAAAGAAGACGGGGAC | |
| OR); | GATGTAATTGTTGTATTTGATGCCAAAGCACCGAGCTTCCGCCACGAAGCTTATGGTGGC | |
| DNA | TACAAGGCAGGACGCGCCCCTCGTCCAGAAGATTTCCCCCGTCAGCTGGCATTAATTAAG | |
| sequence | GAGTTAGTAGACCTTCTCGGCTTAGCGCGTCTGGAAGTTCCGGGTTATGAGGCGGACGAT | |
| (5′ to 3′) | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAACGTTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M90 | MRGMLPLFEPKGRVLLVDGHRLAYRTFHALKGLTTSRGEPVQAVYGFAKSLLKALKEDGD | 167 |
| (H21R/A | DVIVVFDAKAPSFRHEAYGGYKAGRAPTPEDFPRQLALIKELVDLLGLARLEVPGYEADD | |
| 61D/E21 | VLASLAKKAEKEGYEVRILTADKDLYQLLSDRIHVLHPEGYLITPAWLWEKYGLRPDQWA | |
| OR); | DYRALTGDESDNLPGVKGIGEKTARKLLERWGSLEALLKNLDRLKPAIREKILAHMDDLK | |
| Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| (N to C | LREGLGLPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M90 | ATGCGTGGTATGCTGCCGTTGTTCGAGCCTAAAGGCCGCGTACTGTTAGTCGATGGTCAT | 168 |
| (H21R/A | CGTTTGGCCTATCGGACGTTCCATGCACTCAAAGGTCTGACGACCAGTCGTGGCGAACCG | |
| 61D/E21 | GTCCAGGCTGTTTATGGTTTCGCTAAGTCTTTGCTCAAAGCACTGAAAGAAGACGGGGAC | |
| OR); | GATGTAATTGTTGTATTTGATGCCAAAGCACCGAGCTTCCGCCACGAAGCTTATGGTGGC | |
| DNA | TACAAGGCAGGACGCGCCCCTACCCCAGAAGATTTCCCCCGTCAGCTGGCATTAATTAAG | |
| sequence | GAGTTAGTAGACCTTCTCGGCTTAGCGCGTCTGGAAGTTCCGGGTTATGAGGCGGACGAT | |
| (5′ to 3′) | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAACGTTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M91 | MRGMLPLFEPKGRVLLLDGHHLAYRTFHALKGLTTSRGEPVQAVYGFAKSLLKALKEDGD | 169 |
| (V17L/T | AVIVVFDAKAPSFRHEAYGGYKAGRAPRPEDFPROLALIKELVDLLGLARLEVPGYEADD | |
| 88R/E21 | VLASLAKKAEKEGYEVRILTADKDLYQLLSDRIHVLHPEGYLITPAWLWEKYGLRPDQWA | |
| OR); | DYRALTGDESDNLPGVKGIGEKTARKLLERWGSLEALLKNLDRLKPAIREKILAHMDDLK | |
| Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| (N to C | LREGLGLPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M91 | ATGCGTGGTATGCTGCCGTTGTTCGAGCCTAAAGGCCGCGTACTGTTACTTGATGGTCAT | 170 |
| (V17L/T | CACTTGGCCTATCGGACGTTCCATGCACTCAAAGGTCTGACGACCAGTCGTGGCGAACCG | |
| 88R/E21 | GTCCAGGCTGTTTATGGTTTCGCTAAGTCTTTGCTCAAAGCACTGAAAGAAGACGGGGAC | |
| OR); | GCGGTAATTGTTGTATTTGATGCCAAAGCACCGAGCTTCCGCCACGAAGCTTATGGTGGC | |
| DNA | TACAAGGCAGGACGCGCCCCTCGTCCAGAAGATTTCCCCCGTCAGCTGGCATTAATTAAG | |
| sequence | GAGTTAGTAGACCTTCTCGGCTTAGCGCGTCTGGAAGTTCCGGGTTATGAGGCGGACGAT | |
| (5′ to 3′) | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAACGTTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M92 | MRGMLPLFEPKGRVLLLDGHHLAYRTFHALKGLTTSRGEPVQAVYGFAKSLLKALKEDGD | 171 |
| (V17L/A | DVIVVFDAKAPSFRHEAYGGYKAGRAPTPEDFPRQLALIKELVDLLGLARLEVPGYEADD | |
| 61D/E21 | VLASLAKKAEKEGYEVRILTADKDLYQLLSDRIHVLHPEGYLITPAWLWEKYGLRPDQWA | |
| OR); | DYRALTGDESDNLPGVKGIGEKTARKLLERWGSLEALLKNLDRLKPAIREKILAHMDDLK | |
| Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| (N to C | LREGLGLPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M92 | ATGCGTGGTATGCTGCCGTTGTTCGAGCCTAAAGGCCGCGTACTGTTACTTGATGGTCAT | 172 |
| (V17L/A | CACTTGGCCTATCGGACGTTCCATGCACTCAAAGGTCTGACGACCAGTCGTGGCGAACCG | |
| 61D/E21 | GTCCAGGCTGTTTATGGTTTCGCTAAGTCTTTGCTCAAAGCACTGAAAGAAGACGGGGAC | |
| OR); | GATGTAATTGTTGTATTTGATGCCAAAGCACCGAGCTTCCGCCACGAAGCTTATGGTGGC | |
| DNA | TACAAGGCAGGACGCGCCCCTACCCCAGAAGATTTCCCCCGTCAGCTGGCATTAATTAAG | |
| sequence | GAGTTAGTAGACCTTCTCGGCTTAGCGCGTCTGGAAGTTCCGGGTTATGAGGCGGACGAT | |
| (5′ to 3′) | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAACGTTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M93 | MRGMLPLFEPKGRVLLLDGHHLAYRTFHALKGLTTSRGEPVQAVYGFAKSLLKALKEDGD | 173 |
| (V17L/A | DVIVVFDAKAPSFRHEAYGGYKAGRAPRPEDFPRQLALIKELVDLLGLARLEVPGYEADD | |
| 61D/T88 | VLASLAKKAEKEGYEVRILTADKDLYQLLSDRIHVLHPEGYLITPAWLWEKYGLRPDQWA | |
| R); | DYRALTGDESDNLPGVKGIGEKTARKLLEEWGSLEALLKNLDRLKPAIREKILAHMDDLK | |
| Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| (N to C | LREGLGLPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M93 | ATGCGTGGTATGCTGCCGTTGTTCGAGCCTAAAGGCCGCGTACTGTTACTTGATGGTCAT | 174 |
| (V17L/A | CACTTGGCCTATCGGACGTTCCATGCACTCAAAGGTCTGACGACCAGTCGTGGCGAACCG | |
| 61D/T88 | GTCCAGGCTGTTTATGGTTTCGCTAAGTCTTTGCTCAAAGCACTGAAAGAAGACGGGGAC | |
| R); DNA | GATGTAATTGTTGTATTTGATGCCAAAGCACCGAGCTTCCGCCACGAAGCTTATGGTGGC | |
| sequence | TACAAGGCAGGACGCGCCCCTCGTCCAGAAGATTTCCCCCGTCAGCTGGCATTAATTAAG | |
| (5′ to 3′) | GAGTTAGTAGACCTTCTCGGCTTAGCGCGTCTGGAAGTTCCGGGTTATGAGGCGGACGAT | |
| | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M94 | MRGMLPLFEPKGRVLLLDGHRLAYRTFHALKGLTTSRGEPVQAVYGFAKSLLKALKEDGD | 175 |
| (V17L/H | AVIVVFDAKAPSFRHEAYGGYKAGRAPTPEDFPRQLALIKELVDLLGLARLEVPGYEADD | |
| 21R/E21 | VLASLAKKAEKEGYEVRILTADKDLYQLLSDRIHVLHPEGYLITPAWLWEKYGLRPDQWA | |
| OR); | DYRALTGDESDNLPGVKGIGEKTARKLLERWGSLEALLKNLDRLKPAIREKILAHMDDLK | |
| Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| (N to C | LREGLGLPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M94 | ATGCGTGGTATGCTGCCGTTGTTCGAGCCTAAAGGCCGCGTACTGTTACTTGATGGTCAT | 176 |
| (V17L/H | CGTTTGGCCTATCGGACGTTCCATGCACTCAAAGGTCTGACGACCAGTCGTGGCGAACCG | |
| 21R/E21 | GTCCAGGCTGTTTATGGTTTCGCTAAGTCTTTGCTCAAAGCACTGAAAGAAGACGGGGAC | |
| OR); | GCGGTAATTGTTGTATTTGATGCCAAAGCACCGAGCTTCCGCCACGAAGCTTATGGTGGC | |
| DNA | TACAAGGCAGGACGCGCCCCTACCCCAGAAGATTTCCCCCGTCAGCTGGCATTAATTAAG | |
| sequence | GAGTTAGTAGACCTTCTCGGCTTAGCGCGTCTGGAAGTTCCGGGTTATGAGGCGGACGAT | |
| (5′ to 3′) | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAACGTTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M95 | MRGMLPLFEPKGRVLLLDGHRLAYRTFHALKGLTTSRGEPVQAVYGFAKSLLKALKEDGD | 177 |
| (V17L/H | DVIVVFDAKAPSFRHEAYGGYKAGRAPTPEDFPRQLALIKELVDLLGLARLEVPGYEADD | |
| 21R/A61 | VLASLAKKAEKEGYEVRILTADKDLYQLLSDRIHVLHPEGYLITPAWLWEKYGLRPDQWA | |
| D); | DYRALTGDESDNLPGVKGIGEKTARKLLEEWGSLEALLKNLDRLKPAIREKILAHMDDLK | |
| Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| (N to C | LREGLGLPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M95 | ATGCGTGGTATGCTGCCGTTGTTCGAGCCTAAAGGCCGCGTACTGTTACTTGATGGTCAT | 178 |
| (V17L/H | CGTTTGGCCTATCGGACGTTCCATGCACTCAAAGGTCTGACGACCAGTCGTGGCGAACCG | |
| 21R/A61 | GTCCAGGCTGTTTATGGTTTCGCTAAGTCTTTGCTCAAAGCACTGAAAGAAGACGGGGAC | |
| D); DNA | GATGTAATTGTTGTATTTGATGCCAAAGCACCGAGCTTCCGCCACGAAGCTTATGGTGGC | |
| sequence | TACAAGGCAGGACGCGCCCCTACCCCAGAAGATTTCCCCCGTCAGCTGGCATTAATTAAG | |
| (5′ to 3′) | GAGTTAGTAGACCTTCTCGGCTTAGCGCGTCTGGAAGTTCCGGGTTATGAGGCGGACGAT | |
| | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M96 | MRGMLPLFEPKGRVLLVDGHHLAYRTFHALKGLTTSRGEPVQAVYGFAKSLLKALKEDGD | 179 |
| (A61D/T | DVIVVFDAKAPSFRHEAYGGYKAGRAPRPEDFPRQLALIKELVDLLGLARLEVPGYEADD | |
| 88R/E21 | VLASLAKKAEKEGYEVRILTADKDLYQLLSDRIHVLHPEGYLITPAWLWEKYGLRPDQWA | |
| 0R/E507 | DYRALTGDESDNLPGVKGIGEKTARKLLERWGSLEALLKNLDRLKPAIREKILAHMDDLK | |
| T); | LSWDLAKVRTDLPLEVDFAKRREPDRERLRAFLERLEFGSLLHEFGLLESPKALEEAPWP | |
| Protein | PPEGAFVGFVLSRKEPMWADLLALAAARGGRVHRAPEPYKALRDLKEARGLLAKDLSVLA | |
| sequence | LREGLGLPPGDDPMLLAYLLDPSNTTPEGVARRYGGEWTEEAGERAALSERLFANLWGRL | |
| (N to C | EGEERLLWLYREVERPLSAVLAHMEATGVRLDVAYLRALSLEVAEEIARLEAEVFRLAGH | |
| terminus) | PFNLNSRDQLERVLFDELGLPAIGKTTKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M96 | ATGCGTGGTATGCTGCCGTTGTTCGAGCCTAAAGGCCGCGTACTGTTAGTCGATGGTCAT | 180 |
| (A61D/T | CACTTGGCCTATCGGACGTTCCATGCACTCAAAGGTCTGACGACCAGTCGTGGCGAACCG | |
| 88R/E21 | GTCCAGGCTGTTTATGGTTTCGCTAAGTCTTTGCTCAAAGCACTGAAAGAAGACGGGGAC | |
| 0R/E507 | GATGTAATTGTTGTATTTGATGCCAAAGCACCGAGCTTCCGCCACGAAGCTTATGGTGGC | |
| T); DNA | TACAAGGCAGGACGCGCCCCTCGTCCAGAAGATTTCCCCCGTCAGCTGGCATTAATTAAG | |
| sequence | GAGTTAGTAGACCTTCTCGGCTTAGCGCGTCTGGAAGTTCCGGGTTATGAGGCGGACGAT | |
| (5′ to 3′) | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAACGTTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCACCAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M97 | MRGMLPLFEPKGRVLLVDGHRLAYRTFHALKGLTTSRGEPVQAVYGFAKSLLKALKEDGD | 181 |
| (H21R/T | AVIVVFDAKAPSFRHEAYGGYKAGRAPRPEDFPRQLALIKELVDLLGLARLEVPGYEADD | |
| 88R/E21 | VLASLAKKAEKEGYEVRILTADKDLYQLLSDRIHVLHPEGYLITPAWLWEKYGLRPDQWA | |
| OR/E507 | DYRALTGDESDNLPGVKGIGEKTARKLLERWGSLEALLKNLDRLKPAIREKILAHMDDLK | |
| T); | LSWDLAKVRTDLPLEVDFAKRREPDRERLRAFLERLEFGSLLHEFGLLESPKALEEAPWP | |
| Protein | PPEGAFVGFVLSRKEPMWADLLALAAARGGRVHRAPEPYKALRDLKEARGLLAKDLSVLA | |
| sequence | LREGLGLPPGDDPMLLAYLLDPSNTTPEGVARRYGGEWTEEAGERAALSERLFANLWGRL | |
| (N to C | EGEERLLWLYREVERPLSAVLAHMEATGVRLDVAYLRALSLEVAEEIARLEAEVFRLAGH | |
| terminus) | PFNLNSRDQLERVLFDELGLPAIGKTTKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M97 | ATGCGTGGTATGCTGCCGTTGTTCGAGCCTAAAGGCCGCGTACTGTTAGTCGATGGTCAT | 182 |
| (H21R/T | CGTTTGGCCTATCGGACGTTCCATGCACTCAAAGGTCTGACGACCAGTCGTGGCGAACCG | |
| 88R/E21 | GTCCAGGCTGTTTATGGTTTCGCTAAGTCTTTGCTCAAAGCACTGAAAGAAGACGGGGAC | |
| OR/E507 | GCGGTAATTGTTGTATTTGATGCCAAAGCACCGAGCTTCCGCCACGAAGCTTATGGTGGC | |
| T); DNA | TACAAGGCAGGACGCGCCCCTCGTCCAGAAGATTTCCCCCGTCAGCTGGCATTAATTAAG | |
| sequence | GAGTTAGTAGACCTTCTCGGCTTAGCGCGTCTGGAAGTTCCGGGTTATGAGGCGGACGAT | |
| (5′ to 3′) | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAACGTTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCACCAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M98 | MRGMLPLFEPKGRVLLVDGHRLAYRTFHALKGLTTSRGEPVQAVYGFAKSLLKALKEDGD | 183 |
| (H21R/A | DVIVVFDAKAPSFRHEAYGGYKAGRAPTPEDFPRQLALIKELVDLLGLARLEVPGYEADD | |
| 61D/E21 | VLASLAKKAEKEGYEVRILTADKDLYQLLSDRIHVLHPEGYLITPAWLWEKYGLRPDQWA | |
| OR/E507 | DYRALTGDESDNLPGVKGIGEKTARKLLERWGSLEALLKNLDRLKPAIREKILAHMDDLK | |
| T); | LSWDLAKVRTDLPLEVDFAKRREPDRERLRAFLERLEFGSLLHEFGLLESPKALEEAPWP | |
| Protein | PPEGAFVGFVLSRKEPMWADLLALAAARGGRVHRAPEPYKALRDLKEARGLLAKDLSVLA | |
| sequence | LREGLGLPPGDDPMLLAYLLDPSNTTPEGVARRYGGEWTEEAGERAALSERLFANLWGRL | |
| (N to C | EGEERLLWLYREVERPLSAVLAHMEATGVRLDVAYLRALSLEVAEEIARLEAEVFRLAGH | |
| terminus) | PFNLNSRDQLERVLFDELGLPAIGKTTKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M98 | ATGCGTGGTATGCTGCCGTTGTTCGAGCCTAAAGGCCGCGTACTGTTAGTCGATGGTCAT | 184 |
| (H21R/A | CGTTTGGCCTATCGGACGTTCCATGCACTCAAAGGTCTGACGACCAGTCGTGGCGAACCG | |
| 61D/E21 | GTCCAGGCTGTTTATGGTTTCGCTAAGTCTTTGCTCAAAGCACTGAAAGAAGACGGGGAC | |
| 0R/E507 | GATGTAATTGTTGTATTTGATGCCAAAGCACCGAGCTTCCGCCACGAAGCTTATGGTGGC | |
| T); DNA | TACAAGGCAGGACGCGCCCCTACCCCAGAAGATTTCCCCCGTCAGCTGGCATTAATTAAG | |
| sequence | GAGTTAGTAGACCTTCTCGGCTTAGCGCGTCTGGAAGTTCCGGGTTATGAGGCGGACGAT | |
| (5′ to 3′) | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAACGTTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCACCAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M99 | MRGMLPLFEPKGRVLLVDGHRLAYRTFHALKGLTTSRGEPVQAVYGFAKSLLKALKEDGD | 185 |
| (H21R/A | DVIVVFDAKAPSFRHEAYGGYKAGRAPRPEDFPRQLALIKELVDLLGLARLEVPGYEADD | |
| 61D/T88 | VLASLAKKAEKEGYEVRILTADKDLYQLLSDRIHVLHPEGYLITPAWLWEKYGLRPDQWA | |
| R/E507T | DYRALTGDESDNLPGVKGIGEKTARKLLEEWGSLEALLKNLDRLKPAIREKILAHMDDLK | |
| ); Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| (N to C | LREGLGLPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTTKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M99 | ATGCGTGGTATGCTGCCGTTGTTCGAGCCTAAAGGCCGCGTACTGTTAGTCGATGGTCAT | 186 |
| (H21R/A | CGTTTGGCCTATCGGACGTTCCATGCACTCAAAGGTCTGACGACCAGTCGTGGCGAACCG | |
| 61D/T88 | GTCCAGGCTGTTTATGGTTTCGCTAAGTCTTTGCTCAAAGCACTGAAAGAAGACGGGGAC | |
| R/E507T | GATGTAATTGTTGTATTTGATGCCAAAGCACCGAGCTTCCGCCACGAAGCTTATGGTGGC | |
| ); DNA | TACAAGGCAGGACGCGCCCCTCGTCCAGAAGATTTCCCCCGTCAGCTGGCATTAATTAAG | |
| sequence | GAGTTAGTAGACCTTCTCGGCTTAGCGCGTCTGGAAGTTCCGGGTTATGAGGCGGACGAT | |
| (5′ to 3′) | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCACCAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M100 | MRGMLPLFEPKGRVLLLDGHHLAYRTFHALKGLTTSRGEPVQAVYGFAKSLLKALKEDGD | 187 |
| (V17L/T | AVIVVFDAKAPSFRHEAYGGYKAGRAPRPEDFPRQLALIKELVDLLGLARLEVPGYEADD | |
| 88R/E21 | VLASLAKKAEKEGYEVRILTADKDLYQLLSDRIHVLHPEGYLITPAWLWEKYGLRPDQWA | |
| OR/E507 | DYRALTGDESDNLPGVKGIGEKTARKLLERWGSLEALLKNLDRLKPAIREKILAHMDDLK | |
| T); | LSWDLAKVRTDLPLEVDFAKRREPDRERLRAFLERLEFGSLLHEFGLLESPKALEEAPWP | |
| Protein | PPEGAFVGFVLSRKEPMWADLLALAAARGGRVHRAPEPYKALRDLKEARGLLAKDLSVLA | |
| sequence | LREGLGLPPGDDPMLLAYLLDPSNTTPEGVARRYGGEWTEEAGERAALSERLFANLWGRL | |
| (N to C | EGEERLLWLYREVERPLSAVLAHMEATGVRLDVAYLRALSLEVAEEIARLEAEVFRLAGH | |
| terminus) | PFNLNSRDQLERVLFDELGLPAIGKTTKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M100 | ATGCGTGGTATGCTGCCGTTGTTCGAGCCTAAAGGCCGCGTACTGTTACTTGATGGTCAT | 188 |
| (V17L/T | CACTTGGCCTATCGGACGTTCCATGCACTCAAAGGTCTGACGACCAGTCGTGGCGAACCG | |
| 88R/E21 | GTCCAGGCTGTTTATGGTTTCGCTAAGTCTTTGCTCAAAGCACTGAAAGAAGACGGGGAC | |
| 0R/E507 | GCGGTAATTGTTGTATTTGATGCCAAAGCACCGAGCTTCCGCCACGAAGCTTATGGTGGC | |
| T); DNA | TACAAGGCAGGACGCGCCCCTCGTCCAGAAGATTTCCCCCGTCAGCTGGCATTAATTAAG | |
| sequence | GAGTTAGTAGACCTTCTCGGCTTAGCGCGTCTGGAAGTTCCGGGTTATGAGGCGGACGAT | |
| (5′ to 3′) | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAACGTTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCACCAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M101 | MRGMLPLFEPKGRVLLLDGHHLAYRTFHALKGLTTSRGEPVQAVYGFAKSLLKALKEDGD | 189 |
| (V17L/A | DVIVVFDAKAPSFRHEAYGGYKAGRAPTPEDFPRQLALIKELVDLLGLARLEVPGYEADD | |
| 61D/E21 | VLASLAKKAEKEGYEVRILTADKDLYQLLSDRIHVLHPEGYLITPAWLWEKYGLRPDQWA | |
| OR/E507 | DYRALTGDESDNLPGVKGIGEKTARKLLERWGSLEALLKNLDRLKPAIREKILAHMDDLK | |
| T): | LSWDLAKVRTDLPLEVDFAKRREPDRERLRAFLERLEFGSLLHEFGLLESPKALEEAPWP | |
| Protein | PPEGAFVGFVLSRKEPMWADLLALAAARGGRVHRAPEPYKALRDLKEARGLLAKDLSVLA | |
| sequence | LREGLGLPPGDDPMLLAYLLDPSNTTPEGVARRYGGEWTEEAGERAALSERLFANLWGRL | |
| (N to C | EGEERLLWLYREVERPLSAVLAHMEATGVRLDVAYLRALSLEVAEEIARLEAEVFRLAGH | |
| terminus) | PFNLNSRDQLERVLFDELGLPAIGKTTKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M101 | ATGCGTGGTATGCTGCCGTTGTTCGAGCCTAAAGGCCGCGTACTGTTACTTGATGGTCAT | 190 |
| (V17L/A | CACTTGGCCTATCGGACGTTCCATGCACTCAAAGGTCTGACGACCAGTCGTGGCGAACCG | |
| 61D/E21 | GTCCAGGCTGTTTATGGTTTCGCTAAGTCTTTGCTCAAAGCACTGAAAGAAGACGGGGAC | |
| OR/E507 | GATGTAATTGTTGTATTTGATGCCAAAGCACCGAGCTTCCGCCACGAAGCTTATGGTGGC | |
| T); DNA | TACAAGGCAGGACGCGCCCCTACCCCAGAAGATTTCCCCCGTCAGCTGGCATTAATTAAG | |
| sequence | GAGTTAGTAGACCTTCTCGGCTTAGCGCGTCTGGAAGTTCCGGGTTATGAGGCGGACGAT | |
| (5′ to 3′) | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAACGTTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCACCAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M102 | MRGMLPLFEPKGRVLLLDGHHLAYRTFHALKGLTTSRGEPVQAVYGFAKSLLKALKEDGD | 191 |
| (V17L/A | DVIVVFDAKAPSFRHEAYGGYKAGRAPRPEDFPRQLALIKELVDLLGLARLEVPGYEADD | |
| 61D/T88 | VLASLAKKAEKEGYEVRILTADKDLYQLLSDRIHVLHPEGYLITPAWLWEKYGLRPDQWA | |
| R/E507T | DYRALTGDESDNLPGVKGIGEKTARKLLEEWGSLEALLKNLDRLKPAIREKILAHMDDLK | |
| ); Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| N to C | LREGLGLPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTTKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M102 | ATGCGTGGTATGCTGCCGTTGTTCGAGCCTAAAGGCCGCGTACTGTTACTTGATGGTCAT | 192 |
| (V17L/A | CACTTGGCCTATCGGACGTTCCATGCACTCAAAGGTCTGACGACCAGTCGTGGCGAACCG | |
| 61D/T88 | GTCCAGGCTGTTTATGGTTTCGCTAAGTCTTTGCTCAAAGCACTGAAAGAAGACGGGGAC | |
| R/E507T | GATGTAATTGTTGTATTTGATGCCAAAGCACCGAGCTTCCGCCACGAAGCTTATGGTGGC | |
| ); DNA | TACAAGGCAGGACGCGCCCCTCGTCCAGAAGATTTCCCCCGTCAGCTGGCATTAATTAAG | |
| sequence | GAGTTAGTAGACCTTCTCGGCTTAGCGCGTCTGGAAGTTCCGGGTTATGAGGCGGACGAT | |
| (5′ to 3′) | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCACCAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M103 | MRGMLPLFEPKGRVLLLDGHRLAYRTFHALKGLTTSRGEPVQAVYGFAKSLLKALKEDGD | 193 |
| (V17L/H | AVIVVFDAKAPSFRHEAYGGYKAGRAPTPEDFPRQLALIKELVDLLGLARLEVPGYEADD | |
| 21R/E21 | VLASLAKKAEKEGYEVRILTADKDLYQLLSDRIHVLHPEGYLITPAWLWEKYGLRPDQWA | |
| 0R/E507 | DYRALTGDESDNLPGVKGIGEKTARKLLERWGSLEALLKNLDRLKPAIREKILAHMDDLK | |
| T); | LSWDLAKVRTDLPLEVDFAKRREPDRERLRAFLERLEFGSLLHEFGLLESPKALEEAPWP | |
| Protein | PPEGAFVGFVLSRKEPMWADLLALAAARGGRVHRAPEPYKALRDLKEARGLLAKDLSVLA | |
| sequence | LREGLGLPPGDDPMLLAYLLDPSNTTPEGVARRYGGEWTEEAGERAALSERLFANLWGRL | |
| (N to C | EGEERLLWLYREVERPLSAVLAHMEATGVRLDVAYLRALSLEVAEEIARLEAEVFRLAGH | |
| terminus) | PFNLNSRDQLERVLFDELGLPAIGKTTKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M103 | ATGCGTGGTATGCTGCCGTTGTTCGAGCCTAAAGGCCGCGTACTGTTACTTGATGGTCAT | 194 |
| (V17L/H | CGTTTGGCCTATCGGACGTTCCATGCACTCAAAGGTCTGACGACCAGTCGTGGCGAACCG | |
| 21R/E21 | GTCCAGGCTGTTTATGGTTTCGCTAAGTCTTTGCTCAAAGCACTGAAAGAAGACGGGGAC | |
| OR/E507 | GCGGTAATTGTTGTATTTGATGCCAAAGCACCGAGCTTCCGCCACGAAGCTTATGGTGGC | |
| T); DNA | TACAAGGCAGGACGCGCCCCTACCCCAGAAGATTTCCCCCGTCAGCTGGCATTAATTAAGA | |
| sequence | GGTTAGTAGACCTTCTCGGCTTAGCGCGTCTGGAAGTTCCGGGTTATGAGGCGGACGATGT | |
| (5′ to 3′) | CCTTGCATCCTTGGCTAAAAAGGCCGAAAAAGAGGGCTACGAAGTCCGCATCTTGACGGC | |
| | AGACAAAGATCTGTACCAGCTTCTGTCTGACCGTATTCATGTTTTGCACCCTGAAGGCTA | |
| | CTTAATCACTCCGGCCTGGCTCTGGGAAAAGTACGGTCTGCGTCCCGATCAGTGGGCGGA | |
| | TTATCGGGCTTTGACGGGAGATGAGAGCGACAACCTGCCAGGAGTTAAGGGCATTGGTGA | |
| | AAAAACCGCACGTAAGCTGCTTGAACGTTGGGGTTCCCTGGAAGCCTTGTTAAAAAATCT | |
| | GGATCGTCTCAAGCCCGCAATTCGTGAAAAGATCCTGGCTCATATGGACGATCTTAAATT | |
| | AAGTTGGGACCTGGCCAAGGTGCGCACCGATTTACCGCTTGAAGTGGATTTTGCAAAACG | |
| | CCGTGAGCCGGACCGGGAACGTTTACGCGCTTTCTTAGAGCGTCTGGAATTCGGTTCACT | |
| | GCTTCATGAATTCGGTCTGTTAGAGTCTCCTAAAGCACTCGAAGAGGCACCGTGGCCGCC | |
| | CCCAGAAGGTGCTTTTGTTGGCTTCGTACTTTCCCGTAAGGAGCCTATGTGGGCAGATCT | |
| | TCTGGCTTTAGCGGCTGCACGCGGTGGCCGTGTTCACCGGGCCCCTGAGCCATACAAAGC | |
| | GTTACGTGATCTGAAGGAAGCACGTGGCTTGCTGGCAAAAGACCTTTCTGTTTTGGCCCT | |
| | GCGCGAGGGTCTTGGACTGCCGCCAGGCGACGATCCCATGTTATTGGCCTATCTGTTAGA | |
| | CCCTAGCAATACCACACCTGAAGGGGTCGCTCGTCGGTATGGCGGTGAATGGACTGAGGA | |
| | AGCCGGAGAGCGCGCCGCATTGTCCGAACGGCTCTTTGCAAACTTATGGGGTCGTCTGGA | |
| | AGGGGAGGAACGTCTGTTATGGTTGTATCGGGAAGTCGAACGTCCTCTTTCGGCCGTATT | |
| | AGCGCATATGGAGGCAACAGGTGTGCGTTTAGATGTCGCGTACCTTCGGGCCTTATCACT | |
| | GGAAGTTGCAGAGGAAATCGCCCGTCTCGAGGCTGAAGTGTTCCGGTTGGCCGGTCACCC | |
| | GTTTAACCTCAACTCCCGTGACCAGCTGGAACGCGTTTTATTCGATGAGCTTGGGCTTCC | |
| | CGCAATTGGCAAAACCACCAAGACTGGCAAACGCAGTACGAGCGCTGCCGTCCTTGAGGC | |
| | ACTCCGCGAGGCTCACCCTATTGTAGAAAAGATCCTGCAATACCGTGAGTTGACGAAGCT | |
| | TAAAAGCACTTATATTGATCCTCTCCCGGATCTGATCCATCCTCGTACCGGCCGCTTGCA | |
| | CACACGTTTCAACCAGACGGCGACTGCAACCGGCCGTCTGTCTAGCTCGGATCCAAATCT | |
| | CCAGAACATTCCGGTCCGTACACCCTTGGGCCAACGTATCCGCCGGGCGTTTATCGCGGA | |
| | AGAAGGATGGTTACTGGTCGCATTGGACTACTCGCAGATTGAGCTGCGCGTCCTCGCACA | |
| | TCTCTCTGGTGACGAAAATTTAATCCGCGTGTTTCAAGAGGGGCGTGATATTCACACAGA | |
| | AACTGCCTCATGGATGTTCGGTGTCCCACGTGAAGCAGTGGATCCTTTGATGCGCCGTGC | |
| | AGCTAAAACAATTAATTTTGGAGTGCTGTACGGAATGAGCGCTCATCGCTTGAGTCAGGA | |
| | ACTGGCAATCCCCTACGAGGAAGCGCAGGCATTCATCGAACGTTACTTTCAATCGTTTCC | |
| | GAAAGTTCGCGCATGGATCGAGAAGACGCTCGAGGAAGGTCGTCGTCGGGGCTATGTCGA | |
| | AACTCTGTTTGGTCGCCGTCGGTACGTACCAGATCTTGAAGCCCGCGTCAAATCGGTACG | |
| | GGAGGCTGCGGAGCGTATGGCATTTAATATGCCTGTACAGGGTACTGCAGCTGACCTCAT | |
| | GAAACTGGCAATGGTCAAGCTTTTCCCGCGCTTGGAGGAAATGGGCGCACGTATGCTTCT | |
| | GCAGGTCCATGACGAGCTGGTGTTAGAAGCCCCTAAGGAGCGCGCCGAAGCTGTCGCGCG | |
| | CCTCGCTAAAGAAGTGATGGAGGGCGTTTACCCATTGGCCGTACCCCTCGAAGTGGAGGT | |
| | CGGTATTGGAGAAGATTGGTTATCTGCAAAGGAAgcggccgcactcgagatcaaacgggc | |
| | tagccagccagaactcgccccggaagaccccgaggatgtcgagcaccaccaccaccacca | |
| | c | |
| M104 | MRGMLPLFEPKGRVLLLDGHRLAYRTFHALKGLTTSRGEPVQAVYGFAKSLLKALKEDGD | 195 |
| (V17L/H | AVIVVFDAKAPSFRHEAYGGYKAGRAPRPEDFPRQLALIKELVDLLGLARLEVPGYEADD | |
| 21R/T88 | VLASLAKKAEKEGYEVRILTADKDLYQLLSDRIHVLHPEGYLITPAWLWEKYGLRPDQWA | |
| R/E507T | DYRALTGDESDNLPGVKGIGEKTARKLLEEWGSLEALLKNLDRLKPAIREKILAHMDDLK | |
| ); Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| (N to C | LREGLGLPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTTKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M104 | ATGCGTGGTATGCTGCCGTTGTTCGAGCCTAAAGGCCGCGTACTGTTACTTGATGGTCAT | 196 |
| (V17L/H | CGTTTGGCCTATCGGACGTTCCATGCACTCAAAGGTCTGACGACCAGTCGTGGCGAACCG | |
| 21R/T88 | GTCCAGGCTGTTTATGGTTTCGCTAAGTCTTTGCTCAAAGCACTGAAAGAAGACGGGGAC | |
| R/E507T | GCGGTAATTGTTGTATTTGATGCCAAAGCACCGAGCTTCCGCCACGAAGCTTATGGTGGC | |
| ); DNA | TACAAGGCAGGACGCGCCCCTCGTCCAGAAGATTTCCCCCGTCAGCTGGCATTAATTAAG | |
| sequence | GAGTTAGTAGACCTTCTCGGCTTAGCGCGTCTGGAAGTTCCGGGTTATGAGGCGGACGAT | |
| (5′ to 3′) | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCACCAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M105 | MRGMLPLFEPKGRVLLLDGHRLAYRTFHALKGLTTSRGEPVQAVYGFAKSLLKALKEDGD | 197 |
| (V17L/H | DVIVVFDAKAPSFRHEAYGGYKAGRAPTPEDFPRQLALIKELVDLLGLARLEVPGYEADD | |
| 21R/A61 | VLASLAKKAEKEGYEVRILTADKDLYQLLSDRIHVLHPEGYLITPAWLWEKYGLRPDQWA | |
| D/E507T | DYRALTGDESDNLPGVKGIGEKTARKLLEEWGSLEALLKNLDRLKPAIREKILAHMDDLK | |
| ); Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| (N to C | LREGLGLPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTTKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M105 | ATGCGTGGTATGCTGCCGTTGTTCGAGCCTAAAGGCCGCGTACTGTTACTTGATGGTCAT | 198 |
| (V17L/H | CGTTTGGCCTATCGGACGTTCCATGCACTCAAAGGTCTGACGACCAGTCGTGGCGAACCG | |
| 21R/A61 | GTCCAGGCTGTTTATGGTTTCGCTAAGTCTTTGCTCAAAGCACTGAAAGAAGACGGGGAC | |
| D/E507T | GATGTAATTGTTGTATTTGATGCCAAAGCACCGAGCTTCCGCCACGAAGCTTATGGTGGC | |
| ); DNA | TACAAGGCAGGACGCGCCCCTACCCCAGAAGATTTCCCCCGTCAGCTGGCATTAATTAAG | |
| sequence | GAGTTAGTAGACCTTCTCGGCTTAGCGCGTCTGGAAGTTCCGGGTTATGAGGCGGACGAT | |
| (5′ to 3′) | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCACCAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M106 | MRGMLPLFEPKGRVLLLDGHHLAYRTFHALKGLTTSRGEPVQAVYGFAKSLLKALKEDGD | 199 |
| (V17L/A | DVIVVFDAKAPSFRHEAYGGYKAGRAPRPEDFPRQLALIKELVDLLGLARLEVPGYEADD | |
| 61D/T88 | VLASLAKKAEKEGYEVRILTADKDLYQLLSDRIHVLHPEGYLITPAWLWEKYGLRPDQWA | |
| R/E210R | DYRALTGDESDNLPGVKGIGEKTARKLLERWGSLEALLKNLDRLKPAIREKILAHMDDLK | |
| ); Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| (N to C | LREGLGLPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M106 | ATGCGTGGTATGCTGCCGTTGTTCGAGCCTAAAGGCCGCGTACTGTTACTTGATGGTCAT | 200 |
| (V17L/A | CACTTGGCCTATCGGACGTTCCATGCACTCAAAGGTCTGACGACCAGTCGTGGCGAACCG | |
| 61D/T88 | GTCCAGGCTGTTTATGGTTTCGCTAAGTCTTTGCTCAAAGCACTGAAAGAAGACGGGGAC | |
| R/E210R | GATGTAATTGTTGTATTTGATGCCAAAGCACCGAGCTTCCGCCACGAAGCTTATGGTGGC | |
| ); DNA | TACAAGGCAGGACGCGCCCCTCGTCCAGAAGATTTCCCCCGTCAGCTGGCATTAATTAAG | |
| sequence | GAGTTAGTAGACCTTCTCGGCTTAGCGCGTCTGGAAGTTCCGGGTTATGAGGCGGACGAT | |
| (5′ to 3′) | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAACGTTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M107 | MRGMLPLFEPKGRVLLLDGHRLAYRTFHALKGLTTSRGEPVQAVYGFAKSLLKALKEDGD | 201 |
| (V17L/H | DVIVVFDAKAPSFRHEAYGGYKAGRAPTPEDFPRQLALIKELVDLLGLARLEVPGYEADD | |
| 21R/A61 | VLASLAKKAEKEGYEVRILTADKDLYQLLSDRIHVLHPEGYLITPAWLWEKYGLRPDQWA | |
| D/E210R | DYRALTGDESDNLPGVKGIGEKTARKLLERWGSLEALLKNLDRLKPAIREKILAHMDDLK | |
| ); Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| (N to C | LREGLGLPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMR | |
| | RAAKTINFGVLYGMSAHRLSQELAIPYEEAQAFIERYFQSFPKVRAWIEKTLEEGRRRGY | |
| | VETLFGRRRYVPDLEARVKSVREAAERMAFNMPVQGTAADLMKLAMVKLFPRLEEMGARM | |
| | LLQVHDELVLEAPKERAEAVARLAKEVMEGVYPLAVPLEVEVGIGEDWLSAKEAAALEIK | |
| | RASQPELAPEDPEDVEHHHHHH | |
| M107 | ATGCGTGGTATGCTGCCGTTGTTCGAGCCTAAAGGCCGCGTACTGTTACTTGATGGTCAT | 202 |
| (V17L/H | CGTTTGGCCTATCGGACGTTCCATGCACTCAAAGGTCTGACGACCAGTCGTGGCGAACCG | |
| 21R/A61D/ | GTCCAGGCTGTTTATGGTTTCGCTAAGTCTTTGCTCAAAGCACTGAAAGAAGACGGGGAC | |
| E210R); | GATGTAATTGTTGTATTTGATGCCAAAGCACCGAGCTTCCGCCACGAAGCTTATGGTGGC | |
| DNA | TACAAGGCAGGACGCGCCCCTACCCCAGAAGATTTCCCCCGTCAGCTGGCATTAATTAAG | |
| sequence | GAGTTAGTAGACCTTCTCGGCTTAGCGCGTCTGGAAGTTCCGGGTTATGAGGCGGACGAT | |
| (5′ to 3′) | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAACGTTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M108 | MRGMLPLFEPKGRVLLLDGHRLAYRTFHALKGLTTSRGEPVQAVYGFAKSLLKALKEDGD | 203 |
| (V17L/H | DVIVVFDAKAPSFRHEAYGGYKAGRAPRPEDFPRQLALIKELVDLLGLARLEVPGYEADD | |
| 21R/A61 | VLASLAKKAEKEGYEVRILTADKDLYQLLSDRIHVLHPEGYLITPAWLWEKYGLRPDQWA | |
| D/T88R); | DYRALTGDESDNLPGVKGIGEKTARKLLEEWGSLEALLKNLDRLKPAIREKILAHMDDLK | |
| Protein | LSWDLAKVRTDLPLEVDFAKRREPDRERLRAFLERLEFGSLLHEFGLLESPKALEEAPWP | |
| sequence | PPEGAFVGFVLSRKEPMWADLLALAAARGGRVHRAPEPYKALRDLKEARGLLAKDLSVLA | |
| (N to C | LREGLGLPPGDDPMLLAYLLDPSNTTPEGVARRYGGEWTEEAGERAALSERLFANLWGRL | |
| terminus) | EGEERLLWLYREVERPLSAVLAHMEATGVRLDVAYLRALSLEVAEEIARLEAEVFRLAGH | |
| | PFNLNSRDQLERVLFDELGLPAIGKTEKTGKRSTSAAVLEALREAHPIVEKILQYRELTK | |
| | LKSTYID | |
| | PLPDLIHPRTGRLHTRFNQTATATGRLSSSDPNLQNIPVRTPLGQRIRRAFIAEEGWLLV | |
| | ALDYSQIELRVLAHLSGDENLIRVFQEGRDIHTETASWMFGVPREAVDPLMRRAAKTINF | |
| | GVLYGMSAHRLSQELAIPYEEAQAFIERYFQSFPKVRAWIEKTLEEGRRRGYVETLFGRR | |
| | RYVPDLEARVKSVREAAERMAFNMPVQGTAADLMKLAMVKLFPRLEEMGARMLLQVHDEL | |
| | VLEAPKERAEAVARLAKEVMEGVYPLAVPLEVEVGIGEDWLSAKEAAALEIKRASQPELA | |
| | PEDPEDVEHHHHHH | |
| M108 | ATGCGTGGTATGCTGCCGTTGTTCGAGCCTAAAGGCCGCGTACTGTTACTTGATGGTCAT | 204 |
| (V17L/H | CGTTTGGCCTATCGGACGTTCCATGCACTCAAAGGTCTGACGACCAGTCGTGGCGAACCG | |
| 21R/A61 | GTCCAGGCTGTTTATGGTTTCGCTAAGTCTTTGCTCAAAGCACTGAAAGAAGACGGGGAC | |
| D/T88R); | GATGTAATTGTTGTATTTGATGCCAAAGCACCGAGCTTCCGCCACGAAGCTTATGGTGGC | |
| DNA | TACAAGGCAGGACGCGCCCCTCGTCCAGAAGATTTCCCCCGTCAGCTGGCATTAATTAAG | |
| sequence | GAGTTAGTAGACCTTCTCGGCTTAGCGCGTCTGGAAGTTCCGGGTTATGAGGCGGACGAT | |
| (5′ to 3′) | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAAGAGTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCGAAAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M109 | MRGMLPLFEPKGRVLLVDGHRLAYRTFHALKGLTTSRGEPVQAVYGFAKSLLKALKEDGD | 205 |
| (H21R/A | DVIVVFDAKAPSFRHEAYGGYKAGRAPRPEDFPRQLALIKELVDLLGLARLEVPGYEADD | |
| 61D/T88 | VLASLAKKAEKEGYEVRILTADKDLYQLLSDRIHVLHPEGYLITPAWLWEKYGLRPDQWA | |
| R/E210R | DYRALTGDESDNLPGVKGIGEKTARKLLERWGSLEALLKNLDRLKPAIREKILAHMDDLK | |
| /E507T); | LSWDLAKVRTDLPLEVDFAKRREPDRERLRAFLERLEFGSLLHEFGLLESPKALEEAPWP | |
| Protein | PPEGAFVGFVLSRKEPMWADLLALAAARGGRVHRAPEPYKALRDLKEARGLLAKDLSVLA | |
| sequence | LREGLGLPPGDDPMLLAYLLDPSNTTPEGVARRYGGEWTEEAGERAALSERLFANLWGRL | |
| (N to C | EGEERLLWLYREVERPLSAVLAHMEATGVRLDVAYLRALSLEVAEEIARLEAEVFRLAGH | |
| terminus) | PFNLNSRDQLERVLFDELGLPAIGKTTKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M109 | ATGCGTGGTATGCTGCCGTTGTTCGAGCCTAAAGGCCGCGTACTGTTAGTCGATGGTCAT | 206 |
| (H21R/A | CGTTTGGCCTATCGGACGTTCCATGCACTCAAAGGTCTGACGACCAGTCGTGGCGAACCG | |
| 61D/T88 | GTCCAGGCTGTTTATGGTTTCGCTAAGTCTTTGCTCAAAGCACTGAAAGAAGACGGGGAC | |
| R/E210R | GATGTAATTGTTGTATTTGATGCCAAAGCACCGAGCTTCCGCCACGAAGCTTATGGTGGC | |
| /E507T); | TACAAGGCAGGACGCGCCCCTCGTCCAGAAGATTTCCCCCGTCAGCTGGCATTAATTAAG | |
| DNA | GAGTTAGTAGACCTTCTCGGCTTAGCGCGTCTGGAAGTTCCGGGTTATGAGGCGGACGAT | |
| sequence | GTCCTTGCATCCTTGGCTAAAAAGGCCGAAAAAGAGGGCTACGAAGTCCGCATCTTGACG | |
| (5′ to 3′) | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAACGTTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCACCAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M110 | MRGMLPLFEPKGRVLLLDGHHLAYRTFHALKGLTTSRGEPVQAVYGFAKSLLKALKEDGD | 207 |
| (V17L/A | DVIVVFDAKAPSFRHEAYGGYKAGRAPRPEDFPRQLALIKELVDLLGLARLEVPGYEADD | |
| 61D/T88 | VLASLAKKAEKEGYEVRILTADKDLYQLLSDRIHVLHPEGYLITPAWLWEKYGLRPDQWA | |
| R/E210R | DYRALTGDESDNLPGVKGIGEKTARKLLERWGSLEALLKNLDRLKPAIREKILAHMDDLK | |
| /E507T); | LSWDLAKVRTDLPLEVDFAKRREPDRERLRAFLERLEFGSLLHEFGLLESPKALEEAPWP | |
| Protein | PPEGAFVGFVLSRKEPMWADLLALAAARGGRVHRAPEPYKALRDLKEARGLLAKDLSVLA | |
| sequence | LREGLGLPPGDDPMLLAYLLDPSNTTPEGVARRYGGEWTEEAGERAALSERLFANLWGRL | |
| (N to C | EGEERLLWLYREVERPLSAVLAHMEATGVRLDVAYLRALSLEVAEEIARLEAEVFRLAGH | |
| terminus) | PFNLNSRDQLERVLFDELGLPAIGKTTKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M110 | ATGCGTGGTATGCTGCCGTTGTTCGAGCCTAAAGGCCGCGTACTGTTACTTGATGGTCAT | 208 |
| (V17L/A | CACTTGGCCTATCGGACGTTCCATGCACTCAAAGGTCTGACGACCAGTCGTGGCGAACCG | |
| 61D/T88 | GTCCAGGCTGTTTATGGTTTCGCTAAGTCTTTGCTCAAAGCACTGAAAGAAGACGGGGAC | |
| R/E210R | GATGTAATTGTTGTATTTGATGCCAAAGCACCGAGCTTCCGCCACGAAGCTTATGGTGGC | |
| /E507T); | TACAAGGCAGGACGCGCCCCTCGTCCAGAAGATTTCCCCCGTCAGCTGGCATTAATTAAG | |
| DNA | GAGTTAGTAGACCTTCTCGGCTTAGCGCGTCTGGAAGTTCCGGGTTATGAGGCGGACGAT | |
| sequence | GTCCTTGCATCCTTGGCTAAAAAGGCCGAAAAAGAGGGCTACGAAGTCCGCATCTTGACG | |
| (5′ to 3′) | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAACGTTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCACCAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M111 | MRGMLPLFEPKGRVLLLDGHRLAYRTFHALKGLTTSRGEPVQAVYGFAKSLLKALKEDGD | 209 |
| (V17L/H | AVIVVFDAKAPSFRHEAYGGYKAGRAPRPEDFPRQLALIKELVDLLGLARLEVPGYEADD | |
| 21R/T88 | VLASLAKKAEKEGYEVRILTADKDLYQLLSDRIHVLHPEGYLITPAWLWEKYGLRPDQWA | |
| R/E210R | DYRALTGDESDNLPGVKGIGEKTARKLLERWGSLEALLKNLDRLKPAIREKILAHMDDLK | |
| /E507T); | LSWDLAKVRTDLPLEVDFAKRREPDRERLRAFLERLEFGSLLHEFGLLESPKALEEAPWP | |
| Protein | PPEGAFVGFVLSRKEPMWADLLALAAARGGRVHRAPEPYKALRDLKEARGLLAKDLSVLA | |
| sequence | LREGLGLPPGDDPMLLAYLLDPSNTTPEGVARRYGGEWTEEAGERAALSERLFANLWGRL | |
| (N to C | EGEERLLWLYREVERPLSAVLAHMEATGVRLDVAYLRALSLEVAEEIARLEAEVFRLAGH | |
| terminus) | PFNLNSRDQLERVLFDELGLPAIGKTTKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M111 | ATGCGTGGTATGCTGCCGTTGTTCGAGCCTAAAGGCCGCGTACTGTTACTTGATGGTCAT | 210 |
| (V17L/H | CGTTTGGCCTATCGGACGTTCCATGCACTCAAAGGTCTGACGACCAGTCGTGGCGAACCG | |
| 21R/T88 | GTCCAGGCTGTTTATGGTTTCGCTAAGTCTTTGCTCAAAGCACTGAAAGAAGACGGGGAC | |
| R/E210R | GCGGTAATTGTTGTATTTGATGCCAAAGCACCGAGCTTCCGCCACGAAGCTTATGGTGGC | |
| /E507T); | TACAAGGCAGGACGCGCCCCTCGTCCAGAAGATTTCCCCCGTCAGCTGGCATTAATTAAG | |
| DNA | GAGTTAGTAGACCTTCTCGGCTTAGCGCGTCTGGAAGTTCCGGGTTATGAGGCGGACGAT | |
| sequence | GTCCTTGCATCCTTGGCTAAAAAGGCCGAAAAAGAGGGCTACGAAGTCCGCATCTTGACG | |
| (5′ to 3′) | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAACGTTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCACCAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaacgg | |
| | gctagccagccagaactcgccccggaagaccccgaggatgtcgagcaccaccaccaccac | |
| | cac | |
| M112 | MRGMLPLFEPKGRVLLLDGHRLAYRTFHALKGLTTSRGEPVQAVYGFAKSLLKALKEDGD | 211 |
| (V17L/H | DVIVVFDAKAPSFRHEAYGGYKAGRAPTPEDFPRQLALIKELVDLLGLARLEVPGYEADD | |
| 21R/A61 | VLASLAKKAEKEGYEVRILTADKDLYQLLSDRIHVLHPEGYLITPAWLWEKYGLRPDQWA | |
| D/E210R | DYRALTGDESDNLPGVKGIGEKTARKLLERWGSLEALLKNLDRLKPAIREKILAHMDDLK | |
| /E507T); | LSWDLAKVRTDLPLEVDFAKRREPDRERLRAFLERLEFGSLLHEFGLLESPKALEEAPWP | |
| Protein | PPEGAFVGFVLSRKEPMWADLLALAAARGGRVHRAPEPYKALRDLKEARGLLAKDLSVLA | |
| sequence | LREGLGLPPGDDPMLLAYLLDPSNTTPEGVARRYGGEWTEEAGERAALSERLFANLWGRL | |
| (N to C | EGEERLLWLYREVERPLSAVLAHMEATGVRLDVAYLRALSLEVAEEIARLEAEVFRLAGH | |
| terminus) | PFNLNSRDQLERVLFDELGLPAIGKTTKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M112 | ATGCGTGGTATGCTGCCGTTGTTCGAGCCTAAAGGCCGCGTACTGTTACTTGATGGTCAT | 212 |
| (V17L/H | CGTTTGGCCTATCGGACGTTCCATGCACTCAAAGGTCTGACGACCAGTCGTGGCGAACCG | |
| 21R/A61 | GTCCAGGCTGTTTATGGTTTCGCTAAGTCTTTGCTCAAAGCACTGAAAGAAGACGGGGAC | |
| D/E210R | GATGTAATTGTTGTATTTGATGCCAAAGCACCGAGCTTCCGCCACGAAGCTTATGGTGGC | |
| /E507T); | TACAAGGCAGGACGCGCCCCTACCCCAGAAGATTTCCCCCGTCAGCTGGCATTAATTAAG | |
| DNA | GAGTTAGTAGACCTTCTCGGCTTAGCGCGTCTGGAAGTTCCGGGTTATGAGGCGGACGAT | |
| sequence | GTCCTTGCATCCTTGGCTAAAAAGGCCGAAAAAGAGGGCTACGAAGTCCGCATCTTGACG | |
| (5′ to 3′) | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCGATCAGTGGGCG | |
| | GATTATCGGGCTTTGACGGGAGATGAGAGCGACAACCTGCCAGGAGTTAAGGGCATTGGT | |
| | GAAAAAACCGCACGTAAGCTGCTTGAACGTTGGGGTTCCCTGGAAGCCTTGTTAAAAAAT | |
| | CTGGATCGTCTCAAGCCCGCAATTCGTGAAAAGATCCTGGCTCATATGGACGATCTTAAA | |
| | TTAAGTTGGGACCTGGCCAAGGTGCGCACCGATTTACCGCTTGAAGTGGATTTTGCAAAA | |
| | CGCCGTGAGCCGGACCGGGAACGTTTACGCGCTTTCTTAGAGCGTCTGGAATTCGGTTCA | |
| | CTGCTTCATGAATTCGGTCTGTTAGAGTCTCCTAAAGCACTCGAAGAGGCACCGTGGCCG | |
| | CCCCCAGAAGGTGCTTTTGTTGGCTTCGTACTTTCCCGTAAGGAGCCTATGTGGGCAGAT | |
| | CTTCTGGCTTTAGCGGCTGCACGCGGTGGCCGTGTTCACCGGGCCCCTGAGCCATACAAA | |
| | GCGTTACGTGATCTGAAGGAAGCACGTGGCTTGCTGGCAAAAGACCTTTCTGTTTTGGCC | |
| | CTGCGCGAGGGTCTTGGACTGCCGCCAGGCGACGATCCCATGTTATTGGCCTATCTGTTA | |
| | GACCCTAGCAATACCACACCTGAAGGGGTCGCTCGTCGGTATGGCGGTGAATGGACTGAG | |
| | GAAGCCGGAGAGCGCGCCGCATTGTCCGAACGGCTCTTTGCAAACTTATGGGGTCGTCTG | |
| | GAAGGGGAGGAACGTCTGTTATGGTTGTATCGGGAAGTCGAACGTCCTCTTTCGGCCGTA | |
| | TTAGCGCATATGGAGGCAACAGGTGTGCGTTTAGATGTCGCGTACCTTCGGGCCTTATCA | |
| | CTGGAAGTTGCAGAGGAAATCGCCCGTCTCGAGGCTGAAGTGTTCCGGTTGGCCGGTCAC | |
| | CCGTTTAACCTCAACTCCCGTGACCAGCTGGAACGCGTTTTATTCGATGAGCTTGGGCTT | |
| | CCCGCAATTGGCAAAACCACCAAGACTGGCAAACGCAGTACGAGCGCTGCCGTCCTTGAG | |
| | GCACTCCGCGAGGCTCACCCTATTGTAGAAAAGATCCTGCAATACCGTGAGTTGACGAAG | |
| | CTTAAAAGCACTTATATTGATCCTCTCCCGGATCTGATCCATCCTCGTACCGGCCGCTTG | |
| | CACACACGTTTCAACCAGACGGCGACTGCAACCGGCCGTCTGTCTAGCTCGGATCCAAAT | |
| | CTCCAGAACATTCCGGTCCGTACACCCTTGGGCCAACGTATCCGCCGGGCGTTTATCGCG | |
| | GAAGAAGGATGGTTACTGGTCGCATTGGACTACTCGCAGATTGAGCTGCGCGTCCTCGCA | |
| | CATCTCTCTGGTGACGAAAATTTAATCCGCGTGTTTCAAGAGGGGCGTGATATTCACACA | |
| | GAAACTGCCTCATGGATGTTCGGTGTCCCACGTGAAGCAGTGGATCCTTTGATGCGCCGT | |
| | GCAGCTAAAACAATTAATTTTGGAGTGCTGTACGGAATGAGCGCTCATCGCTTGAGTCAG | |
| | GAACTGGCAATCCCCTACGAGGAAGCGCAGGCATTCATCGAACGTTACTTTCAATCGTTT | |
| | CCGAAAGTTCGCGCATGGATCGAGAAGACGCTCGAGGAAGGTCGTCGTCGGGGCTATGTC | |
| | GAAACTCTGTTTGGTCGCCGTCGGTACGTACCAGATCTTGAAGCCCGCGTCAAATCGGTA | |
| | CGGGAGGCTGCGGAGCGTATGGCATTTAATATGCCTGTACAGGGTACTGCAGCTGACCTC | |
| | ATGAAACTGGCAATGGTCAAGCTTTTCCCGCGCTTGGAGGAAATGGGCGCACGTATGCTT | |
| | CTGCAGGTCCATGACGAGCTGGTGTTAGAAGCCCCTAAGGAGCGCGCCGAAGCTGTCGCG | |
| | CGCCTCGCTAAAGAAGTGATGGAGGGCGTTTACCCATTGGCCGTACCCCTCGAAGTGGAG | |
| | GTCGGTATTGGAGAAGATTGGTTATCTGCAAAGGAAgcggccgcactcgagatcaaaggg | |
| | ctagccagccagaactcgccccggaagaccccgaggatgtcgagcaccaccaccaccacc | |
| | ac | |
| M113 | MRGMLPLFEPKGRVLLLDGHRLAYRTFHALKGLTTSRGEPVQAVYGFAKSLLKALKEDGD | 213 |
| (V17L/H | DVIVVFDAKAPSFRHEAYGGYKAGRAPRPEDFPRQLALIKELVDLLGLARLEVPGYEADD | |
| 21R/A61 | VLASLAKKAEKEGYEVRILTADKDLYQLLSDRIHVLHPEGYLITPAWLWEKYGLRPDQWA | |
| D/T88R/ | DYRALTGDESDNLPGVKGIGEKTARKLLEEWGSLEALLKNLDRLKPAIREKILAHMDDLK | |
| E507T); | LSWDLAKVRTDLPLEVDFAKRREPDRERLRAFLERLEFGSLLHEFGLLESPKALEEAPWP | |
| Protein | PPEGAFVGFVLSRKEPMWADLLALAAARGGRVHRAPEPYKALRDLKEARGLLAKDLSVLA | |
| sequence | LREGLGLPPGDDPMLLAYLLDPSNTTPEGVARRYGGEWTEEAGERAALSERLFANLWGRL | |
| (N to C | EGEERLLWLYREVERPLSAVLAHMEATGVRLDVAYLRALSLEVAEEIARLEAEVFRLAGH | |
| terminus) | PFNLNSRDQLERVLFDELGLPAIGKTTKTGKRSTSAAVLEALREAHPIVEKILQYRELTK | |
| | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M113 | ATGCGTGGTATGCTGCCGTTGTTCGAGCCTAAAGGCCGCGTACTGTTACTTGATGGTCAT | 214 |
| (V17L/H | CGTTTGGCCTATCGGACGTTCCATGCACTCAAAGGTCTGACGACCAGTCGTGGCGAACCG | |
| 21R/A61 | GTCCAGGCTGTTTATGGTTTCGCTAAGTCTTTGCTCAAAGCACTGAAAGAAGACGGGGAC | |
| D/T88R/ | GATGTAATTGTTGTATTTGATGCCAAAGCACCGAGCTTCCGCCACGAAGCTTATGGTGGC | |
| E507T); DNA | TACAAGGCAGGACGCGCCCCTCGTCCAGAAGATTTCCCCCGTCAGCTGGCATTAATTAAG | |
| sequence | GAGTTAGTAGACCTTCTCGGCTTAGCGCGTCTGGAAGTTCCGGGTTATGAGGCGGACGAT | |
| (5′ to 3′) | GTCCTTGCATCCTTGGCTAAAAAGGCCGAAAAAGAGGGCTACGAAGTCCGCATCTTGACG | |
| | GCAGACAAAGATCTGTACCAGCTTCTGTCTGAC | |
| | CGTATTCATGTTTTGCACCCTGAAGGCTACTTAATCACTCCGGCCTGGCTCTGGGAAAAG | |
| | TACGGTCTGCGTCCCGATCAGTGGGCGGATTATCGGGCTTTGACGGGAGATGAGAGCGAC | |
| | AACCTGCCAGGAGTTAAGGGCATTGGTGAAAAAACCGCACGTAAGCTGCTTGAAGAGTGG | |
| | GGTTCCCTGGAAGCCTTGTTAAAAAATCTGGATCGTCTCAAGCCCGCAATTCGTGAAAAG | |
| | ATCCTGGCTCATATGGACGATCTTAAATTAAGTTGGGACCTGGCCAAGGTGCGCACCGAT | |
| | TTACCGCTTGAAGTGGATTTTGCAAAACGCCGTGAGCCGGACCGGGAACGTTTACGCGCT | |
| | TTCTTAGAGCGTCTGGAATTCGGTTCACTGCTTCATGAATTCGGTCTGTTAGAGTCTCCT | |
| | AAAGCACTCGAAGAGGCACCGTGGCCGCCCCCAGAAGGTGCTTTTGTTGGCTTCGTACTT | |
| | TCCCGTAAGGAGCCTATGTGGGCAGATCTTCTGGCTTTAGCGGCTGCACGCGGTGGCCGT | |
| | GTTCACCGGGCCCCTGAGCCATACAAAGCGTTACGTGATCTGAAGGAAGCACGTGGCTTG | |
| | CTGGCAAAAGACCTTTCTGTTTTGGCCCTGCGCGAGGGTCTTGGACTGCCGCCAGGCGAC | |
| | GATCCCATGTTATTGGCCTATCTGTTAGACCCTAGCAATACCACACCTGAAGGGGTCGCT | |
| | CGTCGGTATGGCGGTGAATGGACTGAGGAAGCCGGAGAGCGCGCCGCATTGTCCGAACGG | |
| | CTCTTTGCAAACTTATGGGGTCGTCTGGAAGGGGAGGAACGTCTGTTATGGTTGTATCGG | |
| | GAAGTCGAACGTCCTCTTTCGGCCGTATTAGCGCATATGGAGGCAACAGGTGTGCGTTTA | |
| | GATGTCGCGTACCTTCGGGCCTTATCACTGGAAGTTGCAGAGGAAATCGCCCGTCTCGAG | |
| | GCTGAAGTGTTCCGGTTGGCCGGTCACCCGTTTAACCTCAACTCCCGTGACCAGCTGGAA | |
| | CGCGTTTTATTCGATGAGCTTGGGCTTCCCGCAATTGGCAAAACCACCAAGACTGGCAAA | |
| | CGCAGTACGAGCGCTGCCGTCCTTGAGGCACTCCGCGAGGCTCACCCTATTGTAGAAAAG | |
| | ATCCTGCAATACCGTGAGTTGACGAAGCTTAAAAGCACTTATATTGATCCTCTCCCGGAT | |
| | CTGATCCATCCTCGTACCGGCCGCTTGCACACACGTTTCAACCAGACGGCGACTGCAACC | |
| | GGCCGTCTGTCTAGCTCGGATCCAAATCTCCAGAACATTCCGGTCCGTACACCCTTGGGC | |
| | CAACGTATCCGCCGGGCGTTTATCGCGGAAGAAGGATGGTTACTGGTCGCATTGGACTAC | |
| | TCGCAGATTGAGCTGCGCGTCCTCGCACATCTCTCTGGTGACGAAAATTTAATCCGCGTG | |
| | TTTCAAGAGGGGCGTGATATTCACACAGAAACTGCCTCATGGATGTTCGGTGTCCCACGT | |
| | GAAGCAGTGGATCCTTTGATGCGCCGTGCAGCTAAAACAATTAATTTTGGAGTGCTGTAC | |
| | GGAATGAGCGCTCATCGCTTGAGTCAGGAACTGGCAATCCCCTACGAGGAAGCGCAGGCA | |
| | TTCATCGAACGTTACTTTCAATCGTTTCCGAAAGTTCGCGCATGGATCGAGAAGACGCTC | |
| | GAGGAAGGTCGTCGTCGGGGCTATGTCGAAACTCTGTTTGGTCGCCGTCGGTACGTACCA | |
| | GATCTTGAAGCCCGCGTCAAATCGGTACGGGAGGCTGCGGAGCGTATGGCATTTAATATG | |
| | CCTGTACAGGGTACTGCAGCTGACCTCATGAAACTGGCAATGGTCAAGCTTTTCCCGCGC | |
| | TTGGAGGAAATGGGCGCACGTATGCTTCTGCAGGTCCATGACGAGCTGGTGTTAGAAGCC | |
| | CCTAAGGAGCGCGCCGAAGCTGTCGCGCGCCTCGCTAAAGAAGTGATGGAGGGCGTTTAC | |
| | CCATTGGCCGTACCCCTCGAAGTGGAGGTCGGTATTGGAGAAGATTGGTTATCTGCAAAG | |
| | GAAgcggccgcactcgagatcaaacgggctagccagccagaactcgccccggaagacccc | |
| | gaggatgtcgagcaccaccaccaccaccac | |
| M114 | MRGMLPLFEPKGRVLLLDGHRLAYRTFHALKGLTTSRGEPVQAVYGFAKSLLKALKEDGD | 215 |
| (V17L/H | DVIVVFDAKAPSFRHEAYGGYKAGRAPRPEDFPRQLALIKELVDLLGLARLEVPGYEADD | |
| 21R/A61 | VLASLAKKAEKEGYEVRILTADKDLYQLLSDRIHVLHPEGYLITPAWLWEKYGLRPDQWA | |
| D/T88R/ | DYRALTGDESDNLPGVKGIGEKTARKLLERWGSLEALLKNLDRLKPAIREKILAHMDDLK | |
| E210R/E | LSWDLAKVRTDLPLEVDFAKRREPDRERLRAFLERLEFGSLLHEFGLLESPKALEEAPWP | |
| 507T); | PPEGAFVGFVLSRKEPMWADLLALAAARGGRVHRAPEPYKALRDLKEARGLLAKDLSVLA | |
| Protein | LREGLGLPPGDDPMLLAYLLDPSNTTPEGVARRYGGEWTEEAGERAALSERLFANLWGRL | |
| sequence | EGEERLLWLYREVERPLSAVLAHMEATGVRLDVAYLRALSLEVAEEIARLEAEVFRLAGH | |
| (N to C | PFNLNSRDQLERVLFDELGLPAIGKTTKTGKRSTSAAVLEALREAHPIVEKILQYRELTK | |
| terminus) | LKSTYIDPLPDLIHPRTGRLHTRFNQTATATGRLSSSDPNLQNIPVRTPLGQRIRRAFIA | |
| | EEGWLLVALDYSQIELRVLAHLSGDENLIRVFQEGRDIHTETASWMFGVPREAVDPLMRR | |
| | AAKTINFGVLYGMSAHRLSQELAIPYEEAQAFIERYFQSFPKVRAWIEKTLEEGRRRGYV | |
| | ETLFGRRRYVPDLEARVKSVREAAERMAFNMPVQGTAADLMKLAMVKLFPRLEEMGARML | |
| | LQVHDELVLEAPKERAEAVARLAKEVMEGVYPLAVPLEVEVGIGEDWLSAKEAAALEIKR | |
| | ASQPELAPEDPEDVEHHHHHH | |
| M114 | ATGCGTGGTATGCTGCCGTTGTTCGAGCCTAAAGGCCGCGTACTGTTACTTGATGGTCAT | 216 |
| (V17L/H | CGTTTGGCCTATCGGACGTTCCATGCACTCAAAGGTCTGACGACCAGTCGTGGCGAACCG | |
| 21R/A61 | GTCCAGGCTGTTTATGGTTTCGCTAAGTCTTTGCTCAAAGCACTGAAAGAAGACGGGGAC | |
| D/T88R/ | GATGTAATTGTTGTATTTGATGCCAAAGCACCGAGCTTCCGCCACGAAGCTTATGGTGGC | |
| E210R/E507T); | TACAAGGCAGGACGCGCCCCTCGTCCAGAAGATTTCCCCCGTCAGCTGGCATTAATTAAG | |
| DNA | GAGTTAGTAGACCTTCTCGGCTTAGCGCGTCTGGAAGTTCCGGGTTATGAGGCGGACGAT | |
| sequence | GTCCTTGCATCCTTGGCTAAAAAGGCCGAAAAAGAGGGCTACGAAGTCCGCATCTTGACG | |
| (5′ to 3′) | GCAGACAAAGATCTGTACCAGCTTCTGTCTGACCGTATTCATGTTTTGCACCCTGAAGGC | |
| | TACTTAATCACTCCGGCCTGGCTCTGGGAAAAGTACGGTCTGCGTCCCG | |
| | AAACCGCACGTAAGCTGCTTGAACGTTGGGGTTCCCTGGAAGCCTTGTTAAAAAATCTGG | |
| | ATCGTCTCAAGCCCGCATCAGTGGGCGGATTATCGGGCTTTGACGGGAGATGAGAGCGAC | |
| | AACCTGCCAGGAGTTAAGGGCATTGGTGAAACCGCTTGAAGTGGATTTTGCAAAACGCCG | |
| | TGAGCCGGACCGGGAACGTTTACGCGCTTTCTTAGAGCGTCTGGAATAATTCGTGAAAAG | |
| | ATCCTGGCTCATATGGACGATCTTAAATTAAGTTGGGACCTGGCCAAGGTGCGCACCGAT | |
| | TTATCGGTTCACTGCTTCATGAATTCGGTCTGTTAGAGTCTCCTAAAGCACTCGAAGAGG | |
| | CACCGTGGCCGCCCCCAGAGTGGCCGTGTTCACCGGGCCCCTGAGCCATACAAAGCGTTA | |
| | CGTGATCTGAAGGAAGCACGTGGCTTGCTGGCAAAGGTGCTTTTGTTGGCTTCGTACTTT | |
| | CCCGTAAGGAGCCTATGTGGGCAGATCTTCTGGCTTTAGCGGCTGCACGCGAAGACCTTT | |
| | CTGTTTTGGCCCTGCGCGAGGGTCTTGGACTGCCGCCAGGCGACGATCCCATGTTATTGG | |
| | CCTATCTGTTAGACCCTAGCAATACCACACCTGAAGGGGTCGCTCGTCGGTATGGCGGTG | |
| | AATGGACTGAGGAAGCCGGAGAGCGCGCCGCATTGTCCGAACGGCTCTTTGCAAACTTAT | |
| | GGGGTCGTCTGGAAGGGGAGGAACGTCTGTTATGGTTGTATCGGGAAGTCGAACGTCCTC | |
| | TTTCGGCCGTATTAGCGCATATGGAGGCAACAGGTGTGCGTTTAGATGTCGCGTACCTTC | |
| | GGGCCTTATCACTGGAAGTTGCAGAGGAAATCGCCCGTCTCGAGGCTGAAGTGTTCCGGT | |
| | TGGCCGGTCACCCGTTTAACCTCAACTCCCGTGACCAGCTGGAACGCGTTTTATTCGATG | |
| | AGCTTGGGCTTCCCGCAATTGGCAAAACCACCAAGACTGGCAAACGCAGTACGAGCGCTG | |
| | CCGTCCTTGAGGCACTCCGCGAGGCTCACCCTATTGTAGAAAAGATCCTGCAATACCGTG | |
| | AGTTGACGAAGCTTAAAAGCACTTATATTGATCCTCTCCCGGATCTGATCCATCCTCGTA | |
| | CCGGCCGCTTGCACACACGTTTCAACCAGACGGCGACTGCAACCGGCCGTCTGTCTAGCT | |
| | CGGATCCAAATCTCCAGAACATTCCGGTCCGTACACCCTTGGGCCAACGTATCCGCCGGG | |
| | CGTTTATCGCGGAAGAAGGATGGTTACTGGTCGCATTGGACTACTCGCAGATTGAGCTGC | |
| | GCGTCCTCGCACATCTCTCTGGTGACGAAAATTTAATCCGCGTGTTTCAAGAGGGGCGTG | |
| | ATATTCACACAGAAACTGCCTCATGGATGTTCGGTGTCCCACGTGAAGCAGTGGATCCTT | |
| | TGATGCGCCGTGCAGCTAAAACAATTAATTTTGGAGTGCTGTACGGAATGAGCGCTCATC | |
| | GCTTGAGTCAGGAACTGGCAATCCCCTACGAGGAAGCGCAGGCATTCATCGAACGTTACT | |
| | TTCAATCGTTTCCGAAAGTTCGCGCATGGATCGAGAAGACGCTCGAGGAAGGTCGTCGTC | |
| | GGGGCTATGTCGAAACTCTGTTTGGTCGCCGTCGGTACGTACCAGATCTTGAAGCCCGCG | |
| | TCAAATCGGTACGGGAGGCTGCGGAGCGTATGGCATTTAATATGCCTGTACAGGGTACTG | |
| | CAGCTGACCTCATGAAACTGGCAATGGTCAAGCTTTTCCCGCGCTTGGAGGAAATGGGCG | |
| | CACGTATGCTTCTGCAGGTCCATGACGAGCTGGTGTTAGAAGCCCCTAAGGAGCGCGCCG | |
| | AAGCTGTCGCGCGCCTCGCTAAAGAAGTGATGGAGGGCGTTTACCCATTGGCCGTACCCC | |
| | TCGAAGTGGAGGTCGGTATTGGAGAAGATTGGTTATCTGCAAAGGAAgcggccgcactcg | |
| | agatcaaacgggctagccagccagaactcgccccggaagaccccgaggatgtcgagcacc | |
| | accaccaccaccac | |

1. A Taq DNA polymerase mutant comprising a mutation at one or more amino acid positions, wherein the one or more mutations is selected from the group consisting of A141P, A227S, A227Y, A259T, A29P, A341K, A439M, A61D, A705S, A70M, A757S, A77S, A83H, D151S, D547R, E117R, E135A, E159G, E210G, E210R, E255Q, E263S, E296G, E388S, E39R, E404G, E423G, E423I, E465A, E507K, E507T, E524G, E724G, E742G, E742N, E774P, E774S, F27P, F27R, F632L, F73R, F73S, F749A, G115R, G12R, G160N, G160S, G195R, G286S, G395Y, G59F, G59T, G84A, H21R, H75R, K128R, K225R, K247A, K260G, K508R, K531R, K709L, K709R, K831E, L106V, L148P, L233D, L245H, L33P, L352P, L367V, L456P, L55R, L5P, L605P, L606V, L670P, L768P, L96P, M775Y, P194Y, P527Q, P589A, P89I, Q178R, Q582M, Q680R, Q690R, Q754R, Q782R, Q95T, R110M, R2F, R596S, R695S, R801Q, S290P, S383H, S383N, S383P, S543F, S679G, T386Q, T569A, T88R, V103A, V17L, V449A, V607N, V631A, V821D, W167E, W706F, Y161N, Y161R, Y161V, Y182S, Y24H, Y24T, Y339P, Y339V, Y455R, Y78R, and Y78S.

2. The Taq DNA polymerase mutant of 1, wherein the Taq DNA polymerase mutant has a mutation at two or more amino acid positions.

3. The Taq DNA polymerase mutant of 2, wherein the first amino acid position is selected from the group consisting of A61D, E39R, H21R, L367V, R2F, Y182S, and T88R; and the second amino acid position is selected from the group consisting of A61D, E39R, E210R, E507T, H21R, L367V, R2F, Y182S, and T88R; and wherein the mutation at the first amino acid position and the mutation at the second amino acid position are different from one another.

4. The Taq DNA polymerase mutant of 1, wherein the Taq DNA polymerase mutant has a mutation at three or more amino acid positions.

5. The Taq DNA polymerase mutant of 4, wherein the mutation at the first amino acid position is selected from the group consisting of H21R, R2F, T88R, E39R, A61D, L367V, and Y182S; the mutation at the second amino acid position is selected from the group consisting of H21R, R2F, T88R, E39R, A61D, L367V, and Y182S; and the mutation at the third amino acid position is selected from the group consisting of H21R, R2F, T88R, E39R, A61D, L367V, and Y182S; and wherein the mutation at the first amino acid position, the mutation at the second amino acid position, and the mutation at the third amino acid position are different.

6. The Taq DNA polymerase mutant of 5, wherein the mutation at the first amino acid position is H21R; the mutation at the second amino acid is selected from the group consisting of T88R, A61D, and E39R; and the mutation at the third amino acid position is at position L367V or T88R.

7. The Taq DNA Polymerase mutant of 4, wherein the Taq DNA polymerase mutant comprises a mutation at three or more amino acid positions, wherein each mutation is selected from the group consisting of H21R, E39R, A61D, T88R, V17L, A29P, E210R, V631A, E724G, K831E, K709R, Y24H, and F27R.

8. The Taq DNA Polymerase mutant of 7, wherein: the mutation at the first amino acid position is H21R or V17L; the mutation at the second amino acid position is selected from the group consisting of H21R, A29P, T88R, Y24H, and F27R; and the mutation at the third amino acid position is selected from the group consisting of T88R, E210R, V631A, K709R, E724G, and K831E; and wherein the mutation at the first amino acid position, the mutation at the second amino acid position, and the mutation at the third amino acid position are different from one another.

9. The Taq DNA Polymerase mutant of 7 or 8, wherein the mutation at the first amino acid position is H21R or V17L; the mutation at the second amino acid position is H21R or T88R; and the mutation at the third amino acid position is T88R or E210R.

10. The Taq DNA Polymerase mutant of 1, wherein the Taq DNA polymerase mutant comprises a mutation at four or more amino acid positions.

11. The Taq DNA polymerase of 10, wherein: the mutation at a first amino acid position is selected from the group consisting of H21R, R2F, and V17L; the mutation at a second amino acid position is selected from the group consisting of E39R, H21R, A29P, T88R, Y24H, F27R, and A61D; the mutation at a third amino acid position is selected from the group consisting of A61D, T88R, E210R, V631A, K709R, E724G, K831E, Y182S and E39R; and the mutation at a fourth amino acid position is selected from the group consisting of T88R, A61D, L367V, E507T, and E210R; and wherein the mutation at the first amino acid position, the mutation at the second amino acid position, the mutation at the third amino acid position, and/or mutation at the fourth amino acid position are different from one another.

12. The Taq DNA Polymerase mutant of 11, wherein the mutation at the first amino acid position is H21R; the mutation at the second amino acid position is E39R or A61D; the mutation at the third amino acid position is T88R; and the mutation at the fourth amino acid position is E210R.

13. The Taq DNA Polymerase mutant of 1, wherein the Taq DNA polymerase has mutation at five or more amino acid positions.

14. The Taq DNA polymerase mutant of 13, wherein the Taq DNA Polymerase comprises a mutation at five amino acid positions, wherein the mutation at the first amino acid position is H21R or V17L; the mutation at the second amino acid position is selected from the group consisting of A29P, E39R, H21R, Y24H, F27R and A61D; the mutation at the third amino acid position is selected from the group consisting of E39R, A61D, and T88R; the mutation at the fourth amino acid position is T88R, E210R, and A61D; and the mutation at the fifth amino acid position is selected from the group consisting of T88R, E210R, E507T, K709R, V631A, E274G, E724G, and K831E; and wherein the mutation at a first amino acid position, the mutation at a second amino acid position, the mutation at a third amino acid position, the mutation at a fourth amino acid position, and/or the mutation at a fifth amino acid position are different from one another.

15. The Taq DNA polymerase mutant of 14, wherein the mutation at the first amino acid position is V17L; the mutation at the second amino acid position is H21R; the mutation at the third amino acid position is E39R or A61D; the mutation at the fourth amino acid position is T88R; and the mutation at the fifth amino acid position is E210R.

16. The Taq DNA polymerase mutant of 1, wherein the Taq DNA polymerase mutant comprises a mutation at six or more amino acid positions.

17. The Taq DNA polymerase mutant of claim 16, wherein: the mutation at a first amino acid position is V17L; the mutation at a second amino acid position is H21R; the mutation at a third amino acid position is A61D; the mutation at a fourth amino acid position is T88R; the mutation at the fifth amino acid position is E210R; the mutation at the sixth amino acid position is E507T; and wherein the mutation at the first amino acid position, the mutation at the second amino acid position, the mutation at the third amino acid position, and/or mutation at the fourth amino acid position are different from one another.

18. The Taq DNA Polymerase mutant of any of 1 to 17 wherein Taq DNA polymerase mutant exhibits, relative to wild-type DNA polymerase, increased DNA amplification, increased resistance to a PCR inhibitor, and/or increased tolerance to a PCR inhibitor.

19. The Taq DNA polymerase mutant of 18, wherein the Taq DNA polymerase mutant possesses an increased DNA amplification which is at least a 5-fold increase relative to wild-type DNA polymerase.

20. The Taq DNA polymerase mutant of 19, wherein the one or more mutations is selected from the group consisting of L106V, S383N, K260G, T386Q, T88R, E388S, E742N, A259T, W706F, F27P, E774P, E255Q, Y161R, A29P, F749A, E465A, F73R, S543F, G59F, G59T, E210R, A61D, Y78S, F73R, P589A, K709L, Y24H, L245H, F73S, E117R, P89I, R801Q, A705S, S383H, V17L, A141P, F27R, M775Y, E423I, R596S, G84A, E742G, E117R, Y24T, S290P, K531I, G286S. Y78R, Y339P, V631A, A77S, L233D, G12R, K831E, G195R, K709R, and L367V.

21. The Taq DNA polymerase mutant of 18, wherein the PCR inhibitor is selected from the group consisting of humic acid, heparin, hematin, high-salt, ethanol, and guanidine thiocyanate. [others?]

22. The Taq DNA polymerase mutant of 21, wherein the PCR inhibitor is humic acid.

23. The Taq DNA polymerase mutant of any one of the 18 to 22, wherein the DNA amplification increases after a second round of amplification.

24. An isolated nucleic acid molecule comprising a nucleotide sequence encoding the Taq DNA polymerase mutant of any one of 1 to 23.

25. A composition comprising the Taq DNA polymerase mutant of any of one of 1 to 23.

26. A kit comprising the Taq DNA polymerase mutant of any one of claims 1 to 23 and one or more reagents for a DNA synthesis reaction.

27. A method for conducting primer extension, comprising: contacting a Taq DNA polymerase mutant of any one of 1 to 23 with a primer, a polynucleotide template, and nucleoside triphosphates under conditions suitable for a primer extension method, thereby producing an extended primer.

28. A method for synthesizing complementary amplifying complementary deoxyribonucleic acid (cDNA) comprising the steps of: providing the Taq DNA polymerase mutant of any one of 1 to 23; and contacting the Taq DNA polymerase mutant with a nucleic acid template to permit synthesis of cDNA.

29. A method for performing polymerase chain reaction (PCR) on a sample containing humic acid comprising: providing the Taq DNA polymerase mutant of any of 1 to 23; and contacting the Taq DNA polymerase mutant with a nucleic acid template to replicate and amplify the nucleic acid template.

30. The method of 29, wherein the amplification of the nucleic acid template is at least 5-fold increase relative to wild-type DNA polymerase.

31. A method for obtaining a Taq DNA polymerase mutant comprising the steps of: subjecting a polynucleotide encoding a Taq DNA polymerase to PCR to generate a library comprising an array of different polynucleotides; screening the library for increased DNA amplification, increased resistance to a PCR inhibitor, and/or increased tolerance to a PCR inhibitor; selecting one or more Taq DNA polymerases from the screening step; and repeating until a final Taq DNA polymerase is obtained, wherein the final Taq DNA polymerase comprises two or more amino acid mutations, wherein the two or more mutations are selected from the group consisting of A141P, A227S, A227Y, A259T, A29P, A341K, A439M, A61D, A705S, A70M, A757S, A77S, A83H, D151S, D547R, E117R, E135A, E159G, E210G, E210R, E255Q, E263S, E296G, E388S, E39R, E404G, E423G, E423I, E465A, E507K, E507T, E524G, E724G, E742G, E742N, E774P, E774S, F27P, F27R, F632L, F73R, F73S, F749A, G115R, G12R, G160N, G160S, G195R, G286S, G395Y, G59F, G59T, G84A, H21R, H75R, K128R, K225R, K247A, K260G, K508R, K531R, K709L, K709R, K831E, L106V, L148P, L233D, L245H, L33P, L352P, L367V, L456P, L55R, L5P, L605P, L606V, L670P, L768P, L96P, M775Y, P194Y, P527Q, P589A, P89I, Q178R, Q582M, Q680R, Q690R, Q754R, Q782R, Q95T, R110M, R2F, R596S, R695S, R801Q, S290P, S383H, S383N, S383P, S543F, S679G, T386Q, T569A, T88R, V103A, V17L, V449A, V607N, V631A, V821D, W167E, W706F, Y161N, Y161R, Y161V, Y182S, Y24H, Y24T, Y339P, Y339V, Y455R, Y78R, and Y78S.

32. The method of 31, wherein the two or more mutations are selected from the group consisting of H21R, E39R, A61D, T88R, V17L, A29P, E210R, V631A, E724G, K831E, K709R, Y24H, and F27R.

33. The method of 31 or 32, wherein the PCR inhibitor is selected from the group consisting of humic acid, heparin, hematin, high-salt, ethanol, and guanidine thiocyanate.

34. The method of claim 33, wherein the PCR inhibitor is humic acid.

It is to be understood that the foregoing describes preferred embodiments of the technology and that modifications may be made therein without departing from the spirit or scope of the invention as set forth in the appended claims.

1. A Taq DNA polymerase mutant comprising a mutation at one or more amino acid positions, wherein the one or more mutations is selected from the group consisting of A141P, A227S, A227Y, A259T, A29P, A341K, A439M, A61D, A705S, A70M, A757S, A77S, A83H, D151S, D547R, E117R, E135A, E159G, E210G, E210R, E255Q, E263S, E296G, E388S, E39R, E404G, E423G, E423I, E465A, E507K, E507T, E524G, E724G, E742G, E742N, E774P, E774S, F27P, F27R, F632L, F73R, F73S, F749A, G115R, G12R, G160N, G160S, G195R, G286S, G395Y, G59F, G59T, G84A, H21R, H75R, K128R, K225R, K247A, K260G, K508R, K531R, K709L, K709R, K831E, L106V, L148P, L233D, L245H, L33P, L352P, L367V, L456P, L55R, L5P, L605P, L606V, L670P, L768P, L96P, M775Y, P194Y, P527Q, P589A, P89I, Q178R, Q582M, Q680R, Q690R, Q754R, Q782R, Q95T, R110M, R2F, R596S, R695S, R801Q, S290P, S383H, S383N, S383P, S543F, S679G, T386Q, T569A, T88R, V103A, V17L, V449A, V607N, V631A, V821D, W167E, W706F, Y161N, Y161R, Y161V, Y182S, Y24H, Y24T, Y339P, Y339V, Y455R, Y78R, and Y78S.

2-9. (canceled)

10. The Taq DNA Polymerase mutant of claim 1, wherein the Taq DNA polymerase mutant comprises a mutation at four or more amino acid positions.

11. The Taq DNA polymerase of claim 10, wherein:
the mutation at the first amino acid position is selected from the group consisting of H21R, R2F, and V17L;
the mutation at the second amino acid position is selected from the group consisting of E39R, H21R, A29P, T88R, Y24H, F27R, and A61D;
the mutation at the third amino acid position is selected from the group consisting of A61D, T88R, E210R, V631A, K709R, E724G, K831E, Y182S and E39R; and
the mutation at the fourth amino acid position is selected from the group consisting of T88R, A61D, L367V, E507T, and E210R; and
wherein the mutation at the first amino acid position, the mutation at the second amino acid position, the mutation at the third amino acid position, and the mutation at the fourth amino acid position are different from one another.

12. The Taq DNA Polymerase mutant of claim 11, wherein the mutation at the first amino acid position is H21R; the mutation at the second amino acid position is E39R or A61D; the mutation at the third amino acid position is T88R; and the mutation at the fourth amino acid position is E210R.

13. The Taq DNA Polymerase mutant of claim 1, wherein the Taq DNA polymerase has mutation at five or more amino acid positions.

14. The Taq DNA polymerase mutant of claim 13, wherein the Taq DNA Polymerase comprises a mutation at five amino acid positions, wherein,
the mutation at a first amino acid position is H21R or V17L;
the mutation at a second amino acid position is selected from the group consisting of A29P, E39R, H21R, Y24H, F27R and A61D;
the mutation at a third amino acid position is selected from the group consisting of E39R, A61D, and T88R;
the mutation at a fourth amino acid position is T88R, E210R, and A61D; and
the mutation at a fifth amino acid position is selected from the group consisting of T88R, E210R, E507T, K709R, V631A, E274G, E724G, and K831E; and
wherein the mutation at a first amino acid position, the mutation at a second amino acid position, the mutation at a third amino acid position, the mutation at a fourth amino acid position, and/or the mutation at a fifth amino acid position are different from one another.

15. The Taq DNA polymerase mutant of claim 14, wherein the mutation at the first amino acid position is V17L; the mutation at the second amino acid position is H21R; the mutation at the third amino acid position is E39R or A61D; the mutation at the fourth amino acid position is T88R; and the mutation at the fifth amino acid position is E210R.

16. The Taq DNA polymerase mutant of claim 1, wherein the Taq DNA polymerase mutant comprises a mutation at six or more amino acid positions.

17. The Taq DNA polymerase mutant of claim 16, wherein:
the mutation at a first amino acid position is V17L;
the mutation at a second amino acid position is H21R;
the mutation at a third amino acid position is A61D;
the mutation at a fourth amino acid position is T88R;
the mutation at the fifth amino acid position is E210R;
the mutation at the sixth amino acid position is E507T; and
wherein the mutation at the first amino acid position, the mutation at the second amino acid position, the mutation at the third amino acid position, and/or mutation at the fourth amino acid position are different.

18. The Taq DNA Polymerase mutant of claim 1, wherein Taq DNA polymerase mutant exhibits, relative to wild-type DNA polymerase, increased DNA amplification, increased resistance to a PCR inhibitor, and/or increased tolerance to a PCR inhibitor.

19. The Taq DNA polymerase mutant of claim 18, wherein the Taq DNA polymerase mutant possesses an increased DNA amplification which is at least a 5-fold increase relative to wild-type DNA polymerase.

20. The Taq DNA polymerase mutant of claim 19, wherein the one or more mutations is selected from the group consisting of L106V, S383N, K260G, T386Q, T88R, E388S, E742N, A259T, W706F, F27P, E774P, E255Q, Y161R, A29P, F749A, E465A, F73R, S543F, G59F, G59T, E210R, A61D, Y78S, F73R, P589A, K709L, Y24H, L245H, F73S, E117R, P89I, R801Q, A705S, S383H, V17L, A141P, F27R, M775Y, E423I, R596S, G84A, E742G, E117R, Y24T, S290P, K531I, G286S. Y78R, Y339P, V631A, A77S, L233D, G12R, K831E, G195R, K709R, and L367V.

21. The Taq DNA polymerase mutant of claim 18, wherein the PCR inhibitor is selected from the group consisting of humic acid, heparin, hematin, high-salt, ethanol, and guanidine thiocyanate.

22. The Taq DNA polymerase mutant of claim 21, wherein the PCR inhibitor is humic acid.

23. The Taq DNA polymerase mutant of claim 18, wherein the DNA amplification increases after a second round of amplification.

24. An isolated nucleic acid molecule comprising a nucleotide sequence encoding the Taq DNA polymerase mutant of claim 1.

25. A composition comprising the Taq DNA polymerase mutant of claim 1.

26. A kit comprising the Taq DNA polymerase mutant of claim 1 and one or more reagents for a DNA synthesis reaction.

27. A method for conducting primer extension, comprising: contacting a Taq DNA polymerase mutant of claim 1 with a primer, a polynucleotide template, and nucleoside triphosphates under conditions suitable for a primer extension method, thereby producing an extended primer.

28. A method for synthesizing complementary amplifying complementary deoxyribonucleic acid (cDNA) comprising the steps of:
providing the Taq DNA polymerase mutant of claim 1; and
contacting the Taq DNA polymerase mutant with a nucleic acid template to permit synthesis of cDNA.

29. A method for performing polymerase chain reaction (PCR) on a sample containing humic acid comprising: providing the Taq DNA polymerase mutant of claim 1; and contacting the Taq DNA polymerase mutant with a nucleic acid template to replicate and amplify the nucleic acid template.

30-34. (canceled)
